(19) United States
(12) Patent Application Publication
(10) Pub. No.: US 2025/0000915 A1
(43) Pub. Date: Jan. 2, 2025

(54) METHODS OF POPULATING A GASTROINTESTINAL TRACT

(71) Applicant: Seres Therapeutics, Inc., Cambridge, MA (US)

(72) Inventors: Geoffrey VON MALTZAHN, Boston, MA (US); Matthew R. HENN, Somerville, MA (US); David N. COOK, Brooklyn, NY (US); David Arthur BERRY, Brookline, MA (US); Noubar B. AFEYAN, Lexington, MA (US); Brian GOODMAN, Boston, MA (US); Mary-Jane LOMBARDO MCKENZIE, Arlington, MA (US); Marin VULIC, Boston, MA (US)

(73) Assignee: Seres Therapeutics, Inc., Cambridge, MA (US)

(21) Appl. No.: 18/431,885

(22) Filed: Feb. 2, 2024

Related U.S. Application Data
(63) Continuation of application No. 16/051,747, filed on Aug. 1, 2018, now abandoned.
Application No. 16/051,747 is a division of application No. 14/765,810, filed on Aug. 4, 2015, now Pat. No. 10,064,900.
(60) Provisional application No. 61/926,918, filed on Jan. 13, 2014.
(60) Provisional application No. 61/760,606, filed on Feb. 4, 2013.
(60) Provisional application No. 61/760,584, filed on Feb. 4, 2013.
(60) Provisional application No. 61/760,574, filed on Feb. 4, 2013.
(60) Provisional application No. 61/760,585, filed on Feb. 4, 2013.

(51) Int. Cl. A61K 35/741; A61K 9/00; A61K 9/48; A61K 35/37; A61K 35/74; A61K 35/742; A61K 35/744; A61K 35/745; A61K 35/747; A61K 45/06; C12N 1/20

(52) U.S. Cl. CPC A61K 35/741; A61K 9/0053; A61K 9/48; A61K 9/4816; A61K 35/37; A61K 35/74; A61K 35/742; A61K 35/744; A61K 35/745; A61K 35/747; A61K 45/06; C12N 1/20; Y02A 50/30

(57) ABSTRACT

Methods and compositions for populating the gastrointestinal tract of a subject are described. Methods include administering to a subject a therapeutic composition comprising a purified population of spore-forming bacteria produced by providing a fecal material and subjecting the material to a treatment step resulting in purification of spore-forming bacteria. Methods include administering an amount effective to engraft and/or augment in the gastrointestinal tract in order to treat or prevent a dysbiosis in the mammalian subject.

### RELATED APPLICATIONS

This application is a divisional of co-pending U.S. application Ser. No. 14/765,810, filed Aug. 4, 2015, which is the National Stage of International application no. PCT/US14/14738, filed Feb. 4, 2014, which claims the benefit of U.S. Provisional Application No. 61/760,584, filed on Feb. 4, 2013, and U.S. Provisional Application No. 61/760,585, filed on Feb. 4, 2013, and U.S. Provisional Application No. 61/760,574, filed on Feb. 4, 2013, and U.S. Provisional Application No. 61/760,606, filed on Feb. 4, 2013, and U.S. Provisional Application No. 61/926,928, filed on Jan. 13, 2014, each of which is incorporated herein by reference in its entirety.

### REFERENCE TO A SEQUENCE LISTING

This application includes a Sequence Listing submitted electronically as a text file named 36980US_CRF_sequencelisting.txt, created on Jul. 26, 2018, with a size of 4,324,731 bytes. The sequence listing is incorporated by reference.

### BACKGROUND

Mammals are colonized by microbes in the gastrointestinal (GI) tract, on the skin, and in other epithelial and tissue niches such as the oral cavity, eye surface and vagina. The gastrointestinal tract harbors an abundant and diverse microbial community. It is a complex system, providing an environment or niche for a community of many different species or organisms, including diverse strains of bacteria. Hundreds of different species may form a commensal community in the GI tract in a healthy person, and this complement of organisms evolves from the time of birth to ultimately form a functionally mature microbial population by about 3 years of age. Interactions between microbial strains in these populations and between microbes and the host, e.g., the host immune system, shape the community structure, with availability of and competition for resources affecting the distribution of microbes. Such resources may be food, location and the availability of space to grow or a physical structure to which the microbe may attach. For example, host diet is involved in shaping the GI tract flora.

A healthy microbiota provides the host with multiple benefits, including colonization resistance to a broad spectrum of pathogens, essential nutrient biosynthesis and absorption, and immune stimulation that maintains a healthy gut epithelium and an appropriately controlled systemic immunity. In settings of ‘dysbiosis’ or disrupted symbiosis, microbiota functions can be lost or deranged, resulting in increased susceptibility to pathogens, altered metabolic profiles, or induction of proinflammatory signals that can result in local or systemic inflammation or autoimmunity. Thus, the intestinal microbiota plays a significant role in the pathogenesis of many diseases and disorders, including a variety of pathogenic infections of the gut. For instance, subjects become more susceptible to pathogenic infections when the normal intestinal microbiota has been disturbed due to use of broad-spectrum antibiotics. Many of these diseases and disorders are chronic conditions that significantly decrease a subject's quality of life and can be ultimately fatal.

Manufacturers of probiotics have asserted that their preparations of bacteria promote mammalian health by preserving the natural microflora in the GI tract and reinforcing the normal controls on aberrant immune responses. See, e.g., U.S. Pat. No. 8,034,601. Probiotics, however, have been limited to a very narrow group of genera and a correspondingly limited number of species; as such, they do not adequately replace the missing natural microflora of the GI tract in many situations.

Thus, there is a need for methods of populating a subject's gastrointestinal tract with a wider selection of microbiota in order to alter a dysbiosis. There is also a need to design microbial compositions of isolated bacterial strains with a plurality of beneficial properties based on current understanding of bacterial strains and analysis of the properties that would enhance the utility and commercialization of a microbial composition.

Therefore, there exists a need for durable, efficient, and effective compositions and methods for treatment of GI diseases by way of restoring or enhancing microbiota functions and for addressing these and other shortcomings in the art.

### SUMMARY OF THE INVENTION

Disclosed herein are methods of populating the gastrointestinal tract of a mammalian subject, including the step of orally administering to the mammalian subject an effective amount of a therapeutic composition comprising a purified population of spore-forming bacteria produced by the steps of a) providing a fecal material and b) subjecting the material to a treatment step resulting in purification of spore-forming bacteria, wherein the purified population is present in an amount effective to engraft and/or augment in the gastrointestinal tract in order to treat or prevent a dysbiosis in the mammalian subject.

Disclosed herein are methods for populating the gastrointestinal tract of a mammalian subject, comprising the step of orally administering to the subject an effective amount of a therapeutic composition comprising a purified population of spore-forming bacteria in an amount effective to engraft and/or augment in the gastrointestinal tract in order to treat or prevent a dysbiosis in a mammalian subject.

Also described are therapeutic compositions comprising a purified population of spore-forming bacteria or bacteria having a substantial activity of spore-forming bacteria in an amount effective to populate the gastrointestinal tract of a mammalian subject to whom the therapeutic composition is administered. In some embodiments, the population is effective to i) treat a gastrointestinal dysbiosis, and/or ii) engraft at least one type of bacteria present in the therapeutic composition but not present in a mammalian subject prior to treatment; and/or iii) augment at least one type of bacteria not present in the therapeutic composition in a mammalian subject to whom the therapeutic composition is administered.

Also provided are therapeutic compositions comprising a purified population of spore-forming bacteria in an amount effective to populate the gastrointestinal tract of a mammalian subject to whom the therapeutic composition is administered, wherein the purified population is obtained by the steps of a) providing a fecal material comprising spore-forming bacteria and b) subjecting the spore-forming bacteria to a treatment step resulting in purification of the spore-forming bacteria.

Also provided are methods for production of a composition comprising a population of bacterial spores suitable for therapeutic administration to a mammalian subject in need thereof, comprising the steps of: (a) providing a fecal material obtained from a mammalian donor subject; and (b) subjecting the fecal material to at least one purification step under conditions such that a purified population of spore-forming bacteria is produced from the fecal material. Also provided are methods of treating or preventing a dysbiosis in a human subject, comprising administering to the human subject the therapeutic compositions of the invention.

The invention also includes therapeutic compositions comprising a purified bacterial spore population comprising at least about 1×10³, 1×10⁴, 1×10⁵, or 1×10⁶ spores, wherein the composition does not exceed about 1 gram in weight, formulated for oral administration to populate the gastrointestinal trace in a mammalian subject in need thereof, in order to i) engraft in the mammalian recipient and/or ii) augment a bacterial population in the mammalian recipient and thereby treat or prevent a dysbiosis.

The invention includes kits comprising in one or more containers a fecal material collection apparatus and a solvent solution, and instructions for use thereof for generating purified populations of spore-forming bacteria.

The invention includes methods of populating the gastrointestinal tract of a human subject, comprising the step of administering to the human subject a therapeutic composition comprising a purified population of spore-forming bacteria, under conditions such that a microbial population present in the gastrointestinal tract and/or a microbial population outside the gastrointestinal tract is modulated.

The invention also includes methods of populating a bacterial population in the gastrointestinal tract of a human subject, comprising the step of administering to the human subject a therapeutic composition comprising a purified population of spore-forming bacteria, under conditions such that at least i) a subset of the spore-forming bacteria sustainably engraft within the gastrointestinal tract, or ii) at least one type of bacteria not present in the therapeutic composition is augmented within the gastrointestinal tract.

In some embodiments, the invention includes methods of populating the gastrointestinal tract of a mammalian subject, comprising the step of orally administering to the subject an effective amount of a bacterial composition comprising at least a first type of isolated bacteria and a second type of isolated bacteria, wherein the first type and the second type are not identical, under conditions such that the first type of isolated bacteria and the second type of isolated bacteria functionally populate the gastrointestinal tract of the subject.

In other embodiments, the invention includes methods of populating the gastrointestinal tract of a mammalian subject, comprising the step of orally administering to the subject an effective amount of a therapeutic composition comprising a purified population of spore-forming bacteria produced by the steps of a) providing a fecal material and b) subjecting the material to a treatment step resulting in purification of spore-forming bacteria, wherein the purified population is present in an amount effective to treat or prevent a dysbiosis in a mammalian recipient subject to whom the therapeutic composition is administered.

The invention also includes methods of populating the gastrointestinal tract of a mammalian subject, comprising the step of orally administering to the subject an effective amount of a therapeutic composition comprising a purified population of spore-forming bacteria, in an amount effective to treat or prevent a dysbiosis in a mammalian recipient subject to whom the therapeutic composition is administered.

### BRIEF DESCRIPTION OF THE DRAWINGS

FIG. 1A provides a schematic of 16S rRNA gene and denotes the coordinates of hypervariable regions 1-9 (V1-V9), according to an embodiment of the invention. Coordinates of V1-V9 are 69-99, 137-242, 433-497, 576-682, 822-879, 986-1043, 1117-1173, 1243-1294, and 1435-1465 respectively, based on numbering using E. coli system of nomenclature defined by Brosius et al., Complete nucleotide sequence of a 16S ribosomal RNA gene (16S rRNA) from Escherichia coli, PNAS 75 (10): 4801-4805 (1978).

FIG. 1B highlights in bold the nucleotide sequences for each hypervariable region in the exemplary reference E. coli 16S sequence described by Brosius et al. (SEQ ID NO: 828)

FIG. 2 shows a photograph of a CsCl gradient demonstrating the spore separation from other residual habitat material, according to an embodiment of the invention.

FIG. 3 shows three phase contrast images demonstrating the progressive enrichment of spores from a fecal suspension (ethanol treated, CsCl purified spore preparation, and an ethanol treated, CsCl purified, sucrose purified spore preparation).

FIG. 4 shows a set of survival curves demonstrating efficacy of the spore population in a hamster prophylaxis model of C. difficile. (Circles are ethanol-treated spores+Vancomycin; Squares are ethanol-treated, gradient purified spores+Vancomycin; X's are vancomycin control; and Diamonds are the vehicle control.

FIG. 5 provides a set of survival curves demonstrating efficacy of the spore population in a hamster relapse prevention model of C. difficile. (Opaque diamonds are ethanol-treated spores+Vancomycin; squares are ethanol-treated, gradient purified spores+Vancomycin; X's are vancomycin control; and Clear Diamonds are vehicle control.

FIG. 6 demonstrates the cell viability under a variety of ethanol and heat treatments for varying lengths of time.

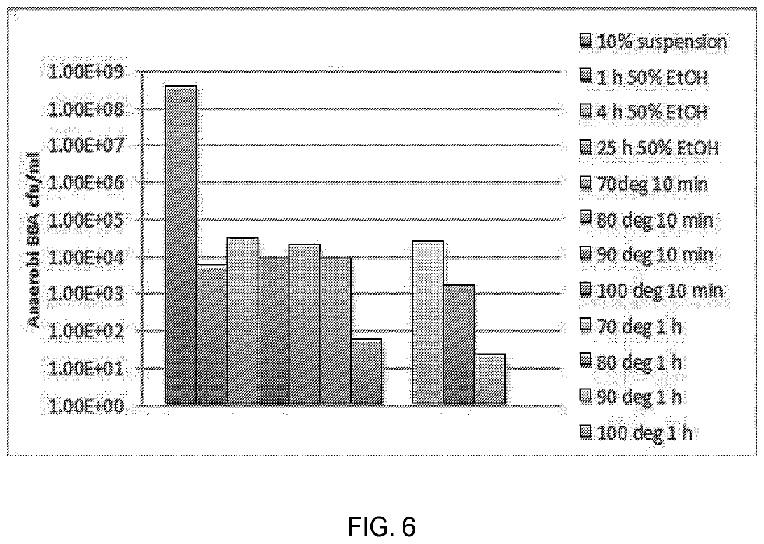

FIG. 7 shows the strong correlation and linear correspondence between the measurement of DPA concentration by a coupled fluorescence assay and the viable spore colony forming units.

FIG. 8 demonstrates the effect on various germination treatments on the ability to cultivate vegetative bacteria from a spore population.

FIG. 9 demonstrates the increase in bacterial diversity from using a germinant treatment to grow vegetative bacteria from spore populations.

FIG. 10 demonstrates the role of heat activation at various temperatures on spores from three different donor fecal samples.

FIG. 11 demonstrates that a lysozyme treatment with heat activation improves germination at most temperatures.

FIG. 12 demonstrates the microbial diversity measured in the ethanol treated spore treatment sample and patient pre- and post-treatment samples. Total microbial diversity is defined using the Chao1 Alpha-Diversity Index and is measured at the same genomic sampling depths to confirm adequate sequence coverage to assay the microbiome in the target samples. The patient pretreatment (line 1) harbored a microbiome that was significantly reduced in total diversity as compared to the ethanol treated spore treatment (line 2) and patient post treatment at days 5 (line 3), 14 (line 4), and 25 (line 5).

FIG. 13 demonstrates how the patient microbial ecology is shifted by treatment with an ethanol treated spore treatment from a dysbiotic state to a state of health. Principle coordinates analysis based on the total diversity and structure of the microbiome (Bray Curtis Beta Diversity) of the patient pre- and post-treatment delineates that the combination of engraftment of the OTUs from the spore treatment and the augmentation of the patient microbial ecology leads to a microbial ecology that is distinct from both the pretreatment microbiome and the ecology of the ethanol treated spore treatment.

FIG. 14 demonstrates the augmentation of bacteroides species in patients treated with the spore population. Comparing the number of Bacteroides colonies from fecal suspensions pre-treatment and in week 4 post treatment reveals an increase of 4 logs or greater. Colonies were enumerated by serial dilution and plating on Bacteroides Bile Esculin agar which is highly selective for the B. fragilis group. Species were determined by 16S full-length sequence identification.

FIG. 15 demonstrates the increase in number of species engrafting and species augmenting in patient's microbiomes after treatment with an ethanol-treated spore population. Relative abundance of species that engrafted or augmented as described were determined based on the number of 16S sequence reads. Each plot is from a different patient treated with the ethanol-treated spore population for recurrent C. difficile.

The figures depict various embodiments of the present invention for purposes of illustration only. One skilled in the art will readily recognize from the following discussion that alternative embodiments of the structures and methods illustrated herein may be employed without departing from the principles of the invention described herein.

### DETAILED DESCRIPTION

### Definitions

“Microbiota” refers to the community of microorganisms that occur (sustainably or transiently) in and on an animal subject, typically a mammal such as a human, including eukaryotes, archaea, bacteria, and viruses (including bacterial viruses i.e., phage).

“Microbiome” refers to the genetic content of the communities of microbes that live in and on the human body, both sustainably and transiently, including eukaryotes, archaea, bacteria, and viruses (including bacterial viruses (i.e., phage)), wherein “genetic content” includes genomic DNA, RNA such as ribosomal RNA, the epigenome, plasmids, and all other types of genetic information.

“Microbial Carriage” or simply “Carriage” refers to the population of microbes inhabiting a niche within or on humans. Carriage is often defined in terms of relative abundance. For example, OTU1 comprises 60% of the total microbial carriage, meaning that OTU1 has a relative abundance of 60% compared to the other OTUs in the sample from which the measurement was made. Carriage is most often based on genomic sequencing data where the relative abundance or carriage of a single OTU or group of OTUs is defined by the number of sequencing reads that are assigned to that OTU/s relative to the total number of sequencing reads for the sample.

“Microbial Augmentation” or simply “augmentation” refers to the establishment or significant increase of a population of microbes that are (i) absent or undetectable (as determined by the use of standard genomic and microbiological techniques) from the administered therapeutic microbial composition, (ii) absent, undetectable, or present at low frequencies in the host niche (for example: gastrointestinal tract, skin, anterior-nares, or vagina) before the delivery of the microbial composition, and (iii) are found after the administration of the microbial composition or significantly increase, for instance 2-fold, 5-fold, 1×10², 1×10³, 1×10⁴, 1×10⁵, 1×10⁶, 1×10⁷, or greater than 1×10⁸, in cases where they were present at low frequencies. The microbes that comprise an augmented ecology can be derived from exogenous sources such as food and the environment, or grow out from micro-niches within the host where they reside at low frequency. The administration of the therapeutic microbial composition induces an environmental shift in the target niche that promotes favorable conditions for the growth of these commensal microbes. In the absence of treatment with a therapeutic microbial composition, the host can be constantly exposed to these microbes; however, sustained growth and the positive health effects associated with the stable population of increased levels of the microbes comprising the augmented ecology are not observed.

“Microbial Engraftment” or simply “engraftment” refers to the establishment of OTUs comprising a therapeutic microbial composition in a target niche that are absent in the treated host prior to treatment. The microbes that comprise the engrafted ecology are found in the therapeutic microbial composition and establish as constituents of the host microbial ecology upon treatment. Engrafted OTUs can establish for a transient period of time, or demonstrate long-term stability in the microbial ecology that populates the host post treatment with a therapeutic microbial composition. The engrafted ecology can induce an environmental shift in the target niche that promotes favorable conditions for the growth of commensal microbes capable of catalyzing a shift from a dysbiotic ecology to one representative of a health state.

“Ecological Niche” or simply “Niche” refers to the ecological space in which an organism or group of organisms occupies. Niche describes how an organism or population or organisms responds to the distribution of resources, physical parameters (e.g., host tissue space) and competitors (e.g., by growing when resources are abundant, and when predators, parasites and pathogens are scarce) and how it in turn alters those same factors (e.g., limiting access to resources by other organisms, acting as a food source for predators and a consumer of prey).

“Dysbiosis” refers to a state of the microbiota or microbiome of the gut or other body area, including mucosal or skin surfaces in which the normal diversity and/or function of the ecological network is disrupted. Any disruption from the preferred (e.g., ideal) state of the microbiota can be considered a dysbiosis, even if such dysbiosis does not result in a detectable decrease in health. This state of dysbiosis may be unhealthy, it may be unhealthy under only certain conditions, or it may prevent a subject from becoming healthier. Dysbiosis may be due to a decrease in diversity, the overgrowth of one or more pathogens or pathobionts, symbiotic organisms able to cause disease only when certain genetic and/or environmental conditions are present in a patient, or the shift to an ecological network that no longer provides a beneficial function to the host and therefore no longer promotes health.

The term “pathobiont” refer to specific bacterial species found in healthy hosts that may trigger immune-mediated pathology and/or disease in response to certain genetic or environmental factors. Chow et al., (2011) Curr Op Immunol. Pathobionts of the intestinal microbiota and inflammatory disease. 23:473-80. Thus, a pathobiont is a pathogen that is mechanistically distinct from an acquired infectious organism. Thus, the term “pathogen” includes both acquired infectious organisms and pathobionts.

The terms “pathogen”, “pathobiont” and “pathogenic” in reference to a bacterium or any other organism or entity includes any such organism or entity that is capable of causing or affecting a disease, disorder or condition of a host organism containing the organism or entity.

“Phylogenetic tree” refers to a graphical representation of the evolutionary relationships of one genetic sequence to another that is generated using a defined set of phylogenetic reconstruction algorithms (e.g. parsimony, maximum likelihood, or Bayesian). Nodes in the tree represent distinct ancestral sequences and the confidence of any node is provided by a bootstrap or Bayesian posterior probability, which measures branch uncertainty.

“Operational taxonomic units,” “OTU” (or plural, “OTUs”) refer to a terminal leaf in a phylogenetic tree and is defined by a nucleic acid sequence, e.g., the entire genome, or a specific genetic sequence, and all sequences that share sequence identity to this nucleic acid sequence at the level of species. In some embodiments the specific genetic sequence may be the 16S sequence or a portion of the 16S sequence. In other embodiments, the entire genomes of two entities are sequenced and compared. In another embodiment, select regions such as multilocus sequence tags (MLST), specific genes, or sets of genes may be genetically compared. In 16S embodiments, OTUs that share ≥97% average nucleotide identity across the entire 16S or some variable region of the 16S are considered the same OTU (see e.g. Claesson M J, Wang Q, O'Sullivan O, Greene-Diniz R, Cole J R, Ross R P, and O'Toole P W. 2010. Comparison of two next-generation sequencing technologies for resolving highly complex microbiota composition using tandem variable 16S rRNA gene regions. Nucleic Acids Res 38: e200. Konstantinidis K T, Ramette A, and Tiedje J M. 2006. The bacterial species definition in the genomic era. Philos Trans R Soc Lond B Biol Sci 361:1929-1940). In embodiments involving the complete genome, MLSTs, specific genes, or sets of genes OTUs that share ≥95% average nucleotide identity are considered the same OTU (see e.g. Achtman M, and Wagner M. 2008. Microbial diversity and the genetic nature of microbial species. Nat. Rev. Microbiol. 6:431-440. Konstantinidis K T, Ramette A, and Tiedje J M. 2006. The bacterial species definition in the genomic era. Philos Trans R Soc Lond B Biol Sci 361:1929-1940). OTUs are frequently defined by comparing sequences between organisms. Generally, sequences with less than 95% sequence identity are not considered to form part of the same OTU. OTUs may also be characterized by any combination of nucleotide markers or genes, in particular highly conserved genes (e.g., “house-keeping” genes), or a combination thereof. Such characterization employs, e.g., WGS data or a whole genome sequence.

Table 1 below shows a List of Operational Taxonomic Units (OTU) with taxonomic assignments made to Genus, Species, and Phylogenetic Clade. Clade membership of bacterial OTUs is based on 16S sequence data. Clades are defined based on the topology of a phylogenetic tree that is constructed from full-length 16S sequences using maximum likelihood methods familiar to individuals with ordinary skill in the art of phylogenetics. Clades are constructed to ensure that all OTUs in a given clade are: (i) within a specified number of bootstrap supported nodes from one another, and (ii) within 5% genetic similarity. OTUs that are within the same clade can be distinguished as genetically and phylogenetically distinct from OTUs in a different clade based on 16S-V4 sequence data, while OTUs falling within the same clade are closely related. OTUs falling within the same clade are evolutionarily closely related and may or may not be distinguishable from one another using 16S-V4 sequence data. Members of the same clade, due to their evolutionary relatedness, play similar functional roles in a microbial ecology such as that found in the human gut. Compositions substituting one species with another from the same clade are likely to have conserved ecological function and therefore are useful in the present invention. All OTUs are denoted as to their putative capacity to form spores and whether they are a Pathogen or Pathobiont (see Definitions for description of “Pathobiont”). NIAID Priority Pathogens are denoted as ‘Category-A’, ‘Category-B’, or ‘Category-C’, and Opportunistic Pathogens are denoted as ‘OP’. OTUs that are not pathogenic or for which their ability to exist as a pathogen is unknown are denoted as ‘N’. The ‘SEQ ID Number’ denotes the identifier of the OTU in the Sequence Listing File and ‘Public DB Accession’ denotes the identifier of the OTU in a public sequence repository.

Table 2 contains bacterial OTUs identified from the 16s analysis of the ethanol treated spore population before and after a CsCl gradient purification.

“Residual habitat products” refers to material derived from the habitat for microbiota within or on a human or animal. For example, microbiota live in feces in the gastrointestinal tract, on the skin itself, in saliva, mucus of the respiratory tract, or secretions of the genitourinary tract (i.e., biological matter associated with the microbial community). Substantially free of residual habitat products means that the bacterial composition no longer contains the biological matter associated with the microbial environment on or in the human or animal subject and is 100% free, 99% free, 98% free, 97% free, 96% free, or 95% free of any contaminating biological matter associated with the microbial community. Residual habitat products can include abiotic materials (including undigested food) or it can include unwanted microorganisms. Substantially free of residual habitat products may also mean that the bacterial composition contains no detectable cells from a human or animal and that only microbial cells are detectable. In one embodiment, substantially free of residual habitat products may also mean that the bacterial composition contains no detectable viral (including bacterial viruses (i.e., phage)), fungal, mycoplasmal contaminants. In another embodiment, it means that fewer than 1×10⁻²%, 1×10⁻³%, 1×10⁻⁴%, 1×10⁻⁵%, 1×10⁻⁶%, 1×10⁻⁷%, 1×10⁻⁸ of the viable cells in the bacterial composition are human or animal, as compared to microbial cells. There are multiple ways to accomplish this degree of purity, none of which are limiting. Thus, contamination may be reduced by isolating desired constituents through multiple steps of streaking to single colonies on solid media until replicate (such as, but not limited to, two) streaks from serial single colonies have shown only a single colony morphology. Alternatively, reduction of contamination can be accomplished by multiple rounds of serial dilutions to single desired cells (e.g., a dilution of 10⁻⁸ or 10⁻⁹), such as through multiple 10-fold serial dilutions. This can further be confirmed by showing that multiple isolated colonies have similar cell shapes and Gram staining behavior. Other methods for confirming adequate purity include genetic analysis (e.g. PCR, DNA sequencing), serology and antigen analysis, enzymatic and metabolic analysis, and methods using instrumentation such as flow cytometry with reagents that distinguish desired constituents from contaminants.

“Clade” refers to the OTUs or members of a phylogenetic tree that are downstream of a statistically valid node in a phylogenetic tree. The clade comprises a set of terminal leaves in the phylogenetic tree that is a distinct monophyletic evolutionary unit and that share some extent of sequence similarity.

16s Sequencing, 16s, 16s-rRNA, 16s-NGS: In microbiology, “16S sequencing” or “16S-rRNA” or “16S” refers to sequence derived by characterizing the nucleotides that comprise the 16S ribosomal RNA gene(s). The bacterial 16S rDNA is approximately 1500 nucleotides in length and is used in reconstructing the evolutionary relationships and sequence similarity of one bacterial isolate to another using phylogenetic approaches. 16S sequences are used for phylogenetic reconstruction as they are in general highly conserved, but contain specific hypervariable regions that harbor sufficient nucleotide diversity to differentiate genera and species of most bacteria.

The “V1-V9 regions” of the 16S rRNA refers to the first through ninth hypervariable regions of the 16S rRNA gene that are used for genetic typing of bacterial samples. These regions in bacteria are defined by nucleotides 69-99, 137-242, 433-497, 576-682, 822-879. 986-1043, 1117-1173, 1243-1294 and 1435-1465 respectively using numbering based on the E. coli system of nomenclature. Brosius et al., Complete nucleotide sequence of a 16S ribosomal RNA gene from Escherichia coli, PNAS 75 (10): 4801-4805 (1978). In some embodiments, at least one of the V1, V2, V3, V4, V5, V6, V7, V8, and V9 regions are used to characterize an OTU. In one embodiment, the V1, V2, and V3 regions are used to characterize an OTU. In another embodiment, the V3, V4, and V5 regions are used to characterize an OTU. In another embodiment, the V4 region is used to characterize an OTU. A person of ordinary skill in the art can identify the specific hypervariable regions of a candidate 16S rRNA by comparing the candidate sequence in question to a reference sequence and identifying the hypervariable regions based on similarity to the reference hypervariable regions, or alternatively, one can employ Whole Genome Shotgun (WGS) sequence characterization of microbes or a microbial community.

The term “subject” refers to any animal subject including humans, laboratory animals (e.g., primates, rats, mice), livestock (e.g., cows, sheep, goats, pigs, turkeys, and chickens), and household pets (e.g., dogs, cats, and rodents). The subject may be suffering from a dysbiosis, including, but not limited to, an infection due to a gastrointestinal pathogen or may be at risk of developing or transmitting to others an infection due to a gastrointestinal pathogen.

The term “phenotype” refers to a set of observable characteristics of an individual entity. As example an individual subject may have a phenotype of “health” or “disease”. Phenotypes describe the state of an entity and all entities within a phenotype share the same set of characteristics that describe the phenotype. The phenotype of an individual results in part, or in whole, from the interaction of the entities genome and/or microbiome with the environment.

The term “Network Ecology” refers to a consortium of OTUs that co-occur in some number of subjects. As used herein, a “network” is defined mathematically by a graph delineating how specific nodes (i.e. OTUs) and edges (connections between specific OTUs) relate to one another to define the structural ecology of a consortium of OTUs. Any given Network Ecology will possess inherent phylogenetic diversity and functional properties. A Network Ecology can also be defined in terms of function where for example the nodes would be comprised of elements such as, but not limited to, enzymes, clusters of orthologous groups (COGS; http: www.ncbi.nlm.nih.gov/books/NBK21090/), or KEGG pathways (www.genome.jp/kegg/).

Network Class, Core Network, Core Network Ecology: The terms “Network Class”, “Core Network” and “Core Network Ecology” refer to a group of network ecologies that in general are computationally determined to comprise ecologies with similar phylogenetic and/or functional characteristics. A Core Network therefore contains important biological features, defined either phylogenetically or functionally, of a group (i.e., a cluster) of related network ecologies. One representation of a Core Network Ecology is a designed consortium of microbes, typically non-pathogenic bacteria, that represents core features of a set of phylogenetically or functionally related network ecologies seen in many different subjects. In many occurrences, a Core Network, while designed as described herein, exists as a Network Ecology observed in one or more subjects. Core Network ecologies are useful for reversing or reducing a dysbiosis in subjects where the underlying, related Network Ecology has been disrupted.

The term “Keystone OTU” refers to one or more OTUs that are common to many network ecologies and are members of networks ecologies that occur in many subjects (i.e. are pervasive). Due to the ubiquitous nature of Keystone OTUs, they are central to the function of network ecologies in healthy subjects and are often missing or at reduced levels in subjects with disease. Keystone OTUs may exist in low, moderate, or high abundance in subjects.

The term “non-Keystone OTU” refers to an OTU that is observed in a Network Ecology and is not a keystone OTU.

The term “Phylogenetic Diversity” refers to the biodiversity present in a given Network Ecology or Core Network Ecology based on the OTUs that comprise the network. Phylogenetic diversity is a relative term, meaning that a Network Ecology or Core Network that is comparatively more phylogenetically diverse than another network contains a greater number of unique species, genera, and taxonomic families. Uniqueness of a species, genera, or taxonomic family is generally defined using a phylogenetic tree that represents the genetic diversity all species, genera, or taxonomic families relative to one another. In another embodiment phylogenetic diversity may be measured using the total branch length or average branch length of a phylogenetic tree.

A “spore” or a population of “spores” includes bacteria (or other single-celled organisms) that are generally viable, more resistant to environmental influences such as heat and bacteriocidal agents than vegetative forms of the same bacteria, and typically capable of germination and out-growth. “Spore-formers” or bacteria “capable of forming spores” are those bacteria containing the genes and other necessary abilities to produce spores under suitable environmental conditions.

A “spore population” refers to a plurality of spores present in a composition. Synonymous terms used herein include spore composition, spore preparation, ethanol treated spore fraction and spore ecology. A spore population may be purified from a fecal donation, e.g. via ethanol or heat treatment, or a density gradient separation or any combination of methods described herein to increase the purity, potency and/or concentration of spores in a sample. Alternatively, a spore population may be derived through culture methods starting from isolated spore former species or spore former OTUs or from a mixture of such species, either in vegetative or spore form.

In one embodiment, the spore preparation comprises spore forming species wherein residual non-spore forming species have been inactivated by chemical or physical treatments including ethanol, detergent, heat, sonication, and the like; or wherein the non-spore forming species have been removed from the spore preparation by various separations steps including density gradients, centrifugation, filtration and/or chromatography; or wherein inactivation and separation methods are combined to make the spore preparation. In yet another embodiment, the spore preparation comprises spore forming species that are enriched over viable non-spore formers or vegetative forms of spore formers. In this embodiment, spores are enriched by 2-fold, 5-fold, 10-fold, 50-fold. 100-fold, 1000-fold, 10,000-fold or greater than 10,000-fold compared to all vegetative forms of bacteria. In yet another embodiment, the spores in the spore preparation undergo partial germination during processing and formulation such that the final composition comprises spores and vegetative bacteria derived from spore forming species.

The term “isolated” encompasses a bacterium or other entity or substance that has been (1) separated from at least some of the components with which it was associated when initially produced (whether in nature or in an experimental setting), and/or (2) produced, prepared, purified, and/or manufactured by the hand of man. Isolated bacteria include those bacteria that are cultured, even if such cultures are not monocultures. Isolated bacteria may be separated from at least about 10%, about 20%, about 30%, about 40%, about 50%, about 60%, about 70%, about 80%, about 90%, or more of the other components with which they were initially associated. In some embodiments, isolated bacteria are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. The terms “purify.” “purifying” and “purified” refer to a bacterium or other material that has been separated from at least some of the components with which it was associated either when initially produced or generated (e.g., whether in nature or in an experimental setting), or during any time after its initial production. A bacterium or a bacterial population may be considered purified if it is isolated at or after production, such as from a material or environment containing the bacterium or bacterial population, or by passage through culture, and a purified bacterium or bacterial population may contain other materials up to about 10%, about 20%, about 30%, about 40%, about 50%, about 60%, about 70%, about 80%, about 90%, or above about 90% and still be considered “isolated.” In some embodiments, purified bacteria and bacterial populations are more than about 80%, about 85%, about 90%, about 91%, about 92%, about 93%, about 94%, about 95%, about 96%, about 97%, about 98%, about 99%, or more than about 99% pure. In the instance of bacterial compositions provided herein, the one or more bacterial types present in the composition can be independently purified from one or more other bacteria produced and/or present in the material or environment containing the bacterial type. Bacterial compositions and the bacterial components thereof are generally purified from residual habitat products.

“Inhibition” of a pathogen encompasses the inhibition of any desired function or activity of the bacterial compositions of the present invention. Demonstrations of pathogen inhibition, such as decrease in the growth of a pathogenic bacterium or reduction in the level of colonization of a pathogenic bacterium are provided herein and otherwise recognized by one of ordinary skill in the art. Inhibition of a pathogenic bacterium's “growth” may include inhibiting the increase in size of the pathogenic bacterium and/or inhibiting the proliferation (or multiplication) of the pathogenic bacterium. Inhibition of colonization of a pathogenic bacterium may be demonstrated by measuring the amount or burden of a pathogen before and after a treatment. An “inhibition” or the act of “inhibiting” includes the total cessation and partial reduction of one or more activities of a pathogen, such as growth, proliferation, colonization, and function.

A “germinant” is a material or composition or physical-chemical process capable of inducing vegetative growth of a bacterium that is in a dormant spore form, or group of bacteria in the spore form, either directly or indirectly in a host organism and/or in vitro.

A “sporulation induction agent” is a material or physical-chemical process that is capable of inducing sporulation in a bacterium, either directly or indirectly, in a host organism and/or in vitro.

To “increase production of bacterial spores” includes an activity or a sporulation induction agent. “Production” includes conversion of vegetative bacterial cells into spores and augmentation of the rate of such conversion, as well as decreasing the germination of bacteria in spore form, decreasing the rate of spore decay in vivo, or ex vivo, or to increasing the total output of spores (e.g. via an increase in volumetric output of fecal material).

The “colonization” of a host organism includes the non-transitory residence of a bacterium or other microscopic organism. As used herein, “reducing colonization” of a host subject's gastrointestinal tract (or any other microbiotal niche) by a pathogenic bacterium includes a reduction in the residence time of the pathogen in the gastrointestinal tract as well as a reduction in the number (or concentration) of the pathogen in the gastrointestinal tract or adhered to the luminal surface of the gastrointestinal tract. Measuring reductions of adherent pathogens may be demonstrated, e.g., by a biopsy sample, or reductions may be measured indirectly, e.g., by measuring the pathogenic burden in the stool of a mammalian host.

A “combination” of two or more bacteria includes the physical co-existence of the two bacteria, either in the same material or product or in physically connected products, as well as the temporal co-administration or co-localization of the two bacteria.

A “cytotoxic” activity or bacterium includes the ability to kill a bacterial cell, such as a pathogenic bacterial cell. A “cytostatic” activity or bacterium includes the ability to inhibit, partially or fully, growth, metabolism, and/or proliferation of a bacterial cell, such as a pathogenic bacterial cell.

Non-comestible products: To be free of “non-comestible products” means that a bacterial composition or other material provided herein does not have a substantial amount of a non-comestible product, e.g., a product or material that is inedible, harmful or otherwise undesired in a product suitable for administration, e.g., oral administration, to a human subject. Non-comestible products are often found in preparations of bacteria from the prior art.

As used herein the term “vitamin” is understood to include any of various fat-soluble or water-soluble organic substances (non-limiting examples include vitamin A, Vitamin B1 (thiamine), Vitamin B2 (riboflavin), Vitamin B3 (niacin or niacinamide), Vitamin B5 (pantothenic acid), Vitamin B6 (pyridoxine, pyridoxal, or pyridoxamine, or pyridoxine hydrochloride), Vitamin B7 (biotin), Vitamin B9 (folic acid), and Vitamin B12 (various cobalamins; commonly cyanocobalamin in vitamin supplements), vitamin C, vitamin D, vitamin E, vitamin K, K1 and K2 (i.e. MK-4, MK-7), folic acid and biotin) essential in minute amounts for normal growth and activity of the body and obtained naturally from plant and animal foods or synthetically made, pro-vitamins, derivatives, analogs.

As used herein, the term “minerals” is understood to include boron, calcium, chromium, copper, iodine, iron, magnesium, manganese, molybdenum, nickel, phosphorus, potassium, selenium, silicon, tin, vanadium, zinc, or combinations thereof.

As used herein, the term “antioxidant” is understood to include any one or more of various substances such as beta-carotene (a vitamin A precursor), vitamin C, vitamin E, and selenium) that inhibit oxidation or reactions promoted by Reactive Oxygen Species (“ROS”) and other radical and non-radical species. Additionally, antioxidants are molecules capable of slowing or preventing the oxidation of other molecules. Non-limiting examples of antioxidants include astaxanthin, carotenoids, coenzyme Q10 (“CoQ10”), flavonoids, glutathione, Goji (wolfberry), hesperidin, lactowolfberry, lignan, lutein, lycopene, polyphenols, selenium, vitamin A, vitamin C, vitamin E, zeaxanthin, or combinations thereof.

### Compositions of the Invention

### Bacterial Compositions

Provided are bacteria and combinations of bacteria of the human gut microbiota with the capacity to meaningfully provide functions of a healthy microbiota when administered to mammalian hosts. Without being limited to a specific mechanism, it is thought that such compositions inhibit the growth, proliferation, and/or colonization of one or a plurality of pathogenic bacteria in the dysbiotic microbiotal niche, so that a healthy, diverse and protective microbiota colonizes and populates the intestinal lumen to establish or reestablish ecological control over pathogens or potential pathogens (e.g., some bacteria are pathogenic bacteria only when present in a dysbiotic environment). In some embodiments, bacterial composition engraft in the host gut are detectable 1, 2, 3, 4, 5, or 6 days, 1 or 2 weeks, 1, 3 or 6 months, 1 year or greater than 1 year after the administration of composition when the bacteria compositions were not present before treatment. In some embodiments, the bacteria compositions delivered shift the gut environment to a state allowing other healthy commensal bacteria to augment the delivered population. In this manner, augmentation of the commensal bacteria not directly delivered increases the overall diversity of the subsequent microbiome with healthy commensal bacteria and improves host health.

Inhibition of pathogens includes those pathogens such as C. difficile, Salmonella spp., enteropathogenic E coli, multi-drug resistant bacteria such as Klebsiella, and E. coli, Carbapenem-resistant Enterobacteriaceae (CRE), extended spectrum beta-lactam resistant Enterococci (ESBL), and vancomycin-resistant Enterococci (VRE). In particular, provided are synergistic combinations that treat, prevent, delay or reduce the symptoms of diseases, disorders and conditions associated with a dysbiosis. Representative diseases, disorders and conditions potentially associated with a dysbiosis, which are suitable for treatment with the compositions and methods as described herein, are provided in Tables 3 and 4.

As used herein, a “type” or more than one “types” of bacteria may be differentiated at the genus level, the species, level, the sub-species level, the strain level or by any other taxonomic method, as described herein and otherwise known in the art.

Bacterial compositions may comprise two types of bacteria (termed “binary combinations” or “binary pairs”) or greater than two types of bacteria. For instance, a bacterial composition may comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, or at least 21, 22, 23, 24, 25, 26, 27, 28, 29 30, 31, 32, 33, 34, 35, 36, 37, 38, 39, or at least 40, at least 50 or greater than 50 types of bacteria, as defined by species or operational taxonomic unit (OTU), or otherwise as provided herein.

In another embodiment, the number of types of bacteria present in a bacterial composition is at or below a known value. For example, in such embodiments the bacterial composition comprises 50 or fewer types of bacteria, such as 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, or 10 or fewer, or 9 or fewer types of bacteria, 8 or fewer types of bacteria, 7 or fewer types of bacteria, 6 or fewer types of bacteria, 5 or fewer types of bacteria, 4 or fewer types of bacteria, or 3 or fewer types of bacteria. In another embodiment, a bacterial composition comprises from 2 to no more than 40, from 2 to no more than 30, from 2 to no more than 20, from 2 to no more than 15, from 2 to no more than 10, or from 2 to no more than 5 types of bacteria. In some embodiments, bacterial compositions are provided with the ability to exclude pathogenic bacteria. Exemplary bacterial compositions are demonstrated to reduce the growth rate of one pathogen, C. difficile, as provided in the Examples, wherein the ability of the bacterial compositions is demonstrated by assessing the antagonism activity of a combination of OTUs or strains towards a given pathogen using in vitro assays.

### Bacterial Compositions Described by Species

Bacterial compositions may be prepared comprising at least two types of isolated bacteria, chosen from the species in Table 1.

In one embodiment, the bacterial composition comprises at least one and preferably more than one of the following: Enterococcus faecalis (previously known as Streptococcus faecalis), Clostridium innocuum, Clostridium ramosum, Bacteroides ovatus, Bacteroides vulgatus, Bacteroides thetaiotaomicron, Escherichia coli (1109 and 1108-1), Clostridium bifermentans, and Blautia producta (previously known as Peptostreptococcus productus). In an alternative embodiment, at least one of the preceding species is not substantially present in the bacterial composition.

In one embodiment, the bacterial composition comprises at least one and preferably more than one of the following: Enterococcus faecalis (previously known as Streptococcus faecalis), Clostridium innocuum, Clostridium ramosum, Bacteroides ovatus, Bacteroides vulgatus, Bacteroides thetaiotaomicron, Escherichia coli (1109 and 1108-1), Clostridium bifermentans, and Blautia producta (previously known as Peptostreptococcus productus). In an alternative embodiment, at least one of the preceding species is not substantially present in the bacterial composition.

In another embodiment, the bacterial composition comprises at least one and preferably more than one of the following: Acidaminococcus intestinalis, Bacteroides ovatus, two strains of Bifidobacterium adolescentis, two strains of Bifidobacterium longum, Blautia producta, Clostridium cocleatum, Collinsella aerofaciens, two strains of Dorea longicatena, Escherichia coli, Eubacterium desmolans, Eubacterium eligens, Eubacterium limosum, four strains of Eubacterium rectale, Eubacterium ventriosum, Faecalibacterium prausnitzii, Lachnospira pectinoshiza, Lactobacillus casei, Lactobacillus casei/paracasei, Paracateroides distasonis, Raoultella sp., one strain of Roseburia (chosen from Roseburia faecalis or Roseburia faecis), Roseburia intestinalis, two strains of Ruminococcus torques, two strains of Ruminococcus obeum, and Streptococcus mitis. In an alternative embodiment, at least one of the preceding species is not substantially present in the bacterial composition.

In yet another embodiment, the bacterial composition comprises at least one and preferably more than one of the following: Barnesiella intestinihominis; Lactobacillus reuteri; a species characterized as one of Enterococcus hirae, Enterococcus faecium, or Enterococcus durans; a species characterized as one of Anaerostipes caccae or Clostridium indolis; a species characterized as one of Staphylococcus warneri or Staphylococcus pasteuri; and Adlercreutzia equolifaciens. In an alternative embodiment, at least one of the preceding species is not substantially present in the bacterial composition.

In other embodiments, the bacterial composition comprises at least one and preferably more than one of the following: Clostridium absonum, Clostridium argentinense, Clostridium baratii, Clostridium bartlettii, Clostridium bifermentans, Clostridium botulinum, Clostridium butyricum, Clostridium cadaveris, Clostridium camis, Clostridium celatum, Clostridium chauvoei, Clostridium clostridioforme, Clostridium cochlearium, Clostridium difficile, Clostridium fallax, Clostridium felsineum, Clostridium ghonii, Clostridium glycolicum, Clostridium haemolyticum, Clostridium hastiforme, Clostridium histolyticum, Clostridium indolis, Clostridium innocuum, Clostridium irregulare, Clostridium limosum, Clostridium malenominatum, Clostridium novyi, Clostridium oroticum, Clostridium paraputrificum, Clostridium perfringens, Clostridium piliforme, Clostridium putrefaciens, Clostridium putrificum, Clostridium ramosum, Clostridium sardiniense, Clostridium sartagoforme, Clostridium scindens, Clostridium septicum, Clostridium sordellii, Clostridium sphenoides, Clostridium spiroforme, Clostridium sporogenes, Clostridium subterminale, Clostridium symbiosum, Clostridium tertium, Clostridium tetani, Clostridium welchii, and Clostridium villosum. In an alternative embodiment, at least one of the preceding species is not substantially present in the bacterial composition.

In one embodiment, the bacterial composition comprises at least one and preferably more than one of the following: Clostridium innocuum, Clostridium bifermentans, Clostridium butyricum, Bacteroides fragilis, Bacteroides thetaiotaomicron, Bacteroides uniformis, three strains of Escherichia coli, and Lactobacillus sp. In an alternative embodiment, at least one of the preceding species is not substantially present in the bacterial composition.

In one embodiment, the bacterial composition comprises at least one and preferably more than one of the following: Clostridium bifermentans, Clostridium innocuum, Clostridium butyricum, three strains of Escherichia coli, three strains of Bacteroides, and Blautia producta. In an alternative embodiment, at least one of the preceding species is not substantially present in the bacterial composition.

In one embodiment, the bacterial composition comprises at least one and preferably more than one of the following: Bacteroides sp., Escherichia coli, and non pathogenic Clostridia, including Clostridium innocuum, Clostridium bifermentans and Clostridium ramosum. In an alternative embodiment, at least one of the preceding species is not substantially present in the bacterial composition.

In one embodiment, the bacterial composition comprises at least one and preferably more than one of the following: Bacteroides species, Escherichia coli and non-pathogenic Clostridia, such as Clostridium butyricum, Clostridium bifermentans and Clostridium innocuum. In an alternative embodiment, at least one of the preceding species is not substantially present in the bacterial composition.

In one embodiment, the bacterial composition comprises at least one and preferably more than one of the following: Bacteroides caccae, Bacteroides capillosus, Bacteroides coagulans, Bacteroides distasonis, Bacteroides eggerthii, Bacteroides forsythus, Bacteroides fragilis, Bacteroides fragilis-ryhm, Bacteroides gracilis, Bacteroides levii, Bacteroides macacae, Bacteroides merdae, Bacteroides ovatus, Bacteroides pneumosintes, Bacteroides putredinis, Bacteroides pyogenes, Bacteroides splanchnicus, Bacteroides stercoris, Bacteroides tectum, Bacteroides thetaiotaomicron, Bacteroides uniformis, Bacteroides ureolyticus, and Bacteroides vulgatus. In an alternative embodiment, at least one of the preceding species is not substantially present in the bacterial composition.

In one embodiment, the bacterial composition comprises at least one and preferably more than one of the following: Bacteroides, Eubacteria, Fusobacteria, Propionibacteria, Lactobacilli, anaerobic cocci, Ruminococcus, Escherichia coli, Gemmiger, Desulfomonas, and Peptostreptococcus. In an alternative embodiment, at least one of the preceding species is not substantially present in the bacterial composition.

In one embodiment, the bacterial composition comprises at least one and preferably more than one of the following: Bacteroides fragilis ss. Vulgatus, Eubacterium aerofaciens, Bacteroides fragilis ss. Thetaiotaomicron, Blautia producta (previously known as Peptostreptococcus productus II), Bacteroides fragilis ss. Distasonis, Fusobacterium prausnitzii, Coprococcus eutactus, Eubacterium aerofaciens III, Blautia producta (previously known as Peptostreptococcus productus I), Ruminococcus bromii, Bifidobacterium adolescentis, Gemmiger formicilis, Bifidobacterium longum, Eubacterium siraeum, Ruminococcus torques, Eubacterium rectale III-H, Eubacterium rectale IV, Eubacterium eligens, Bacteroides eggerthii, Clostridium leptum, Bacteroides fragilis ss. A, Eubacterium biforme, Bifidobacterium infantis, Eubacterium rectale III-F, Coprococcus comes, Bacteroides capillosus, Ruminococcus albus, Eubacterium formicigenerans, Eubacterium hallii, Eubacterium ventriosum I, Fusobacterium russii, Ruminococcus obeum, Eubacterium rectale II, Clostridium ramosum I, Lactobacillus leichmanii, Ruminococcus cailidus, Butyrivibrio crossotus, Acidaminococcus fermentans, Eubacterium ventriosum, Bacteroides fragilis ss. fragilis, Bacteroides AR, Coprococcus catus, Eubacterium hadrum, Eubacterium cylindroides, Eubacterium ruminantium, Eubacterium CH-1, Staphylococcus epidermidis, Peptostreptococcus BL, Eubacterium limosum, Bacteroides praeacutus, Bacteroides L, Fusobacterium mortiferum I, Fusobacterium naviforme, Clostridium innocuum, Clostridium ramosum, Propionibacterium acnes, Ruminococcus flavefaciens, Ruminococcus AT, Peptococcus AU-1, Eubacterium AG, -AK, -AL, -AL-1, -AN, Bacteroides fragilis ss. ovatus, -ss. d, -ss. f; Bacteroides L-1, L-5; Fusobacterium nucleatum, Fusobacterium mortiferum, Escherichia coli, Streptococcus morbiliorum, Peptococcus magnus, Peptococcus G, AU-2; Streptococcus intermedius, Ruminococcus lactaris, Ruminococcus CO Gemmiger X, Coprococcus BH, -CC; Eubacterium tenue, Eubacterium ramulus, Eubacterium AE, -AG-H, -AG-M, -AJ, -BN-1; Bacteroides clostridiiformis ss. clostridliformis, Bacteroides coagulans, Bacteroides orails, Bacteroides ruminicola ss. brevis, -ss. ruminicola, Bacteroides splanchnicus, Desuifomonas pigra, Bacteroides L-4, -N-i; Fusobacterium H, Lactobacillus G, and Succinivibrio A. In an alternative embodiment, at least one of the preceding species is not substantially present in the bacterial composition.

### Bacterial Compositions Described by Operational Taxonomic Unit (OTUs)

Bacterial compositions may be prepared comprising at least two types of isolated bacteria, chosen from Table 1.

In one embodiment, the OTUs can be characterized by one or more of the variable regions of the 16S sequence (V1-V9). These regions in bacteria are defined by nucleotides 69-99, 137-242, 433-497, 576-682, 822-879, 986-1043, 1117-1173, 1243-1294 and 1435-1465 respectively using numbering based on the E. coli system of nomenclature. (See, e.g., Brosius et al., Complete nucleotide sequence of a 16S ribosomal RNA gene from Escherichia coli, PNAS 75 (10): 4801-4805 (1978)). In some embodiments, at least one of the V1, V2, V3, V4, V5, V6, V7, V8, and V9 regions are used to characterize an OTU. In one embodiment, the V1, V2, and V3 regions are used to characterize an OTU. In another embodiment, the V3. V4, and V5 regions are used to characterize an OTU. In another embodiment, the V4 region is used to characterize an OTU.

### Bacterial Compositions Exclusive of Certain Bacterial Species or Strains

In one embodiment, the bacterial composition does not comprise at least one of Enterococcus faecalis (previously known as Streptococcus faecalis), Clostridium innocuum, Clostridium ramosum, Bacteroides ovatus, Bacteroides vulgatus, Bacteroides thetaiotaomicron, Escherichia coli (1109 and 1108-1). Clostridium bifermentans, and Blautia producta (previously known as Peptostreptococcus productus).

In another embodiment, the bacterial composition does not comprise at least one of Acidaminococcus intestinalis, Bacteroides ovatus, two species of Bifidobacterium adolescentis, two species of Bifidobacterium longum, Collinsella aerofaciens, two species of Dorea longicatena, Escherichia coli, Eubacterium eligens, Eubacterium limosum, four species of Eubacterium rectale, Eubacterium ventriosium, Faecalibacterium prausnitzii, Lactobacillus casei, Lactobacillus paracasei, Paracateroides distasonis, Raoultella sp., one species of Roseburia (chosen from Roseburia faecalis or Roseburia faecis), Roseburia intestinalis, two species of Ruminococcus torques, and Streptococcus mitis.

In yet another embodiment, the bacterial composition does not comprise at least one of Barnesiella intestinihominis; Lactobacillus reuteri; a species characterized as one of Enterococcus hirae, Enterococcus faecium, or Enterococcus durans; a species characterized as one of Anaerostipes caccae or Clostridium indolis; a species characterized as one of Staphylococcus warneri or Staphylococcus pasteuri; and Adlercreutzia equolifaciens.

In other embodiments, the bacterial composition does not comprise at least one of Clostridium absonum, Clostridium argentinense, Clostridium baratii, Clostridium bifermentans, Clostridium botulinum, Clostridium butyricum, Clostridium cadaveris, Clostridium camis, Clostridium celatum, Clostridium chauvoei, Clostridium clostridioforme, Clostridium cochlearium, Clostridium difficile, Clostridium fallax, Clostridium felsineum, Clostridium ghonii, Clostridium glycolicum, Clostridium haemolyticum, Clostridium hastiforme, Clostridium histolyticum, Clostridium indolis, Clostridium innocuum, Clostridium irregulare, Clostridium limosum, Clostridium malenominatum, Clostridium novyi, Clostridium oroticum, Clostridium paraputrificum, Clostridium perfringens, Clostridium piliforme, Clostridium putrefaciens, Clostridium putrificum, Clostridium ramosum, Clostridium sardiniense, Clostridium sartagoforme, Clostridium scindens, Clostridium septicum, Clostridium sordellii, Clostridium sphenoides, Clostridium spiroforme, Clostridium sporogenes, Clostridium subterminale, Clostridium symbiosum, Clostridium tertium, Clostridium tetani, Clostridium welchii, and Clostridium villosum.

In another embodiment, the bacterial composition does not comprise at least one of Clostridium innocuum, Clostridium bifermentans, Clostridium butyricum, Bacteroides fragilis, Bacteroides thetaiotaomicron, Bacteroides uniformis, three strains of Escherichia coli, and Lactobacillus sp.

In another embodiment, the bacterial composition does not comprise at least one of Clostridium bifermentans, Clostridium innocuum, Clostridium butyricum, three strains of Escherichia coli, three strains of Bacteroides, and Blautia producta (previously known as Peptostreptococcus productus).

In another embodiment, the bacterial composition does not comprise at least one of Bacteroides sp., Escherichia coli, and non pathogenic Clostridia, including Clostridium innocuum, Clostridium bifermentans and Clostridium ramosum.

In another embodiment, the bacterial composition does not comprise at least one of more than one Bacteroides species, Escherichia coli and non-pathogenic Clostridia, such as Clostridium butyricum, Clostridium bifermentans and Clostridium innocuum.

In another embodiment, the bacterial composition does not comprise at least one of Bacteroides caccae, Bacteroides capillosus, Bacteroides coagulans, Bacteroides distasonis, Bacteroides eggerthii, Bacteroides forsythus, Bacteroides fragilis, Bacteroides fragilis-ryhm, Bacteroides gracilis, Bacteroides levii, Bacteroides macacae, Bacteroides merdae, Bacteroides ovatus, Bacteroides pneumosintes, Bacteroides putredinis, Bacteroides pyogenes, Bacteroides splanchnicus, Bacteroides stercoris, Bacteroides tectum, Bacteroides thetaiotaomicron, Bacteroides uniformis, Bacteroides ureolyticus, and Bacteroides vulgatus.

In another embodiment, the bacterial composition does not comprise at least one of Bacteroides, Eubacteria, Fusobacteria, Propionibacteria, Lactobacilli, anaerobic cocci, Ruminococcus, Escherichia coli, Gemmiger, Desulfomonas, and Peptostreptococcus.

In another embodiment, the bacterial composition does not comprise at least one of Bacteroides fragilis ss. Vulgatus, Eubacterium aerofaciens, Bacteroides fragilis ss. Thetaiotaomicron, Blautia producta (previously known as Peptostreptococcus productus II), Bacteroides fragilis ss. Distasonis, Fusobacterium prausnitzii, Coprococcus eutactus, Eubacterium aerofaciens III, Blautia producta (previously known as Peptostreptococcus productus I), Ruminococcus bromil, Bifidobacterium adolescentis, Gemmiger formicilis, Bifidobacterium longum, Eubacterium siraeum, Ruminococcus torques, Eubacterium rectale III-H, Eubacterium rectale IV, Eubacterium eligens, Bacteroides eggerthii, Clostridium leptum, Bacteroides fragilis ss. A, Eubacterium biforme, Bifidobacterium infantis, Eubacterium rectale III-F, Coprococcus comes, Bacteroides capillosus, Ruminococcus albus, Eubacterium formicigenerans, Eubacterium hallii, Eubacterium ventriosum I, Fusobacterium russii, Ruminococcus obeum, Eubacterium rectale II, Clostridium ramosum I, Lactobacillus leichmanii, Ruminococcus cailidus, Butyrivibrio crossotus, Acidaminococcus fermentans, Eubacterium ventriosum, Bacteroides fragilis ss. fragilis, Bacteroides AR, Coprococcus catus, Eubacterium hadrum, Eubacterium cylindroides, Eubacterium ruminantium, Eubacterium CH-1, Staphylococcus epidermidis, Peptostreptococcus BL, Eubacterium limosum, Bacteroides praeacutus, Bacteroides L, Fusobacterium mortiferum I, Fusobacterium naviforme, Clostridium innocuum, Clostridium ramosum, Propionibacterium acnes, Ruminococcus flavefaciens, Ruminococcus AT, Peptococcus AU-1, Eubacterium AG, -AK, -AL, -AL-1, -AN; Bacteroides fragilis ss. ovatus, -ss. d, -ss. f; Bacteroides L-1, L-5; Fusobacterium nucleatum, Fusobacterium mortiferum, Escherichia coli. Streptococcus morbiliorum, Peptococcus magnus, Peptococcus G, AU-2; Streptococcus intermedius, Ruminococcus lactaris, Ruminococcus CO Gemmiger X, Coprococcus BH, -CC; Eubacterium tenue, Eubacterium ramulus, Eubacterium AE, -AG-H, -AG-M, -AJ, -BN-1; Bacteroides clostridiiformis ss. clostridliformis, Bacteroides coagulans, Bacteroides orails, Bacteroides ruminicola ss. brevis, -ss. ruminicola, Bacteroides splanchnicus, Desuifomonas pigra, Bacteroides L-4, -N-i; Fusobacterium H, Lactobacillus G, and Succinivibrio A.

### Inhibition of Bacterial Pathogens

In some embodiments, the bacterial composition provides a protective or therapeutic effect against infection by one or more GI pathogens of interest.

A list of exemplary bacterial pathogens is provided in Table 1, demarcated by the pathogen status column.

In some embodiments, the pathogenic bacterium or pathobiont is selected from the group categorized as pathogens or pathobionts in Table 1. In another embodiment, the pathogenic bacterium is selected from the group consisting of Yersinia, Vibrio, Treponema, Streptococcus, Staphylococcus, Shigella, Salmonella, Rickettsia, Orientia, Pseudomonas, Neisseria, Mycoplasma, Mycobacterium, Listeria, Leptospira, Legionella, Klebsiella, Helicobacter, Haemophilus, Francisella, Escherichia, Ehrlichia, Enterococcus, Coxiella, Corynebacterium, Clostridium, Chlamydia, Chlamydophila, Campylobacter, Burkholderia, Brucella, Borrelia, Bordetella, Bifidobacterium, Bacillus, multi-drug resistant bacteria, extended spectrum beta-lactam resistant Enterococci (ESBL), Carbapenem-resistant Enterobacteriaceae (CRE), and vancomycin-resistant Enterococci (VRE).

In some embodiments, these pathogens include, but are not limited to, Aeromonas hydrophila, Campylobacter fetus, Plesiomonas shigelloides, Bacillus cereus, Campylobacter jejuni, Clostridium botulinum, Clostridium difficile, Clostridium perfringens, enteroaggregative Escherichia coli, enterohemorrhagic Escherichia coli, enteroinvasive Escherichia coli, enterotoxigenic Escherichia coli (such as, but not limited to, LT and/or ST), Escherichia coli 0157: H7, Helicobacter pylori, Klebsiella pneumonia, Lysteria monocytogenes, Plesiomonas shigelloides, Salmonella spp., Salmonella typhi, Salmonella paratyphi, Shigella spp., Staphylococcus spp., Staphylococcus aureus, vancomycin-resistant enterococcus spp., Vibrio spp., Vibrio cholerae, Vibrio parahaemolyticus, Vibrio vulnificus, and Yersinia enterocolitica.

In one embodiment, the pathogen of interest is at least one pathogen chosen from Clostridium difficile, Salmonella spp., pathogenic Escherichia coli, vancomycin-resistant Enterococcus spp., and extended spectrum beta-lactam resistant Enterococci (ESBL).

### Purified Spore Populations

In some embodiments, the bacterial compositions comprise purified spore populations. Purified spore populations contain combinations of commensal bacteria of the human gut microbiota with the capacity to meaningfully provide functions of a healthy microbiota when administered to a mammalian subject. Without being limited to a specific mechanism, it is thought that such compositions inhibit the growth of a pathogen such as C. difficile, Salmonella spp., enteropathogenic E. coli, and vancomycin-resistant Enterococcus spp., so that a healthy, diverse and protective microbiota can be maintained or, in the case of pathogenic bacterial infections such as C. difficile infection, repopulate the intestinal lumen to reestablish ecological control over potential pathogens. Spore forming bacteria often exist in both a spore form and a vegetative form in a given population of cells. The biological activity and resistance to environmental stress can vary in each form and as a result both forms might be advantageous for different purposes. Bacterial compositions and methods described allow one to purify compositions containing spores to produce bacterial compositions that are resistant to environmental stresses such as but not limited to aerobic conditions, lack of nutrients, low pH, and temperature variations to populate the gut. In vegetative form, the proper bacterial compositions disclosed herein can produce beneficial health effects.

Disclosed herein are therapeutic compositions containing non-pathogenic, germination-competent bacterial spores, for the prevention, control, and treatment of gastrointestinal diseases, disorders and conditions and for general nutritional health. These compositions are advantageous in being suitable for safe administration to humans and other mammalian subjects and are efficacious in numerous gastrointestinal diseases, disorders and conditions and in general nutritional health. While spore-based compositions are known, these are generally prepared according to various techniques such as lyophilization or spray-drying of liquid bacterial cultures, resulting in poor efficacy, instability, substantial variability and lack of adequate safety.

It has now been found that populations of bacterial spores can be obtained from biological materials obtained from mammalian subjects, including humans. These populations are formulated into compositions as provided herein, and administered to mammalian subjects using the methods as provided herein.

Provided herein are therapeutic compositions containing a purified population of bacterial spores. As used herein, the terms “purify”, “purified” and “purifying” refer to the state of a population (e.g., a plurality of known or unknown amount and/or concentration) of desired bacterial spores, that have undergone one or more processes of purification, e.g., a selection or an enrichment of the desired bacterial spore, or alternatively a removal or reduction of residual habitat products as described herein. In some embodiments, a purified population has no detectable undesired activity or, alternatively, the level or amount of the undesired activity is at or below an acceptable level or amount. In other embodiments, a purified population has an amount and/or concentration of desired bacterial spores at or above an acceptable amount and/or concentration. In other embodiments, the purified population of bacterial spores is enriched as compared to the starting material (e.g., a fecal material) from which the population is obtained. This enrichment may be by 10%, 20%, 30%, 40%, 50%, 60%, 70%, 80%, 90%, 95%, 96%, 97%, 98%, 99%, 99.9%, 99.99%, 99.999%, 99.9999%, or greater than 99.9999% as compared to the starting material.

In certain embodiments, the purified populations of bacterial spores have reduced or undetectable levels of one or more pathogenic activities, such as toxicity, an infection of the mammalian recipient subject, an immunomodulatory activity, an autoimmune response, a metabolic response, or an inflammatory response or a neurological response. Such a reduction in a pathogenic activity may be by 10%, 20%, 30%, 40%, 50%, 60%, 70%, 80%, 90%, 95%, 96%, 97%, 98%, 99%, 99.9%, 99.99%, 99.999%, 99.9999%, or greater than 99.9999% as compared to the starting material. In other embodiments, the purified populations of bacterial spores have reduced sensory components as compared to fecal material, such as reduced odor, taste, appearance, and umami.

Provided are purified populations of bacterial spores that are substantially free of residual habitat products. In certain embodiments, this means that the bacterial spore composition no longer contains a substantial amount of the biological matter associated with the microbial community while living on or in the human or animal subject, and the purified population of spores may be 100% free, 99% free, 98% free, 97% free, 96% free, or 95% free of any contamination of the biological matter associated with the microbial community. Substantially free of residual habitat products may also mean that the bacterial spore composition contains no detectable cells from a human or animal, and that only microbial cells are detectable, in particular, only desired microbial cells are detectable. In another embodiment, it means that fewer than 1×10⁻²%, 1×10⁻³%, 1×10⁻⁴%, 1×10⁻⁵%, 1×10.6%. 1×10⁻⁷%, 1×10⁻⁸% of the cells in the bacterial composition are human or animal, as compared to microbial cells. In another embodiment, the residual habitat product present in the purified population is reduced at least a certain level from the fecal material obtained from the mammalian donor subject, e.g., reduced by at least about 10%, 20%, 30%, 40%, 50%, 60%, 70%, 80%, 90%, 95%, 96%, 97%, 98%, 99%, 99.9%, 99.99%, 99.999%, 99.9999%, or greater than 99.9999%.

In one embodiment, substantially free of residual habitat products or substantially free of a detectable level of a pathogenic material means that the bacterial composition contains no detectable viral (including bacterial viruses (i.e., phage)), fungal, or mycoplasmal or toxoplasmal contaminants, or a eukaryotic parasite such as a helminth. Alternatively, the purified spore populations are substantially free of an acellular material, e.g., DNA, viral coat material, or non-viable bacterial material.

As described herein, purified spore populations can be demonstrated by genetic analysis (e.g., PCR, DNA sequencing), serology and antigen analysis, and methods using instrumentation such as flow cytometry with reagents that distinguish desired bacterial spores from non-desired, contaminating materials.

Exemplary biological materials include fecal materials such as feces or materials isolated from the various segments of the small and large intestines. Fecal materials are obtained from a mammalian donor subject, or can be obtained from more than one donor subject, e.g., 2, 3, 4, 5, 6, 7, 8, 9, 10, 15, 20, 25, 30, 35, 40, 45, 50, 75, 100, 200, 300, 400, 500, 750, 1000 or from greater than 1000 donors, where such materials are then pooled prior to purification of the desired bacterial spores.

In alternative embodiments, the desired bacterial spores are purified from a single fecal material sample obtained from a single donor, and after such purification are combined with purified spore populations from other purifications, either from the same donor at a different time, or from one or more different donors, or both.

Preferred bacterial genera include Acetonema, Alkaliphilus, Alicyclobacillus, Amphibacillus, Ammonifex, Anaerobacter, Anaerofustis, Anaerostipes, Anaerotruncus, Anoxybacillus, Bacillus, Blautia, Brevibacillus, Bryantella, Caldicellulosiruptor, Caloramator, Candidatus, Carboxydibrachium, Carboxydothermus, Clostridium, Cohnella, Coprococcus, Dendrosporobacter Desulfitobacterium, Desulfosporosinus, Desulfotomaculum, Dorea, Eubacterium, Faecalibacterium, Filifactor, Geobacillus, Halobacteroides, Heliobacillus, Heliobacterium, Heliophilum, Heliorestis, Lachnoanaerobaculum, Lysinibacillus, Moorella, Oceanobacillus, Orenia (S.), Oxalophagus, Oxobacter, Paenibacillus, Pelospora, Pelotomaculum, Propionispora, Roseburia, Ruminococcus, Sarcina, Sporobacterium, Sporohalobacter, Sporolactobacillus, Sporomusa, Sporosarcina, Sporotomaculum, Subdoligranulum, Symbiobacterium, Syntrophobotulus, Syntrophospora, Terribacillus, Thermoanaerobacter, and Thermosinus.

Preferred bacterial species are provided at Table 1, demarcated as spore former or non-spore former. Where specific strains of a species are provided, one of skill in the art will recognize that other strains of the species or species in the same clade can be substituted for the named strain.

In some embodiments, spore-forming bacteria are identified by the presence of nucleic acid sequences that modulate sporulation. In particular, signature sporulation genes are highly conserved across members of distantly related genera including Clostridium and Bacillus. Traditional approaches of forward genetics have identified many, if not all, genes that are essential for sporulation (spo). The developmental program of sporulation is governed in part by the successive action of four compartment-specific sigma factors (appearing in the order σF, σE, σG and σK), whose activities are confined to the forespore (σF and σG) or the mother cell (GE and ok).

Provided are spore populations containing more than one type of bacterium. As used herein, a “type” or more than one “types” of bacteria may be differentiated at the genus level, the species, level, the sub-species level, the strain level or by any other taxonomic method, as described herein and otherwise known in the art.

In some embodiments, all or essentially all of the bacterial spores present in a purified population are obtained from a fecal material treated as described herein or otherwise known in the art. In alternative embodiments, one or more than one bacterial spores or types of bacterial spores are generated in culture and combined to form a purified spore population. In other alternative embodiments, one or more of these culture-generated spore populations are combined with a fecal material-derived spore population to generate a hybrid spore population. Bacterial compositions may contain at least two types of these preferred bacteria, including strains of the same species. For instance, a bacterial composition may comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 or more than 20 types of bacteria, as defined by species or operational taxonomic unit (OTU) encompassing such species.

Thus, provided herein are methods for production of a composition containing a population of bacterial spores suitable for therapeutic administration to a mammalian subject in need thereof. And the composition is produced by generally following the steps of: (a) providing a fecal material obtained from a mammalian donor subject; and (b) subjecting the fecal material to at least one purification treatment or step under conditions such that a population of bacterial spores is produced from the fecal material. The composition is formulated such that a single oral dose contains at least about 1×10⁴ colony forming units of the bacterial spores, and a single oral dose will typically contain about 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, or greater than 1×10¹⁵ CFUs of the bacterial spores. The presence and/or concentration of a given type of bacteria spore may be known or unknown in a given purified spore population. If known, for example the concentration of spores of a given strain, or the aggregate of all strains, is e.g., 1×10⁴. 1×10⁶, 1×10⁶, 1×10⁷, 1×10⁸, 1×10⁹, 1×10¹⁰, 1×10¹¹, 1×10¹², 1×10¹³, 1×10¹⁴, 1×1016, or greater than 1×10¹⁵ viable bacterial spores per gram of composition or per administered dose.

In some formulations, the composition contains at least about 10%, 20%, 30%, 40%, 50%, 60%, 70%, 80%, 90% or greater than 90% spores on a mass basis. In some formulations, the administered dose does not exceed 200, 300, 400, 500, 600, 700, 800, 900 milligrams or 1, 1.1, 1.2, 1.3, 1.4, 1.5, 1.6, 1.7, 1.8, or 1.9 grams in mass.

The bacterial spore compositions are generally formulated for oral or gastric administration, typically to a mammalian subject. In particular embodiments, the composition is formulated for oral administration as a solid, semi-solid, gel, or liquid form, such as in the form of a pill, tablet, capsule, or lozenge. In some embodiments, such formulations contain or are coated by an enteric coating to protect the bacteria through the stomach and small intestine, although spores are generally resistant to the stomach and small intestines.

The bacterial spore compositions may be formulated to be effective in a given mammalian subject in a single administration or over multiple administrations. For example, a single administration is substantially effective to reduce Cl. difficile and/or Cl. difficile toxin content in a mammalian subject to whom the composition is administered. Substantially effective means that Cl. difficile and/or Cl. difficile toxin content in the subject is reduced by at least 10%, 20%, 30%, 40%, 50%, 60%, 70%, 80%, 90%, 95%, 98%, 99% or greater than 99% following administration of the composition.

### Methods of the Invention

### In Vitro Assays Substantiating Protective Effect of Bacterial Compositions

In one embodiment, provided is an In Vitro Assay utilizing competition between the bacterial compositions or subsets thereof and C. difficile. Exemplary embodiments of this Assay are provided herein and in the Examples.

In another embodiment, provided is an In Vitro Assay utilizing 10% (wt/vol) Sterile-Filtered Feces. Provided is an in vitro assay to test for the protective effect of the bacterial compositions and to screen in vitro for combinations of microbes that inhibit the growth of a pathogen. The assay can operate in automated high-throughput or manual modes. Under either system, human or animal feces may be re-suspended in an anaerobic buffer solution, such as pre-reduced PBS or other suitable buffer, the particulate removed by centrifugation, and filter sterilized. This 10% sterile-filtered feces material serves as the base media for the in vitro assay. To test a bacterial composition, an investigator may add it to the sterile-filtered feces material for a first incubation period and then may inoculate the incubated microbial solution with the pathogen of interest for a second incubation period. The resulting titer of the pathogen may be quantified by any number of methods such as those described below, and the change in the amount of pathogen is compared to standard controls including the pathogen cultivated in the absence of the bacterial composition. The assay is conducted using at least one control. Feces from a healthy subject may be used as a positive control. As a negative control, antibiotic-treated feces or heat-treated feces may be used. Various bacterial compositions may be tested in this material and the bacterial compositions optionally compared to the positive and/or negative controls. The ability to inhibit the growth of the pathogen may be measured by plating the incubated material on C. difficile selective media and counting colonies. After competition between the bacterial composition and C. difficile, each well of the in vitro assay plate is serially diluted ten-fold six times, and plated on selective media, such as but not limited to cycloserine cefoxitin mannitol agar (CCMA) or cycloserine cefoxitin fructose agar (CCFA), and incubated. Colonies of C. difficile are then counted to calculate the concentration of viable cells in each well at the end of the competition. Colonies of C. difficile are confirmed by their characteristic diffuse colony edge morphology as well as fluorescence under UV light.

In another embodiment, the in vitro assay utilizes Antibiotic-Treated Feces. In an alternative embodiment, and instead of using 10% sterile-filtered feces, human or animal feces may be resuspended in an anaerobic buffer solution, such as pre-reduced PBS or other suitable buffer. The resuspended feces is treated with an antibiotic, such as clindamycin, or a cocktail of several antibiotics in order to reduce the ability of feces from a healthy subject to inhibit the growth of C. difficile; this material is termed the antibiotic-treated matrix. While not being bound by any mechanism, it is believed that beneficial bacteria in healthy subjects protects them from infection by competing out C. difficile. Treating feces with antibiotics kills or reduces the population of those beneficial bacteria, allowing C. difficile to grow in this assay matrix. Antibiotics in addition to clindamycin that inhibit the normal flora include ceftriaxone and piperacillin-tazobactam and may be substituted for the clindamycin. The antibiotic-treated matrix is centrifuged, the supernatant removed, and the pelleted material resuspended in filter-sterilized, diluted feces in order to remove any residual antibiotic. This washed antibiotic-treated matrix may be used in the in vitro assay described above in lieu of the 10% sterile-filtered feces.

Alternatively, the ability to inhibit the growth of the pathogen may be measured by quantitative PCR (qPCR). Standard techniques may be followed to generate a standard curve for the pathogen of interest. Genomic DNA may be extracted from samples using commercially-available kits, such as the Mo Bio Powersoil®-htp 96 Well Soil DNA Isolation Kit (Mo Bio Laboratories, Carlsbad, CA), the Mo Bio Powersoil® DNA Isolation Kit (Mo Bio Laboratories, Carlsbad, CA), or the QIAamp DNA Stool Mini Kit (QIAGEN, Valencia, CA) according to the manufacturer's instructions. The qPCR may be conducted using HotMasterMix (5PRIME, Gaithersburg, MD) and primers specific for the pathogen of interest, and may be conducted on a MicroAmp+Fast Optical 96-well Reaction Plate with Barcode (0.1 mL) (Life Technologies, Grand Island, NY) and performed on a BioRad C1000™ Thermal Cycler equipped with a CFX96™ Real-Time System (BioRad, Hercules, CA), with fluorescent readings of the FAM and ROX channels. The Cq value for each well on the FAM channel is determined by the CFX Manager™ software version 2.1. The log₁₀ (cfu/ml) of each experimental sample is calculated by inputting a given sample's Cq value into linear regression model generated from the standard curve comparing the Cq values of the standard curve wells to the known log₁₀ (cfu/ml) of those samples. The skilled artisan may employ alternative qPCR modes.

### In Vitro Assays Substantiating Protective Effect of Bacterial Compositions

Also provided are in vivo assays establishing the protective effect of bacterial compositions. Provided is an in vivo mouse model to test for the protective effect of the bacterial compositions against C. difficile. In this model (based on Chen, et al., A mouse model of Clostridium difficile associated disease, Gastroenterology 135 (6): 1984-1992 (2008)), mice are made susceptible to C. difficile by a 7 day treatment (days −12 to −5 of experiment) with 5 to 7 antibiotics (including kanamycin, colistin, gentamycin, metronidazole and vancomycin and optionally including ampicillin and ciprofloxacin) delivered via their drinking water, followed by a single dose with Clindamycin on day −3, then challenged three days later on day 0 with 10⁴ spores of C. difficile via oral gavage (i.e., oro-gastric lavage). Bacterial compositions may be given either before (prophylactic treatment) or after (therapeutic treatment) C. difficile gavage. Further, bacterial compositions may be given after (optional) vancomycin treatment (see below) to assess their ability to prevent recurrence and thus suppress the pathogen in vivo. The outcomes assessed each day from day −1 to day 6 (or beyond, for prevention of recurrence) are weight, clinical signs, mortality and shedding of C. difficile in the feces. Weight loss, clinical signs of disease, and C. difficile shedding are typically observed without treatment. Vancomycin provided by oral gavage on days −1 to 4 protects against these outcomes and serves as a positive control. Clinical signs are subjective, and scored each day by the same experienced observer. Animals that lose greater than or equal to 25% of their body weight are euthanized and counted as infection-related mortalities. Feces are gathered from mouse cages (5 mice per cage) each day, and the shedding of C. difficile spores is detected in the feces using a selective plating assay as described for the in vitro assay above, or via qPCR for the toxin gene as described herein. The effects of test materials including 10% suspension of human feces (as a positive control), bacterial compositions, or PBS (as a negative vehicle control), are determined by introducing the test article in a 0.2 mL volume into the mice via oral gavage on day −1, one day prior to C. difficile challenge, on day 1, 2 and 3 as treatment or post-vancomycin treatment on days 5, 6, 7 and 8. Vancomycin, as discussed above, is given on days 1 to 4 as another positive control. Alternative dosing schedules and routes of administration (e.g. rectal) may be employed, including multiple doses of test article, and 10⁵ to 10¹⁰ of a given organism or composition may be delivered.

### Methods for Determining 16S Sequences

OTUs can be defined either by full 16S sequencing of the rRNA gene, by sequencing of a specific hypervariable region of this gene (i.e. V1, V2, V3, V4, V5, V6, V7, V8, or V9), or by sequencing of any combination of hypervariable regions from this gene (e.g. V1-3 or V3-5). The bacterial 16S rDNA is approximately 1500 nucleotides in length and is used in reconstructing the evolutionary relationships and sequence similarity of one bacterial isolate to another using phylogenetic approaches. 16S sequences are used for phylogenetic reconstruction as they are in general highly conserved, but contain specific hypervariable regions that harbor sufficient nucleotide diversity to differentiate genera and species of most microbes.

Using well known techniques, in order to determine the full 16S sequence or the sequence of any hypervariable region of the 16S sequence, genomic DNA is extracted from a bacterial sample, the 16S rDNA (full region or specific hypervariable regions) amplified using polymerase chain reaction (PCR), the PCR products cleaned, and nucleotide sequences delineated to determine the genetic composition of 16S gene or subdomain of the gene. If full 16S sequencing is performed, the sequencing method used may be, but is not limited to, Sanger sequencing. If one or more hypervariable regions are used, such as the V4 region, the sequencing can be, but is not limited to being, performed using the Sanger method or using a next-generation sequencing method, such as an Illumina (sequencing by synthesis) method using barcoded primers allowing for multiplex reactions.

OTUs can be defined by a combination of nucleotide markers or genes, in particular highly conserved genes (e.g., “house-keeping” genes), or a combination thereof, full-genome sequence, or partial genome sequence generated using amplified genetic products, or whole genome sequence (WGS). Using well defined methods DNA extracted from a bacterial sample will have specific genomic regions amplified using PCR and sequenced to determine the nucleotide sequence of the amplified products. In the whole genome shotgun (WGS) method, extracted DNA will be directly sequenced without amplification. Sequence data can be generated using any sequencing technology including, but not limited to Sanger, Illumina, 454 Life Sciences, Ion Torrent, ABI, Pacific Biosciences, and/or Oxford Nanopore.

### Methods for Preparing a Bacterial Composition for Administration to a Subject

Methods for producing bacterial compositions can include three main processing steps, combined with one or more mixing steps. The steps include organism banking, organism production, and preservation.

For banking, the strains included in the bacterial composition may be (1) isolated directly from a specimen or taken from a banked stock, (2) optionally cultured on a nutrient agar or broth that supports growth to generate viable biomass, and (3) the biomass optionally preserved in multiple aliquots in long-term storage.

In embodiments that use a culturing step, the agar or broth can contain nutrients that provide essential elements and specific factors that enable growth. An example would be a medium composed of 20 g/L glucose, 10 g/L yeast extract, 10 g/L soy peptone, 2 g/L citric acid, 1.5 g/L sodium phosphate monobasic, 100 mg/L ferric ammonium citrate, 80 mg/L magnesium sulfate, 10 mg/L hemin chloride, 2 mg/L calcium chloride, 1 mg/L menadione. A variety of microbiological media and variations are well known in the art (e.g. R. M. Atlas, Handbook of Microbiological Media (2010) CRC Press). Medium can be added to the culture at the start, may be added during the culture, or may be intermittently/continuously flowed through the culture. The strains in the bacterial composition may be cultivated alone, as a subset of the bacterial composition, or as an entire collection comprising the bacterial composition. As an example, a first strain may be cultivated together with a second strain in a mixed continuous culture, at a dilution rate lower than the maximum growth rate of either cell to prevent the culture from washing out of the cultivation.

The inoculated culture is incubated under favorable conditions for a time sufficient to build biomass. For bacterial compositions for human use, this is often at 37° C. temperature, pH, and other parameter with values similar to the normal human niche. The environment can be actively controlled, passively controlled (e.g., via buffers), or allowed to drift. For example, for anaerobic bacterial compositions (e.g., gut microbiota), an anoxic/reducing environment can be employed. This can be accomplished by addition of reducing agents such as cysteine to the broth, and/or stripping it of oxygen. As an example, a culture of a bacterial composition can be grown at 37° C., pH 7, in the medium above, pre-reduced with 1 g/L cysteine HCl.

When the culture has generated sufficient biomass, it can be preserved for banking. The organisms can be placed into a chemical milieu that protects from freezing (adding ‘cryoprotectants’), drying (‘lyoprotectants’), and/or osmotic shock (‘osmoprotectants’), dispensing into multiple (optionally identical) containers to create a uniform bank, and then treating the culture for preservation. Containers are generally impermeable and have closures that assure isolation from the environment. Cryopreservation treatment is accomplished by freezing a liquid at ultra-low temperatures (e.g., at or below −80° C.). Dried preservation removes water from the culture by evaporation (in the case of spray drying or ‘cool drying’) or by sublimation (e.g., for freeze drying, spray freeze drying). Removal of water improves long-term bacterial composition storage stability at temperatures elevated above cryogenic. If the bacterial composition comprises spore forming species and results in the production of spores, the final composition can be purified by additional means, such as density gradient centrifugation preserved using the techniques described above. Bacterial composition banking can be done by culturing and preserving the strains individually, or by mixing the strains together to create a combined bank. As an example of cryopreservation, a bacterial composition culture can be harvested by centrifugation to pellet the cells from the culture medium, the supernate decanted and replaced with fresh culture broth containing 15% glycerol. The culture can then be aliquoted into 1 mL cryotubes, sealed, and placed at −80° C. for long-term viability retention. This procedure achieves acceptable viability upon recovery from frozen storage.

Organism production can be conducted using similar culture steps to banking, including medium composition and culture conditions. It can be conducted at larger scales of operation, especially for clinical development or commercial production. At larger scales, there can be several subcultivations of the bacterial composition prior to the final cultivation. At the end of cultivation, the culture is harvested to enable further formulation into a dosage form for administration. This can involve concentration, removal of undesirable medium components, and/or introduction into a chemical milieu that preserves the bacterial composition and renders it acceptable for administration via the chosen route. For example, a bacterial composition can be cultivated to a concentration of 10¹⁰ CFU/mL, then concentrated 20-fold by tangential flow microfiltration; the spent medium can be exchanged by diafiltering with a preservative medium consisting of 2% gelatin, 100 mM trehalose, and 10 mM sodium phosphate buffer. The suspension can then be freeze-dried to a powder and titrated.

After drying, the powder can be blended to an appropriate potency, and mixed with other cultures and/or a filler such as microcrystalline cellulose for consistency and ease of handling, and the bacterial composition formulated as provided herein.

### Methods of Treating a Subject

In some embodiments, the compositions disclosed herein are administered to a patient or a user (sometimes collectively referred to as a “subject”). As used herein “administer” and “administration” encompasses embodiments in which one person directs another to consume a bacterial composition in a certain manner and/or for a certain purpose, and also situations in which a user uses a bacteria composition in a certain manner and/or for a certain purpose independently of or in variance to any instructions received from a second person. Non-limiting examples of embodiments in which one person directs another to consume a bacterial composition in a certain manner and/or for a certain purpose include when a physician prescribes a course of conduct and/or treatment to a patient, when a parent commands a minor user (such as a child) to consume a bacterial composition, when a trainer advises a user (such as an athlete) to follow a particular course of conduct and/or treatment, and when a manufacturer, distributer, or marketer recommends conditions of use to an end user, for example through advertisements or labeling on packaging or on other materials provided in association with the sale or marketing of a product.

The bacterial compositions offer a protective and/or therapeutic effect against infection by one or more GI pathogens of interest and can be administered after an acute case of infection has been resolved in order to prevent relapse, during an acute case of infection as a complement to antibiotic therapy if the bacterial composition is not sensitive to the same antibiotics as the GI pathogen, or to prevent infection or reduce transmission from disease carriers. In some embodiments a bacterial compositions administered to the gut are used to treat disorders that are not gut-related. Altering the gut microbiome can have effects on the skin and vagina of mammalian hosts (Levkovich et al, Probiotic bacteria induce a ‘glow of health’, PlosOne, Vol 8, Issue 1, e53867. 2013) and can treat distal bacterial infections including mastitis (Jimenez et al. Oral Administration of Lactobacillus strains isolate from breast milk as an alternative for the treatment of Infectious mastitis during lactation. Applied and Environmental Microbiology (74) No. 15. 2008). Because the gut serves as a reservoir for bacteria for the rest of the body, the bacterial compositions described herein can be used to treat non-GI related health problems.

The present bacterial compositions can be useful in a variety of clinical situations. For example, the bacterial compositions can be administered as a complementary treatment to antibiotics when a patient is suffering from an acute infection, to reduce the risk of recurrence after an acute infection has subsided, or when a patient will be in close proximity to others with or at risk of serious gastrointestinal infections (physicians, nurses, hospital workers, family members of those who are ill or hospitalized).

The present bacterial compositions can be administered to animals, including humans, laboratory animals (e.g., primates, rats, mice), livestock (e.g., cows, sheep, goats, pigs, turkeys, chickens), and household pets (e.g., dogs, cats, rodents).

In the present method, the bacterial composition can be administered enterically, in other words, by a route of access to the gastrointestinal tract. This includes oral administration, rectal administration (including enema, suppository, or colonoscopy), by an oral or nasal tube (nasogastric, nasojejunal, oral gastric, or oral jejunal), as detailed more fully herein.

### Pretreatment Protocols

Prior to administration of the bacterial composition, the patient can optionally have a pretreatment protocol to prepare the gastrointestinal tract to receive the bacterial composition. In certain embodiments, the pretreatment protocol is advisable, such as when a patient has an acute infection with a highly resilient pathogen. In other embodiments, the pretreatment protocol is entirely optional, such as when the pathogen causing the infection is not resilient, or the patient has had an acute infection that has been successfully treated but where the physician is concerned that the infection may recur. In these instances, the pretreatment protocol can enhance the ability of the bacterial composition to affect the patient's microbiome.

As one way of preparing the patient for administration of the microbial ecosystem, at least one antibiotic can be administered to alter the bacteria in the patient. As another way of preparing the patient for administration of the microbial ecosystem, a standard colon-cleansing preparation can be administered to the patient to substantially empty the contents of the colon, such as used to prepare a patient for a colonoscopy. By “substantially emptying the contents of the colon,” this application means removing at least 75%, at least 80%, at least 90%, at least 95%, or about 100% of the contents of the ordinary volume of colon contents. Antibiotic treatment can precede the colon-cleansing protocol.

If a patient has received an antibiotic for treatment of an infection, or if a patient has received an antibiotic as part of a specific pretreatment protocol, in one embodiment, the antibiotic can be stopped in sufficient time to allow the antibiotic to be substantially reduced in concentration in the gut before the bacterial composition is administered. In one embodiment, the antibiotic can be discontinued 1, 2, or 3 days before the administration of the bacterial composition. In another embodiment, the antibiotic can be discontinued 3, 4, 5, 6, or 7 antibiotic half-lives before administration of the bacterial composition. In another embodiment, the antibiotic can be chosen so the constituents in the bacterial composition have an MIC50 that is higher than the concentration of the antibiotic in the gut.

MIC50 of a bacterial composition or the elements in the composition can be determined by methods well known in the art. Reller et al., Antimicrobial Susceptibility Testing: A Review of General Principles and Contemporary Practices, Clinical Infectious Diseases 49 (11): 1749-1755 (2009). In such an embodiment, the additional time between antibiotic administration and administration of the bacterial composition is not necessary. If the pretreatment protocol is part of treatment of an acute infection, the antibiotic can be chosen so that the infection is sensitive to the antibiotic, but the constituents in the bacterial composition are not sensitive to the antibiotic.

### Routes of Administration

The bacterial compositions of the invention are suitable for administration to mammals and non-mammalian animals in need thereof. In certain embodiments, the mammalian subject is a human subject who has one or more symptoms of a dysbiosis.

When a mammalian subject is suffering from a disease, disorder or condition characterized by an aberrant microbiota, the bacterial compositions described herein are suitable for treatment thereof. In some embodiments, the mammalian subject has not received antibiotics in advance of treatment with the bacterial compositions. For example, the mammalian subject has not been administered at least two doses of vancomycin, metronidazole and/or or similar antibiotic compound within one week prior to administration of the therapeutic composition. In other embodiments, the mammalian subject has not previously received an antibiotic compound in the one month prior to administration of the therapeutic composition. In other embodiments, the mammalian subject has received one or more treatments with one or more different antibiotic compounds and such treatment(s) resulted in no improvement or a worsening of symptoms.

In some embodiments, the gastrointestinal disease, disorder or condition is diarrhea caused by C. difficile including recurrent C. difficile infection, ulcerative colitis, colitis, Crohn's disease, or irritable bowel disease. Beneficially, the therapeutic composition is administered only once prior to improvement of the disease, disorder or condition. In some embodiments, the therapeutic composition is administered at intervals greater than two days, such as once every three, four, five or six days, or every week or less frequently than every week. In other embodiments, the preparation can be administered intermittently according to a set schedule, e.g., once a day, once weekly, or once monthly, or when the subject relapses from the primary illness. In another embodiment, the preparation may be administered on a long-term basis to subjects who are at risk for infection with or who may be carriers of these pathogens, including subjects who will have an invasive medical procedure (such as surgery), who will be hospitalized, who live in a long-term care or rehabilitation facility, who are exposed to pathogens by virtue of their profession (livestock and animal processing workers), or who could be carriers of pathogens (including hospital workers such as physicians, nurses, and other health care professionals).

In certain embodiments, the bacterial composition is administered enterically. This preferentially includes oral administration, or by an oral or nasal tube (including nasogastric, nasojejunal, oral gastric, or oral jejunal). In other embodiments, administration includes rectal administration (including enema, suppository, or colonoscopy). The bacterial composition can be administered to at least one region of the gastrointestinal tract, including the mouth, esophagus, stomach, small intestine, large intestine, and rectum. In some embodiments, it is administered to all regions of the gastrointestinal tract. The bacterial compositions can be administered orally in the form of medicaments such as powders, capsules, tablets, gels or liquids. The bacterial compositions can also be administered in gel or liquid form by the oral route or through a nasogastric tube, or by the rectal route in a gel or liquid form, by enema or instillation through a colonoscope or by a suppository.

If the composition is administered colonoscopically and, optionally, if the bacterial composition is administered by other rectal routes (such as an enema or suppository) or even if the subject has an oral administration, the subject can have a colon-cleansing preparation. The colon-cleansing preparation can facilitate proper use of the colonoscope or other administration devices, but even when it does not serve a mechanical purpose, it can also maximize the proportion of the bacterial composition relative to the other organisms previously residing in the gastrointestinal tract of the subject. Any ordinarily acceptable colon-cleansing preparation may be used such as those typically provided when a subject undergoes a colonoscopy.

### Dosages and Schedule for Administration

In some embodiments, the bacteria and bacterial compositions are provided in a dosage form. In certain embodiments, the dosage form is designed for administration of at least one OTU or combination thereof disclosed herein, wherein the total amount of bacterial composition administered is selected from 0.1 ng to 10 g, 10 ng to 1 g, 100 ng to 0.1 g, 0.1 mg to 500 mg, 1 mg to 100 mg, or from 10-15 mg. In other embodiments, the bacterial composition is consumed at a rate of from 0.1 ng to 10 g a day, 10 ng to 1 g a day, 100 ng to 0.1 g a day. 0.1 mg to 500 mg a day, 1 mg to 100 mg a day, or from 10-15 mg a day, or more.

In certain embodiments, the treatment period is at least 1 day, at least 2 days, at least 3 days, at least 4 days, at least 5 days, at least 6 days, at least 1 week, at least 2 weeks, at least 3 weeks, at least 4 weeks, at least 1 month, at least 2 months, at least 3 months, at least 4 months, at least 5 months, at least 6 months, or at least 1 year. In some embodiments the treatment period is from 1 day to 1 week, from 1 week to 4 weeks, from 1 month, to 3 months, from 3 months to 6 months, from 6 months to 1 year, or for over a year.

In one embodiment, 10⁵ and 10¹² microorganisms total can be administered to the patient in a given dosage form. In another embodiment, an effective amount can be provided in from 1 to 500 ml or from 1 to 500 grams of the bacterial composition having from 10⁷ to 10¹¹ bacteria per ml or per gram, or a capsule, tablet or suppository having from 1 mg to 1000 mg lyophilized powder having from 10⁷ to 10¹¹ bacteria. Those receiving acute treatment can receive higher doses than those who are receiving chronic administration (such as hospital workers or those admitted into long-term care facilities).

Any of the preparations described herein can be administered once on a single occasion or on multiple occasions, such as once a day for several days or more than once a day on the day of administration (including twice daily, three times daily, or up to five times daily). In another embodiment, the preparation can be administered intermittently according to a set schedule, e.g., once weekly, once monthly, or when the patient relapses from the primary illness. In one embodiment, the preparation can be administered on a long-term basis to individuals who are at risk for infection with or who may be carriers of these pathogens, including individuals who will have an invasive medical procedure (such as surgery), who will be hospitalized, who live in a long-term care or rehabilitation facility, who are exposed to pathogens by virtue of their profession (livestock and animal processing workers), or who could be carriers of pathogens (including hospital workers such as physicians, nurses, and other health care professionals).

### Patient Selection

Particular bacterial compositions can be selected for individual patients or for patients with particular profiles. For example, 16S sequencing can be performed for a given patient to identify the bacteria present in his or her microbiota. The sequencing can either profile the patient's entire microbiome using 16S sequencing (to the family, genera, or species level), a portion of the patient's microbiome using 16S sequencing, or it can be used to detect the presence or absence of specific candidate bacteria that are biomarkers for health or a particular disease state, such as markers of multi-drug resistant organisms or specific genera of concern such as Escherichia. Based on the biomarker data, a particular composition can be selected for administration to a patient to supplement or complement a patient's microbiota in order to restore health or treat or prevent disease. In another embodiment, patients can be screened to determine the composition of their microbiota to determine the likelihood of successful treatment.

### Combination Therapy

The bacterial compositions can be administered with other agents in a combination therapy mode, including anti-microbial agents and prebiotics. Administration can be sequential, over a period of hours or days, or simultaneous.

In one embodiment, the bacterial compositions are included in combination therapy with one or more anti-microbial agents, which include anti-bacterial agents, anti-fungal agents, anti-viral agents and anti-parasitic agents.

Anti-bacterial agents can include cephalosporin antibiotics (cephalexin, cefuroxime, cefadroxil, cefazolin, cephalothin, cefaclor, cefamandole, cefoxitin, cefprozil, and ceftobiprole); fluoroquinolone antibiotics (cipro, Levaquin, floxin, tequin, avelox, and norflox); tetracycline antibiotics (tetracycline, minocycline, oxytetracycline, and doxycycline); penicillin antibiotics (amoxicillin, ampicillin, penicillin V, dicloxacillin, carbenicillin, vancomycin, and methicillin); and carbapenem antibiotics (ertapenem, doripenem, imipenem/cilastatin, and meropenem).

Anti-viral agents can include Abacavir, Acyclovir, Adefovir, Amprenavir, Atazanavir, Cidofovir, Darunavir, Delavirdine, Didanosine, Docosanol, Efavirenz, Elvitegravir, Emtricitabine, Enfuvirtide, Etravirine, Famciclovir, Foscarnet, Fomivirsen, Ganciclovir, Indinavir, Idoxuridine, Lamivudine, Lopinavir Maraviroc, MK-2048, Nelfinavir, Nevirapine, Penciclovir, Raltegravir, Rilpivirine, Ritonavir, Saquinavir, Stavudine, Tenofovir Trifluridine, Valaciclovir, Valganciclovir, Vidarabine, Ibacitabine, Amantadine, Oseltamivir, Rimantidine, Tipranavir, Zalcitabine, Zanamivir and Zidovudine.

Examples of antifungal compounds include, but are not limited to polyene antifungals such as natamycin, rimocidin, filipin, nystatin, amphotericin B, candicin, and hamycin; imidazole antifungals such as miconazole, ketoconazole, clotrimazole, econazole, omoconazole, bifonazole, butoconazole, fenticonazole, isoconazole, oxiconazole, sertaconazole, sulconazole, and tioconazole; triazole antifungals such as fluconazole, itraconazole, isavuconazole, ravuconazole, posaconazole, voriconazole, terconazole, and albaconazole; thiazole antifungals such as abafungin; allylamine antifungals such as terbinafine, naftifine, and butenafine; and echinocandin antifungals such as anidulafungin, caspofungin, and micafungin. Other compounds that have antifungal properties include, but are not limited to polygodial, benzoic acid, ciclopirox, tolnaftate, undecylenic acid, flucytosine or 5-fluorocytosine, griseofulvin, and haloprogin.

In one embodiment, the bacterial compositions are included in combination therapy with one or more corticosteroids, mesalazine, mesalamine, sulfasalazine, sulfasalazine derivatives, immunosuppressive drugs, cyclosporin A, mercaptopurine, azathiopurine, prednisone, methotrexate, antihistamines, glucocorticoids, epinephrine, theophylline, cromolyn sodium, anti-leukotrienes, anti-cholinergic drugs for rhinitis, anti-cholinergic decongestants, mast-cell stabilizers, monoclonal anti-IgE antibodies, vaccines, and combinations thereof.

A prebiotic is a selectively fermented ingredient that allows specific changes, both in the composition and/or activity in the gastrointestinal microbiota that confers benefits upon host well-being and health. Prebiotics can include complex carbohydrates, amino acids, peptides, or other essential nutritional components for the survival of the bacterial composition. Prebiotics include, but are not limited to, amino acids, biotin, fructooligosaccharide, galactooligosaccharides, inulin, lactulose, mannan oligosaccharides, oligofructose-enriched inulin, oligofructose, oligodextrose, tagatose, trans-galactooligosaccharide, and xylooligosaccharides.

### Methods for Testing Bacterial Compositions for Populating Effect

### In Vivo Assay for Determining Whether a Bacterial Composition Populates a Subject's Gastrointestinal Tract

In order to determine that the bacterial composition populates the gastrointestinal tract of a subject, an animal model, such as a mouse model, can be used. The model can begin by evaluating the microbiota of the mice. Qualitative assessments can be accomplished using 16S profiling of the microbial community in the feces of normal mice. It can also be accomplished by full genome sequencing, whole genome shotgun sequencing (WGS), or traditional microbiological techniques. Quantitative assessments can be conducted using quantitative PCR (qPCR), described below, or by using traditional microbiological techniques and counting colony formation.

Optionally, the mice can receive an antibiotic treatment to mimic the condition of dysbiosis. Antibiotic treatment can decrease the taxonomic richness, diversity, and evenness of the community, including a reduction of abundance of a significant number of bacterial taxa. Dethlefsen et al., The pervasive effects of an antibiotic on the human gut microbiota, as revealed by deep 16S rRNA sequencing, PLOS Biology 6(11):3280 (2008). At least one antibiotic can be used, and antibiotics are well known. Antibiotics can include aminoglycoside antibiotic (amikacin, arbekacin, gentamicin, kanamycin, neomycin, netilmicin, paromomycin, rhodostreptomycin, streptomycin, tobramycin, and apramycin), amoxicillin, ampicillin, Augmentin (an amoxicillin/clavulanate potassium combination), cephalosporin (cefaclor, defadroxil, cefazolin, cefixime, fefoxitin, cefprozil, ceftazimdime, cefuroxime, cephalexin), clavulanate potassium, clindamycin, colistin, gentamycin, kanamycin, metronidazole, or vancomycin. As an individual, nonlimiting specific example, the mice can be provided with drinking water containing a mixture of the antibiotics kanamycin, colistin, gentamycin, metronidazole and vancomycin at 40 mg/kg, 4.2 mg/kg, 3.5 mg/kg, 21.5 mg/kg, and 4.5 mg/kg (mg per average mouse body weight), respectively, for 7 days. Alternatively, mice can be administered ciprofloxacin at a dose of 15-20 mg/kg (mg per average mouse body weight), for 7 days.

If the mice are provided with an antibiotic, a wash out period of from one day to three days may be provided with no antibiotic treatment and no bacterial composition treatment.

Subsequently, the test bacterial composition is administered to the mice by oral gavage. The test bacterial composition may be administered in a volume of 0.2 ml containing 10⁴ CFUs of each type of bacteria in the bacterial composition. Dose-response may be assessed by using a range of doses, including, but not limited to 10², 10³, 10⁴, 10⁵, 10⁶, 10⁷, 10⁸, 10⁹, and/or 10¹⁰.

The mice can be evaluated using 16S sequencing, full genome sequencing, whole genome shotgun sequencing (WGS), or traditional microbiological techniques to determine whether the test bacterial composition has populated the gastrointestinal tract of the mice. For example only, one day, three days, one week, two weeks, and one month after administration of the bacterial composition to the mice, 16S profiling is conducted to determine whether the test bacterial composition has populated the gastrointestinal tract of the mice. Quantitative assessments, including qPCR and traditional microbiological techniques such as colony counting, can additionally or alternatively be performed, at the same time intervals.

Furthermore, the number of sequence counts that correspond exactly to those in the bacterial composition over time can be assessed to determine specifically which components of the bacterial composition reside in the gastrointestinal tract over a particular period of time. In one embodiment, the strains of the bacterial composition persist for a desired period of time. In another embodiment, the components of the bacterial composition persist for a desired period of time, while also increasing the ability of other microbes (such as those present in the environment, food, etc.) to populate the gastrointestinal tract, further increasing overall diversity, as discussed below.

### Ability of Bacterial Compositions to Populate Different Regions of the Gastrointestinal Tract

The present bacterial compositions can also be assessed for their ability to populate different regions on the gastrointestinal tract. In one embodiment, a bacterial composition can be chosen for its ability to populate one or more than one region of the gastrointestinal tract, including, but not limited to the stomach, the small intestine (duodenum, jejunum, and ileum), the large intestine (the cecum, the colon (the ascending, transverse, descending, and sigmoid colon), and the rectum).

An in vivo study can be conducted to determine which regions of the gastrointestinal tract a given bacterial composition will populate. A mouse model similar to the one described above can be conducted, except instead of assessing the feces produced by the mice, particular regions of the gastrointestinal tract can be removed and studied individually. For example, at least one particular region of the gastrointestinal tract can be removed and a qualitative or quantitative determination can be performed on the contents of that region of the gastrointestinal tract. In another embodiment, the contents can optionally be removed and the qualitative or quantitative determination may be conducted on the tissue removed from the mouse.

qPCR

As one quantitative method for determining whether a bacterial composition populates the gastrointestinal tract, quantitative PCR (qPCR) can be performed. Standard techniques can be followed to generate a standard curve for the bacterial composition of interest, either for all of the components of the bacterial composition collectively, individually, or in subsets (if applicable). Genomic DNA can be extracted from samples using commercially-available kits, such as the Mo Bio Powersoil®-htp 96 Well Soil DNA Isolation Kit (Mo Bio Laboratories, Carlsbad, CA), the Mo Bio Powersoil® DNA Isolation Kit (Mo Bio Laboratories, Carlsbad, CA), or the QIAamp DNA Stool Mini Kit (QIAGEN, Valencia, CA) according to the manufacturer's instructions.

In some embodiments, qPCR can be conducted using HotMasterMix (5PRIME, Gaithersburg, MD) and primers specific for the bacterial composition of interest, and may be conducted on a MicroAmp® Fast Optical 96-well Reaction Plate with Barcode (0.1 mL) (Life Technologies, Grand Island, NY) and performed on a BioRad C1000™ Thermal Cycler equipped with a CFX96™ Real-Time System (BioRad, Hercules, CA), with fluorescent readings of the FAM and ROX channels. The Cq value for each well on the FAM channel is determined by the CFX Manager™ software version 2.1. The log₁₀ (cfu/ml) of each experimental sample is calculated by inputting a given sample's Cq value into linear regression model generated from the standard curve comparing the Cq values of the standard curve wells to the known log₁₀ (cfu/ml) of those samples. The skilled artisan may employ alternative qPCR modes.

### Methods for Characterization of Bacterial Compositions

In certain embodiments, provided are methods for testing certain characteristics of bacterial compositions. For example, the sensitivity of bacterial compositions to certain environmental variables is determined, e.g., in order to select for particular desirable characteristics in a given composition, formulation and/or use. For example, the constituents in the bacterial composition can be tested for pH resistance, bile acid resistance, and/or antibiotic sensitivity, either individually on a constituent-by-constituent basis or collectively as a bacterial composition comprised of multiple bacterial constituents (collectively referred to in this section as bacterial composition).

### pH Sensitivity Testing

If a bacterial composition will be administered other than to the colon or rectum (i.e., for example, an oral route), optionally testing for pH resistance enhances the selection of bacterial compositions that will survive at the highest yield possible through the varying pH environments of the distinct regions of the GI tract. Understanding how the bacterial compositions react to the pH of the GI tract also assists in formulation, so that the number of bacteria in a dosage form can be increased if beneficial and/or so that the composition may be administered in an enteric-coated capsule or tablet or with a buffering or protective composition. As the pH of the stomach can drop to a pH of 1 to 2 after a high-protein meal for a short time before physiological mechanisms adjust it to a pH of 3 to 4 and often resides at a resting pH of 4 to 5, and as the pH of the small intestine can range from a pH of 6 to 7.4, bacterial compositions can be prepared that survive these varying pH ranges (specifically wherein at least 1%, 5%, 10%, 15%, 20%, 25%, 30%, 40%, 50%, 60%, 70%, 80%, 90%, or as much as 100% of the bacteria can survive gut transit times through various pH ranges). This can be tested by exposing the bacterial composition to varying pH ranges for the expected gut transit times through those pH ranges. Therefore, as a nonlimiting example only, 18-hour cultures of bacterial compositions can be grown in standard media, such as gut microbiota medium (“GMM”, see Goodman et al., Extensive personal human gut microbiota culture collections characterized and manipulated in gnotobiotic mice, PNAS 108(15): 6252-6257 (2011)) or another animal-products-free medium, with the addition of pH adjusting agents for a pH of 1 to 2 for 30 minutes, a pH of 3 to 4 for 1 hour, a pH of 4 to 5 for 1 to 2 hours, and a pH of 6 to 7.4 for 2.5 to 3 hours. An alternative method for testing stability to acid is described in U.S. Pat. No. 4,839,281. Survival of bacteria may be determined by culturing the bacteria and counting colonies on appropriate selective or non-selective media.

### Bile Acid Sensitivity Testing

Additionally, in some embodiments, testing for bile-acid resistance enhances the selection of bacterial compositions that will survive exposures to bile acid during transit through the GI tract. Bile acids are secreted into the small intestine and can, like pH, affect the survival of bacterial compositions. This can be tested by exposing the bacterial compositions to bile acids for the expected gut exposure time to bile acids. For example, bile acid solutions can be prepared at desired concentrations using 0.05 mM Tris at pH 9 as the solvent. After the bile acid is dissolved, the pH of the solution may be adjusted to 7.2 with 10% HCl. Bacterial compositions can be cultured in 2.2 ml of a bile acid composition mimicking the concentration and type of bile acids in the patient, 1.0 ml of 10% sterile-filtered feces media and 0.1 ml of an 18-hour culture of the given strain of bacteria. Incubations may be conducted for from 2.5 to 3 hours or longer. An alternative method for testing stability to bile acid is described in U.S. Pat. No. 4,839,281. Survival of bacteria may be determined by culturing the bacteria and counting colonies on appropriate selective or non-selective media.

### Antibiotic Sensitivity Testing

As a further optional sensitivity test, bacterial compositions can be tested for sensitivity to antibiotics. In one embodiment, bacterial compositions can be chosen so that the bacterial constituents are sensitive to antibiotics such that if necessary they can be eliminated or substantially reduced from the patient's gastrointestinal tract by at least one antibiotic targeting the bacterial composition.

### Adherence to Gastrointestinal Cells

The bacterial compositions may optionally be tested for the ability to adhere to gastrointestinal cells. A method for testing adherence to gastrointestinal cells is described in U.S. Pat. No. 4,839,281.

### Methods for Purifying Spores

### Solvent Treatments

To purify the bacterial spores, the fecal material is subjected to one or more solvent treatments. A solvent treatment is a miscible solvent treatment (either partially miscible or fully miscible) or an immiscible solvent treatment. Miscibility is the ability of two liquids to mix with each to form a homogeneous solution. Water and ethanol, for example, are fully miscible such that a mixture containing water and ethanol in any ratio will show only one phase. Miscibility is provided as a wt/wt %, or weight of one solvent in 100 g of final solution. If two solvents are fully miscible in all proportions, their miscibility is 100%. Provided as fully miscible solutions with water are alcohols, e.g., methanol, ethanol, isopropanol, butanol, etc. The alcohols can be provided already combined with water; e.g., a solution containing 10%, 20%, 25%, 30%, 35%, 40%, 45%, 50%, 55%, 60%, 65%, 70%, 75%, 89%, 85%, 90%, 95% or greater than 95% Other solvents are only partially miscible, meaning that only some portion will dissolve in water. Diethyl ether, for example, is partially miscible with water. Up to 7 grams of diethyl ether will dissolve in 93 g of water to give a 7% (wt/wt %) solution. If more diethyl ether is added, a two phase solution will result with a distinct diethyl ether layer above the water. Other miscible materials include ethers, dimethoxyethane, or tetrahydrofuran In contrast, an oil such as an alkane and water are immiscible and form two phases. Further, immiscible treatments are optionally combined with a detergent, either an ionic detergent or a non-ionic detergent. Exemplary detergents include Triton X-100, Tween 20, Tween 80, Nonidet P40, a pluronic, or a polyol.

### Chromatography Treatments

To purify spore populations, the fecal materials are subjected to one or more chromatographic treatments, either sequentially or in parallel. In a chromatographic treatment, a solution containing the fecal material is contacted with a solid medium containing a hydrophobic interaction chromatographic (HIC) medium or an affinity chromatographic medium. In an alternative embodiment, a solid medium capable of absorbing a residual habitat product present in the fecal material is contacted with a solid medium that adsorbs a residual habitat product. In certain embodiments, the HIC medium contains sepharose or a derivatized sepharose such as butyl sepharose, octyl sepharose, phenyl sepharose, or butyl-s sepharose. In other embodiments, the affinity chromatographic medium contains material derivatized with mucin type I, II, III, IV, V, or VI, or oligosaccharides derived from or similar to those of mucins type I, II, III, IV. V. or VI. Alternatively, the affinity chromatographic medium contains material derivatized with antibodies that recognize spore-forming bacteria.

### Mechanical Treatments

Provided herein is the physical disruption of the fecal material, particularly by one or more mechanical treatment such as blending, mixing, shaking, vortexing, impact pulverization, and sonication. As provided herein, the mechanical disrupting treatment substantially disrupts a non-spore material present in the fecal material and does not substantially disrupt a spore present in the fecal material. Mechanical treatments optionally include filtration treatments, where the desired spore populations are retained on a filter while the undesirable (non-spore) fecal components to pass through, and the spore fraction is then recovered from the filter medium. Alternatively, undesirable particulates and eukaryotic cells may be retained on a filter while bacterial cells including spores pass through. In some embodiments the spore fraction retained on the filter medium is subjected to a diafiltration step, wherein the retained spores are contacted with a wash liquid, typically a sterile saline-containing solution or other diluent, in order to further reduce or remove the undesirable fecal components.

### Thermal Treatments

Provided herein is the thermal disruption of the fecal material. Generally, the fecal material is mixed in a saline-containing solution such as phosphate-buffered saline (PBS) and subjected to a heated environment, such as a warm room, incubator, water-bath, or the like, such that efficient heat transfer occurs between the heated environment and the fecal material. Preferably the fecal material solution is mixed during the incubation to enhance thermal conductivity and disrupt particulate aggregates. Thermal treatments can be modulated by the temperature of the environment and/or the duration of the thermal treatment. For example, the fecal material or a liquid comprising the fecal material is subjected to a heated environment, e.g., a hot water bath of at least about 20, 25, 30, 35, 40, 45, 50, 55; 60, 65, 70, 75, 80, 85, 90, 95, 100 or greater than 100 degrees Celsius, for at least about 1, 5, 10, 15, 20, 30, 45 seconds, or 1, 2, 3, 4, 5, 6, 7, 8, 9, 10, 15, 20, 25, 30, 40, or 50 minutes, or 1, 2, 3, 4, 5, 6, 7, 8, 9, 10 or more than 10 hours. In certain embodiments the thermal treatment occurs at two different temperatures, such as 30 seconds in a 100 degree Celsius environment followed by 10 minutes in a 50 degree Celsius environment. In preferred embodiments the temperature and duration of the thermal treatment are sufficient to kill or remove pathogenic materials while not substantially damaging or reducing the germination-competency of the spores.

### Irradiation Treatments

Provided are methods of treating the fecal material or separated contents of the fecal material with ionizing radiation, typically gamma irradiation, ultraviolet irradiation or electron beam irradiation provided at an energy level sufficient to kill pathogenic materials while not substantially damaging the desired spore populations. For example, ultraviolet radiation at 254 nm provided at an energy level below about 22,000 microwatt seconds per cm² will not generally destroy desired spores.

### Centrifugation and Density Separation Treatments

Provided are methods of separating desired spore populations from the other components of the fecal material by centrifugation. A solution containing the fecal material is subjected to one or more centrifugation treatments, e.g., at about 1000× g, 2000× g, 3000× g, 4000× g, 5000× g, 6000× g, 7000× g, 8000× g or greater than 8000× g. Differential centrifugation separates desired spores from undesired non-spore material; at low forces the spores are retained in solution, while at higher forces the spores are pelleted while smaller impurities (e.g., virus particles, phage) are retained in solution. For example, a first low force centrifugation pellets fibrous materials; a second, higher force centrifugation pellets undesired eukaryotic cells, and a third, still higher force centrifugation pellets the desired spores while small contaminants remain in suspension. In some embodiments density or mobility gradients or cushions (e.g., step cushions), such as Percoll, Ficoll, Nycodenz, Histodenz or sucrose gradients, are used to separate desired spore populations from other materials in the fecal material.

Also provided herein are methods of producing spore populations that combine two or more of the treatments described herein in order to synergistically purify the desired spores while killing or removing undesired materials and/or activities from the spore population. It is generally desirable to retain the spore populations under non-germinating and non-growth promoting conditions and media, in order to minimize the growth of pathogenic bacteria present in the spore populations and to minimize the germination of spores into vegetative bacterial cells.

### Pharmaceutical Compositions and Formulations of the Invention

### Formulations

Provided are formulations for administration to humans and other subjects in need thereof. Generally the bacterial compositions are combined with additional active and/or inactive materials in order to produce a final product, which may be in single dosage unit or in a multi-dose format.

In some embodiments, the composition comprises at least one carbohydrate. A “carbohydrate” refers to a sugar or polymer of sugars. The terms “saccharide,” “polysaccharide,” “carbohydrate,” and “oligosaccharide” may be used interchangeably. Most carbohydrates are aldehydes or ketones with many hydroxyl groups, usually one on each carbon atom of the molecule. Carbohydrates generally have the molecular formula CₙH₂ₙOₙ. A carbohydrate can be a monosaccharide, a disaccharide, trisaccharide, oligosaccharide, or polysaccharide. The most basic carbohydrate is a monosaccharide, such as glucose, sucrose, galactose, mannose, ribose, arabinose, xylose, and fructose. Disaccharides are two joined monosaccharides. Exemplary disaccharides include sucrose, maltose, cellobiose, and lactose. Typically, an oligosaccharide includes between three and six monosaccharide units (e.g., raffinose, stachyose), and polysaccharides include six or more monosaccharide units. Exemplary polysaccharides include starch, glycogen, and cellulose. Carbohydrates can contain modified saccharide units, such as 2′-deoxyribose wherein a hydroxyl group is removed, 2′-fluororibose wherein a hydroxyl group is replace with a fluorine, or N-acetylglucosamine, a nitrogen-containing form of glucose (e.g., 2′-fluororibose, deoxyribose, and hexose). Carbohydrates can exist in many different forms, for example, conformers, cyclic forms, acyclic forms, stereoisomers, tautomers, anomers, and isomers.

In some embodiments, the composition comprises at least one lipid. As used herein, a “lipid” includes fats, oils, triglycerides, cholesterol, phospholipids, fatty acids in any form including free fatty acids. Fats, oils and fatty acids can be saturated, unsaturated (cis or trans) or partially unsaturated (cis or trans). In some embodiments, the lipid comprises at least one fatty acid selected from lauric acid (12:0), myristic acid (14:0), palmitic acid (16:0), palmitoleic acid (16:1), margaric acid (17:0), heptadecenoic acid (17:1), stearic acid (18:0), oleic acid (18:1), linoleic acid (18:2), linolenic acid (18:3), octadecatetraenoic acid (18:4), arachidic acid (20:0), eicosenoic acid (20:1), eicosadienoic acid (20:2), eicosatetraenoic acid (20:4), eicosapentaenoic acid (20:5) (EPA), docosanoic acid (22:0), docosenoic acid (22:1), docosapentaenoic acid (22:5), docosahexaenoic acid (22:6) (DHA), and tetracosanoic acid (24:0). In other embodiments, the composition comprises at least one modified lipid, for example, a lipid that has been modified by cooking.

In some embodiments, the composition comprises at least one supplemental mineral or mineral source. Examples of minerals include, without limitation: chloride, sodium, calcium, iron, chromium, copper, iodine, zinc, magnesium, manganese, molybdenum, phosphorus, potassium, and selenium. Suitable forms of any of the foregoing minerals include soluble mineral salts, slightly soluble mineral salts, insoluble mineral salts, chelated minerals, mineral complexes, non-reactive minerals such as carbonyl minerals, and reduced minerals, and combinations thereof.

In certain embodiments, the composition comprises at least one supplemental vitamin. The at least one vitamin can be fat-soluble or water soluble vitamins. Suitable vitamins include but are not limited to vitamin C, vitamin A, vitamin E, vitamin B12, vitamin K, riboflavin, niacin, vitamin D, vitamin B6, folic acid, pyridoxine, thiamine, pantothenic acid, and biotin. Suitable forms of any of the foregoing are salts of the vitamin, derivatives of the vitamin, compounds having the same or similar activity of the vitamin, and metabolites of the vitamin.

In other embodiments, the composition comprises an excipient. Non-limiting examples of suitable excipients include a buffering agent, a preservative, a stabilizer, a binder, a compaction agent, a lubricant, a dispersion enhancer, a disintegration agent, a flavoring agent, a sweetener, and a coloring agent.

In another embodiment, the excipient is a buffering agent. Non-limiting examples of suitable buffering agents include sodium citrate, magnesium carbonate, magnesium bicarbonate, calcium carbonate, and calcium bicarbonate.

In some embodiments, the excipient comprises a preservative. Non-limiting examples of suitable preservatives include antioxidants, such as alpha-tocopherol and ascorbate, and antimicrobials, such as parabens, chlorobutanol, and phenol.

In other embodiments, the composition comprises a binder as an excipient. Non-limiting examples of suitable binders include starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C₁₂-C₁₈ fatty acid alcohol, polyethylene glycol, polyols, saccharides, oligosaccharides, and combinations thereof.

In another embodiment, the composition comprises a lubricant as an excipient. Non-limiting examples of suitable lubricants include magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, and light mineral oil.

In other embodiments, the composition comprises a dispersion enhancer as an excipient. Non-limiting examples of suitable dispersants include starch, alginic acid, polyvinylpyrrolidones, guar gum, kaolin, bentonite, purified wood cellulose, sodium starch glycolate, isoamorphous silicate, and microcrystalline cellulose as high HLB emulsifier surfactants.

In some embodiments, the composition comprises a disintegrant as an excipient. In other embodiments, the disintegrant is a non-effervescent disintegrant. Non-limiting examples of suitable non-effervescent disintegrants include starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pecitin, and tragacanth. In another embodiment, the disintegrant is an effervescent disintegrant. Non-limiting examples of suitable effervescent disintegrants include sodium bicarbonate in combination with citric acid, and sodium bicarbonate in combination with tartaric acid.

In another embodiment, the excipient comprises a flavoring agent. Flavoring agents can be chosen from synthetic flavor oils and flavoring aromatics; natural oils; extracts from plants, leaves, flowers, and fruits; and combinations thereof. In some embodiments the flavoring agent is selected from cinnamon oils; oil of wintergreen; peppermint oils; clover oil; hay oil; anise oil; eucalyptus; vanilla; citrus oil such as lemon oil, orange oil, grape and grapefruit oil; and fruit essences including apple, peach, pear, strawberry, raspberry, cherry, plum, pineapple, and apricot.

In other embodiments, the excipient comprises a sweetener. Non-limiting examples of suitable sweeteners include glucose (corn syrup), dextrose, invert sugar, fructose, and mixtures thereof (when not used as a carrier); saccharin and its various salts such as the sodium salt; dipeptide sweeteners such as aspartame; dihydrochalcone compounds, glycyrrhizin; Stevia Rebaudiana (Stevioside); chloro derivatives of sucrose such as sucralose; and sugar alcohols such as sorbitol, mannitol, sylitol, and the like. Also contemplated are hydrogenated starch hydrolysates and the synthetic sweetener 3,6-dihydro-6-methyl-1,2,3-oxathiazin-4-one-2,2-dioxide, particularly the potassium salt (acesulfame-K), and sodium and calcium salts thereof.

In yet other embodiments, the composition comprises a coloring agent. Non-limiting examples of suitable color agents include food, drug and cosmetic colors (FD&C), drug and cosmetic colors (D&C), and external drug and cosmetic colors (Ext. D&C). The coloring agents can be used as dyes or their corresponding lakes.

The weight fraction of the excipient or combination of excipients in the formulation is usually about 99% or less, such as about 95% or less, about 90% or less, about 85% or less, about 80% or less, about 75% or less, about 70% or less, about 65% or less, about 60% or less, about 55% or less, 50% or less, about 45% or less, about 40% or less, about 35% or less, about 30% or less, about 25% or less, about 20% or less, about 15% or less, about 10% or less, about 5% or less, about 2% or less, or about 1% or less of the total weight of the composition.

The bacterial compositions disclosed herein can be formulated into a variety of forms and administered by a number of different means. The compositions can be administered orally, rectally, or parenterally, in formulations containing conventionally acceptable carriers, adjuvants, and vehicles as desired. The term “parenteral” as used herein includes subcutaneous, intravenous, intramuscular, or intrastemal injection and infusion techniques. In an exemplary embodiment, the bacterial composition is administered orally.

Solid dosage forms for oral administration include capsules, tablets, caplets, pills, troches, lozenges, powders, and granules. A capsule typically comprises a core material comprising a bacterial composition and a shell wall that encapsulates the core material. In some embodiments, the core material comprises at least one of a solid, a liquid, and an emulsion. In other embodiments, the shell wall material comprises at least one of a soft gelatin, a hard gelatin, and a polymer. Suitable polymers include, but are not limited to: cellulosic polymers such as hydroxypropyl cellulose, hydroxyethyl cellulose, hydroxypropyl methyl cellulose (HPMC), methyl cellulose, ethyl cellulose, cellulose acetate, cellulose acetate phthalate, cellulose acetate trimellitate, hydroxypropylmethyl cellulose phthalate, hydroxypropylmethyl cellulose succinate and carboxymethylcellulose sodium; acrylic acid polymers and copolymers, such as those formed from acrylic acid, methacrylic acid, methyl acrylate, ammonio methylacrylate, ethyl acrylate, methyl methacrylate and/or ethyl methacrylate (e.g., those copolymers sold under the trade name “Eudragit”): vinyl polymers and copolymers such as polyvinyl pyrrolidone, polyvinyl acetate, polyvinylacetate phthalate, vinylacetate crotonic acid copolymer, and ethylene-vinyl acetate copolymers; and shellac (purified lac). In yet other embodiments, at least one polymer functions as taste-masking agents.

Tablets, pills, and the like can be compressed, multiply compressed, multiply layered, and/or coated. The coating can be single or multiple. In one embodiment, the coating material comprises at least one of a saccharide, a polysaccharide, and glycoproteins extracted from at least one of a plant, a fungus, and a microbe. Non-limiting examples include corn starch, wheat starch, potato starch, tapioca starch, cellulose, hemicellulose, dextrans, maltodextrin, cyclodextrins, inulins, pectin, mannans, gum arabic, locust bean gum, mesquite gum, guar gum, gum karaya, gum ghatti, tragacanth gum, funori, carrageenans, agar, alginates, chitosans, or gellan gum. In some embodiments the coating material comprises a protein. In another embodiment, the coating material comprises at least one of a fat and an oil. In other embodiments, the at least one of a fat and an oil is high temperature melting. In yet another embodiment, the at least one of a fat and an oil is hydrogenated or partially hydrogenated. In one embodiment, the at least one of a fat and an oil is derived from a plant. In other embodiments, the at least one of a fat and an oil comprises at least one of glycerides, free fatty acids, and fatty acid esters. In some embodiments, the coating material comprises at least one edible wax. The edible wax can be derived from animals, insects, or plants. Non-limiting examples include beeswax, lanolin, bayberry wax, carnauba wax, and rice bran wax. Tablets and pills can additionally be prepared with enteric coatings.

Alternatively, powders or granules embodying the bacterial compositions disclosed herein can be incorporated into a food product. In some embodiments, the food product is a drink for oral administration. Non-limiting examples of a suitable drink include fruit juice, a fruit drink, an artificially flavored drink, an artificially sweetened drink, a carbonated beverage, a sports drink, a liquid diary product, a shake, an alcoholic beverage, a caffeinated beverage, infant formula and so forth. Other suitable means for oral administration include aqueous and nonaqueous solutions, emulsions, suspensions and solutions and/or suspensions reconstituted from non-effervescent granules, containing at least one of suitable solvents, preservatives, emulsifying agents, suspending agents, diluents, sweeteners, coloring agents, and flavoring agents.

In some embodiments, the food product can be a solid foodstuff. Suitable examples of a solid foodstuff include without limitation a food bar, a snack bar, a cookie, a brownie, a muffin, a cracker, an ice cream bar, a frozen yogurt bar, and the like.

In other embodiments, the compositions disclosed herein are incorporated into a therapeutic food. In some embodiments, the therapeutic food is a ready-to-use food that optionally contains some or all essential macronutrients and micronutrients. In another embodiment, the compositions disclosed herein are incorporated into a supplementary food that is designed to be blended into an existing meal. In one embodiment, the supplemental food contains some or all essential macronutrients and micronutrients. In another embodiment, the bacterial compositions disclosed herein are blended with or added to an existing food to fortify the food's protein nutrition. Examples include food staples (grain, salt, sugar, cooking oil, margarine), beverages (coffee, tea, soda, beer, liquor, sports drinks), snacks, sweets and other foods.

In one embodiment, the formulations are filled into gelatin capsules for oral administration. An example of an appropriate capsule is a 250 mg gelatin capsule containing from 10 (up to 100 mg) of lyophilized powder (10⁸ to 10¹¹ bacteria), 160 mg microcrystalline cellulose, 77.5 mg gelatin, and 2.5 mg magnesium stearate. In an alternative embodiment, from 10⁵ to 10¹² bacteria may be used, 10⁶ to 10⁷, 10⁶ to 10⁷, or 10⁸ to 10¹⁰, with attendant adjustments of the excipients if necessary. In an alternative embodiment, an enteric-coated capsule or tablet or with a buffering or protective composition can be used.

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W. H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pennsylvania: Mack Publishing Company, 1990); Carey and Sundberg Advanced Organic Chemistry 3^{rd} Ed. (Plenum Press) Vols A and B (1992).

### Example 1: Species Identification

The identity of the bacterial species which grew up from a complex fraction can be determined in multiple ways. First, individual colonies can be picked into liquid media in a 96 well format, grown up and saved as 15% glycerol stocks at −80° C. Aliquots of the cultures can be placed into cell lysis buffer and colony PCR methods can be used to amplify and sequence the 16S rDNA gene (Example 2 and described above). Alternatively, colonies may be streaked to purity in several passages on solid media. Well separated colonies are streaked onto the fresh plates of the same kind and incubated for 48-72 hours at 37° C. The process is repeated multiple times in order to ensure purity. Pure cultures can be analyzed by phenotypic- or sequence-based methods, including 16S rDNA amplification and sequencing as described in Examples 2 and 3. Sequence characterization of pure isolates or mixed communities e.g. plate scrapes and spore fractions can also include whole genome shotgun sequencing. The latter is valuable to determine the presence of genes associated with sporulation, antibiotic resistance, pathogenicity, and virulence. Colonies can also be scraped from plates en masse and sequenced using a massively parallel sequencing method as described in Examples 2 and 3, such that individual 16S signatures can be identified in a complex mixture. Optionally, the sample can be sequenced prior to germination (if appropriate DNA isolation procedures are used to Isye and release the DNA from spores) in order to compare the diversity of germinable species with the total number of species in a spore sample. As an alternative or complementary approach to 16S analysis, MALDI-TOF-mass spec can also be used for species identification (as reviewed in Anaerobe 22:123).

### Example 2: 16s Sequencing to Determine Operational Taxonomic Unit (OTU

### Method for Determining 16S Sequence

OTUs can be defined either by full 16S sequencing of the rRNA gene, by sequencing of a specific hypervariable region of this gene (i.e. V1, V2, V3, V4, V5, V6, V7, V8, or V9), or by sequencing of any combination of hypervariable regions from this gene (e.g. V1-3 or V3-5). The bacterial 16S rDNA is approximately 1500 nucleotides in length and is used in reconstructing the evolutionary relationships and sequence similarity of one bacterial isolate to another using phylogenetic approaches. 16S sequences are used for phylogenetic reconstruction as they are in general highly conserved, but contain specific hypervariable regions that harbor sufficient nucleotide diversity to differentiate genera and species of most microbes.

Using well known techniques, in order to determine the full 16S sequence or the sequence of any hypervariable region of the 16S sequence, genomic DNA is extracted from a bacterial sample, the 16S rDNA (full region or specific hypervariable regions) amplified using polymerase chain reaction (PCR), the PCR products cleaned, and nucleotide sequences delineated to determine the genetic composition of 16S gene or subdomain of the gene. If full 16S sequencing is performed, the sequencing method used may be, but is not limited to, Sanger sequencing. If one or more hypervariable regions are used, such as the V4 region, the sequencing may be, but is not limited to being, performed using the Sanger method or using a next-generation sequencing method, such as an Illumina (sequencing by synthesis) method using barcoded primers allowing for multiplex reactions.

In addition to the 16S rRNA gene, one can define an OTU by sequencing a selected set of genes that are known to be marker genes for a given species or taxonomic group of OTUs. These genes may alternatively be assayed using a PCR-based screening strategy. As example, various strains of pathogenic Escherichia coli can be distinguished using DNAs from the genes that encode heat-labile (LTI, LTIIa, and LTIIb) and heat-stable (STI and STII) toxins, verotoxin types 1, 2, and 2e (VT1, VT2, and VT2e, respectively), cytotoxic necrotizing factors (CNF1 and CNF2), attaching and effacing mechanisms (eaeA), enteroaggregative mechanisms (Eagg), and enteroinvasive mechanisms (Einv). The optimal genes to utilize for taxonomic assignment of OTUs by use of marker genes will be familiar to one with ordinary skill of the art of sequence based taxonomic identification.

### Genomic DNA Extraction

Genomic DNA is extracted from pure microbial cultures using a hot alkaline lysis method. 1 μl of microbial culture is added to 9 μl of Lysis Buffer (25 mM NaOH, 0.2 mM EDTA) and the mixture is incubated at 95° C. for 30 minutes. Subsequently, the samples are cooled to 4° C. and neutralized by the addition of 10 μl of Neutralization Buffer (40 mM Tris-HCl) and then diluted 10-fold in Elution Buffer (10 mM Tris-HCl). Alternatively, genomic DNA is extracted from pure microbial cultures using commercially available kits such as the Mo Bio Ultraclean® Microbial DNA Isolation Kit (Mo Bio Laboratories, Carlsbad, CA) or by standard methods known to those skilled in the art.

### Amplification of 16S Sequences for Downstream Sanger Sequencing

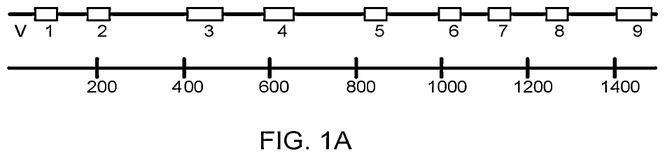

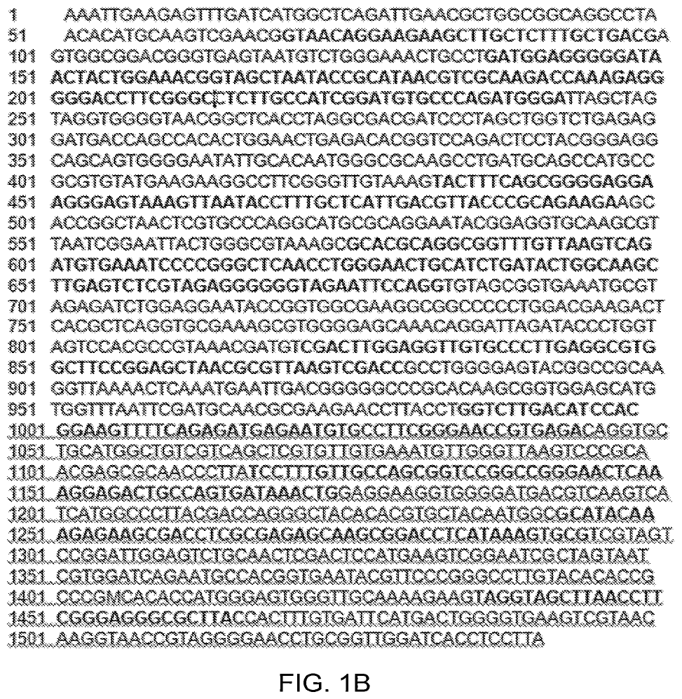

To amplify bacterial 16S rDNA (FIG. 1A), 2 μl of extracted gDNA is added to a 20 μl final volume PCR reaction. For full-length 16 sequencing the PCR reaction also contains 1× HotMasterMix (5PRIME, Gaithersburg, MD), 250 nM of 27f (AGRGTTTGATCMTGGCTCAG (SEQ ID NO: 2033), IDT, Coralville, IA), and 250 nM of 1492r (TACGGYTACCTTGTTAYGACTT (SEQ ID NO: 2034), IDT, Coralville, (A), with PCR Water (Mo Bio Laboratories, Carlsbad, CA) for the balance of the volume. Alternatively, other universal bacterial primers or thermostable polymerases known to those skilled in the art are used. For example primers are available to those skilled in the art for the sequencing of the “V1-V9 regions” of the 16S rRNA (FIG. 1A). These regions refer to the first through ninth hypervariable regions of the 16S rRNA gene that are used for genetic typing of bacterial samples. These regions in bacteria are defined by nucleotides 69-99, 137-242, 433-497, 576-682, 822-879, 986-1043, 1117-1173, 1243-1294 and 1435-1465 respectively using numbering based on the E. coli system of nomenclature. Brosius et al., Complete nucleotide sequence of a 16S ribosomal RNA gene from Escherichia coli, PNAS 75 (10): 4801-4805 (1978). In some embodiments, at least one of the V1, V2, V3, V4, V5, V6, V7, V8, and V9 regions are used to characterize an OTU. In one embodiment, the V1, V2, and V3 regions are used to characterize an OTU. In another embodiment, the V3, V4, and V5 regions are used to characterize an OTU. In another embodiment, the V4 region is used to characterize an OTU. A person of ordinary skill in the art can identify the specific hypervariable regions of a candidate 16S rRNA (in FIG. 1A) by comparing the candidate sequence in question to the reference sequence (FIG. 1B) and identifying the hypervariable regions based on similarity to the reference hypervariable regions.

The PCR can be performed on commercially available thermocyclers such as a BioRad MyCycler™ Thermal Cycler (BioRad, Hercules, CA). The reactions are run at 94° C. for 2 minutes followed by 30 cycles of 94° C. for 30 seconds, 51° C. for 30 seconds, and 68° C. for 1 minute 30 seconds, followed by a 7 minute extension at 72° C. and an indefinite hold at 4° C. Following PCR, gel electrophoresis of a portion of the reaction products is used to confirm successful amplification of a ˜1.5 kb product.

To remove nucleotides and oligonucleotides from the PCR products, 2 μl of HT ExoSap-IT (Affymetrix, Santa Clara, CA) is added to 5 μl of PCR product followed by a 15 minute incubation at 37° C. and then a 15 minute inactivation at 80° C.

### Amplification of 16S Sequences for Downstream Characterization by Massively Parallel Sequencing Technologies

Amplification performed for downstream sequencing by short read technologies such as Illumina require amplification using primers known to those skilled in the art that additionally include a sequence-based barcoded tag. As example, to amplify the 16s hypervariable region V4 region of bacterial 16S rDNA, 2 μl of extracted gDNA is added to a 20 μl final volume PCR reaction. The PCR reaction also contains 1× HotMasterMix (5PRIME, Gaithersburg, MD), 200 nM of V4_515f_adapt (AATGATACGGCGACCACCGAGATCTACACTATGGTAATTGTGTGCCAGCMGCC GCGGTAA (SEQ ID NO: 2035), IDT, Coralville, IA), and 200 nM of barcoded 806 rbc (CAAGCAGAAGACGGCATACGAGAT_12bpGolayBarcode_AGTCAGTCAGCCGG ACTACHVGGGTWTCTAAT (SEQ ID NOS 2036 and 2037, respectively), IDT, Coralville, IA), with PCR Water (Mo Bio Laboratories, Carlsbad, CA) for the balance of the volume. These primers incorporate barcoded adapters for Illumina sequencing by synthesis. Optionally, identical replicate, triplicate, or quadruplicate reactions may be performed. Alternatively other universal bacterial primers or thermostable polymerases known to those skilled in the art are used to obtain different amplification and sequencing error rates as well as results on alternative sequencing technologies.

The PCR amplification is performed on commercially available thermocyclers such as a BioRad MyCycler™ Thermal Cycler (BioRad, Hercules, CA). The reactions are run at 94° C. for 3 minutes followed by 25 cycles of 94° C. for 45 seconds, 50° C. for 1 minute, and 72° C. for 1 minute 30 seconds, followed by a 10 minute extension at 72° C. and a indefinite hold at 4° C. Following PCR, gel electrophoresis of a portion of the reaction products is used to confirm successful amplification of a ˜1.5 kb product. PCR cleanup is performed as specified in the previous example.

Sanger Sequencing of Target Amplicons from Pure Homogeneous Samples

To detect nucleic acids for each sample, two sequencing reactions are performed to generate a forward and reverse sequencing read. For full-length 16s sequencing primers 27f and 1492r are used. 40 ng of ExoSap-IT-cleaned PCR products are mixed with 25 pmol of sequencing primer and Mo Bio Molecular Biology Grade Water (Mo Bio Laboratories, Carlsbad, CA) to 15 μl total volume. This reaction is submitted to a commercial sequencing organization such as Genewiz (South Plainfield, NJ) for Sanger sequencing.

Massively Parallel Sequencing of Target Amplicons from Heterogeneous Samples

DNA Quantification & Library Construction. The cleaned PCR amplification products are quantified using the Quant-IT™ PicoGreen® dsDNA Assay Kit (Life Technologies, Grand Island, NY) according to the manufacturer's instructions. Following quantification, the barcoded cleaned PCR products are combined such that each distinct PCR product is at an equimolar ratio to create a prepared Illumina library.

Nucleic Acid Detection. The prepared library can be sequenced on Illumina HiSeq or MiSeq sequencers (Illumina, San Diego, CA) with cluster generation, template hybridization, iso-thermal amplification, linearization, blocking and denaturization and hybridization of the sequencing primers performed according to the manufacturer's instructions. 16SV4SeqFw (TATGGTAATTGTGTGCCAGCMGCCGCGGTAA) (SEQ ID NO: 2038), 16SV4SeqRev (AGTCAGTCAGCCGGACTACHVGGGTWTCTAAT) (SEQ ID NO: 2037), and 16SV4Index (ATTAGAWACCCBDGTAGTCCGGCTGACTGACT) (SEQ ID NO: 2039) (IDT, Coralville, IA) are used for sequencing. Other sequencing technologies can be used such as but not limited to 454, Pacific Biosciences, Helicos, Ion Torrent, and Nanopore using protocols that are standard to someone skilled in the art of genomic sequencing.

### Example 3: Sequence Read Annotation

### Primary Read Annotation

Nucleic acid sequences can be analyzed and annotated to define taxonomic assignments using sequence similarity and phylogenetic placement methods or a combination of the two strategies. A similar approach can be used to annotate protein names, transcription factor names, and any other classification schema for nucleic acid sequences. Sequence similarity based methods include those familiar to individuals skilled in the art including, but not limited to BLAST, BLASTx, tBLASTn, tBLASTx, RDP-classifier, DNAclust, and various implementations of these algorithms such as Qiime or Mothur. These methods rely on mapping a sequence read to a reference database and selecting the match with the best score and e-value. Common databases include, but are not limited to the Human Microbiome Project, NCBI non-redundant database, Greengenes, RDP, and Silva. Phylogenetic methods can be used in combination with sequence similarity methods to improve the calling accuracy of an annotation or taxonomic assignment. Here tree topologies and nodal structure are used to refine the resolution of the analysis. In this approach we analyze nucleic acid sequences using one of numerous sequence similarity approaches and leverage phylogenetic methods that are well known to those skilled in the art, including but not limited to maximum likelihood phylogenetic reconstruction (see e.g. Liu K, Linder C R, and Warnow T. 2011. RAxML and FastTree: Comparing Two Methods for Large-Scale Maximum Likelihood Phylogeny Estimation. PLOS ONE 6: e27731. McGuire G, Denham M C, and Balding D J. 2001. Models of sequence evolution for DNA sequences containing gaps. Mol. Biol. Evol 18:481-490. Wróbel B. 2008. Statistical measures of uncertainty for branches in phylogenetic trees inferred from molecular sequences by using model-based methods. J. Appl. Genet. 49:49-67.) Sequence reads are placed into a reference phylogeny comprised of appropriate reference sequences. Annotations are made based on the placement of the read in the phylogenetic tree. The certainty or significance of the OTU annotation is defined based on the OTU's sequence similarity to a reference nucleic acid sequence and the proximity of the OTU sequence relative to one or more reference sequences in the phylogeny. As an example, the specificity of a taxonomic assignment is defined with confidence at the the level of Family, Genus, Species, or Strain with the confidence determined based on the position of bootstrap supported branches in the reference phylogenetic tree relative to the placement of the OTU sequence being interrogated.

### Clade Assignments

The ability of 16S-V4 OTU identification to assign an OTU as a specific species depends in part on the resolving power of the 16S-V4 region of the 16S gene for a particular species or group of species. Both the density of available reference 16S sequences for different regions of the tree as well as the inherent variability in the 16S gene between different species will determine the definitiveness of a taxonomic annotation. Given the topological nature of a phylogenetic tree and the fact that tree represents hierarchical relationships of OTUs to one another based on their sequence similarity and an underlying evolutionary model, taxonomic annotations of a read can be rolled up to a higher level using a clade-based assignment procedure (Table 1). Using this approach, clades are defined based on the topology of a phylogenetic tree that is constructed from full-length 16S sequences using maximum likelihood or other phylogenetic models familiar to individuals with ordinary skill in the art of phylogenetics. Clades are constructed to ensure that all OTUs in a given clade are: (i) within a specified number of bootstrap supported nodes from one another (generally, 1-5 bootstraps), and (ii) within a 5% genetic similarity. OTUs that are within the same clade can be distinguished as genetically and phylogenetically distinct from OTUs in a different clade based on 16S-V4 sequence data. OTUs falling within the same clade are evolutionarily closely related and may or may not be distinguishable from one another using 16S-V4 sequence data. The power of clade based analysis is that members of the same clade, due to their evolutionary relatedness, are likely to play similar functional roles in a microbial ecology such as that found in the human gut. Compositions substituting one species with another from the same clade are likely to have conserved ecological function and therefore are useful in the present invention.

Notably, 16S sequences of isolates of a given OTU are phylogenetically placed within their respective clades, sometimes in conflict with the microbiological-based assignment of species and genus that may have preceded 16S-based assignment. Discrepancies between taxonomic assignment based on microbiological characteristics versus genetic sequencing are known to exist from the literature.

### Example 4: Germinating Spores

Mixtures of bacteria can include species that are in spore form. Germinating a spore fraction increases the number of viable bacteria that will grow on various media types. To germinate a population of spores, the sample is moved to the anaerobic chamber, resuspended in prereduced PBS, mixed and incubated for 1 hour at 37 C to allow for germination. Germinants can include amino-acids (e.g., alanine, glycine), sugars (e.g., fructose), nucleosides (e.g., inosine), bile salts (e.g., cholate and taurocholate), metal cations (e.g., Mg2+, Ca2+), fatty acids, and long-chain alkyl amines (e.g., dodecylamine, Germination of bacterial spores with alkyl primary amines” J. Bacteriology, 1961). Mixtures of these or more complex natural mixtures, such as rumen fluid or Oxgall, can be used to induce germination. Oxgall is dehydrated bovine bile composed of fatty acids, bile acids, inorganic salts, sulfates, bile pigments, cholesterol, mucin, lecithin, glycuronic acids, porphyrins, and urea. The germination can also be performed in a growth medium like prereduced BHIS/oxgall germination medium, in which BHIS (Brain heart infusion powder (37 g/L), yeast extract (5 g/L), L-cysteine HCl (1 g/L)) provides peptides, amino acids, inorganic ions and sugars in the complex BHI and yeast extract mixtures and Oxgall provides additional bile acid germinants.

In addition, pressure may be used to germinate spores. The selection of germinants can vary with the microbe being sought. Different species require different germinants and different isolates of the same species can require different germinants for optimal germination. Finally, it is important to dilute the mixture prior to plating because some germinants are inhibitory to growth of the vegetative-state microorganisms. For instance, it has been shown that alkyl amines must be neutralized with anionic lipophiles in order to promote optimal growth. Bile acids can also inhibit growth of some organisms despite promoting their germination, and must be diluted away prior to plating for viable cells.

For example, BHIS/oxgall solution is used as a germinant and contains 0.5×BHIS medium with 0.25% oxgall (dehydrated bovine bile) where 1×BHIS medium contains the following per L of solution: 6 g Brain Heart Infusion from solids, 7 g peptic digest of animal tissue, 14.5 g of pancreatic digest of casein, 5 g of yeast extract, 5 g sodium chloride, 2 g glucose, 2.5 g disodium phosphate, and 1 g cysteine. Additionally, Ca-DPA is a germinant and contains 40 mM CaCl2, and 40 mM dipicolinic acid (DPA). Rumen fluid (Bar Diamond, Inc.) is also a germinant. Simulated gastric fluid (Ricca Chemical) is a germinant and is 0.2% (w/v) Sodium Chloride in 0.7% (v/v) Hydrochloric Acid. Mucin medium is a germinant and prepared by adding the following items to 1 L of distilled sterile water: 0.4 g KH₂PO₄, 0.53 g Na₂HPO₄, 0.3 g NH₄Cl, 0.3 g NaCl, 0.1 g MgCl₂×6H₂O, 0.11 g CaCl₂), 1 ml alkaline trace element solution, 1 ml acid trace element solution, 1 ml vitamin solution, 0.5 mg resazurin, 4 g NaHCO₃, 0.25 g Na₂S×9H₂O. The trace element and vitamin solutions prepared as described previously (Stams et al., 1993). All compounds were autoclaved, except the vitamins, which were filter-sterilized. The basal medium was supplemented with 0.7% (v/v) clarified, sterile rumen fluid and 0.25% (v/V) commercial hog gastric mucin (Type III; Sigma), purified by ethanol precipitation as described previously (Miller & Hoskins, 1981). This medium is referred herein as mucin medium.

Fetal Bovine Serum (Gibco) can be used as a germinant and contains 5% FBS heat inactivated, in Phosphate Buffered Saline (PBS, Fisher Scientific) containing 0.137M Sodium Chloride, 0.0027M Potassium Chloride, 0.0119M Phosphate Buffer. Thioglycollate is a germinant as described previously (Kamiya et al Journal of Medical Microbiology 1989) and contains 0.25M (pH10) sodium thioglycollate. Dodecylamine solution containing 1 mM dodecylamine in PBS is a germinant. A sugar solution can be used as a germinant and contains 0.2% fructose, 0.2% glucose, and 0.2% mannitol. Amino acid solution can also be used as a germinant and contains 5 mM alanine, 1 mM arginine, 1 mM histidine, 1 mM lysine, 1 mM proline, 1 mM asparagine, 1 mM aspartic acid, 1 mM phenylalanine. A germinant mixture referred to herein as Germix 3 can be a germinant and contains 5 mM alanine, 1 mM arginine, 1 mM histidine, 1 mM lysine, 1 mM proline, 1 mM asparagine, 1 mM aspartic acid, 1 mM phenylalanine, 0.2% taurocholate, 0.2% fructose, 0.2% mannitol, 0.2% glucose, 1 mM inosine, 2.5 mM Ca-DPA, and 5 mM KCl. BHIS medium+DPA is a germinant mixture and contains BHIS medium and 2 mM Ca-DPA. Escherichia coli spent medium supernatant referred to herein as EcSN is a germinant and is prepared by growing E. coli MG1655 in SweetB/Fos inulin medium anaerobically for 48 hr, spinning down cells at 20,000 rcf for 20 minutes, collecting the supernatant and heating to 60 C for 40 min. Finally, the solution is filter sterilized and used as a germinant solution.

### Example 5: Selection of Media for Growth

Provided are appropriate media to support growth, including preferred carbon sources. For example, some organisms prefer complex sugars such as cellobiose over simple sugars. Examples of media used in the isolation of sporulating organisms include EYA, BHI, BHIS, and GAM (see below for complete names and references). Multiple dilutions are plated out to ensure that some plates will have well isolated colonies on them for analysis, or alternatively plates with dense colonies may scraped and suspended in PBS to generate a mixed diverse community.

Plates are incubated anaerobically or aerobically at 37 C for 48-72 or more hours, targeting anaerobic or aerobic spore formers, respectively.

Solid plate media include:

Gifu Anaerobic Medium (GAM, Nissui) without dextrose supplemented with fructooligosaccharides/inulin (0.4%), mannitol (0.4%), inulin (0.4%), or fructose (0.4%), or a combination thereof.

Sweet GAM [Gifu Anaerobic Medium (GAM, Nissui)] modified, supplemented with glucose, cellobiose, maltose, L-arabinose, fructose, fructooligosaccharides/inulin, mannitol and sodium lactate)

Brucella Blood Agar (BBA, Atlas, Handbook of Microbiological Media, 4th ed, ASM Press, 2010)

PEA sheep blood (Anaerobe Systems; 5% Sheep Blood Agar with Phenylethyl Alcohol)

Egg Yolk Agar (EYA) (Atlas, Handbook of Microbiological Media, 4th ed, ASM Press, 2010)

Sulfite polymyxin milk agar (Mevissen-Verhage et al., J. Clin. Microbiol. 25:285-289 (1987))

Mucin agar (Derrien et al., IJSEM 54:1469-1476 (2004))

Polygalacturonate agar (Jensen & Canale-Parola, Appl. Environ. Microbiol. 52:880-997 (1986))

M2GSC (Atlas, Handbook of Microbiological Media, 4th ed, ASM Press, 2010)

M2 agar (Atlas, Handbook of Microbiological Media, 4th ed, ASM Press, 2010) supplemented with starch (1%), mannitol (0.4%), lactate (1.5 g/L) or lactose (0.4%)

Sweet B—Brain Heart Infusion agar (Atlas, Handbook of Microbiological Media, 4th ed, ASM Press, 2010) supplemented with yeast extract (0.5%), hemin, cysteine (0.1%), maltose (0.1%), cellobiose (0.1%), soluble starch (sigma, 1%), MOPS (50 mM, pH 7).

PY-salicin (peptone-yeast extract agar supplemented with salicin) (Atlas, Handbook of Microbiological Media, 4th ed, ASM Press, 2010).

Modified Brain Heart Infusion (M-BHI) [sweet and sour] contains the following per L: 37.5 g Brain Heart Infusion powder (Remel), 5 g yeast extract, 2.2 g meat extract, 1.2 g liver extract, 1 g cystein HCl, 0.3 g sodium thioglycolate, 10 mg hemin, 2 g soluble starch, 2 g FOS/Inulin, 1 g cellobiose, 1 g L-arabinose, 1 g mannitol, 1 Na-lactate, 1 mL Tween 80, 0.6 g MgSO4×7H2O, 0.6 g CaCl2, 6 g (NH4)2SO4, 3 g KH2PO4, 0.5 g K2HPO4, 33 mM Acetic acid, 9 mM propionic acid, 1 mM Isobutyric acid, 1 mM isovaleric acid, 15 g agar, and after autoclaving add 50 mL of 8% NaHCO₃ solution and 50 mL 1M MOPS-KOH (pH 7).

Noack-Blaut Eubacterium agar (See Noack et al. J. Nutr. (1998) 128:1385-1391)

BHIS az1/ge2-BHIS az/ge agar (Reeves et. al. Infect. Immun. 80:3786-3794 (2012)) [Brain Heart Infusion agar (Atlas, Handbook of Microbiological Media, 4th ed, ASM Press, 2010) supplemented with yeast extract 0.5%, cysteine 0.1%, 0.1% cellobiose, 0.1% inulin, 0.1% maltose, aztreonam 1 mg/L, gentamycin 2 mg/L]

BHIS CInM az1/ge2-BHIS CInM [Brain Heart Infusion agar (Atlas, Handbook of Microbiological Media, 4th ed, ASM Press, 2010) supplemented with yeast extract 0.5%, cysteine 0.1%, 0.1% cellobiose, 0.1% inulin, 0.1% maltose, aztreonam 1 mg/L, gentamycin 2 mg/L]

### Example 6: Purification and Isolation of a Spore Forming Fraction from Feces

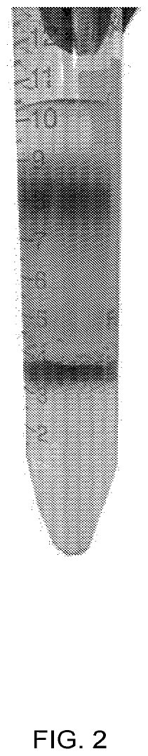

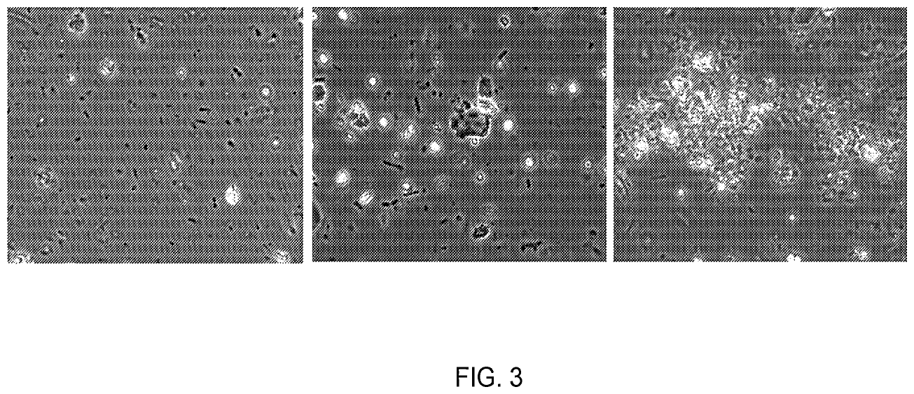

To purify and selectively isolate efficacious spores from fecal material a donation is first blended with saline using a homogenization device (e.g., laboratory blender) to produce a 20% slurry (w/v). 100% ethanol is added for an inactivation treatment that lasts 10 seconds to 1 hour. The final alcohol concentration can range from 30-90%, preferably 50-70%. High speed centrifugation (3200 rcf for 10 min) is performed to remove solvent and the pellet is retained and washed. Subsequently, once the washed pellet is resuspended, a low speed centrifugation step (200 rcf for 4 min) is performed to remove large particulate vegetative matter and the supernatant containing the spores is retained. High speed centrifugation (3200 rcf for 10 min) is performed on the supernatant to concentrate the spore material. The pellet is then washed and resuspended to generate a 20% slurry. This is the ethanol treated spore preparation. The concentrated slurry is then separated with a density based gradient e.g. a CsCl gradient, sucrose gradient or combination of the two generating a ethanol treated, gradient-purified spore preparation. For example, a CsCl gradient is performed by loading a 20% volume of spore suspension on top a 80% volume of a stepwise CsCl gradient (w/v) containing the steps of 64%, 50%, 40% CsCl (w/v) and centrifuging for 20 min at 3200 rcf. The spore fraction is then run on a sucrose step gradient with steps of 67%, 50%, 40%, and 30% (w/v). When centrifuged in a swinging bucket rotor for 10 min at 3200 rcf. The spores run roughly in the 30% and 40% sucrose fractions. The lower spore fraction (FIG. 2) is then removed and washed to produce a concentrated ethanol treated, gradient-purified spore preparation. Taking advantage of the refractive properties of spores observed by phase contrast microscopy (spores are bright and refractive while germinated spores and vegetative cells are dark) one can see an enrichment of the spore fraction from a fecal bacterial cell suspension (FIG. 3, left) compared to an ethanol treated, CsCl gradient purified, spore preparation (FIG. 3, center), and to an ethanol treated, CsCl gradient purified, sucrose gradient purified, spore preparation (FIG. 3, right).

Furthermore, growth of spores after treatment with a germinant can also be used to quantify a viable spore population. Briefly, samples were incubated with a germinant (Oxgall, 0.25% for up to 1 hour), diluted and plated anaerobically on BBA (Brucella Blood Agar) or similar media (e.g. see Examples 4 and 5). Individual colonies were picked and DNA isolated for full-length 16S sequencing to identify the species composition (e.g. see Examples 2 and 3). Analysis revealed that 22 species were observed in total (Table 2) with a vast majority present in both the material purified with the gradient and without the gradient, indicating no or inconsequential shift in the ecology as a result of gradient purification. Spore yield calculations demonstrate an efficient recovery of 38% of the spores from the initial fecal material as measured by germination and plating of spores on BBA or measuring DPA count in the sample.

### Example 7: Bacterial Compositions Prevent C. difficile Infection in a Mouse Model

To test the therapeutic potential of the bacterial composition such as but not limited to a spore population, a prophylactic mouse model of C. difficile infection (model based on Chen, et al., A mouse model of Clostridium difficile associated disease, Gastroenterology 135 (6): 1984-1992) was used. Two cages of five mice each were tested for each arm of the experiment. All mice received an antibiotic cocktail consisting of 10% glucose, kanamycin (0.5 mg/ml), gentamicin (0.044 mg/ml), colistin (1062.5 U/ml), metronidazole (0.269 mg/ml), ciprofloxacin (0.156 mg/ml), ampicillin (0.1 mg/ml) and Vancomycin (0.056 mg/ml) in their drinking water on days −14 through −5 and a dose of 10 mg/kg Clindamycin by oral gavage on day −3. On day −1, they received either the test article or vehicle control via oral gavage. On day 0 they were challenged by administration of approximately 4.5 log 10 cfu of C. difficile (ATCC 43255) via oral gavage. Optionally a positive control group received vancomycin from day −1 through day 3 in addition to the antibiotic protocol and C. difficile challenge specified above. Feces were collected from the cages for analysis of bacterial carriage, mortality was assessed every day from day 0 to day 6 and the weight and subsequent weight change of the animal was assessed with weight loss being associated with C. difficile infection. Mortality and reduced weight loss of the test article compared to the vehicle were used to assess the success of the test article. Additionally, a C. difficile symptom scoring was performed each day from day −1 through day 6. Clinical Score was based on a 0-4 scale by combining scores for Appearance (0-2 pts based on normal, hunched, piloerection, or lethargic), and Clinical Signs (0-2 points based on normal, wet tail, cold-to-the-touch, or isolation from other animals).

In a naive control arm, animals were challenged with C. difficile. In the vancomycin positive control arm animals were dosed with C. difficile and treated with vancomycin from day −1 through day 3. The negative control was gavaged with PBS alone and no bacteria. The test arms of the experiment tested 1×, 0.1×, 0.01× dilutions derived from a single donor preparation of ethanol treated spores (e.g. see example AAAF) or the heat treated feces prepared by treating a 20% slurry for 30 min at 80 C. Dosing for CFU counts was determined from the final ethanol treated spores and dilutions of total spores were administered at 1×, 0.1×, 0.01× of the spore mixture for the ethanol treated fraction and a 1× dose for the heat treated fraction.

Weight loss and mortality were assessed on day 3. The negative control, treated with C. difficile only, exhibits 20% mortality and weight loss on Day 3, while the positive control of 10% human fecal suspension displays no mortality or weight loss on Day 3 (Table 5). Table 5 contains the mortality and weight change of mice treated with a donor fecal suspension and an ethanol and/or heat-treated spore preparation at various dilutions. EtOH-treated feces prevents mortality and weight loss at three dilutions, while the heat-treated fraction was protective at the only dose tested. These data indicate that the spore fraction is efficacious in preventing C. difficile infection in the mouse.

### Example 8: Prophylactic and Relapse Prevention Hamster Models

Previous studies with hamsters using toxigenic and nontoxigenic strains of C. difficile demonstrated the utility of the hamster model in examining relapse post antibiotic treatment and the effects of prophylaxis treatments with cecal flora in C. difficile infection (Wilson et al. 1981, Wilson et al. 1983, Borriello et al. 1985) and more broadly gastrointestinal infectious disease. To demonstrate prophylactic use of a bacterial composition such as but not limited to a spore population, spore preparation, vegetative cell population, to ameliorate C. difficile infection, the following hamster model is used. In a prophylactic model, Clindamycin (10 mg/kg s.c.) is given on day −5, the bacterial composition or control is administered on day −3, and C. difficile challenge occurs on day 0. In the positive control arm, vancomycin is then administered on day 1-5 (and vehicle control is delivered on day −3). Feces are collected on day −5, −4, −1, 1, 3, 5, 7, 9 and fecal samples are assessed for pathogen carriage and reduction by microbiological methods, 16S sequencing approaches or other methods utilized by one skilled in the art. Mortality is assessed throughout the experiment through 21 days post C. difficile challenge. The percentage survival curves show that ethanol treated spores and ethanol treated, gradient-purified spores better protect the hamsters compared to the Vancomycin control, and vehicle control.

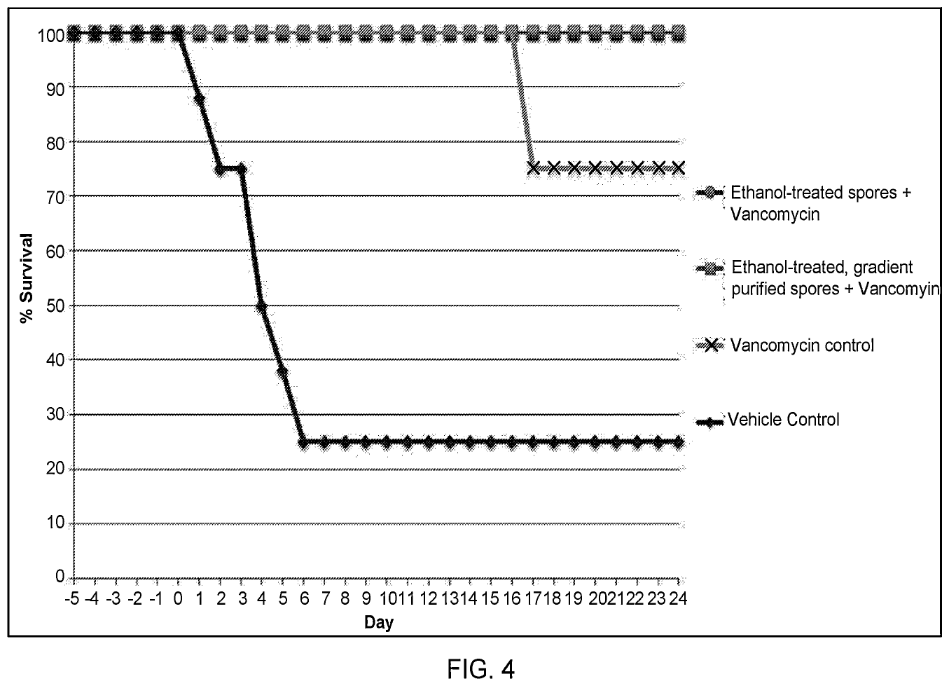

FIG. 4 shows a prophylaxis model with the ethanol treated spore preparation and the ethanol treated, gradient-purified spore preparation.

In the relapse prevention model, hamsters are challenged with toxigenic C. difficile strains on day 0, and treated with clindamycin by oral gavage on day 1, and vancomycin dosing day 2-6. Test or control treatment was then administered on day 7, 8, and 9. The groups of hamsters for each arm consist of 8 hamsters per group. Fecal material is collected on day −1, 1, 3, 5, 7, 10 and 13 and hamster mortality is assessed throughout. Survival curves are used to assess the success of the test article e.g. ethanol treated or ethanol treated, gradient purified spores versus the control treatment in preventing hamster death. The survival curves demonstrate maximum efficacy for the ethanol treated, gradient-purified spores followed by the ethanol treated spores. Both treatments improved survival percentage over vancomycin treatment alone.

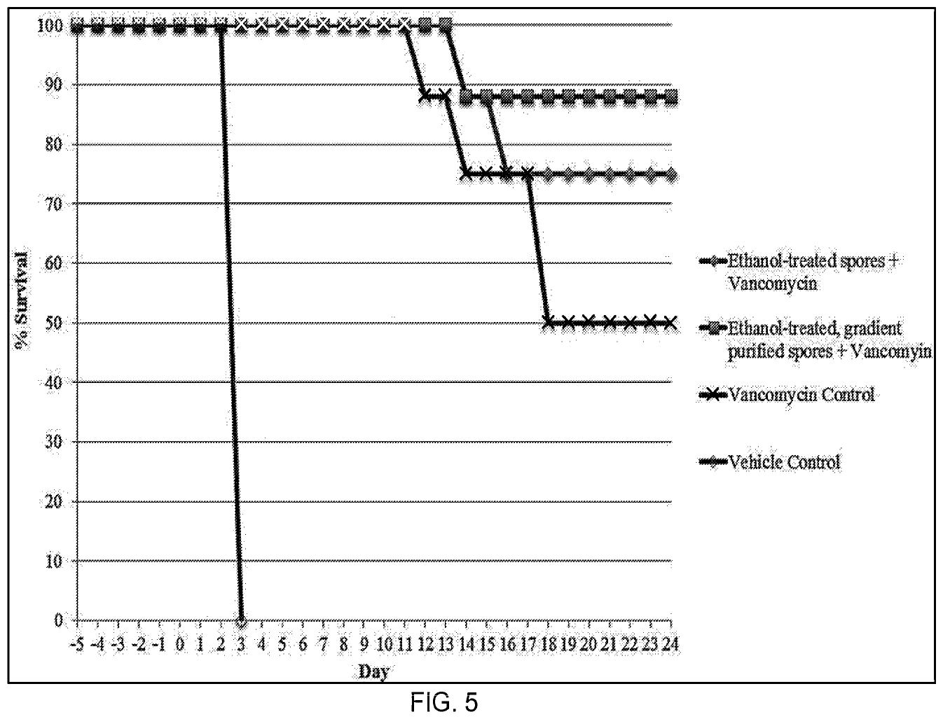

FIG. 5 shows the relapse prevention model with ethanol treated spores and ethanol treated, gradient purified spores

### Example 9: Clinical Treatment of Recurrent C. difficile in Patients

To assess the efficacy of test articles like bacterial composition including but not limited to a ethanol treated spore preparations (e.g. see Example 6) to treat recurrent C. difficile in human patients, the following procedure was performed to take feces from a healthy donor, inactivate via the ethanol treated spore preparation protocol described below, and treat recurrent C. difficile in patients presenting with this indication. Non-related donors were screened for general health history for absence of chronic medical conditions (including inflammatory bowel disease; irritable bowel syndrome; Celiac disease; or any history of gastrointestinal malignancy or polyposis), absence of risk factors for transmissible infections, antibiotic non-use in the previous 6 months, and negative results in laboratory assays for blood-borne pathogens (HIV, HTLV, HCV, HBV, CMV, HAV and Treponema pallidum) and fecal bacterial pathogens (Salmonella, Shigella, Yersinia, Campylobacter, E. coli 0157), ova and parasites, and other infectious agents (Giardia, Cryptosporidium Cyclospora, Isospora) prior to stool donation.

Donor stool was frozen shortly after donation and sampled for testing. At the time of use, approximately 75 g of donor stool was thawed and resuspended in 500 mL of non-bacteriostatic normal saline and mixed in a single use glass or plastic blender. The resulting slurry was sequentially passed through sterile, disposable mesh screens that remove particles of size 600, 300 and 200 microns. The slurry was then centrifuged briefly (200 rcf for 4 min) to separate fibrous and particulate materials, and the supernatant (containing bacterial cells and spores) was transferred to a fresh container. Ethanol was added to a final concentration of 50% and the resulting ˜1500 ml slurry was incubated at room temperature for 1 hr with continuous mixing to inactivate vegetative bacterial cells. Midway through inactivation the slurry was transferred to a new bottle to ensure complete contact with the ethanol. The solid matter was pelleted in a centrifuge and washed 3 times with normal saline to remove residual ethanol. The final pellet was resuspended in 100% sterile, USP glycerol at a minimum volume, and filled into approximately 30 size 0 delayed release capsules (hypromellose DRcaps, Capsugel, Inc.) at 0.65 mL suspension each. The capsules were immediately capped and placed onto an aluminum freezing block held at −80° C. via dry ice to freeze. The frozen capsules were in turn over-capsulated with size 00 DRcaps to enhance capsule stability, labeled, and placed into <−65° C. storage immediately. The final product was stored at <−65° C. until the day and time of use. Encapsulated product may be stored for indefinitely at <−65° C. On the day of dosing capsules were warmed on wet ice for 1 to 2 hours to improve tolerability, and were then dosed with water ad libitium.

Patient 1 is a 45-year old woman with a history of C. difficile infection and diarrhea for at least 1 year prior to treatment. She has been previously treated with multiple courses of antibiotics followed each time by recurrence of C. difficile-associated diarrhea.

Patient 2 is an 81-year old female who has experienced recurrent C. difficile infection for 6 months prior to treatment despite adequate antibiotic therapy following each recurrence.

24 hours prior to starting oral treatment, CDAD antibiotic therapy was discontinued. Each patient received a colon preparation procedure intended to reduce the competing microbial burden in the gastrointestinal tract and to facilitate repopulation by the spore forming organisms in the investigational product.

On the morning of the first treatment day, the patients received a dose of delayed release capsules containing the investigational product with water ad libitum. Patients were requested to avoid food for 1 hour thereafter. The next day, the patient returned to the clinic to receive an additional dose. Patients were asked to avoid food for 4 hours prior to receiving their second dose and for 1 hour following dosing.

Both patients were followed closely for evidence of relapse or adverse symptoms following treatment. Patients were contacted by phone on Day 2, Day 4, and Weeks 1, 2 and 4 and each was queried about her general status and the condition of her CDAD and related symptoms. Stool samples were collected at baseline and Weeks 1, 2, 4 and 8 post-treatment to assess changes in the gut microbiota via 16S sequencing and spore count with methods explained previously (e.g. see Examples 2 and 3). Through 4 weeks post treatment, each patient has gradually improved with no evidence of C. difficile recurrence.

Six other patients with recurrent C. difficile-associated diarrhea were treated in a similar fashion, with no CDI recurrence and no requirement for resumption of antibiotics (total of 8 patients). Additionally, there were no treatment-related serious adverse events.

The above protocol could be modified to deliver other bacterial compositions e.g. vegetative cells, spore preparations, and combinations thereof.

### Example 10: Enrichment and Purification of Bacteria

To purify individual bacterial strains, dilution plates were selected in which the density enables distinct separation of single colonies. Colonies were picked with a sterile implement (either a sterile loop or toothpick) and re-streaked to BBA or other solid media. Plates were incubated at 37° C. for 3-7 days. One or more well-isolated single colonies of the major morphology type were re-streaked. This process was repeated at least three times until a single, stable colony morphology is observed. The isolated microbe was then cultured anaerobically in liquid media for 24 hours or longer to obtain a pure culture of 10⁶-10¹⁰ cfu/ml. Liquid growth medium might include Brain Heart Infusion-based medium (Atlas, Handbook of Microbiological Media, 4th ed, ASM Press, 2010) supplemented with yeast extract, hemin, cysteine, and carbohydrates (for example, maltose, cellobiose, soluble starch) or other media described previously (e.g. see example AAAE). The culture was centrifuged at 10,000×g for 5 min to pellet the bacteria, the spent culture media was removed, and the bacteria were resuspended in sterile PBS. Sterile 75% glycerol was added to a final concentration of 20%. An aliquot of glycerol stock was titered by serial dilution and plating. The remainder of the stock was frozen on dry ice for 10-15 min and then placed at −80 C for long term storage.

### Example 11: Cell Bank Preparation

Cell banks (RCBs) of bacterial strains were prepared as follows. Bacterial strains were struck from −80° C. frozen glycerol stocks to Brucella blood agar with Hemin or Vitamin K (Atlas, Handbook of Microbiological Media, 4th ed, ASM Press, 2010), M2GSC (Atlas, Handbook of Microbiological Media, 4th ed, ASM Press, 2010) or other solid growth media and incubated for 24 to 48 h at 37° C. in an anaerobic chamber with a gas mixture of H₂:CO₂:N₂ of 10:10:80. Single colonies were then picked and used to inoculate 250 ml to 1 L of Wilkins-Chalgren broth, Brain-Heart Infusion broth, M2GSC broth or other growth media, and grown to mid to late exponential phase or into the stationary phase of growth. Alternatively, the single colonies may be used to inoculate a pilot culture of 10 ml, which were then used to inoculate a large volume culture. The growth media and the growth phase at harvest were selected to enhance cell titer, sporulation (if desired) and phenotypes that might be associated desired in vitro or in vivo. Optionally, Cultures were grown static or shaking, depending which yielded maximal cell titer. The cultures were then concentrated 10 fold or more by centrifugation at 5000 rpm for 20 min, and resuspended in sterile phosphate buffered saline (PBS) plus 15% glycerol. 1 ml aliquots were transferred into 1.8 ml cryovials which were then frozen on dry ice and stored at −80 C. The identity of a given cell bank was confirmed by PCR amplification of the 16S rDNA gene, followed by Sanger direct cycle sequencing, and comparison to a curated rDNA database to determine a taxonomic ID. Each bank was confirmed to yield colonies of a single morphology upon streaking to Brucella blood agar or M2GSC agar. When more than one morphology was observed, colonies were confirmed to be the expected species by PCR and sequencing analysis of the 16S rDNA gene. Variant colony morphologies can be observed within pure cultures, and in a variety of bacteria the mechanisms of varying colony morphologies have been well described (van der Woude, Clinical Microbiology Reviews, 17:518, 2004), including in Clostridium species (Wadsworth-KTL Anaerobic Bacteriology Manual, 6th Ed, Jousimie-Somer, et al 2002). For obligate anaerobes, RCBs were confirmed to lack aerobic colony forming units at a limit of detection of 10 cfu/ml.

### Example 12: Titer Determination

The number of viable cells per ml was determined on the freshly harvested, washed and concentrated culture by plating serial dilutions of the RCB to Brucella blood agar or other solid media, and varied from 10⁶ to 10¹⁰ cfu/ml. The impact of freezing on viability was determined by titering the banks after one or two freeze-thaw cycles on dry ice or at −80° C., followed by thawing in an anaerobic chamber at room temperature. Some strains displayed a 1-3 log drop in viable cfu/ml after the 1st and/or 2nd freeze thaw, while the viability of others were unaffected.

### Example 13: Preparation of Bacterial Compositions

Individual strains were typically thawed on ice and combined in an anaerobic chamber to create mixtures, followed by a second freeze at −80° C. to preserve the mixed samples. When making combinations of strains for in vitro or in vivo assays, the cfu in the final mixture was estimated based on the second freeze-thaw titer of the individual strains. For experiments in rodents, strains may be combined at equal counts in order to deliver between 1e4 and 1e10 per strain. Additionally, some bacteria may not grow to sufficient titer to yield cell banks that allowed the production of compositions where all bacteria were present at 1e10.

### Example 14: Provision of Gut Microbiome Sample Material

Fresh gut microbiome samples e.g. fecal samples were obtained from healthy human donors who have been screened for general good health and for the absence of infectious diseases, and meet inclusion and exclusion criteria, inclusion criteria include being in good general health, without significant medical history, physical examination findings, or clinical laboratory abnormalities, regular bowel movements with stool appearance typically Type 2, 3, 4, 5 or 6 on the Bristol Stool Scale, and having a BMI ≥18 kg/m2 and ≤25 kg/m2. Exclusion criteria generally included significant chronic or acute medical conditions including renal, hepatic, pulmonary, gastrointestinal, cardiovascular, genitourinary, endocrine, immunologic, metabolic, neurologic or hematological disease, a family history of, inflammatory bowel disease including Crohn's disease and ulcerative colitis, Irritable bowel syndrome, colon, stomach or other gastrointestinal malignancies, or gastrointestinal polyposis syndromes, or recent use of yogurt or commercial probiotic materials in which an organism(s) is a primary component. Samples were collected directly using a commode specimen collection system, which contains a plastic support placed on the toilet seat and a collection container that rests on the support. Gut microbiome samples e.g. feces were deposited into the container, and the lid was then placed on the container and sealed tightly. The sample was then delivered on ice within 1-4 hours for processing. Samples were mixed with a sterile disposable tool, and 2-4 g aliquots were weighed and placed into tubes and flash frozen in a dry ice/ethanol bath. Aliquots are frozen at −80 degrees Celsius until use.

Optionally, the microbiome sample was suspended in a solution, and/or fibrous and/or particulate materials were removed. A frozen aliquot containing a known weight of sample was removed from storage at −80 degrees Celsius and allowed to thaw at room temperature. Sterile 1×PBS was added to create a 10% w/v suspension, and vigorous vortexing was performed to suspend the sample until the material appeared homogeneous. The sample was then left to sit for 10 minutes at room temperature to sediment fibrous and particulate matter. The suspension above the sediment was then carefully removed into a new tube and contains a purified spore population. Optionally, the suspension was then centrifuged at a low speed, e.g., 1000× g, for 5 minutes to pellet particulate matter including fibers. The pellet was discarded and the supernatant, which contained vegetative organisms and spores, was removed into a new tube. The supernatant was then centrifuged at 6000×g for 10 minutes to pellet the vegetative organisms and spores. The pellet was then resuspended in 1×PBS with vigorous vortexing until the sample material appears homogenous.

### Example 15: Bacterial Compositions Populating the Gut in a Mouse Model

Two bacterial compositions were evaluated in a mouse model to demonstrate the ability to populate the gastrointestinal tract. Bacteria were grown as described in Example 13. Compositions were pre-made under anaerobic conditions and suspended in PBS+15% glycerol and stored at ≥−70° C. prior to use.

Groups of mice (10 females/group; 5 per cage) were pre-treated on Days −14 to −5 with an antibiotic cocktail consisting of 10% glucose, kanamycin (0.5 mg/ml), gentamicin (0.044 mg/ml), colistin (1062.5 U/ml), metronidazole (0.269 mg/ml), ciprofloxacin (0.156 mg/ml), ampicillin (0.1 mg/ml) and vancomycin (0.056 mg/ml) in their drinking water. On Day −3 they received 10 mg/kg Clindamycin by oral gavage. On Day −1, they were dosed with a microbial compositions by oral gavage in a volume of 0.2 mL (Table 6). Microbial compositions comprised approximately equal numbers of each OTU and were dosed at approximately 1×10⁹, 1×10⁸ and 1×10⁷ per OTU for each composition (e.g. microbial composition 1, comprising 15 strains, was dosed at approximately 1.5×10¹⁰, 1.5×10⁹, and 1.5×10⁸ total CFU). Fecal samples were collected from each cage on Day −1 (approximately 1 hour before dosing) and on Days 2, 3 and 4 post-dosing. Feces were stored frozen prior to processing and sequencing. Weight gain of mice treated with either microbial compositions was similar to that of naive, control mice.

In parallel, groups of animals treated with the same microbial compositions on Day −1 were challenged on Day 0 with approximately 10⁴.5 spores of Clostridium difficile (ATCC 43255) via oral gavage. Mortality for C. difficile challenged animals was assessed every day from Day 0 to Day 6 and the weight and subsequent weight change of the animal was assessed with weight loss being associated with C. difficile infection. Mortality and reduced weight loss of the test article compared to the empty vehicle was used to assess the success of the test article.

Fecal samples were processed by isolating and sequencing DNA according to Examples 2 and 3. The OTU assignment of fecal samples from Days −1, 2, 3 and 4 was determined by analyzing 16S-V4 sequence reads and assigning OTUs as described in Example 2. Clades were assigned as described in Example 2. Total read counts were determined for each OTU or each clade by summing the results from cages of the same experimental group. Samples with 10 or fewer sequence reads for a given OTU or clade were considered to be below background and were not included in the summation process. Results are shown by OTU (Table 7) and by clade (Table 8).

Upon examining the OTU data in Table 7, several patterns emerge. First, there are a group of OTUs with no sequence reads on Day −1 that populate large numbers of sequence reads on Days 2, 3, or 4; this group includes Cl. butyricum, Cl. hylemonae, Cl. orbiscindens, Cl. symbiosum, and L. bacterium_5_1_57FAA. Cl. disporicum is comparable to this group as it has sequence reads on Day −1 that are very close to background (10 and 29 in compositions 1 and 2, respectively), which subsequently increase their population by as much as 1000-fold on Days 2, 3 or 4. Second, there are OTUs such as Co. aerofaciens, C. comes, R. bromii, B. producta, Cl. bolteae, Cl. mayombei, Cl. innocuum, Cl. tertium and R. gnavus which are not detectable at the OTU level in either the Day −1 sample or in subsequent samples. In composition 2. Co. aerofaciens is detected transiently on Day 2 in the 1×10⁸ and 1×10⁷ dose groups; E. rectale in the same experimental groups is detected on Day 3, suggesting a possible relationship between transient population by Co. aerofaciens followed by E. rectale in these groups of mice. A striking observation is that the observed population of OTU sequence reads is not highly dose dependent. Overall, the data is consistent with a model whereby OTUs populate rapidly following oral administration.

The clade-based analysis in Table 8 was performed to more thoroughly evaluate the population of the GI tract. Clade-based analysis obscures some of the details afforded by an OTU analysis. For instance, Cl. tertium and Cl. butyricum are members of the same clade and thus a clade-based analysis cannot distinguish the dynamics of these individual OTUs. However, clade-based analysis has the compensatory benefit that it is sensitive to measuring population changes that can be missed by an OTU-based analysis. The ability of 16S-V4 OTU identification to assign an OTU as a specific species depends in part on the resolving power of the 16S-V4 region for a particular species or group of species. Both the density of available reference 16S sequences for different regions of the tree as well as the inherent variability in the 16S gene between different species will determine the definitiveness of a taxonomic annotation. So in some cases, the population of a species can be followed using clade-based assignments when OTU based-detection is insensitive in a complex population. For instance, the clade-based analysis in Table 2B supports the case that R. bromii, B. producta, Cl. innocuum, and R. gnavus were able to populate since each OTU is a sole member of a clade in the microbial compositions and sequence reads went from undetectable on Day −1 to well above background on Days 2, 3 or 4. 16S V4 sequencing and clade-based analysis could not determine whether Cl. tertium or Cl. bolteae populated due to the fact that other members of their clades (Cl. butyricum and Cl. symbiosum, respectively) were present and shown to populate at the OTU level in the mice.

In the mice challenged in parallel with C. difficile, animals were significantly protected as shown in Table 9. Mice gavaged with vehicle (phosphate buffered saline) experienced 100% mortality while microbial compositions 1 and 2 protected at all dose levels with between 0 and 10% mortality by Day 6, the last day of the experiment. In addition, weight loss in animals treated with microbial compositions 1 and 2 was minimal compared to animals receiving the vehicle gavage. These data confirm that population of the gastrointestinal tract with microbial compositions confers a clinical benefit by restoring a state of dysbiosis so that animals can resist infection by a pathogen.

### Example 16: Quantification of Spore Concentrations Using DPA Assay

Methods to assess spore concentration in complex mixtures typically require the separation and selection of spores and subsequent growth of individual species to determine the colony forming units. The art does not teach how to quantitatively germinate all the spores in a complex mixture as there are many species for which appropriate germinants have not been identified. Furthermore, sporulation is thought to be a stochastic process as a result of evolutionary selection, meaning that not all spores from a single species germinate with same response to germinant concentration, time and other environmental conditions. Alternatively, a key metabolite of bacterial spores, dipicolinic acid (DPA) has been developed to quantify spores particles in a sample and avoid interference from fecal contaminants. The assay utilizes the fact that DPA chelates Terbium 3+ to form a luminescent complex (Fichtel et al, FEMS Microbiology Ecology, 2007; Kort et al, Applied and Environmental Microbiology, 2005; Shafaat and Ponce, Applied and Environmental Microbiology, 2006; Yang and Ponce, International Journal of Food Microbiology, 2009; Hindle and Hall, Analyst, 1999). A time-resolved fluorescence assay detects terbium luminescence in the presence of DPA giving a quantitative measurement of DPA concentration in a solution.

To perform the assay 1 mL of the spore standard to be measured was transferred to a 2 mL microcentrifuge tube. The samples were centrifuged at 13000 RCF for 10 min and the sample is washed in 1 mL sterile deionized H₂O. Wash an additional time by repeating the centrifugation. Transfer the 1 mL solution to hungate tubes and autoclave samples on a steam cycle for 30 min at 250 C. Add 100 μL of 30 uM TbCl₃ solution (400 mM sodium acetate, pH 5.0, 30 μM TbCl₃) to the sample. Make serial dilutions of the autoclaved material and measure the fluorescence of each sample by exciting with 275 nm light and measuring the emission wavelength of 543 nm for an integration time of 1.25 ms and a 0.1 ms delay.

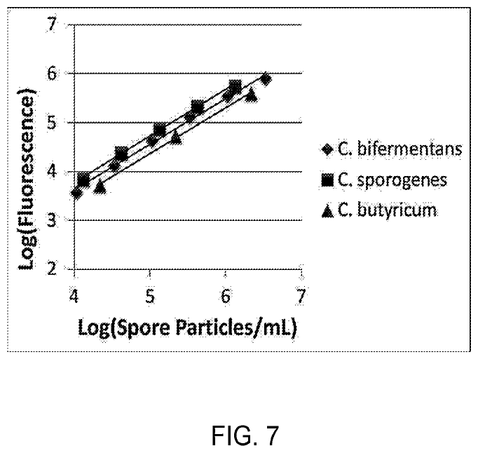

Purified spores are produced as described previously (e.g. see www.epa.gov/pesticides/methods/MB-28-00.pdf). Serial dilutions of purified spores from C. bifermentans, C. sporogenes, and C. butyricum cultures were prepared and measured by plating on BBA media and incubating overnight at 37° C. to determine CFU/ml. FIG. 7 shows the linear correspondence across different spore producing bacteria across several logs demonstrating the DPA assay as means to assess spore content (linear range of DPA assay compared to CFU counts/ml).

The discrepancy for complex spore populations between spore counts measured by germinable spore CFU and by DPA has important implications for determining the potency of an ethanol treated spore preparation for clinical use. Table 10 below shows spore content data from 3 different ethanol treated spore preparations used to successfully treat 3 patients suffering from recurrent C. difficile infection. The spore content of each spore preparation is characterized using the two described methods.

What is immediately apparent is that spore content varies greatly per 30 capsules. As measured by germinable SCFU, spore content varies by greater than 10,000-fold. As measured by DPA, spore content varies by greater than 100-fold. In the absence of the DPA assay, it would be difficult to set a minimum dose for administration to a patient. For instance, without data from the DPA assay, one would conclude that a minimum effective dose of spores is 4×10⁵ or less using the SCFU assay (e.g. Preparation 1, See Table 10 below). If that SCFU dose was used to normalize dosing in a clinical setting, however, then the actual spore doses given to patients would be much lower for other ethanol treated spore preparations as measured as by the DPA assay (See Table 11 below).

It becomes immediately obvious from the variability of SCFU and DPA counts across various donations that using SCFU as the measure of potency would lead to significant underdosing in certain cases. For instance, setting a dose specification of 4×10⁵ SCFU (the apparent effective dose from donor Preparation 1) for product Preparation 3 would lead to a potential underdosing of more than 100-fold. This can be rectified only by setting potency specifications based on the DPA assay which better reflects total spore counts in an ethanol treated spore preparation. The unexpected finding of this work is that the DPA assay is uniquely suited to set potency and determine dosing for an ethanol treated spore preparation.

### Example 17: Demonstration of Enhanced Growth with a Germinant

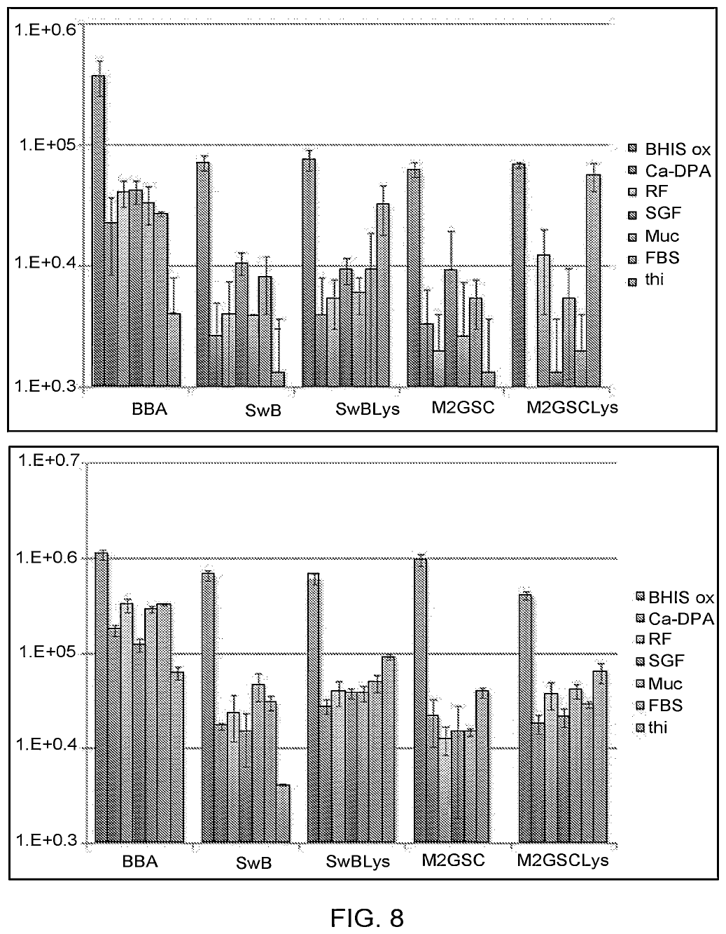

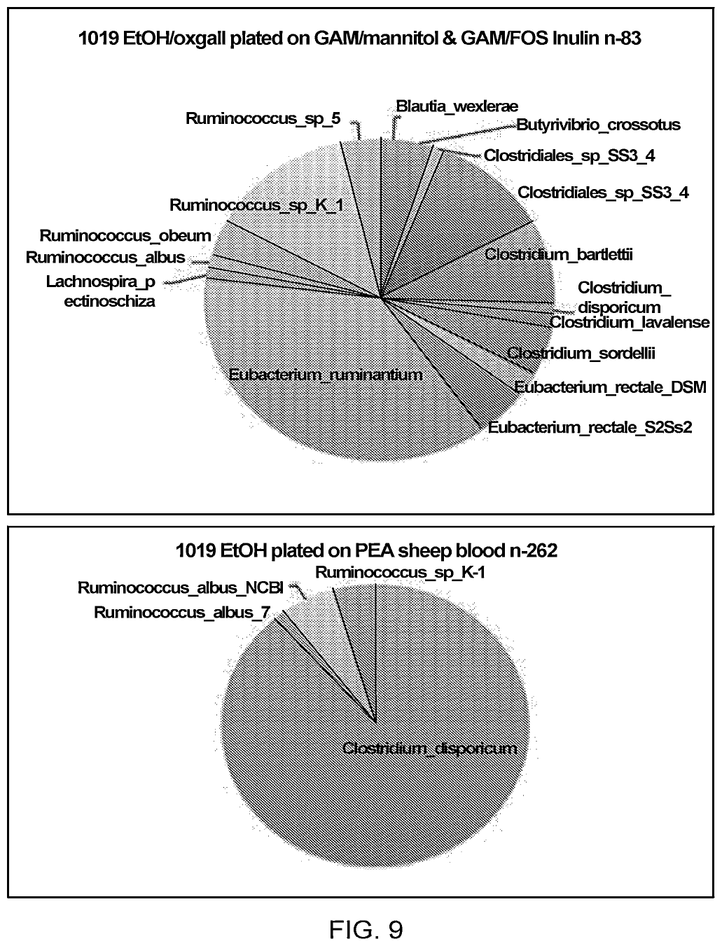

To enhance the ethanol treated spores germination capability and demonstrate spore viability, spores from three different donors were germinated by various treatments and plated on various media. Germination with BHIS/oxgall (BHIS ox), Ca-DPA, rumen fluid (RF), simulated gastric fluid (SGF), mucin medium (Muc), fetal bovine serum (FBS), or thioglycollate (Thi) for 1 hour at 37 C in anaerobic conditions was performed as described previously (e.g. see Examples 4 and 5) with samples derived from two independent donors (FIG. 8). The spore-germinant mixture was serially diluted and plated on different plate media including BBA, Sweet B, Sweet B+lysozyme (2 ug/ml), M2GSC and M2GSC+lysozyme (2 ug/ml) as previously described (e.g. see Examples 4 and 5) to determine spore germination. Colony forming units were tallied and titers were determined using standard techniques by one skilled in the art. As FIG. 8 shows, maximum colony forming units are derived from BHI-oxgall treatment. This germination treatment also greatly increases the diversity as measured by the number of OTUs identified when samples were submitted for 16S sequencing (e.g. see Examples 2 and 3) compared to plating without a germination step (FIG. 9).

FIG. 8 shows ifferent germinant treatments have variable effects on CFU counts from donor A (upper left) and donor B (lower right). The Y-Axes are spore CFU per ml.

FIG. 9 shows germinates greatly increase the diversity of cultured spore forming OTUs.

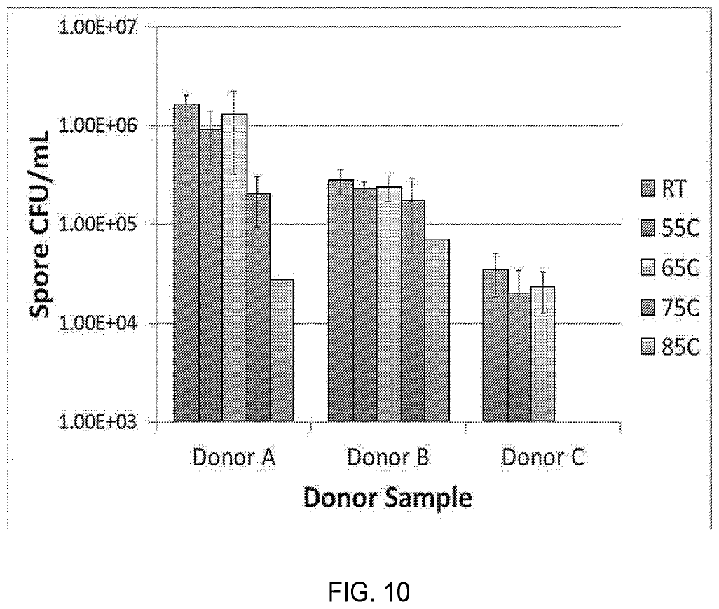

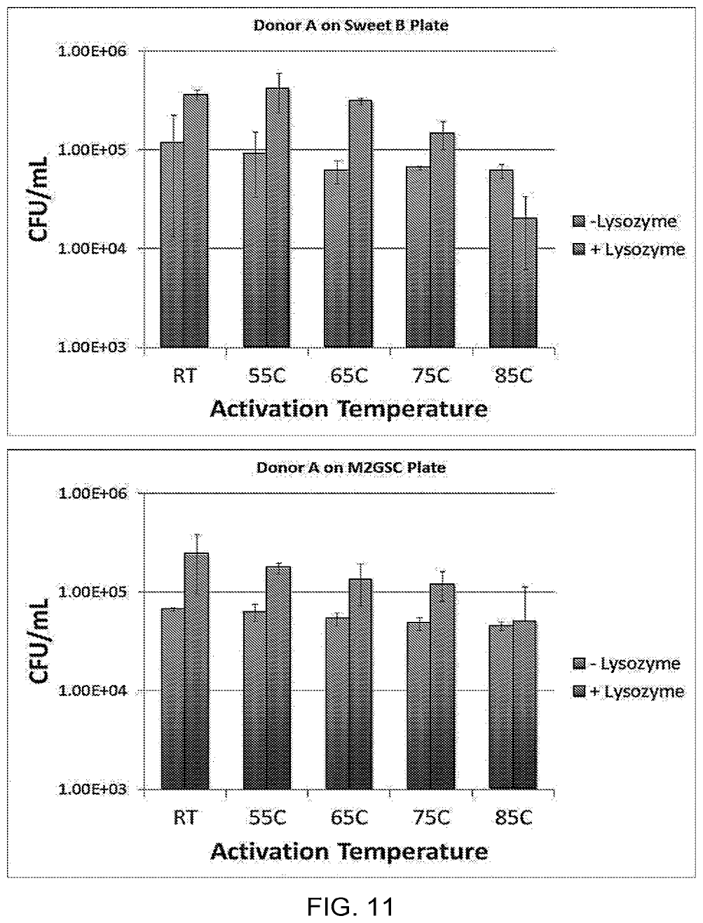

To test the effect of heat activation to promote germination, ethanol treated fecal samples were treated for 15 min at room temperature, 55° C., 65° C., 75° C. or 85° C. from three different donors and germinated subsequently with BHIS+Oxgall for 1 hr at 37° C. then plated on BBA media (FIG. 10) as previously described (e.g. see Examples 4 and 5). Pretreatment at room temperature produced equal if not more spores than the elevated temperatures in all three donors. The temperature of germinating was also examined by incubating samples at room temperature or 37° C. for 1 hr in anaerobic conditions before plating on BBA. No difference in the number of CFUs was observed between the two conditions. Lysozyme addition to the plates (2 ug/ml) was also tested on a single donor sample by the testing of various activation temperature followed by an incubation in the presence or absence of lysozyme. The addition of lysozyme had a small effect when plated on Sweet B or M2GSC media but less so than treatment with BHIS oxgall without lysozyme for 1 hr (FIG. 11).

FIG. 10 shows heat activation as a germination treatment with BHIS+oxgall.

FIG. 11 shows the effect of lysozyme slightly enhances germination

Germination time was also tested by treating a 10% suspension of a single donor ethanol treated feces (e.g. see Example AAAF) incubated in either BHIS, taurocholate, oxgall, or germix for 0, 15, 30, or 60 minutes and subsequently plated on BHIS, EYA, or BBA media (e.g. see Examples AAAD and AAAE). 60 minutes resulted in the most CFU units across all various combinations germinates and plate media tested. Furthermore, this protocol and germination treatment can be applied to mixtures of bacteria and spore forming species to enrich the population of spore-forming bacterial growth. In this manner, spore formers growth can be enhanced in a mixed population by addition of a germinate or germination treatment step as described above.

### Example 18: Construction of an In Vitro Assay to Screen for Combinations of Microbes Inhibitory to the Growth of Clostridium difficile

The in vitro assay is a high-throughput assay designed to screen for combinations of microbes that inhibit the growth of a pathogen or pathobiont. Feces from a human donor is resuspended 10% w/v in pre-reduced PBS under anaerobic conditions and then is pelleted in an Eppendorf Centrifuge 5810R, Rotor F34-6-38 at 8,500 rpm (9289 rcf) for 5 min to remove cells and particulates. The supernatant is then filter-sterilized using a 0.22 μm filter to produce the background media for in vitro competitions. The 10% sterile-filtered feces media is then added in 178 μL aliquots to wells of a 96-well plate. 20 μL of candidate bacterial strains or combinations of strains are added to each well. The in vitro assay plate is then incubated anaerobically at 37° C. for 3 hr. before inoculation of Clostridium difficile ATCC 9689. The C. difficile inoculum is prepared by inoculating 5 mL BHI (Brain Heart Infusion Broth) and incubating 37° C. for 18 hr. The overnight culture is then diluted 100 fold in PBS before inoculating 2 μL into each well to produce a final dilution of 10,000 fold from the overnight culture. In vitro assay plates are then incubated anaerobically for 24 hr. before assaying for viable counts of C. difficile by plating serial dilutions on C. difficile selective agar plates with cycloserine cefoxitin mannitol agar (CCMA).

Colonies of C. difficile are confirmed by their characteristic diffuse colony edge morphology as well as fluorescence under UV light.

### Example 19: Quantification of Pathogen Using Quantitative PCR (qPCR

### Standard Curve Preparation

The standard curve is generated from wells containing pathogenic C. difficile grown in 10% sterile-filtered feces media from Example 2 by selective spot plating. Serial dilutions of duplicate cultures are performed in sterile phosphate-buffered saline. Genomic DNA is then extracted from the standard curve samples along with the other wells.

### Genomic DNA Extraction

Genomic DNA can be extracted from 50 μl of each sample using the Mo Bio Powersoil®-htp 96 Well Soil DNA Isolation Kit (Mo Bio Laboratories, Carlsbad, CA) according to the manufacturer's instructions with two exceptions: the bead beating is performed for 2×4:40 minutes using a BioSpec Mini-Beadbeater-96 (BioSpec Products, Bartlesville, OK) and the DNA is eluted in 50 μl of Solution C6. Alternatively the genomic DNA could be isolated using the Mo Bio Powersoil® DNA Isolation Kit (Mo Bio Laboratories, Carlsbad, CA) or the QIAamp DNA Stool Mini Kit (QIAGEN, Valencia, CA) according to the manufacturer's instructions.

qPCR Composition and Conditions

The qPCR contains 1× HotMasterMix (5PRIME, Gaithersburg, MD), 900 nM of Wr-tcdB-F (AGCAGTTGAATATAGTGGTTTAGTTAGAGTTG (SEQ ID NO: 2040), IDT, Coralville, IA), 900 nM of Wr-tcdB-R (CATGCTTTTTTAGTTTCTGGATTGAA, (SEQ ID NO: 2041) IDT, Coralville, IA), 250 nM of We-tcdB-P (6FAM-CATCCAGTCTCAATTGTATATGTTTCTCCA-MGB (SEQ ID NO: 2042), Life Technologies, Grand Island, NY), and PCR Water (Mo Bio Laboratories, Carlsbad, CA) to 18 μl (Primers adapted from: Wroblewski, D. et al, Rapid Molecular Characterization of Clostridium difficile and Assessment of Populations of C. difficile in Stool Specimens, Journal of Clinical Microbiology 47:2142-2148 (2009)). This reaction mixture is aliquoted to wells of a MicroAmp® Fast Optical 96-well Reaction Plate with Barcode (0.1 mL) (Life Technologies, Grand Island, NY). To this reaction mixture, 2 μl of extracted genomic DNA is added. The qPCR is performed on a BioRad C1000™ Thermal Cycler equipped with a CFX96™ Real-Time System (BioRad, Hercules, CA). The thermocycling conditions are 95° C. for 2 minutes followed by 45 cycles of 95° C. for 3 seconds, 60° C. for 30 seconds, and fluorescent readings of the FAM and ROX channels.

### Data Analysis

The Cq value for each well on the FAM channel is determined by the CFX Manager™ software version 2.1. The log₁₀ (cfu/ml) of each experimental sample is calculated by inputting a given sample's Cq value into linear regression model generated from the standard curve comparing the Cq values of the standard curve wells to the known log₁₀ (cfu/ml) of those samples.

### Example 20. Construction of an In Vitro Assay to Screen for Combinations of Microbes Inhibitory to the Growth of Pathogenic E. coli

The in vitro assay is used to screen for combinations of bacteria inhibitory to the growth of E. coli by modifying the media used for growth of the pathogen inoculum. One of several choices of media is used for growth of the pathogen such as Reinforced Clostridial Media (RCM), Brain Heart Infusion Broth (BHI) or Luria Bertani Broth (LB) (also known as Lysogeny Broth). E. coli is quantified by using alternative selective media specific for E. coli or using qPCR probes specific for the pathogen. For example, aerobic growth on MacConkey lactose medium selects for enteric Gram negatives, including E. coli. qPCR is conducted using probes specific for the shiga toxin of pathogenic E. coli.

### Example 21. Construction of an In Vitro Assay to Screen for Combinations of Microbes Inhibitory to the Growth of Vancomycin-Resistant Enterococcus (VRE)

The in vitro assay is used to screen for combinations of bacteria inhibitory to the growth of Vancomycin-Resistant Enterococcus spp. (VRE) by modifying the media used for growth of the pathogen inoculum. Several choices of media are used for growth of the pathogen such as Reinforced Clostridial Media (RCM), Brain Heart Infusion Broth (BHI) or Luria Bertani Broth (LB). VRE is quantified by using alternative selective media specific for VRE or using qPCR probes specific for the pathogen. For example, m-Enterococcus agar containing sodium azide is selective for Enterococcus spp. and a small number of other species. Probes specific to the van genes conferring vancomycin resistance are used in the qPCR.

### Example 22: Prophylactic Use and Treatment in a Mouse Model of Vancomycin Resistant Enterococcus (VRE) Colonization

The emergence and spread of highly antibiotic-resistant bacteria represent a major clinical challenge (Snitkin et al Science Translational Medicine, 2012). In recent years, the numbers of infections caused by organisms such as methicillin-resistant Staphylococcus aureus, carbapenem-resistant Enterobacteriaceae, vancomycin-resistant Enterococcus (VRE), and Clostridium difficile have increased markedly, and many of these strains are acquiring resistance to the few remaining active antibiotics. Most infections produced by highly antibiotic-resistant bacteria are acquired during hospitalizations, and preventing patient-to-patient transmission of these pathogens is one of the major challenges confronting hospitals and clinics. Most highly antibiotic-resistant bacterial strains belong to genera that colonize mucosal surfaces, usually at low densities. The highly complex microbiota that normally colonizes mucosal surfaces inhibits expansion of and domination by bacteria such as Enterobacteriaceae and Enterococcaceae. Destruction of the normal flora by antibiotic administration, however, disinhibition antibiotic-resistant members of these bacterial families, leading to their expansion to very high densities (Ubeda et al Journal of Clinical Investigation 2010). High-density colonization by these organisms can be calamitous for the susceptible patient, resulting in bacteremia and sepsis (Taur et al, Clinical Infectious Disease, 2012).

To test prophylactic use and treatment of a bacterial composition test article, a VRE infection mouse model is used as previously described (Ubeda et al, Infectious Immunity 2013, Ubeda et al, Journal of clinical investigation, 2010). Briefly, experiments are done with 7-week-old C57BL/6J female mice purchased from Jackson Laboratory, housed with irradiated food, and provided with acidified water. Mice are individually housed to avoid contamination between mice due to coprophagia. For experimental infections with VRE, mice are treated with ampicillin (0.5 g/liter) in their drinking water, which is changed every 3 days.

In the treatment model, on day 1, mice are infected by means of oral gavage with 10⁸ CFU of the vancomycin-resistant Enterococcus faecium strain purchased from ATCC (ATCC 700221). One day after infection (day 1), antibiotic treatment is stopped and VRE levels are determined at different time points by plating serial dilutions of fecal pellets on Enterococcosel agar plates (Difco) with vancomycin (8 ug/ml; Sigma). VRE colonies are identified by appearance and confirmed by Gram staining or other methods previously described (e.g. see Examples 1-3). In addition, as previously described (Ubeda et al, Journal of Clinical Investigation 2010), PCR of the vanA gene, which confers resistance to vancomycin, confirms the presence of VRE in infected mice. The bacterial composition test article such as but not limited to an ethanol treated, gradient purified spore preparation (as described herein), fecal suspension, or antibiotic treatment is delivered in PBS on days 1-3 while the negative control contains only PBS and is also delivered on days 1-3 by oral gavage. Fresh fecal stool pellets are obtained daily for the duration of the experiment from days −7 to day 10. The samples are immediately frozen and stored at −80° C. DNA was extracted using standard techniques and analyzed with 16S or comparable methods (e.g. see Examples 2 and 3).

In the colonization model, ampicillin is administered as described above for day −7 to day 1, treatment with the test article or vehicle control is administered on day 0-2 and the VRE resistant bacteria at 10⁸ CFU are administered on day 14. Fecal samples are taken throughout the experiment daily from −7 to day 21 and submitted for 16S sequencing as previously described (e.g. see Examples 2 and 3).

In both models titers of VRE in feces are used to evaluate the success of the test article versus the negative control. Furthermore, microbiota composition is assessed for the ability of the bacterial composition test article to induce a healthy microbiome.

### Example 23: Prophylactic Use and Treatment of a Mouse Model of Carbapenem Resistant Klebsiella (CRKB) Colonization

The emergence of Klebsiella pneumoniae strains with decreased susceptibility to carbapenems is a significant threat to hospitalized patients. Resistance to carbapenems in these organisms is most frequently mediated by K. pneumoniae carbapenemase (KPC), a class A beta-lactamase that also confers resistance to broad-spectrum cephalosporins and commercially available beta-lactam/beta-lactamase inhibitor combinations (Queenan et al, Clinical Microbiology Review, 2007). KPC-producing K. pneumoniae (KPC-Kp) strains often harbor resistance determinants against several other classes of antimicrobials, including aminoglycosides and fluoroquinolones, resulting in truly multidrug-resistant (MDR) organisms (Hirsch et al, Journal of Antimicrobial Chemotherapy, 2009). Considering the limited antimicrobial options, infections caused by KPC-Kp pose a tremendous therapeutic challenge and are associated with poor clinical outcomes

A treatment protocol in a mouse model as previously described (e.g. Perez et al, Antimicrobial Agents Chemotherapy, 2011) is used to evaluate the bacterial composition (test article) for treating carbapenem resistant Klebsiella and reducing carriage in the GI tract. Female CF1 mice (Harlan Sprague-Dawley, Indianapolis, IN) are used and are individually housed and weighed between 25 and 30 g.

The thoroughly characterized strain of K. pneumoniae, VA-367 (8, 9, 25) is used in this study. This clinical isolate is genetically related to the KPC-Kp strain circulating in the Eastern United States. Characterization of the resistance mechanisms in K. pneumoniae VA-367 with PCR and DNA sequence analysis revealed the presence of bla_{KPC-3}, bla_{TEM-1}, blas_{HV-11}, and blas_{HV-12} as well as qnrB19 and aac (6′)-lb. Additionally, PCR and DNA sequencing revealed disruptions in the coding sequences of the following outer membrane protein genes: ompK35, ompK36, and ompK37. Antibiotic susceptibility testing (AST) was performed with the agar dilution method and interpreted according to current recommendations from the Clinical and Laboratory Standards Institute (CLSI). A modified Hodge test were performed, according to a method described previously (e.g. see Anderson et al, Journal of Clinical Microbiology, 2007) with ertapenem, meropenem, and imipenem. Tigecycline and polymyxin E were evaluated by Etest susceptibility assays (AB bioM′erieux, Solna, Sweden). Results for tigecycline were interpreted as suggested by the U.S. Food and Drug Administration (FDA) and according to CLSI recommendations (criteria for Pseudomonas) for polymyxin E.

Mice (10 per group) are assigned to either a bacterial composition (test article), ethanol treated, spore preparation (e.g. see Example 6), antibiotic clindamycin, piperacillin-tazobactam, tigecycline, ertapenem, cefepime, ciprofloxacin, or combination thereof or control group receiving only the vehicle. They are administered the test article daily from day −10 to day 0, On day 0, 10³ CFU of KPC-Kp VA-367 diluted in 0.5 ml phosphate-buffered saline (PBS) was administered by oral gavage using a stainless-steel feeding tube (Perfektum; Popper & Sons, New Hyde Park, NY). Stool samples were collected 1, 4, 6, and 11 days after the administration of KPC-Kp in order to measure the concentration of carbapenem-resistant K. pneumoniae. Stool samples (100 mg diluted in 800 ml of PBS) are plated onto MacConkey agar with and without 0.5 ug/ml of imipenem, and the number of CFU per gram of stool was determined. Alternatively other methods may be used to measure the levels of carbapenem-resistant K. pneumoniae e.g. pcr, antigen testing, as one who's skilled in the art could perform.

Stool samples were collected after 5 days of treatment to assess the effects of the antibiotics on the stool microflora and to measure antibiotic levels in stool. To assess the effects on the microflora, fresh stool samples as previously described (e.g. see Examples 2 and 3). Additional experiments are performed to examine whether the administration the bacterial composition (test article) resulted in the elimination or persistence of colonization with KPC-Kp VA-367.

Mice are treated with subcutaneous clindamycin to reduce the normal intestinal flora 1 day before receiving 10⁴ CFU of KPC-Kp VA-367 by oral gavage, and the mice continued to receive subcutaneous clindamycin every other day for 7 days. Concurrently, for 7 days after oral gavage with KPC-Kp, mice received oral gavage of normal saline (control group), or the bacterial composition as specified. An additional dose of subcutaneous clindamycin was administered 20 days after the administration of KPC-Kp VA-367 to assess whether low levels of carbapenem-resistant K. pneumoniae were present that could be augmented by the elimination of the anaerobic microflora. Stool samples were collected at baseline and at 3, 6, 8, 11, 16, and 21 days after KPC-Kp VA-367 was given by gavage. The bacterial composition will be examined by the reduction of CRKB in feces.

### Example 24: Identification of Keystone OTUs and Functions

The human body is an ecosystem in which the microbiota, and the microbiome, play a significant role in the basic healthy function of human systems (e.g. metabolic, immunological, and neurological). The microbiota and resulting microbiome comprise an ecology of microorganisms that co-exist within single subjects interacting with one another and their host (i.e., the mammalian subject) to form a dynamic unit with inherent biodiversity and functional characteristics. Within these networks of interacting microbes (i.e. ecologies), particular members can contribute more significantly than others; as such these members are also found in many different ecologies, and the loss of these microbes from the ecology can have a significant impact on the functional capabilities of the specific ecology. Robert Paine coined the concept “Keystone Species” in 1969 (see Paine RT. 1969. A note on trophic complexity and community stability. The American Naturalist 103:91-93) to describe the existence of such lynchpin species that are integral to a given ecosystem regardless of their abundance in the ecological community. Paine originally describe the role of the starfish Pisaster ochraceus in marine systems and since the concept has been experimentally validated in numerous ecosystems.

Keystone OTUs and/or Functions are computationally-derived by analysis of network ecologies elucidated from a defined set of samples that share a specific phenotype. Keystone OTUs and/or Functions are defined as all Nodes within a defined set of networks that meet two or more of the following criteria. Using Criterion 1, the node is frequently observed in networks, and the networks in which the node is observed are found in a large number of individual subjects; the frequency of occurrence of these Nodes in networks and the pervasiveness of the networks in individuals indicates these Nodes perform an important biological function in many individuals. Using Criterion 2, the node is frequently observed in networks, and each the networks in which the node is observed contain a large number of Nodes—these Nodes are thus “super-connectors”, meaning that they form a nucleus of a majority of networks and as such have high biological significance with respect to their functional contributions to a given ecology. Using Criterion 3, the node is found in networks containing a large number of Nodes (i.e. they are large networks), and the networks in which the node is found occur in a large number of subjects; these networks are potentially of high interest as it is unlikely that large networks occurring in many individuals would occur by chance alone strongly suggesting biological relevance. Optionally, the required thresholds for the frequency at which a node is observed in network ecologies, the frequency at which a given network is observed across subject samples, and the size of a given network to be considered a Keystone node are defined by the 50th, 70th, 80th, or 90th percentiles of the distribution of these variables. Optionally, the required thresholds are defined by the value for a given variable that is significantly different from the mean or median value for a given variable using standard parametric or non-parametric measures of statistical significance. In another embodiment a Keystone node is defined as one that occurs in a sample phenotype of interest such as but not limited to “health” and simultaneously does not occur in a sample phenotype that is not of interest such as but not limited to “disease.” Optionally, a Keystone Node is defined as one that is shown to be significantly different from what is observed using permuted test datasets to measure significance.

### Example 25: Microbial Population (Engraftment and Augmentation) and Reduction of Pathogen Carriage in Patients Treated with Spore Compositions

Complementary genomic and microbiological methods were used to characterize the composition of the microbiota from Patient 1, 2, 3, 4, and 5, 6, 7, 8, 9, and 10 at pretreatment (pretreatment) and on up to 4 weeks post-treatment.

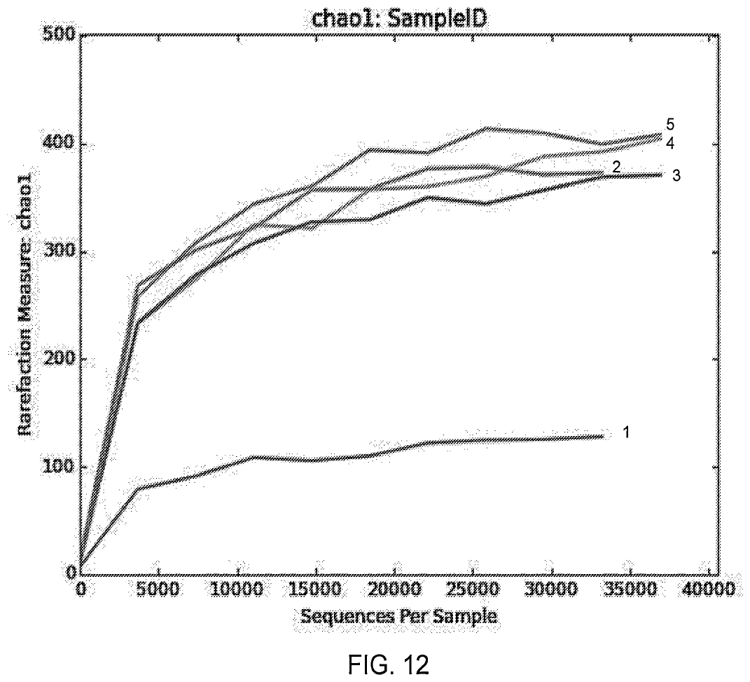

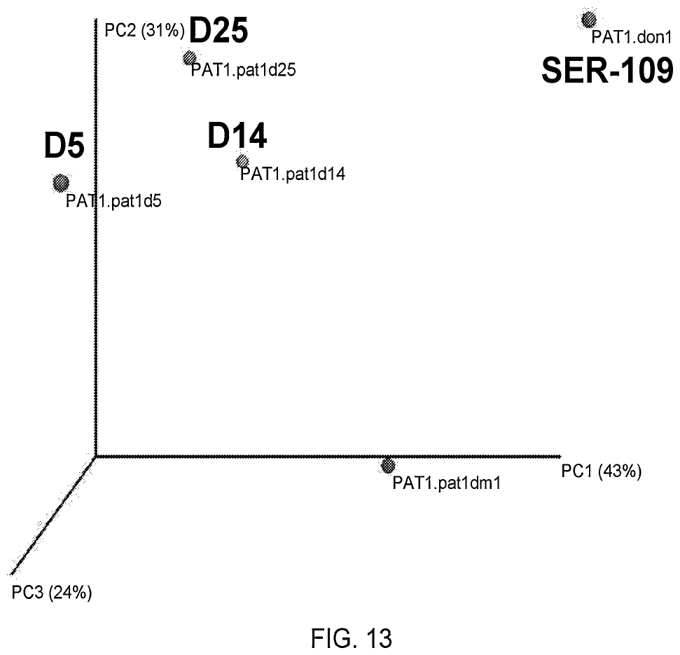

To determine the OTUs that engraft from treatment with an ethanol treated spore preparation in the patients and how their microbiome changed in response, the microbiome was characterized by 16S-V4 sequencing prior to treatment (pretreatment) with an ethanol treated spore preparation and up to 25 days after receiving treatment. Alternatively, one might use a bacterial composition in the vegetative state, or a mixture of vegetative bacteria and bacterial spores. As example, the treatment of patient 1 with an ethanol treated spore preparation led to microbial population via the engraftment of OTUs from the spore treatment and augmentation in the microbiome of the patient (FIG. 12 and FIG. 13). By day 25 following treatment, the total microbial carriage was dominated by species of the following taxonomic groups: Bacteroides, Sutterella, Ruminococcus, Blautia, Eubacterium, Gemmiger/Faecalibacterium, and the non-sporeforming Lactobacillus (see Tables 2 and 12 for specific OTUs). The first two genera represent OTUs that do not form spores while the latter taxonomic groups represent OTUs that are believed to form spores.

Table 12 shows bacterial OTUs associated with engraftment and ecological augmentation and establishment of a more diverse microbial ecology in patients treated with an ethanol treated spore preparation. OTUs that comprise an augmented ecology are not present in the patient prior to treatment and/or exist at extremely low frequencies such that they do not comprise a significant fraction of the total microbial carriage and are not detectable by genomic and/or microbiological assay methods. OTUs that are members of the engrafting and augmented ecologies were identified by characterizing the OTUs that increase in their relative abundance post treatment and that respectively are: (i) present in the ethanol treated spore preparation and absent in the patient pretreatment (engrafting OTUs), or (ii) absent in the ethanol treated spore preparation, but increase in their relative abundance through time post treatment with the preparation due to the formation of favorable growth conditions by the treatment (augmenting OTUs). Notably, the latter OTUs can grow from low frequency reservoirs in the patient, or be introduced from exogenous sources such as diet. OTUs that comprise a “core” augmented or engrafted ecology can be defined by the percentage of total patients in which they are observed to engraft and/or augment; the greater this percentage the more likely they are to be part of a core ecology responsible for catalyzing a shift away from a dysbiotic ecology. The dominant OTUs in an ecology can be identified using several methods including but not limited to defining the OTUs that have the greatest relative abundance in either the augmented or engrafted ecologies and defining a total relative abundance threshold. As example, the dominant OTUs in the augmented ecology of Patient-1 were identified by defining the OTUs with the greatest relative abundance, which together comprise 60% of the microbial carriage in this patient's augmented ecology.

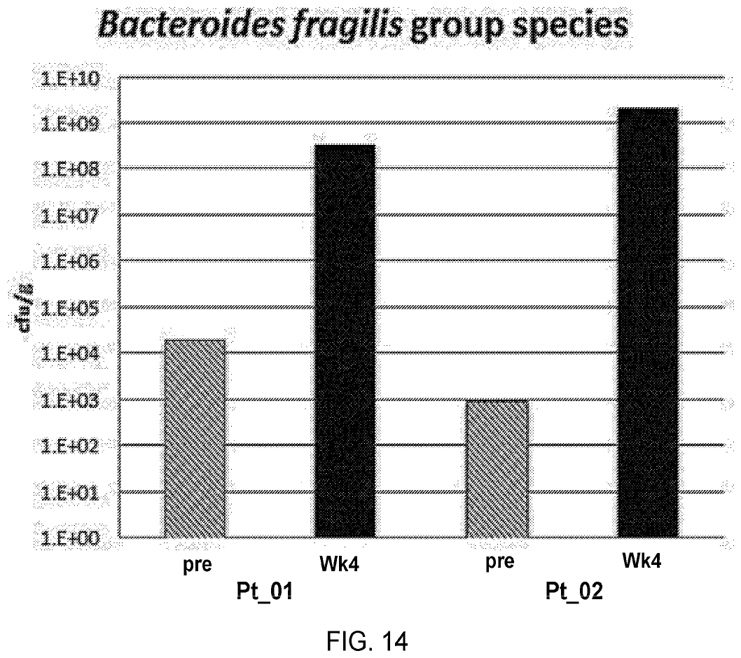

Patient treatment with the ethanol treated spore preparation leads to the population of a microbial ecology that has greater diversity than prior to treatment (FIG. 12). Genomic-based microbiome characterization confirmed engraftment of a range of OTUs that were absent in the patient pretreatment (Table 12). These OTUs comprised both bacterial species that were capable and not capable of forming spores, and OTUs that represent multiple phylogenetic clades. Organisms absent in Patient 1 pre-treatment either engraft directly from the ethanol treated spore fraction or are augmented by the creation of a gut environment favoring a healthy, diverse microbiota. Furthermore, Bacteroides fragilis group species were increased by 4 and 6 logs in patients 1 and 2 (FIG. 14).

FIG. 12 shows the microbial diversity measured in the ethanol treated spore treatment sample and patient pre- and post-treatment samples. Total microbial diversity is defined using the Chao1 Alpha-Diversity Index and is measured at different genomic sampling depths to confirm adequate sequence coverage to assay the microbiome in the target samples. The patient pretreatment (purple) harbored a microbiome that was significantly reduced in total diversity as compared to the ethanol treated spore treatment (red) and patient post treatment at days 5 (blue), 14 (orange), and 25 (green).

FIG. 13 shows patient microbial ecology is shifted by treatment with an ethanol treated spore treatment from a dysbiotic state to a state of health. Principle Coordinates Analysis based on the total diversity and structure of the microbiome (Bray-Curtis Beta-Diversity) of the patient pre- and post-treatment delineates that the engraftment of OTUs from the spore treatment and the augmentation of the patient microbial ecology leads to a microbial ecology that is distinct from both the pretreatment microbiome and the ecology of the ethanol treated spore treatment (Table 12).

FIG. 14 shows the augmentation of Bacteroides species in patients. Comparing the number of Bacteroides fragilis groups species per cfu/g of feces pre-treatment and in week 4 post treatment reveals an increase of 4 logs or greater. The ability of 16S-V4 OTU identification to assign an OTU as a specific species depends in part on the resolution of the 16S-V4 region of the 16S gene for a particular species or group of species. Both the density of available reference 16S sequences for different regions of the tree as well as the inherent variability in the 16S gene between different species will determine the definitiveness of a taxonomic annotation to a given sequence read. Given the topological nature of a phylogenetic tree and that the tree represents hierarchical relationships of OTUs to one another based on their sequence similarity and an underlying evolutionary model, taxonomic annotations of a read can be rolled up to a higher level using a clade-based assignment procedure (Table 1). Using this approach, clades are defined based on the topology of a phylogenetic tree that is constructed from full-length 16S sequences using maximum likelihood or other phylogenetic models familiar to individuals with ordinary skill in the art of phylogenetics. Clades are constructed to ensure that all OTUs in a given clade are: (i) within a specified number of bootstrap supported nodes from one another (generally, 1-5 bootstraps), and (ii) within a 5% genetic similarity. OTUs that are within the same clade can be distinguished as genetically and phylogenetically distinct from OTUs in a different clade based on 16S-V4 sequence data. OTUs falling within the same clade are evolutionarily closely related and may or may not be distinguishable from one another using 16S-V4 sequence data. The power of clade based analysis is that members of the same clade, due to their evolutionary relatedness, play similar functional roles in a microbial ecology such as that found in the human gut. Compositions substituting one species with another from the same clade are likely to have conserved ecological function and therefore are useful in the present invention.

Stool samples were aliquoted and resuspended 10× vol/wt in either 100% ethanol (for genomic characterization) or PBS containing 15% glycerol (for isolation of microbes) and then stored at −80° C. until needed for use. For genomic 16S sequence analysis colonies picked from plate isolates had their full-length 16S sequence characterized as described in Examples 2 and 3, and primary stool samples were prepared targeting the 16S-V4 region using the method for heterogeneous samples described herein.

Notably, 16S sequences of isolates of a given OTU are phylogenetically placed within their respective clades despite that the actual taxonomic assignment of species and genus may suggest they are taxonomically distinct from other members of the clades in which they fall. Discrepancies between taxonomic names given to an OTU is based on microbiological characteristics versus genetic sequencing are known to exist from the literature. The OTUs footnoted in this table are known to be discrepant between the different methods for assigning a taxonomic name.

Engraftment of OTUs from the ethanol treated spore preparation treatment into the patient as well as the resulting augmentation of the resident microbiome led to a significant decrease in and elimination of the carriage of pathogenic species other than C. difficile in the patient. 16S-V4 sequencing of primary stool samples demonstrated that at pretreatment, 20% of reads were from the genus Klebsiella and an additional 19% were assigned to the genus Fusobacterium. These striking data are evidence of a profoundly dysbiotic microbiota associated with recurrent C. difficile infection and chronic antibiotic use. In healthy individuals, Klebsiella is a resident of the human microbiome in only about 2% of subjects based on an analysis of HMP database (www.hmpdacc.org), and the mean relative abundance of Klebsiella is only about 0.09% in the stool of these people. Its surprising presence at 20% relative abundance in Patient 1 before treatment is an indicator of a proinflammatory gut environment enabling a “pathobiont” to overgrow and outcompete the commensal organisms normally found in the gut. Similarly, the dramatic overgrowth of Fusobacterium indicates a profoundly dysbiotic gut microbiota. One species of Fusobacterium, F. nucleatum (an OTU phylogenetically indistinguishable from Fusobacterium sp. 3_1_33 based on 16S-V4), has been termed “an emerging gut pathogen” based on its association with IBD, Crohn's disease, and colorectal cancer in humans and its demonstrated causative role in the development of colorectal cancer in animal models [Allen-Vercoe, Gut Microbes (2011) 2:294-8]. Importantly, neither Klebsiella nor Fusobacterium was detected in the 16S-V4 reads by Day 25 (Table 13).

To further characterize the colonization of the gut by Klebsiella and other Enterobacteriaceae and to speciate these organisms, pretreatment and Day 25 fecal samples stored at −80 C as PBS-glycerol suspensions were plated on a variety of selective media including MacConkey lactose media (selective for gram negative enterobacteria) and Simmons Citrate Inositol media (selective for Klebsiella spp) [Van Cregten et al, J. Clin. Microbiol. (1984) 20:936-41]. Enterobacteria identified in the patient samples included K. pneumoniae, Klebsiella sp. Co_9935 and E. coli. Strikingly, each Klebsiella species was reduced by 2-4 logs whereas E. coli, a normal commensal organism present in a healthy microbiota, was reduced by less than 1 log (Table 14 below). This decrease in Klebsiella spp. carriage is consistent across multiple patients. Four separate patients were evaluated for the presence of Klebsiella spp. pre treatment and 4 weeks post treatment. Klebsiella spp. were detected by growth on selective Simmons Citrate Inositol media as previously described. Serial dilution and plating, followed by determining cfu/mL titers of morphologically distinct species and 16S full length sequence identification of representatives of those distinct morphological classes, allowed calculation of titers of specific species.

The genus Bacteroides is an important member of the gastrointestinal microbiota; 100% of stool samples from the Human Microbiome Project contain at least one species of Bacteroides with total relative abundance in these samples ranging from 0.96% to 93.92% with a median relative abundance of 52.67% (www.hmpdacc.org reference data set HMSMCP). Bacteroides in the gut has been associated with amino acid fermentation and degradation of complex polysaccharides. Its presence in the gut is enhanced by diets rich in animal-derived products as found in the typical western diet [David, L. A. et al, Nature (2013) doi: 10.1038/nature12820]. Strikingly, prior to treatment, fewer than 0.008% of the 16S-V4 reads from Patient 1 mapped to the genus Bacteroides strongly suggesting that Bacteroides species were absent or that viable Bacteroides were reduced to an extremely minor component of the patient's gut microbiome. Post treatment, ≥42% of the 16S-V4 reads could be assigned to the genus Bacteroides within 5 days of treatment and by Day 25 post treatment 59.48% of the patients gut microbiome was comprised of Bacteroides. These results were confirmed microbiologically by the absence of detectable Bacteroides in the pretreatment sample plated on two different Bacteroides selective media: Bacteroides Bile Esculin (BBE) agar which is selective for Bacteroides fragilis group species [Livingston, S. J. et al J. Clin. Microbiol (1978). 7:448-453] and Polyamine Free Arabinose (PFA) agar [Noack et al. J. Nutr. (1998) 128:1385-1391; modified by replacing glucose with arabinose]. The highly selective BBE agar had a limit of detection of <2×10³ cfu/g, while the limit of detection for Bacteroides on PFA agar was approximately 2×10⁷ cfu/g due to the growth of multiple non-Bacteroides species in the pretreatment sample on that medium. Colony counts of Bacteroides species on Day 25 were up to 2×10¹⁰ cfu/g, consistent with the 16S-V4 sequencing, demonstrating a profound reconstitution of the gut microbiota in Patient 1 (Table 15 below).

The significant abundance of Bacteroides in Patient 1 on Day 25 (and as early as Day 5 as shown by 16S-V4 sequencing) is remarkable. Viable Bacteroides fragilis group species were not present in the ethanol treated spore population based on microbiological plating (limit of detection of 10 cfu/ml). Thus, administration of the ethanol treated spore population to Patient 1 resulted in microbial population of the patient's GI tract, not only due to the engraftment of bacterial species such as but not limited to spore forming species, but also the restoration of high levels of non-spore forming species commonly found in healthy individuals through the creation of a niche that allowed for the repopulation of Bacteroides species. These organisms were most likely either present at extremely low abundance in the GI tract of Patient 1, or present in a reservoir in the GI tract from which they could rebound to high titer. Those species may also be reinoculated via oral uptake from food following treatment. We term this healthy repopulation of the gut with OTUs that are not present in the bacterial composition such as but not limited to a spore population or ethanol treated spore population, “Augmentation.” Augmentation is an important phenomenon in that it shows the ability to use an ethanol treated spore ecology or other bacterial composition to restore a healthy microbiota by seeding a diverse array or commensal organisms beyond the actual component organisms in the bacterial composition such as but not limited to an ethanol treated spore population itself; specifically the spore composition treatment itself and the engraftment of OTUs from the spore composition create a niche that enables the outgrowth of OTUs required to shift a dysbiotic microbiome to a microbial ecology that is associated with health. The diversity of Bacteroides species and their approximate relative abundance in the gut of Patient 1 is shown in Table 16, comprising at least 8 different species.

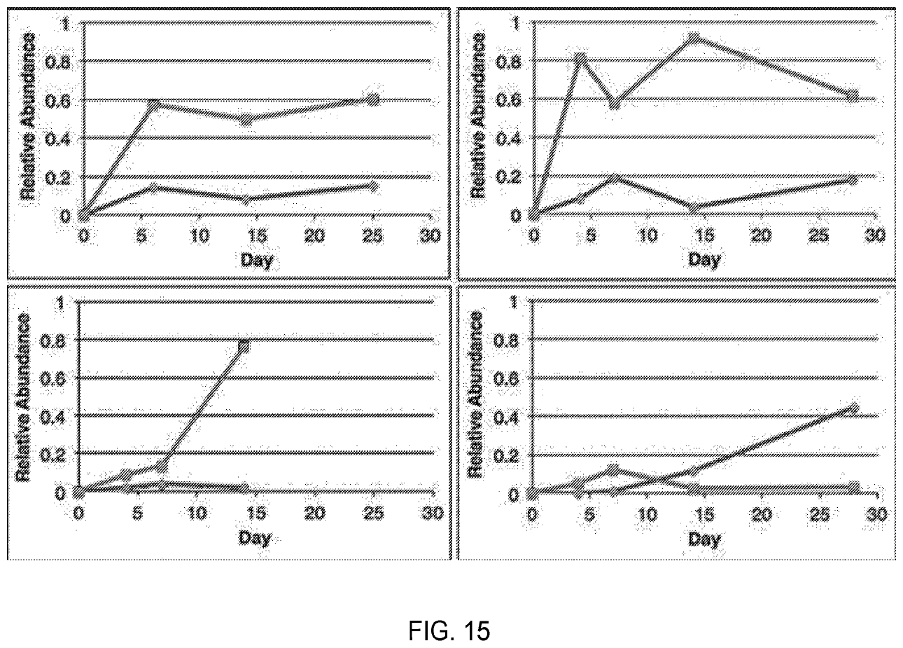

FIG. 15 shows species engrafting versus species augmenting in patients microbiomes after treatment with a bacterial composition such as but not limited to an ethanol-treated spore population. Relative abundance of species that engrafted or augmented as described were determined based on the number of 16S sequence reads. Each plot is from a different patient treated with the bacterial composition such as but not limited to an ethanol-treated spore population for recurrent C. difficile.

The impact of the bacterial composition such as but not limited to an ethanol treated spore population treatment on carriage of imipenem resistant Enterobacteriaceae was assessed by plating pretreatment and Day 28 clinical samples from Patients 2, 4 and 5 on MacConkey lactose plus 1 μg/mL of imipenem. Resistant organisms were scored by morphology, enumerated and DNA was submitted for full length 16S rDNA sequencing as described above. Isolates were identified as Morganella morganii, Providencia rettgeri and Proteus pennerii. Each of these are gut commensal organisms; overgrowth can lead to bacteremia and/or urinary tract infections requiring aggressive antibiotic treatment and, in some cases, hospitalization [Kim, B-N, et al Scan J. Inf Dis (2003) 35:98-103; Lee, I—K and Liu, J-W J. Microbiol Immunol Infect (2006) 39:328-334; O'Hara et al, Clin Microbiol Rev (2000) 13:534]. The titer of organisms at pretreatment and Day 28 by patient is shown in Table 17. Importantly, administration of the bacterial composition such as but not limited to an ethanol treated spore preparation resulted in greater than 100-fold reduction in 4 of 5 cases of Enterobacteriaceae carriage with multiple imipenem resistant organisms (See Table 17 which shows titers (in cfu/g) of imipenem-resistant M. morganii, P. rettgeri and P. pennerii from Patients 2, 4 & 5).

In addition to speciation and enumeration, multiple isolates of each organism from Patient 4 were grown overnight in 96-well trays containing a 2-fold dilution series of imipenem in order to quantitatively determine the minimum inhibitory concentration (MIC) of antibiotic. Growth of organisms was detected by light scattering at 600 nm on a SpectraMax M5e plate reader. In the clinical setting, these species are considered resistant to imipenem if they have an MIC of 1 μg/mL or greater. M. morganii isolates from pretreatment samples from Patient D had MICs of 2-4 ug/mL and P. pennerii isolates had MICs of 4-8 ug/mL. Thus, the bacterial composition, such as but not limited to, an ethanol treated spores administered to Patient 4 caused the clearance of 2 imipenem resistant organisms (Table 12). While this example specifically uses a spore preparation, the methods herein describe how one skilled in the art would use a more general bacterial composition to achieve the same effects. The specific example should not be viewed as a limitation of the scope of this disclosure.

### Example 26: Identifying the Core Ecology from the Bacterial Combination

Ten different bacterial compositions were made by the ethanol treated spore preparation methods from 6 different donors (as described in Example 6). The spore preparations were used to treat 10 patients, each suffering from recurrent C. difficile infection. Patients were identified using the inclusion/exclusion criteria described in Example 9, and donors were identified using the criteria described in Example 10. None of the patients experienced a relapse of C. difficile in the 4 weeks of follow up after treatment, whereas the literature would predict that 70-80% of subjects would experience a relapse following cessation of antibiotic [Van Nood, et al, NEJM (2013)]. Thus, the ethanol treated spore preparations derived from multiple different donors and donations showed remarkable clinical efficacy. These ethanol treated spore preparations are a subset of the bacterial compositions described herein and the results should not be viewed as a limitation on the scope of the broader set of bacterial compositions.

To define the Core Ecology underlying the remarkable clinical efficacy of the bacterial compositions e.g. ethanol treated spore preparations, the following analysis was carried out. The OTU composition of the spore preparation was determined by 16S-V4 rDNA sequencing and computational assignment of OTUs per Example 2. A requirement to detect at least ten sequence reads in the ethanol treated spore preparation was set as a conservative threshold to define only OTUs that were highly unlikely to arise from errors during amplification or sequencing. Methods routinely employed by those familiar to the art of genomic-based microbiome characterization use a read relative abundance threshold of 0.005% (see e.g. Bokulich, A. et al. 2013. Quality-filtering vastly improves diversity estimates from Illumina amplicon sequencing. Nature Methods 10:57-59), which would equate to ≥2 reads given the sequencing depth obtained for the samples analyzed in this example, as cut-off which is substantially lower than the ≥10 reads used in this analysis. All taxonomic and clade assignments were made for each OTU as described in Example 3. The resulting list of OTUs, clade assignments, and frequency of detection in the spore preparations are shown in Table 18. Table 18 shows OTUs detected by a minimum of ten 16S-V4 sequence reads in at least a one ethanol treated spore preparation (pan-microbiome). OTUs that engraft in a treated patients and the percentage of patients in which they engraft are denoted, as are the clades, spore forming status, and Keystone OTU status. Starred OTUs occur in ≥80% of the ethanol preps and engraft in ≥50% of the treated patients.

Next, it was reasoned that for an OTU to be considered a member of the Core Ecology of the bacterial composition, that OTU must be shown to engraft in a patient. Engraftment is important for two reasons. First, engraftment is a sine qua non of the mechanism to reshape the microbiome and eliminate C. difficile colonization. OTUs that engraft with higher frequency are highly likely to be a component of the Core Ecology of the spore preparation or broadly speaking a set bacterial composition. Second, OTUs detected by sequencing a bacterial composition (as in Table 18 may include non-viable cells or other contaminant DNA molecules not associated with the composition. The requirement that an OTU must be shown to engraft in the patient eliminates OTUs that represent non-viable cells or contaminating sequences. Table 18 also identifies all OTUs detected in the bacterial composition that also were shown to engraft in at least one patient post-treatment. OTUs that are present in a large percentage of the bacterial composition e.g. ethanol spore preparations analyzed and that engraft in a large number of patients represent a subset of the Core Ecology that are highly likely to catalyze the shift from a dysbiotic disease ecology to a healthy microbiome.

A third lens was applied to further refine insights into the Core Ecology of the bacterial composition e.g. spore preparation. Computational-based, network analysis has enabled the description of microbial ecologies that are present in the microbiota of a broad population of healthy individuals. These network ecologies are comprised of multiple OTUs, some of which are defined as Keystone OTUs. Keystone OTUs are computationally defined as described in Example 24. Keystone OTUs form a foundation to the microbially ecologies in that they are found and as such are central to the function of network ecologies in healthy subjects. Keystone OTUs associated with microbial ecologies associated with healthy subjects are often are missing or exist at reduced levels in subjects with disease. Keystone OTUs may exist in low, moderate, or high abundance in subjects. Table 18 further notes which of the OTUs in the bacterial composition e.g. spore preparation are Keystone OTUs exclusively associated with individuals that are healthy and do not harbor disease.

There are several important findings from this data. A relatively small number of species, 16 in total, are detected in all of the spore preparations from 6 donors and 10 donations. This is surprising because the HMP database (www.hmpdacc.org) describes the enormous variability of commensal species across healthy individuals. The presence of a small number of consistent OTUs lends support to the concept of a Core Ecology. The engraftment data further supports this conclusion. A regression analysis shows a significant correlation between frequency of detection in a spore preparation and frequency of engraftment in a donor: R=0.43 (p<0.001). While this may seem obvious, there is no a priori requirement that an OTU detected frequently in the bacterial composition e.g. spore preparation will or should engraft. For instance, Lutispora thermophila, a spore former found in all ten spore preparations, did not engraft in any of the patients. Bilophila wadsworthia, a gram negative anaerobe, is present in 9 of 10 donations, yet it does not engraft in any patient, indicating that it is likely a non-viable contaminant in the ethanol treated spore preparation. Finally, it is worth noting the high preponderance of previously defined Keystone OTUs among the most frequent OTUs in the spore preparations.

These three factors—prevalence in the bacterial composition such as but not limited to a spore preparation, frequency of engraftment, and designation as a Keystone OTUs—enabled the creation of a “Core Ecology Score” (CES) to rank individual OTUs. CES was defined as follows:
40% weighting for presence of OTU in spore preparation
multiplier of 1 for presence in 1-3 spore preparations
multiplier of 2.5 for presence in 4-8 spore preparations
multiplier of 5 for presences in ≥9 spore preparations
40% weighting for engraftment in a patient
multiplier of 1 for engraftment in 1-4 patients
multiplier of 2.5 for engraftment in 5-6 patients
multiplier of 5 for engraftment in ≥7 patients
20% weighting to Keystone OTUs
multiplier of 1 for a Keystone OTU
multiplier of 0 for a non-Keystone OTU

Using this guide, the CES has a maximum possible score of 5 and a minimum possible score of 0.8. As an example, an OTU found in 8 of the 10 bacterial composition such as but not limited to a spore preparations that engrafted in 3 patients and was a Keystone OTU would be assigned the follow CES:

$$CES = ( 0.4 \times 2.5 ) + ( 0.4 \times 1 ) + ( 0.2 \times 1 ) = 1.6$$

Table 19 ranks the top 20 OTUs by CES with the further requirement that an OTU must be shown to engraft to be a considered an element of a core ecology.

### Example 27: Defining Efficacious Subsets of the Core Ecology

The number of organisms in the human gastrointestinal tract, as well as the diversity between healthy individuals, is indicative of the functional redundancy of a healthy gut microbiome ecology (see The Human Microbiome Consortia. 2012 Structure, function and diversity of the healthy human microbiome. Nature 486:207-214). This redundancy makes it highly likely that subsets of the Core Ecology describe therapeutically beneficial components of the bacterial composition such as but not limited to an ethanol treated spore preparation and that such subsets may themselves be useful compositions for populating the GI tract and for the treatment of C. difficile infection given the ecologies functional characteristics. Using the CES, individual OTUs can be prioritized for evaluation as an efficacious subset of the Core Ecology.

Another aspect of functional redundancy is that evolutionarily related organisms (i.e. those close to one another on the phylogenetic tree, e.g. those grouped into a single clade) will also be effective substitutes in the Core Ecology or a subset thereof for treating C. difficile.

To one skilled in the art, the selection of appropriate OTU subsets for testing in vitro (e.g. see Example 28 below) or in vivo (e.g. see Examples 7 or 8) is straightforward. Subsets may be selected by picking any 2, 3, 4, 5, 6, 7, 8, 9, 10, or more than 10 OTUs from Table 18, with a particular emphasis on those with higher CES, such as the OTUs described Table 19. In addition, using the clade relationships defined in Example 2 and Table 1 above, related OTUs can be selected as substitutes for OTUs with acceptable CES values. These organisms can be cultured anaerobically in vitro using the appropriate media (selected from those described in Example 5 above), and then combined in a desired ratio. A typical experiment in the mouse C. difficile model utilizes at least 10⁴ and preferably at least 10⁵, 10⁶, 10⁷, 10⁸, 10⁹ or more than 10⁹ colony forming units of a each microbe in the composition. Variations in the culture yields may sometimes mean that organisms are combined in unequal ratios, e.g. 1:10, 1:100, 1:1,000, 1:10,000, 1:100,000, or greater than 1:100,000. What is important in these compositions is that each strain be provided in a minimum amount so that the strain's contribution to the efficacy of the Core Ecology subset can be measured. Using the principles and instructions described here, it is straightforward for one of skill in the art to make clade-based substitutions to test the efficacy of subsets of the Core Ecology. Table 18 describes the clades for each OTU detected in a spore preparation and Table 1 describes the OTUs that can be used for substitutions based on clade relationships.

### Example 28: Testing Subsets of the Core Ecology in the Mouse Model

Several subsets of the Core Ecology were tested in the C. difficile mouse model. The negative control was phosphate buffered saline and the positive control was a 10% human fecal suspension. The subsets are described in Table 20 (Subsets of the Core Ecology tested in the C. difficile mouse model).

Two cages of five mice each were tested for each arm of the experiment. All mice received an antibiotic cocktail consisting of 10% glucose, kanamycin (0.5 mg/ml), gentamicin (0.044 mg/ml), colistin (1062.5 U/ml), metronidazole (0.269 mg/ml), ciprofloxacin (0.156 mg/ml), ampicillin (0.1 mg/ml) and Vancomycin (0.056 mg/ml) in their drinking water on days −14 through-5 and a dose of 10 mg/kg Clindamycin by oral gavage on day −3. On day −1, they received either the test articles or control articles via oral gavage. On day 0, they were challenged by administration of approximately 4.5 log 10 cfu of C. difficile (ATCC 43255) via oral gavage. Mortality was assessed every day from day 0 to day 6 and the weight and subsequent weight change of the animal was assessed with weight loss being associated with C. difficile infection. Mortality and reduced weight loss of the test article compared to the empty vehicle was used to assess the success of the test article. Additionally, a C. difficile symptom scoring was performed each day from day −1 through day 6. Symptom scoring was based on Appearance (0-2 pts based on normal, hunched, piloerection, or lethargic), Respiration (0-2 pts based on normal, rapid or shallow, with abdominal breathing), Clinical Signs (0-2 points based on normal, wet tail, cold-to-the-touch, or isolation from other animals).

In addition to compiling the cumulative mortality for each arm, the average minimum relative weight is calculated as the mean of each mouse's minimum weight relative to Day −1 and the average maximum clinical score is calculated as the mean of each mouse's maximum combined clinical score with a score of 4 assigned in the case of death. The results are reported in Table 21 below (Results of bacterial compositions tested in a C. difficile mouse model).

### Example 29. Defining Subsets of the Core Ecology in the In Vitro C. difficile Inhibition Assay

Vials of −80° C. glycerol stock banks were thawed and diluted to 1e8 CFU/mL. Selected strains and their clade assignment are given in Table 22. Each strain was then diluted 10× (to a final concentration of 1e7 CFU/mL of each strain) into 200 μL of PBS+15% glycerol in the wells of a 96-well plate. Plates were then frozen at −80° C. When needed for the assay, plates were removed from −80° C. and thawed at room temperature under anaerobic conditions when testing in a in vitro C. difficile inhibition assay (CivSim).

An overnight culture of Clostridium difficile is grown under anaerobic conditions in SweetB-Fosin or other suitable media for the growth of C. difficile. SweetB-FosIn is a complex media composed of brain heart infusion, yeast extract, cysteine, cellobiose, maltose, soluble starch, and fructooligosaccharides/inulin, and hemin, and is buffered with MOPs. After 24 hr of growth the culture is diluted 100,000 fold into a complex media such as SweetB-FosIn which is suitable for the growth of a wide variety of anaerobic bacterial species. The diluted C. difficile mixture is then aliquoted to wells of a 96-well plate (180 uL to each well). 20 μL of a subset Core Ecology is then added to each well at a final concentration of 1e6 CFU/mL of each species. Alternatively the assay can be tested each species at different initial concentrations (1e9 CFU/mL, 1e8 CFU/mL, 1e7 CFU/mL, 1e5 CFU/mL, 1e4 CFU/mL, 1e3 CFU/mL, 1e2 CFU/mL). Control wells only inoculated with C. difficile are included for a comparison to the growth of C. difficile without inhibition. Additional wells are used for controls that either inhibit or do not inhibit the growth of C. difficile. One example of a positive control that inhibits growth is a combination of Blautia producta, Clostridium bifermentans and Escherichia coli. One example of a control that shows reduced inhibition of C. difficile growth is a combination of Bacteroides thetaiotaomicron, Bacteroides ovatus and Bacteroides vulgatus. Plates are wrapped with parafilm and incubated for 24 hr at 37° C. under anaerobic conditions. After 24 hr the wells containing C. difficile alone are serially diluted and plated to determine titer. The 96-well plate is then frozen at −80 C before quantifying C. difficile by qPCR assay.

A standard curve is generated from a well on each assay plate containing only pathogenic C. difficile grown in SweetB+FosIn media and quantified by selective spot plating. Serial dilutions of the culture are performed in sterile phosphate-buffered saline. Genomic DNA is extracted from the standard curve samples along with the other wells.

Genomic DNA is extracted from 5 μl of each sample using a dilution, freeze/thaw, and heat lysis protocol. 5 μL of thawed samples is added to 45 μL of UltraPure water (Life Technologies, Carlsbad, CA) and mixed by pipetting. The plates with diluted samples are frozen at −20° C. until use for qPCR which includes a heated lysis step prior to amplification. Alternatively the genomic DNA is isolated using the Mo Bio Powersoil®-htp 96 Well Soil DNA Isolation Kit (Mo Bio Laboratories, Carlsbad, CA), Mo Bio Powersoil® DNA Isolation Kit (Mo Bio Laboratories, Carlsbad, CA), or the QIAamp DNA Stool Mini Kit (QIAGEN, Valencia, CA) according to the manufacturer's instructions.

The qPCR reaction mixture contains 1× SsoAdvanced Universal Probes Supermix, 900 nM of Wr-tcdB-F primer (AGCAGTTGAATATAGTGGTTTAGTTAGAGTTG (SEQ ID NO: 2040), IDT, Coralville, IA), 900 nM of Wr-tcdB-R primer (CATGCTTTTTTAGTTTCTGGATTGAA (SEQ ID NO: 2041), IDT, Coralville, IA), 250 nM of Wr-tcdB-P probe (6FAM-CATCCAGTCTCAATTGTATATGTTTCTCCA-MGB (SEQ ID NO: 2042), Life Technologies, Grand Island, NY), and Molecular Biology Grade Water (Mo Bio Laboratories, Carlsbad, CA) to 18 μl (Primers adapted from: Wroblewski, D. et al, Rapid Molecular Characterization of Clostridium difficile and Assessment of Populations of C. difficile in Stool Specimens, Journal of Clinical Microbiology 47:2142-2148 (2009)). This reaction mixture is aliquoted to wells of a Hard-shell Low-Profile Thin Wall 96-well Skirted PCR Plate (BioRad, Hercules, CA). To this reaction mixture, 2 μl of diluted, frozen, and thawed samples are added and the plate sealed with a Microseal ‘B’ Adhesive Seal (BioRad, Hercules, CA). The qPCR is performed on a BioRad C1000™ Thermal Cycler equipped with a CFX96™ Real-Time System (BioRad, Hercules, CA). The thermocycling conditions are 95° C. for 15 minutes followed by 45 cycles of 95° C. for 5 seconds, 60° C. for 30 seconds, and fluorescent readings of the FAM channel. Alternatively, the qPCR is performed with other standard methods known to those skilled in the art.

The Cq value for each well on the FAM channel is determined by the CFX Manager™ 3.0 software. The log₁₀ (cfu/mL) of C. difficile each experimental sample is calculated by inputting a given sample's Cq value into a linear regression model generated from the standard curve comparing the Cq values of the standard curve wells to the known log₁₀ (cfu/mL) of those samples. The log inhibition is calculated for each sample by subtracting the log₁₀ (cfu/mL) of C. difficile in the sample from the log₁₀ (cfu/mL) of C. difficile in the sample on each assay plate used for the generation of the standard curve that has no additional bacteria added. The mean log inhibition is calculated for all replicates for each composition.

A histogram of the range and standard deviation of each composition is plotted. Ranges or standard deviations of the log inhibitions that are distinct from the overall distribution are examined as possible outliers. If the removal of a single log inhibition datum from one of the binary pairs that is identified in the histograms would bring the range or standard deviation in line with those from the majority of the samples, that datum is removed as an outlier, and the mean log inhibition is recalculated.

The pooled variance of all samples evaluated in the assay is estimated as the average of the sample variances weighted by the sample's degrees of freedom. The pooled standard error is then calculated as the square root of the pooled variance divided by the square root of the number of samples. Confidence intervals for the null hypothesis are determined by multiplying the pooled standard error to the z score corresponding to a given percentage threshold. Mean log inhibitions outside the confidence interval are considered to be inhibitory if positive or stimulatory if negative with the percent confidence corresponding to the interval used. Ternary combinations with mean log inhibition greater than 0.312 are reported as ++++ (≥ 99% confidence interval (C.I.) of the null hypothesis), those with mean log inhibition between 0.221 and 0.312 as +++ (95%<C.I.<99%), those with mean log inhibition between 0.171 and 0.221 as ++ (90%<C.I.<95%), those with mean log inhibition between 0.113 and 0.171 as + (80%<C.I.<90%), those with mean log inhibition between −0.113 and −0.171 as − (80%<C.I.<90%), those with mean log inhibition between −0.171 and −0.221 as −− (90%<C.I.<95%), those with mean log inhibition between −0.221 and −0.312 as −−− (95%<C.I.<99%), and those with mean log inhibition less than −0.312 as −−−− (99%<C.I.).

Table 22 below shows OTUs and their clade assignments tested in ternary combinations with results in the in vitro inhibition assay The CivSim shows that many ternary combinations inhibit C. difficile. 39 of 56 combinations show inhibition with a confidence interval >80%; 36 of 56 with a C.I.>90%; 36 of 56 with a C.I.>95%; 29 of 56 with a C.I. of >99%. Non-limiting but exemplary ternary combinations include those with mean log reduction greater than 0.171, e.g. any combination shown in Table 22 with a score of ++++, such as Collinsella aerofaciens, Coprococcus comes, and Blautia producta. Equally important, the CivSim assay describes ternary combinations that do not effectively inhibit C. difficile. 5 of 56 combinations promote growth with >80% confidence; 2 of 56 promote growth with >90% confidence; 1 of 56, Coprococcus comes, Clostridium symbiosum and Eubacterium rectale, promote growth with >95% confidence. 12 of 56 combinations are neutral in the assay, meaning they neither promote nor inhibit C. difficile growth to the limit of measurement.

It is straightforward for one of skill in the art to use the in vitro competition assay described below to determine efficacious subsets of the Core Ecology derived from the bacterial composition shown to be efficacious in treating C. difficile in humans.

### Use of In Vitro Competition Assay to Test Potential Bacterial Competitor Consortia for Functionality

An in vitro assay is performed to test the ability of a chosen species or combination of species to inhibit the growth of a pathogen such as Clostridium difficile in media that is otherwise suitable for growth of the pathogen. A liquid media suitable for growth of the pathogen is chosen, such as Brain Heart Infusion Broth (BHI) for C. difficile (see Example 5). The potential competitor species or a combination of competitor species are inoculated into 3 mL of the media and incubated anaerobically for 24 hr at 37° C. After incubation the cells are pelleted in a centrifuge at 10,000 rcf for 5 min. Supernatant is removed and filtered through a 0.22 μm filter to remove all cells. C. difficile or another pathogen of interest is then inoculated into the filtered spent supernatant and grown anaerobically at 37° C. for 24 hr. A control culture in fresh media is incubated in parallel. After incubation, the titer of C. difficile is determined by serially diluting and plating to Brucella Blood Agar (BBA) plates and incubated anaerobically for 24 hr at 37° C. Colonies are counted to determine the final titer of the pathogen after incubation in competitor conditioned media and control media. The percent reduction in final titer is calculated and considered inhibitory if a statistically significant reduction in growth is measured. Alternatively, the inhibition of pathogen growth is monitored by OD₆₀₀ measurement of the test and control cultures.

### Example 30: Testing of Bacterial Composition Against Salmonella

The in vitro assay is used to screen for combinations of bacteria inhibitory to the growth of Salmonella spp. by modifying the media used for growth of the pathogen inoculum. Several choices of media are used for growth of the pathogen such as Reinforced Clostridial Media (RCM), Brain Heart Infusion Broth (BHI) or Luria Bertani Broth (LB). Salmonella spp. are quantified by using alternative selective media specific for Salmonella spp. or using qPCR probes specific for the pathogen. For example, MacConkey agar is used to select for Salmonella spp. and the invA gene is targeted with qPCR probes; this gene encodes an invasion protein carried by many pathogenic Salmonella spp. and is used in invading eukaryotic cells.

### Example 31: Method of Preparing the Bacterial Composition for Administration to a Patient

Two strains for the bacterial composition are independently cultured and mixed together before administration. Both strains are independently be grown at 37° C., pH 7, in a GMM or other animal-products-free medium, pre-reduced with 1 g/L cysteine ŸHCl. After each strain reaches a sufficient biomass, it is preserved for banking by adding 15% glycerol and then frozen at −80° C. in 1 ml cryotubes.

Each strain is then be cultivated to a concentration of 10¹⁰ CFU/mL, then concentrated 20-fold by tangential flow microfiltration; the spent medium is exchanged by diafiltering with a preservative medium consisting of 2% gelatin, 100 mM trehalose, and 10 mM sodium phosphate buffer, or other suitable preservative medium. The suspension is freeze-dried to a powder and titrated.

After drying, the powder is blended with microcrystalline cellulose and magnesium stearate and formulated into a 250 mg gelatin capsule containing 10 mg of lyophilized powder (10⁸ to 10¹¹ bacteria), 160 mg microcrystalline cellulose, 77.5 mg gelatin, and 2.5 mg magnesium stearate.

### Example 32: Method of Treating a Subject with a Bacterial Composition

A patient has suffered from recurrent bouts of C. difficile. In the most recent acute phase of illness, the patient is treated with an antibiotic sufficient to ameliorate the symptoms of the illness. In order to prevent another relapse of C. difficile, the patient is administered one of the present bacterial compositions. Specifically, the patient is administered Bacillus circulans and Roseburia inulinivorans at a dose of 10⁸ bacteria total in a lyophilized form, specifically in a 250 mg gelatin capsule containing 10 mg of lyophilized bacteria, 160 mg microcrystalline cellulose, 77.5 mg gelatin, and 2.5 mg magnesium stearate. The patient takes the capsule by mouth and resumes a normal diet after 4, 8, 12, or 24 hours. In another embodiment, the patient may take the capsule by mouth before, during, or immediately after a meal.

Feces is collected before and at 1 day, 3 days, 1 week, and 1 month after administration. The presence of C. difficile is found in the feces before administration of the bacterial composition, but feces collections after administration show reducing (such as at least 50% less, 60%, 70%, 80%, 90%, or 95%) to no detectable levels of C. difficile, as measured by qPCR, as described above. ELISA for toxin protein or traditional microbiological identification techniques may also be used.

As another measure of patient success, a positive response may be defined as absence of diarrhea, which itself is defined as 3 or more loose or watery stools per day for at least 2 consecutive days or 8 or more loose or watery stools in 48 hours, or persisting diarrhea (due to other causes) with repeating (three times) negative stool tests for toxins of C. difficile.

Treatment failure is defined as persisting diarrhea with a positive C. difficile toxin stool test or no reduction in levels of C. difficile, as measured by qPCR sequencing. ELISA or traditional microbiological identification techniques may also be used.

### Example 33: Method of Treating a Subject with a Bacterial Composition

A patient has suffered from recurrent bouts of C. difficile. In the most recent acute phase of illness, the patient is treated with an antibiotic sufficient to ameliorate the symptoms of the illness. In order to prevent another relapse of C. difficile, the patient is administered one of the present bacterial compositions. Specifically, the patient is administered a bacterial composition containing two bacterial types from Table 1, at a dose of 10⁸ bacteria total in a lyophilized form formulated in an enteric coated capsule. Example of the patient or samples derived from the patient is expected to demonstrate at least one measure of success as described herein (reducing levels of C. difficile as measured by qPCR, ELISA, or traditional microbiological identification; absence of diarrhea; persisting diarrhea with repeating (three times) negative stool tests for toxins of C. difficile.

Other embodiments will be apparent to those skilled in the art from consideration of the specification and practice of the embodiments. Consider the specification and examples as exemplary only, with a true scope and spirit being indicated by the following claims.

Unless otherwise indicated, all numbers expressing quantities of ingredients, reaction conditions, and so forth used in the specification, including claims, are to be understood as being modified in all instances by the term “about.” Accordingly, unless otherwise indicated to the contrary, the numerical parameters are approximations and may vary depending upon the desired properties sought to be obtained. At the very least, and not as an attempt to limit the application of the doctrine of equivalents to the scope of the claims, each numerical parameter should be construed in light of the number of significant digits and ordinary rounding approaches.

Unless otherwise indicated, the term “at least” preceding a series of elements is to be understood to refer to every element in the series.

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

TABLE 1

| | SEQ ID | Public DB | | Spore | Pathogen |
|---|---|---|---|---|---|
| OTU | Number | Accession | Clade | Former | Status |
| Eubacterium saburreum | 858 | AB525414 | clade_178 | Y | N |
| Eubacterium sp. oral clone IR009 | 866 | AY349376 | clade_178 | Y | N |
| Lachnospiraceae bacterium ICM62 | 1061 | HQ616401 | clade_178 | Y | N |
| Lachnospiraceae bacterium MSX33 | 1062 | HQ616384 | clade_178 | Y | N |
| Lachnospiraceae bacterium oral taxon 107 | 1063 | ADDS01000069 | clade_178 | Y | N |
| Alicyclobacillus acidocaldarius | 122 | NR_074721 | clade_179 | Y | N |
| Clostridium baratii | 555 | NR_029229 | clade_223 | Y | N |
| Clostridium colicanis | 576 | FJ957863 | clade_223 | Y | N |
| Clostridium paraputrificum | 611 | AB536771 | clade_223 | Y | N |
| Clostridium sardiniense | 621 | NR_041006 | clade_223 | Y | N |
| Eubacterium budayi | 837 | NR_024682 | clade_223 | Y | N |
| Eubacterium moniliforme | 851 | HF558373 | clade_223 | Y | N |
| Eubacterium multiforme | 852 | NR_024683 | clade_223 | Y | N |
| Eubacterium nitritogenes | 853 | NR_024684 | clade_223 | Y | N |
| Anoxybacillus flavithermus | 173 | NR_074667 | clade_238 | Y | N |
| Bacillus aerophilus | 196 | NR_042339 | clade_238 | Y | N |
| Bacillus aestuarii | 197 | GQ980243 | clade_238 | Y | N |
| Bacillus amyloliquefaciens | 199 | NR_075005 | clade_238 | Y | N |
| Bacillus anthracis | 200 | AAEN01000020 | clade_238 | Y | Category-A |
| Bacillus atrophaeus | 201 | NR_075016 | clade_238 | Y | OP |
| Bacillus badius | 202 | NR_036893 | clade_238 | Y | OP |
| Bacillus cereus | 203 | ABDJ01000015 | clade_238 | Y | OP |
| Bacillus circulans | 204 | AB271747 | clade_238 | Y | OP |
| Bacillus firmus | 207 | NR_025842 | clade_238 | Y | OP |
| Bacillus flexus | 208 | NR_024691 | clade_238 | Y | OP |
| Bacillus fordii | 209 | NR_025786 | clade_238 | Y | OP |
| Bacillus halmapalus | 211 | NR_026144 | clade_238 | Y | OP |
| Bacillus herbersteinensis | 213 | NR_042286 | clade_238 | Y | OP |
| Bacillus idriensis | 215 | NR_043268 | clade_238 | Y | OP |
| Bacillus lentus | 216 | NR_040792 | clade_238 | Y | OP |
| Bacillus licheniformis | 217 | NC_006270 | clade_238 | Y | OP |
| Bacillus megaterium | 218 | GU252124 | clade_238 | Y | OP |
| Bacillus nealsonii | 219 | NR_044546 | clade_238 | Y | OP |
| Bacillus niabensis | 220 | NR_043334 | clade_238 | Y | OP |
| Bacillus niacini | 221 | NR_024695 | clade_238 | Y | OP |
| Bacillus pocheonensis | 222 | NR_041377 | clade_238 | Y | OP |
| Bacillus pumilus | 223 | NR_074977 | clade_238 | Y | OP |
| Bacillus safensis | 224 | JQ624766 | clade_238 | Y | OP |
| Bacillus simplex | 225 | NR_042136 | clade_238 | Y | OP |
| Bacillus sonorensis | 226 | NR_025130 | clade_238 | Y | OP |
| Bacillus sp. 10403023 MM10403188 | 227 | CAET01000089 | clade_238 | Y | OP |
| Bacillus sp. 2_A_57_CT2 | 230 | ACWD01000095 | clade_238 | Y | OP |
| Bacillus sp. 2008724126 | 228 | GU252108 | clade_238 | Y | OP |
| Bacillus sp. 2008724139 | 229 | GU252111 | clade_238 | Y | OP |
| Bacillus sp. 7_16AIA | 231 | FN397518 | clade_238 | Y | OP |
| Bacillus sp. AP8 | 233 | JX101689 | clade_238 | Y | OP |
| Bacillus sp. B27(2008) | 234 | EU362173 | clade_238 | Y | OP |
| Bacillus sp. BT1B_CT2 | 235 | ACWC01000034 | clade_238 | Y | OP |
| Bacillus sp. GB1.1 | 236 | FJ897765 | clade_238 | Y | OP |
| Bacillus sp. GB9 | 237 | FJ897766 | clade_238 | Y | OP |
| Bacillus sp. HU19.1 | 238 | FJ897769 | clade_238 | Y | OP |
| Bacillus sp. HU29 | 239 | FJ897771 | clade_238 | Y | OP |
| Bacillus sp. HU33.1 | 240 | FJ897772 | clade_238 | Y | OP |
| Bacillus sp. JC6 | 241 | JF824800 | clade_238 | Y | OP |
| Bacillus sp. oral taxon F79 | 248 | HM099654 | clade_238 | Y | OP |
| Bacillus sp. SRC_DSF1 | 243 | GU797283 | clade_238 | Y | OP |
| Bacillus sp. SRC_DSF10 | 242 | GU797292 | clade_238 | Y | OP |
| Bacillus sp. SRC_DSF2 | 244 | GU797284 | clade_238 | Y | OP |
| Bacillus sp. SRC_DSF6 | 245 | GU797288 | clade_238 | Y | OP |
| Bacillus sp. tc09 | 249 | HQ844242 | clade_238 | Y | OP |
| Bacillus sp. zh168 | 250 | FJ851424 | clade_238 | Y | OP |
| Bacillus sphaericus | 251 | DQ286318 | clade_238 | Y | OP |
| Bacillus sporothermodurans | 252 | NR_026010 | clade_238 | Y | OP |
| Bacillus subtilis | 253 | EU627588 | clade_238 | Y | OP |
| Bacillus thermoamylovorans | 254 | NR_029151 | clade_238 | Y | OP |
| Bacillus thuringiensis | 255 | NC_008600 | clade_238 | Y | OP |
| Bacillus weihenstephanensis | 256 | NR_074926 | clade_238 | Y | OP |
| Geobacillus kaustophilus | 933 | NR_074989 | clade_238 | Y | N |
| Geobacillus stearothermophilus | 936 | NR_040794 | clade_238 | Y | N |
| Geobacillus thermodenitrificans | 938 | NR_074976 | clade_238 | Y | N |
| Geobacillus thermoglucosidasius | 939 | NR_043022 | clade_238 | Y | N |
| Lysinibacillus sphaericus | 1193 | NR_074883 | clade_238 | Y | N |
| Clostridiales sp. SS3_4 | 543 | AY305316 | clade_246 | Y | N |
| Clostridium beijerinckii | 557 | NR_074434 | clade_252 | Y | N |
| Clostridium botulinum | 560 | NC_010723 | clade_252 | Y | Category-A |
| Clostridium butyricum | 561 | ABDT01000017 | clade_252 | Y | N |
| Clostridium chauvoei | 568 | EU106372 | clade_252 | Y | N |
| Clostridium favososporum | 582 | X76749 | clade_252 | Y | N |
| Clostridium histolyticum | 592 | HF558362 | clade_252 | Y | N |
| Clostridium isatidis | 597 | NR_026347 | clade_252 | Y | N |
| Clostridium limosum | 602 | FR870444 | clade_252 | Y | N |
| Clostridium sartagoforme | 622 | NR_026490 | clade_252 | Y | N |
| Clostridium septicum | 624 | NR_026020 | clade_252 | Y | N |
| Clostridium sp. 7_2_43FAA | 626 | ACDK01000101 | clade_252 | Y | N |
| Clostridium sporogenes | 645 | ABKW02000003 | clade_252 | Y | N |
| Clostridium tertium | 653 | Y18174 | clade_252 | Y | N |
| Clostridium carnis | 564 | NR_044716 | clade_253 | Y | N |
| Clostridium celatum | 565 | X77844 | clade_253 | Y | N |
| Clostridium disporicum | 579 | NR_026491 | clade_253 | Y | N |
| Clostridium gasigenes | 585 | NR_024945 | clade_253 | Y | N |
| Clostridium quinii | 616 | NR_026149 | clade_253 | Y | N |
| Clostridium hylemonae | 593 | AB023973 | clade_260 | Y | N |
| Clostridium scindens | 623 | AF262238 | clade_260 | Y | N |
| Lachnospiraceae bacterium 5_1_57FAA | 1054 | ACTR01000020 | clade_260 | Y | N |
| Clostridium glycyrrhizinilyticum | 588 | AB233029 | clade_262 | Y | N |
| Clostridium nexile | 607 | X73443 | clade_262 | Y | N |
| Coprococcus comes | 674 | ABVR01000038 | clade_262 | Y | N |
| Lachnospiraceae bacterium 1_1_57FAA | 1048 | ACTM01000065 | clade_262 | Y | N |
| Lachnospiraceae bacterium 1_4_56FAA | 1049 | ACTN01000028 | clade_262 | Y | N |
| Lachnospiraceae bacterium 8_1_57FAA | 1057 | ACWQ01000079 | clade_262 | Y | N |
| Ruminococcus lactaris | 1663 | ABOU02000049 | clade_262 | Y | N |
| Ruminococcus torques | 1670 | AAVP02000002 | clade_262 | Y | N |
| Paenibacillus lautus | 1397 | NR_040882 | clade_270 | Y | N |
| Paenibacillus polymyxa | 1399 | NR_037006 | clade_270 | Y | N |
| Paenibacillus sp. HGF5 | 1402 | AEXS01000095 | clade_270 | Y | N |
| Paenibacillus sp. HGF7 | 1403 | AFDH01000147 | clade_270 | Y | N |
| Eubacterium sp. oral clone JI012 | 868 | AY349379 | clade_298 | Y | N |
| Alicyclobacillus contaminans | 124 | NR_041475 | clade_301 | Y | N |
| Alicyclobacillus herbarius | 126 | NR_024753 | clade_301 | Y | N |
| Alicyclobacillus pomorum | 127 | NR_024801 | clade_301 | Y | N |
| Blautia coccoides | 373 | AB571656 | clade_309 | Y | N |
| Blautia glucerasea | 374 | AB588023 | clade_309 | Y | N |
| Blautia glucerasei | 375 | AB439724 | clade_309 | Y | N |
| Blautia hansenii | 376 | ABYU02000037 | clade_309 | Y | N |
| Blautia luti | 378 | AB691576 | clade_309 | Y | N |
| Blautia producta | 379 | AB600998 | clade_309 | Y | N |
| Blautia schinkii | 380 | NR_026312 | clade_309 | Y | N |
| Blautia sp. M25 | 381 | HM626178 | clade_309 | Y | N |
| Blautia stercoris | 382 | HM626177 | clade_309 | Y | N |
| Blautia wexlerae | 383 | EF036467 | clade_309 | Y | N |
| Bryantella formatexigens | 439 | ACCL02000018 | clade_309 | Y | N |
| Clostridium coccoides | 573 | EF025906 | clade_309 | Y | N |
| Eubacterium cellulosolvens | 839 | AY178842 | clade_309 | Y | N |
| Lachnospiraceae bacterium 6_1_63FAA | 1056 | ACTV01000014 | clade_309 | Y | N |
| Ruminococcus hansenii | 1662 | M59114 | clade_309 | Y | N |
| Ruminococcus obeum | 1664 | AY169419 | clade_309 | Y | N |
| Ruminococcus sp. 5_1_39BFAA | 1666 | ACII01000172 | clade_309 | Y | N |
| Ruminococcus sp. K_1 | 1669 | AB222208 | clade_309 | Y | N |
| Syntrophococcus sucromutans | 1911 | NR_036869 | clade_309 | Y | N |
| Bacillus alcalophilus | 198 | X76436 | clade_327 | Y | N |
| Bacillus clausii | 205 | FN397477 | clade_327 | Y | OP |
| Bacillus gelatini | 210 | NR_025595 | clade_327 | Y | OP |
| Bacillus halodurans | 212 | AY144582 | clade_327 | Y | OP |
| Bacillus sp. oral taxon F26 | 246 | HM099642 | clade_327 | Y | OP |
| Clostridium innocuum | 595 | M23732 | clade_351 | Y | N |
| Clostridium sp. HGF2 | 628 | AENW01000022 | clade_351 | Y | N |
| Clostridium perfringens | 612 | ABDW01000023 | clade_353 | Y | Category-B |
| Sarcina ventriculi | 1687 | NR_026146 | clade_353 | Y | N |
| Clostridium bartlettii | 556 | ABEZ02000012 | clade_354 | Y | N |
| Clostridium bifermentans | 558 | X73437 | clade_354 | Y | N |
| Clostridium ghonii | 586 | AB542933 | clade_354 | Y | N |
| Clostridium glycolicum | 587 | FJ384385 | clade_354 | Y | N |
| Clostridium mayombei | 605 | FR733682 | clade_354 | Y | N |
| Clostridium sordellii | 625 | AB448946 | clade_354 | Y | N |
| Clostridium sp. MT4 E | 635 | FJ159523 | clade_354 | Y | N |
| Eubacterium tenue | 872 | M59118 | clade_354 | Y | N |
| Clostridium argentinense | 553 | NR_029232 | clade_355 | Y | N |
| Clostridium sp. JC122 | 630 | CAEV01000127 | clade_355 | Y | N |
| Clostridium sp. NMBHI_1 | 636 | JN093130 | clade_355 | Y | N |
| Clostridium subterminale | 650 | NR_041795 | clade_355 | Y | N |
| Clostridium sulfidigenes | 651 | NR_044161 | clade_355 | Y | N |
| Dorea formicigenerans | 773 | AAXA02000006 | clade_360 | Y | N |
| Dorea longicatena | 774 | AJ132842 | clade_360 | Y | N |
| Lachnospiraceae bacterium 2_1_46FAA | 1050 | ADLB01000035 | clade_360 | Y | N |
| Lachnospiraceae bacterium 2_1_58FAA | 1051 | ACTO01000052 | clade_360 | Y | N |
| Lachnospiraceae bacterium 4_1_37FAA | 1053 | ADCR01000030 | clade_360 | Y | N |
| Lachnospiraceae bacterium 9_1_43BFAA | 1058 | ACTX01000023 | clade_360 | Y | N |
| Ruminococcus gnavus | 1661 | X94967 | clade_360 | Y | N |
| Ruminococcus sp. ID8 | 1668 | AY960564 | clade_360 | Y | N |
| Blautia hydrogenotrophica | 377 | ACBZ01000217 | clade_368 | Y | N |
| Lactonifactor longoviformis | 1147 | DQ100449 | clade_368 | Y | N |
| Robinsoniella peoriensis | 1633 | AF445258 | clade_368 | Y | N |
| Eubacterium infirmum | 849 | U13039 | clade_384 | Y | N |
| Eubacterium sp. WAL 14571 | 864 | FJ687606 | clade_384 | Y | N |
| Erysipelotrichaceae bacterium 5_2_54FAA | 823 | ACZW01000054 | clade_385 | Y | N |
| Eubacterium biforme | 835 | ABYT01000002 | clade_385 | Y | N |
| Eubacterium cylindroides | 842 | FP929041 | clade_385 | Y | N |
| Eubacterium dolichum | 844 | L34682 | clade_385 | Y | N |
| Eubacterium sp. 3_1_31 | 861 | ACTL01000045 | clade_385 | Y | N |
| Eubacterium tortuosum | 873 | NR_044648 | clade_385 | Y | N |
| Bulleidia extructa | 441 | ADFR01000011 | clade_388 | Y | N |
| Solobacterium moorei | 1739 | AECQ01000039 | clade_388 | Y | N |
| Coprococcus catus | 673 | EU266552 | clade_393 | Y | N |
| Lachnospiraceae bacterium oral taxon F15 | 1064 | HM099641 | clade_393 | Y | N |
| Clostridium cochlearium | 574 | NR_044717 | clade_395 | Y | N |
| Clostridium malenominatum | 604 | FR749893 | clade_395 | Y | N |
| Clostridium tetani | 654 | NC_004557 | clade_395 | Y | N |
| Acetivibrio ethanolgignens | 6 | FR749897 | clade_396 | Y | N |
| Anaerosporobacter mobilis | 161 | NR_042953 | clade_396 | Y | N |
| Bacteroides pectinophilus | 288 | ABVQ01000036 | clade_396 | Y | N |
| Clostridium aminovalericum | 551 | NR_029245 | clade_396 | Y | N |
| Clostridium phytofermentans | 613 | NR_074652 | clade_396 | Y | N |
| Eubacterium hallii | 848 | L34621 | clade_396 | Y | N |
| Eubacterium xylanophilum | 875 | L34628 | clade_396 | Y | N |
| Ruminococcus callidus | 1658 | NR_029160 | clade_406 | Y | N |
| Ruminococcus champanellensis | 1659 | FP929052 | clade_406 | Y | N |
| Ruminococcus sp. 18P13 | 1665 | AJ515913 | clade_406 | Y | N |
| Ruminococcus sp. 9SE51 | 1667 | FM954974 | clade_406 | Y | N |
| Anaerostipes caccae | 162 | ABAX03000023 | clade_408 | Y | N |
| Anaerostipes sp. 3_2_56FAA | 163 | ACWB01000002 | clade_408 | Y | N |
| Clostridiales bacterium 1_7_47FAA | 541 | ABQR01000074 | clade_408 | Y | N |
| Clostridiales sp. SM4_1 | 542 | FP929060 | clade_408 | Y | N |
| Clostridiales sp. SSC_2 | 544 | FP929061 | clade_408 | Y | N |
| Clostridium aerotolerans | 546 | X76163 | clade_408 | Y | N |
| Clostridium aldenense | 547 | NR_043680 | clade_408 | Y | N |
| Clostridium algidixylanolyticum | 550 | NR_028726 | clade_408 | Y | N |
| Clostridium amygdalinum | 552 | AY353957 | clade_408 | Y | N |
| Clostridium asparagiforme | 554 | ACCJ01000522 | clade_408 | Y | N |
| Clostridium bolteae | 559 | ABCC02000039 | clade_408 | Y | N |
| Clostridium celerecrescens | 566 | JQ246092 | clade_408 | Y | N |
| Clostridium citroniae | 569 | ADLJ01000059 | clade_408 | Y | N |
| Clostridium clostridiiformes | 571 | M59089 | clade_408 | Y | N |
| Clostridium clostridioforme | 572 | NR_044715 | clade_408 | Y | N |
| Clostridium hathewayi | 590 | AY552788 | clade_408 | Y | N |
| Clostridium indolis | 594 | AF028351 | clade_408 | Y | N |
| Clostridium lavalense | 600 | EF564277 | clade_408 | Y | N |
| Clostridium saccharolyticum | 620 | CP002109 | clade_408 | Y | N |
| Clostridium sp. M62_1 | 633 | ACFX02000046 | clade_408 | Y | N |
| Clostridium sp. SS2_1 | 638 | ABGC03000041 | clade_408 | Y | N |
| Clostridium sphenoides | 643 | X73449 | clade_408 | Y | N |
| Clostridium symbiosum | 652 | ADLQ01000114 | clade_408 | Y | N |
| Clostridium xylanolyticum | 658 | NR_037068 | clade_408 | Y | N |
| Eubacterium hadrum | 847 | FR749933 | clade_408 | Y | N |
| Lachnospiraceae bacterium 3_1_57FAA_CT1 | 1052 | ACTP01000124 | clade_408 | Y | N |
| Lachnospiraceae bacterium 5_1_63FAA | 1055 | ACTS01000081 | clade_408 | Y | N |
| Lachnospiraceae bacterium A4 | 1059 | DQ789118 | clade_408 | Y | N |
| Lachnospiraceae bacterium DJF VP30 | 1060 | EU728771 | clade_408 | Y | N |
| Lachnospiraceae genomosp. C1 | 1065 | AY278618 | clade_408 | Y | N |
| Clostridium difficile | 578 | NC_013315 | clade_409 | Y | OP |
| Eubacterium sp. AS15b | 862 | HQ616364 | clade_428 | Y | N |
| Eubacterium sp. OBRC9 | 863 | HQ616354 | clade_428 | Y | N |
| Eubacterium sp. oral clone OH3A | 871 | AY947497 | clade_428 | Y | N |
| Eubacterium yurii | 876 | AEES01000073 | clade_428 | Y | N |
| Clostridium acetobutylicum | 545 | NR_074511 | clade_430 | Y | N |
| Clostridium algidicarnis | 549 | NR_041746 | clade_430 | Y | N |
| Clostridium cadaveris | 562 | AB542932 | clade_430 | Y | N |
| Clostridium carboxidivorans | 563 | FR733710 | clade_430 | Y | N |
| Clostridium estertheticum | 580 | NR_042153 | clade_430 | Y | N |
| Clostridium fallax | 581 | NR_044714 | clade_430 | Y | N |
| Clostridium felsineum | 583 | AF270502 | clade_430 | Y | N |
| Clostridium frigidicarnis | 584 | NR_024919 | clade_430 | Y | N |
| Clostridium kluyveri | 598 | NR_074165 | clade_430 | Y | N |
| Clostridium magnum | 603 | X77835 | clade_430 | Y | N |
| Clostridium putrefaciens | 615 | NR_024995 | clade_430 | Y | N |
| Clostridium sp. HPB_46 | 629 | AY862516 | clade_430 | Y | N |
| Clostridium tyrobutyricum | 656 | NR_044718 | clade_430 | Y | N |
| Sutterella parvirubra | 1899 | AB300989 | clade_432 | Y | N |
| Acetanaerobacterium elongatum | 4 | NR_042930 | clade_439 | Y | N |
| Clostridium cellulosi | 567 | NR_044624 | clade_439 | Y | N |
| Ethanoligenens harbinense | 832 | AY675965 | clade_439 | Y | N |
| Eubacterium rectale | 856 | FP929042 | clade_444 | Y | N |
| Eubacterium sp. oral clone GI038 | 865 | AY349374 | clade_444 | Y | N |
| Lachnobacterium bovis | 1045 | GU324407 | clade_444 | Y | N |
| Roseburia cecicola | 1634 | GU233441 | clade_444 | Y | N |
| Roseburia faecalis | 1635 | AY804149 | clade_444 | Y | N |
| Roseburia faecis | 1636 | AY305310 | clade_444 | Y | N |
| Roseburia hominis | 1637 | AJ270482 | clade_444 | Y | N |
| Roseburia intestinalis | 1638 | FP929050 | clade_444 | Y | N |
| Roseburia inulinivorans | 1639 | AJ270473 | clade_444 | Y | N |
| Brevibacillus brevis | 410 | NR_041524 | clade_448 | Y | N |
| Brevibacillus laterosporus | 414 | NR_037005 | clade_448 | Y | N |
| Bacillus coagulans | 206 | DQ297928 | clade_451 | Y | OP |
| Sporolactobacillus inulinus | 1752 | NR_040962 | clade_451 | Y | N |
| Kocuria palustris | 1041 | EU333884 | clade_453 | Y | N |
| Nocardia farcinica | 1353 | NC_006361 | clade_455 | Y | N |
| Bacillus sp. oral taxon F28 | 247 | HM099650 | clade_456 | Y | OP |
| Catenibacterium mitsuokai | 495 | AB030224 | clade_469 | Y | N |
| Clostridium sp. TM_40 | 640 | AB249652 | clade_469 | Y | N |
| Coprobacillus cateniformis | 670 | AB030218 | clade_469 | Y | N |
| Coprobacillus sp. 29_1 | 671 | ADKX01000057 | clade_469 | Y | N |
| Clostridium rectum | 618 | NR_029271 | clade_470 | Y | N |
| Eubacterium nodatum | 854 | U13041 | clade_476 | Y | N |
| Eubacterium saphenum | 859 | NR_026031 | clade_476 | Y | N |
| Eubacterium sp. oral clone JH012 | 867 | AY349373 | clade_476 | Y | N |
| Eubacterium sp. oral clone JS001 | 870 | AY349378 | clade_476 | Y | N |
| Faecalibacterium prausnitzii | 880 | ACOP02000011 | clade_478 | Y | N |
| Gemmiger formicilis | 932 | GU562446 | clade_478 | Y | N |
| Subdoligranulum variabile | 1896 | AJ518869 | clade_478 | Y | N |
| Clostridiaceae bacterium JC13 | 532 | JF824807 | clade_479 | Y | N |
| Clostridium sp. MLG055 | 634 | AF304435 | clade_479 | Y | N |
| Erysipelotrichaceae bacterium 3_1_53 | 822 | ACTJ01000113 | clade_479 | Y | N |
| Clostridium cocleatum | 575 | NR_026495 | clade_481 | Y | N |
| Clostridium ramosum | 617 | M23731 | clade_481 | Y | N |
| Clostridium saccharogumia | 619 | DQ100445 | clade_481 | Y | N |
| Clostridium spiroforme | 644 | X73441 | clade_481 | Y | N |
| Coprobacillus sp. D7 | 672 | ACDT01000199 | clade_481 | Y | N |
| Clostridiales bacterium SY8519 | 535 | AB477431 | clade_482 | Y | N |
| Clostridium sp. SY8519 | 639 | AP012212 | clade_482 | Y | N |
| Eubacterium ramulus | 855 | AJ011522 | clade_482 | Y | N |
| Erysipelothrix inopinata | 819 | NR_025594 | clade_485 | Y | N |
| Erysipelothrix rhusiopathiae | 820 | ACLK01000021 | clade_485 | Y | N |
| Erysipelothrix tonsillarum | 821 | NR_040871 | clade_485 | Y | N |
| Holdemania filiformis | 1004 | Y11466 | clade_485 | Y | N |
| Mollicutes bacterium pACH93 | 1258 | AY297808 | clade_485 | Y | N |
| Coxiella burnetii | 736 | CP000890 | clade_486 | Y | Category-B |
| Clostridium hiranonis | 591 | AB023970 | clade_487 | Y | N |
| Clostridium irregulare | 596 | NR_029249 | clade_487 | Y | N |
| Clostridium orbiscindens | 609 | Y18187 | clade_494 | Y | N |
| Clostridium sp. NML 04A032 | 637 | EU815224 | clade_494 | Y | N |
| Flavonifractor plautii | 886 | AY724678 | clade_494 | Y | N |
| Pseudoflavonifractor capillosus | 1591 | AY136666 | clade_494 | Y | N |
| Ruminococcaceae bacterium D16 | 1655 | ADDX01000083 | clade_494 | Y | N |
| Acetivibrio cellulolyticus | 5 | NR_025917 | clade_495 | Y | N |
| Clostridium aldrichii | 548 | NR_026099 | clade_495 | Y | N |
| Clostridium clariflavum | 570 | NR_041235 | clade_495 | Y | N |
| Clostridium stercorarium | 647 | NR_025100 | clade_495 | Y | N |
| Clostridium straminisolvens | 649 | NR_024829 | clade_495 | Y | N |
| Clostridium thermocellum | 655 | NR_074629 | clade_495 | Y | N |
| Fusobacterium nucleatum | 901 | ADVK01000034 | clade_497 | Y | N |
| Eubacterium barkeri | 834 | NR_044661 | clade_512 | Y | N |
| Eubacterium callanderi | 838 | NR_026330 | clade_512 | Y | N |
| Eubacterium limosum | 850 | CP002273 | clade_512 | Y | N |
| Anaerotruncus colihominis | 164 | ABGD02000021 | clade_516 | Y | N |
| Clostridium methylpentosum | 606 | ACEC01000059 | clade_516 | Y | N |
| Clostridium sp. YIT 12070 | 642 | AB491208 | clade_516 | Y | N |
| Hydrogenoanaerobacterium saccharovorans | 1005 | NR_044425 | clade_516 | Y | N |
| Ruminococcus albus | 1656 | AY445600 | clade_516 | Y | N |
| Ruminococcus flavefaciens | 1660 | NR_025931 | clade_516 | Y | N |
| Clostridium haemolyticum | 589 | NR_024749 | clade_517 | Y | N |
| Clostridium novyi | 608 | NR_074343 | clade_517 | Y | N |
| Clostridium sp. LMG 16094 | 632 | X95274 | clade_517 | Y | N |
| Eubacterium ventriosum | 874 | L34421 | clade_519 | Y | N |
| Bacteroides galacturonicus | 280 | DQ497994 | clade_522 | Y | N |
| Eubacterium eligens | 845 | CP001104 | clade_522 | Y | N |
| Lachnospira multipara | 1046 | FR733699 | clade_522 | Y | N |
| Lachnospira pectinoschiza | 1047 | L14675 | clade_522 | Y | N |
| Lactobacillus rogosae | 1114 | GU269544 | clade_522 | Y | N |
| Bacillus horti | 214 | NR_036860 | clade_527 | Y | OP |
| Bacillus sp. 9_3AIA | 232 | FN397519 | clade_527 | Y | OP |
| Eubacterium brachy | 836 | U13038 | clade_533 | Y | N |
| Filifactor alocis | 881 | CP002390 | clade_533 | Y | N |
| Filifactor villosus | 882 | NR_041928 | clade_533 | Y | N |
| Clostridium leptum | 601 | AJ305238 | clade_537 | Y | N |
| Clostridium sp. YIT 12069 | 641 | AB491207 | clade_537 | Y | N |
| Clostridium sporosphaeroides | 646 | NR_044835 | clade_537 | Y | N |
| Eubacterium coprostanoligenes | 841 | HM037995 | clade_537 | Y | N |
| Ruminococcus bromii | 1657 | EU266549 | clade_537 | Y | N |
| Eubacterium siraeum | 860 | ABCA03000054 | clade_538 | Y | N |
| Clostridium viride | 657 | NR_026204 | clade_540 | Y | N |
| Oscillibacter sp. G2 | 1386 | HM626173 | clade_540 | Y | N |
| Oscillibacter valericigenes | 1387 | NR_074793 | clade_540 | Y | N |
| Oscillospira guilliermondii | 1388 | AB040495 | clade_540 | Y | N |
| Butyrivibrio crossotus | 455 | ABWN01000012 | clade_543 | Y | N |
| Clostridium sp. L2_50 | 631 | AAYW02000018 | clade_543 | Y | N |
| Coprococcus eutactus | 675 | EF031543 | clade_543 | Y | N |
| Coprococcus sp. ART55_1 | 676 | AY350746 | clade_543 | Y | N |
| Eubacterium ruminantium | 857 | NR_024661 | clade_543 | Y | N |
| Collinsella aerofaciens | 659 | AAVN02000007 | clade_553 | Y | N |
| Alkaliphilus metalliredigenes | 137 | AY137848 | clade_554 | Y | N |
| Alkaliphilus oremlandii | 138 | NR_043674 | clade_554 | Y | N |
| Clostridium sticklandii | 648 | L04167 | clade_554 | Y | N |
| Turicibacter sanguinis | 1965 | AF349724 | clade_555 | Y | N |
| Fulvimonas sp. NML 060897 | 892 | EF589680 | clade_557 | Y | N |
| Desulfitobacterium frappieri | 753 | AJ276701 | clade_560 | Y | N |
| Desulfitobacterium hafniense | 754 | NR_074996 | clade_560 | Y | N |
| Desulfotomaculum nigrificans | 756 | NR_044832 | clade_560 | Y | N |
| Lutispora thermophila | 1191 | NR_041236 | clade_564 | Y | N |
| Brachyspira pilosicoli | 405 | NR_075069 | clade_565 | Y | N |
| Eggerthella lenta | 778 | AF292375 | clade_566 | Y | N |
| Streptomyces albus | 1888 | AJ697941 | clade_566 | Y | N |
| Chlamydiales bacterium NS11 | 505 | JN606074 | clade_567 | Y | N |
| Anaerofustis stercorihominis | 159 | ABIL02000005 | clade_570 | Y | N |
| Butyricicoccus pullicaecorum | 453 | HH793440 | clade_572 | Y | N |
| Eubacterium desmolans | 843 | NR_044644 | clade_572 | Y | N |
| Papillibacter cinnamivorans | 1415 | NR_025025 | clade_572 | Y | N |
| Sporobacter termitidis | 1751 | NR_044972 | clade_572 | Y | N |
| Deferribacteres sp. oral clone JV006 | 744 | AY349371 | clade_575 | Y | N |
| Clostridium colinum | 577 | NR_026151 | clade_576 | Y | N |
| Clostridium lactatifermentans | 599 | NR_025651 | clade_576 | Y | N |
| Clostridium piliforme | 614 | D14639 | clade_576 | Y | N |
| Saccharomonospora viridis | 1671 | X54286 | clade_579 | Y | N |
| Thermobifida fusca | 1921 | NC_007333 | clade_579 | Y | N |
| Leptospira licerasiae | 1164 | EF612284 | clade_585 | Y | OP |
| Moorella thermoacetica | 1259 | NR_075001 | clade_590 | Y | N |
| Thermoanaerobacter pseudethanolicus | 1920 | CP000924 | clade_590 | Y | N |
| Flexistipes sinusarabici | 888 | NR_074881 | clade_591 | Y | N |
| Gloeobacter violaceus | 942 | NR_074282 | clade_596 | Y | N |
| Eubacterium sp. oral clone JN088 | 869 | AY349377 | clade_90 | Y | N |
| Clostridium oroticum | 610 | FR749922 | clade_96 | Y | N |
| Clostridium sp. D5 | 627 | ADBG01000142 | clade_96 | Y | N |
| Eubacterium contortum | 840 | FR749946 | clade_96 | Y | N |
| Eubacterium fissicatena | 846 | FR749935 | clade_96 | Y | N |
| Corynebacterium coyleae | 692 | X96497 | clade_100 | N | N |
| Corynebacterium mucifaciens | 711 | NR_026396 | clade_100 | N | N |
| Corynebacterium ureicelerivorans | 733 | AM397636 | clade_100 | N | N |
| Corynebacterium appendicis | 684 | NR_028951 | clade_102 | N | N |
| Corynebacterium genitalium | 698 | ACLJ01000031 | clade_102 | N | N |
| Corynebacterium glaucum | 699 | NR_028971 | clade_102 | N | N |
| Corynebacterium imitans | 703 | AF537597 | clade_102 | N | N |
| Corynebacterium riegelii | 719 | EU848548 | clade_102 | N | N |
| Corynebacterium sp. L_2012475 | 723 | HE575405 | clade_102 | N | N |
| Corynebacterium sp. NML 93_0481 | 724 | GU238409 | clade_102 | N | N |
| Corynebacterium sundsvallense | 728 | Y09655 | clade_102 | N | N |
| Corynebacterium tuscaniae | 730 | AY677186 | clade_102 | N | N |
| Prevotella maculosa | 1504 | AGEK01000035 | clade_104 | N | N |
| Prevotella oris | 1513 | ADDV01000091 | clade_104 | N | N |
| Prevotella salivae | 1517 | AB108826 | clade_104 | N | N |
| Prevotella sp. ICM55 | 1521 | HQ616399 | clade_104 | N | N |
| Prevotella sp. oral clone AA020 | 1528 | AY005057 | clade_104 | N | N |
| Prevotella sp. oral clone GI032 | 1538 | AY349396 | clade_104 | N | N |
| Prevotella sp. oral taxon G70 | 1558 | GU432179 | clade_104 | N | N |
| Prevotella corporis | 1491 | L16465 | clade_105 | N | N |
| Bacteroides sp. 4_1_36 | 312 | ACTC01000133 | clade_110 | N | N |
| Bacteroides sp. AR20 | 315 | AF139524 | clade_110 | N | N |
| Bacteroides sp. D20 | 319 | ACPT01000052 | clade_110 | N | N |
| Bacteroides sp. F_4 | 322 | AB470322 | clade_110 | N | N |
| Bacteroides uniformis | 329 | AB050110 | clade_110 | N | N |
| Prevotella nanceiensis | 1510 | JN867228 | clade_127 | N | N |
| Prevotella sp. oral taxon 299 | 1548 | ACWZ01000026 | clade_127 | N | N |
| Prevotella bergensis | 1485 | ACKS01000100 | clade_128 | N | N |
| Prevotella buccalis | 1489 | JN867261 | clade_129 | N | N |
| Prevotella timonensis | 1564 | ADEF01000012 | clade_129 | N | N |
| Prevotella oralis | 1512 | AEPE01000021 | clade_130 | N | N |
| Prevotella sp. SEQ072 | 1525 | JN867238 | clade_130 | N | N |
| Leuconostoc carnosum | 1177 | NR_040811 | clade_135 | N | N |
| Leuconostoc gasicomitatum | 1179 | FN822744 | clade_135 | N | N |
| Leuconostoc inhae | 1180 | NR_025204 | clade_135 | N | N |
| Leuconostoc kimchii | 1181 | NR_075014 | clade_135 | N | N |
| Edwardsiella tarda | 777 | CP002154 | clade_139 | N | N |
| Photorhabdus asymbiotica | 1466 | Z76752 | clade_139 | N | N |
| Psychrobacter arcticus | 1607 | CP000082 | clade_141 | N | N |
| Psychrobacter cibarius | 1608 | HQ698586 | clade_141 | N | N |
| Psychrobacter cryohalolentis | 1609 | CP000323 | clade_141 | N | N |
| Psychrobacter faecalis | 1610 | HQ698566 | clade_141 | N | N |
| Psychrobacter nivimaris | 1611 | HQ698587 | clade_141 | N | N |
| Psychrobacter pulmonis | 1612 | HQ698582 | clade_141 | N | N |
| Pseudomonas aeruginosa | 1592 | AABQ07000001 | clade_154 | N | N |
| Pseudomonas sp. 2_1_26 | 1600 | ACWU01000257 | clade_154 | N | N |
| Corynebacterium confusum | 691 | Y15886 | clade_158 | N | N |
| Corynebacterium propinquum | 712 | NR_037038 | clade_158 | N | N |
| Corynebacterium pseudodiphtheriticum | 713 | X84258 | clade_158 | N | N |
| Bartonella bacilliformis | 338 | NC_008783 | clade_159 | N | N |
| Bartonella grahamii | 339 | CP001562 | clade_159 | N | N |
| Bartonella henselae | 340 | NC_005956 | clade_159 | N | N |
| Bartonella quintana | 341 | BX897700 | clade_159 | N | N |
| Bartonella tamiae | 342 | EF672728 | clade_159 | N | N |
| Bartonella washoensis | 343 | FJ719017 | clade_159 | N | N |
| Brucella abortus | 430 | ACBJ01000075 | clade_159 | N | Category-B |
| Brucella canis | 431 | NR_044652 | clade_159 | N | Category-B |
| Brucella ceti | 432 | ACJD01000006 | clade_159 | N | Category-B |
| Brucella melitensis | 433 | AE009462 | clade_159 | N | Category-B |
| Brucella microti | 434 | NR_042549 | clade_159 | N | Category-B |
| Brucella ovis | 435 | NC_009504 | clade_159 | N | Category-B |
| Brucella sp. 83_13 | 436 | ACBQ01000040 | clade_159 | N | Category-B |
| Brucella sp. BO1 | 437 | EU053207 | clade_159 | N | Category-B |
| Brucella suis | 438 | ACBK01000034 | clade_159 | N | Category-B |
| Ochrobactrum anthropi | 1360 | NC_009667 | clade_159 | N | N |
| Ochrobactrum intermedium | 1361 | ACQA01000001 | clade_159 | N | N |
| Ochrobactrum pseudintermedium | 1362 | DQ365921 | clade_159 | N | N |
| Prevotella genomosp. C2 | 1496 | AY278625 | clade_164 | N | N |
| Prevotella multisaccharivorax | 1509 | AFJE01000016 | clade_164 | N | N |
| Prevotella sp. oral clone IDR_CEC_0055 | 1543 | AY550997 | clade_164 | N | N |
| Prevotella sp. oral taxon 292 | 1547 | GQ422735 | clade_164 | N | N |
| Prevotella sp. oral taxon 300 | 1549 | GU409549 | clade_164 | N | N |
| Prevotella marshii | 1505 | AEEI01000070 | clade_166 | N | N |
| Prevotella sp. oral clone IK053 | 1544 | AY349401 | clade_166 | N | N |
| Prevotella sp. oral taxon 781 | 1554 | GQ422744 | clade_166 | N | N |
| Prevotella stercorea | 1562 | AB244774 | clade_166 | N | N |
| Prevotella brevis | 1487 | NR_041954 | clade_167 | N | N |
| Prevotella ruminicola | 1516 | CP002006 | clade_167 | N | N |
| Prevotella sp. sp24 | 1560 | AB003384 | clade_167 | N | N |
| Prevotella sp. sp34 | 1561 | AB003385 | clade_167 | N | N |
| Prevotella albensis | 1483 | NR_025300 | clade_168 | N | N |
| Prevotella copri | 1490 | ACBX02000014 | clade_168 | N | N |
| Prevotella oulorum | 1514 | L16472 | clade_168 | N | N |
| Prevotella sp. BI_42 | 1518 | AJ581354 | clade_168 | N | N |
| Prevotella sp. oral clone P4PB_83 P2 | 1546 | AY207050 | clade_168 | N | N |
| Prevotella sp. oral taxon G60 | 1557 | GU432133 | clade_168 | N | N |
| Prevotella amnii | 1484 | AB547670 | clade_169 | N | N |
| Bacteroides caccae | 268 | EU136686 | clade_170 | N | N |
| Bacteroides finegoldii | 277 | AB222699 | clade_170 | N | N |
| Bacteroides intestinalis | 283 | ABJL02000006 | clade_171 | N | N |
| Bacteroides sp. XB44A | 326 | AM230649 | clade_171 | N | N |
| Bifidobacteriaceae genomosp. C1 | 345 | AY278612 | clade_172 | N | N |
| Bifidobacterium adolescentis | 346 | AAXD02000018 | clade_172 | N | N |
| Bifidobacterium angulatum | 347 | ABYS02000004 | clade_172 | N | N |
| Bifidobacterium animalis | 348 | CP001606 | clade_172 | N | N |
| Bifidobacterium breve | 350 | CP002743 | clade_172 | N | N |
| Bifidobacterium catenulatum | 351 | ABXY01000019 | clade_172 | N | N |
| Bifidobacterium dentium | 352 | CP001750 | clade_172 | N | OP |
| Bifidobacterium gallicum | 353 | ABXB03000004 | clade_172 | N | N |
| Bifidobacterium infantis | 354 | AY151398 | clade_172 | N | N |
| Bifidobacterium kashiwanohense | 355 | AB491757 | clade_172 | N | N |
| Bifidobacterium longum | 356 | ABQQ01000041 | clade_172 | N | N |
| Bifidobacterium pseudocatenulatum | 357 | ABXX02000002 | clade_172 | N | N |
| Bifidobacterium pseudolongum | 358 | NR_043442 | clade_172 | N | N |
| Bifidobacterium scardovii | 359 | AJ307005 | clade_172 | N | N |
| Bifidobacterium sp. HM2 | 360 | AB425276 | clade_172 | N | N |
| Bifidobacterium sp. HMLN12 | 361 | JF519685 | clade_172 | N | N |
| Bifidobacterium sp. M45 | 362 | HM626176 | clade_172 | N | N |
| Bifidobacterium sp. MSX5B | 363 | HQ616382 | clade_172 | N | N |
| Bifidobacterium sp. TM_7 | 364 | AB218972 | clade_172 | N | N |
| Bifidobacterium thermophilum | 365 | DQ340557 | clade_172 | N | N |
| Leuconostoc citreum | 1178 | AM157444 | clade_175 | N | N |
| Leuconostoc lactis | 1182 | NR_040823 | clade_175 | N | N |
| Alicyclobacillus acidoterrestris | 123 | NR_040844 | clade_179 | N | N |
| Alicyclobacillus cycloheptanicus | 125 | NR_024754 | clade_179 | N | N |
| Acinetobacter baumannii | 27 | ACYQ01000014 | clade_181 | N | N |
| Acinetobacter calcoaceticus | 28 | AM157426 | clade_181 | N | N |
| Acinetobacter genomosp. C1 | 29 | AY278636 | clade_181 | N | N |
| Acinetobacter haemolyticus | 30 | ADMT01000017 | clade_181 | N | N |
| Acinetobacter johnsonii | 31 | ACPL01000162 | clade_181 | N | N |
| Acinetobacter junii | 32 | ACPM01000135 | clade_181 | N | N |
| Acinetobacter lwoffii | 33 | ACPN01000204 | clade_181 | N | N |
| Acinetobacter parvus | 34 | AIEB01000124 | clade_181 | N | N |
| Acinetobacter schindleri | 36 | NR_025412 | clade_181 | N | N |
| Acinetobacter sp. 56A1 | 37 | GQ178049 | clade_181 | N | N |
| Acinetobacter sp. CIP 101934 | 38 | JQ638573 | clade_181 | N | N |
| Acinetobacter sp. CIP 102143 | 39 | JQ638578 | clade_181 | N | N |
| Acinetobacter sp. M16_22 | 41 | HM366447 | clade_181 | N | N |
| Acinetobacter sp. RUH2624 | 42 | ACQF01000094 | clade_181 | N | N |
| Acinetobacter sp. SH024 | 43 | ADCH01000068 | clade_181 | N | N |
| Lactobacillus jensenii | 1092 | ACQD01000066 | clade_182 | N | N |
| Alcaligenes faecalis | 119 | AB680368 | clade_183 | N | N |
| Alcaligenes sp. CO14 | 120 | DQ643040 | clade_183 | N | N |
| Alcaligenes sp. S3 | 121 | HQ262549 | clade_183 | N | N |
| Oligella ureolytica | 1366 | NR_041998 | clade_183 | N | N |
| Oligella urethralis | 1367 | NR_041753 | clade_183 | N | N |
| Eikenella corrodens | 784 | ACEA01000028 | clade_185 | N | N |
| Kingella denitrificans | 1019 | AEWV01000047 | clade_185 | N | N |
| Kingella genomosp. P1 oral cone MB2_C20 | 1020 | DQ003616 | clade_185 | N | N |
| Kingella kingae | 1021 | AFHS01000073 | clade_185 | N | N |
| Kingella oralis | 1022 | ACJW02000005 | clade_185 | N | N |
| Kingella sp. oral clone ID059 | 1023 | AY349381 | clade_185 | N | N |
| Neisseria elongata | 1330 | ADBF01000003 | clade_185 | N | N |
| Neisseria genomosp. P2 oral clone MB5_P15 | 1332 | DQ003630 | clade_185 | N | N |
| Neisseria sp. oral clone JC012 | 1345 | AY349388 | clade_185 | N | N |
| Neisseria sp. SMC_A9199 | 1342 | FJ763637 | clade_185 | N | N |
| Simonsiella muelleri | 1731 | ADCY01000105 | clade_185 | N | N |
| Corynebacterium glucuronolyticum | 700 | ABYP01000081 | clade_193 | N | N |
| Corynebacterium pyruviciproducens | 716 | FJ185225 | clade_193 | N | N |
| Rothia aeria | 1649 | DQ673320 | clade_194 | N | N |
| Rothia dentocariosa | 1650 | ADDW01000024 | clade_194 | N | N |
| Rothia sp. oral taxon 188 | 1653 | GU470892 | clade_194 | N | N |
| Corynebacterium accolens | 681 | ACGD01000048 | clade_195 | N | N |
| Corynebacterium macginleyi | 707 | AB359393 | clade_195 | N | N |
| Corynebacterium pseudogenitalium | 714 | ABYQ01000237 | clade_195 | N | N |
| Corynebacterium tuberculostearicum | 729 | ACVP01000009 | clade_195 | N | N |
| Lactobacillus casei | 1074 | CP000423 | clade_198 | N | N |
| Lactobacillus paracasei | 1106 | ABQV01000067 | clade_198 | N | N |
| Lactobacillus zeae | 1143 | NR_037122 | clade_198 | N | N |
| Prevotella dentalis | 1492 | AB547678 | clade_205 | N | N |
| Prevotella sp. oral clone ASCG10 | 1529 | AY923148 | clade_206 | N | N |
| Prevotella sp. oral clone HF050 | 1541 | AY349399 | clade_206 | N | N |
| Prevotella sp. oral clone ID019 | 1542 | AY349400 | clade_206 | N | N |
| Prevotella sp. oral clone IK062 | 1545 | AY349402 | clade_206 | N | N |
| Prevotella genomosp. P9 oral clone MB7_G16 | 1499 | DQ003633 | clade_207 | N | N |
| Prevotella sp. oral clone AU069 | 1531 | AY005062 | clade_207 | N | N |
| Prevotella sp. oral clone CY006 | 1532 | AY005063 | clade_207 | N | N |
| Prevotella sp. oral clone FL019 | 1534 | AY349392 | clade_207 | N | N |
| Actinomyces genomosp. C1 | 56 | AY278610 | clade_212 | N | N |
| Actinomyces genomosp. C2 | 57 | AY278611 | clade_212 | N | N |
| Actinomyces genomosp. P1 oral clone MB6_C03 | 58 | DQ003632 | clade_212 | N | N |
| Actinomyces georgiae | 59 | GU561319 | clade_212 | N | N |
| Actinomyces israelii | 60 | AF479270 | clade_212 | N | N |
| Actinomyces massiliensis | 61 | AB545934 | clade_212 | N | N |
| Actinomyces meyeri | 62 | GU561321 | clade_212 | N | N |
| Actinomyces odontolyticus | 66 | ACYT01000123 | clade_212 | N | N |
| Actinomyces orihominis | 68 | AJ575186 | clade_212 | N | N |
| Actinomyces sp. CCUG 37290 | 71 | AJ234058 | clade_212 | N | N |
| Actinomyces sp. ICM34 | 75 | HQ616391 | clade_212 | N | N |
| Actinomyces sp. ICM41 | 76 | HQ616392 | clade_212 | N | N |
| Actinomyces sp. ICM47 | 77 | HQ616395 | clade_212 | N | N |
| Actinomyces sp. ICM54 | 78 | HQ616398 | clade_212 | N | N |
| Actinomyces sp. oral clone IP081 | 87 | AY349366 | clade_212 | N | N |
| Actinomyces sp. oral taxon 178 | 91 | AEUH01000060 | clade_212 | N | N |
| Actinomyces sp. oral taxon 180 | 92 | AEPP01000041 | clade_212 | N | N |
| Actinomyces sp. TeJ5 | 80 | GU561315 | clade_212 | N | N |
| Haematobacter sp. BC14248 | 968 | GU396991 | clade_213 | N | N |
| Paracoccus denitrificans | 1424 | CP000490 | clade_213 | N | N |
| Paracoccus marcusii | 1425 | NR_044922 | clade_213 | N | N |
| Grimontia hollisae | 967 | ADAQ01000013 | clade_216 | N | N |
| Shewanella putrefaciens | 1723 | CP002457 | clade_216 | N | N |
| Afipia genomosp. 4 | 111 | EU117385 | clade_217 | N | N |
| Rhodopseudomonas palustris | 1626 | CP000301 | clade_217 | N | N |
| Methylobacterium extorquens | 1223 | NC_010172 | clade_218 | N | N |
| Methylobacterium podarium | 1224 | AY468363 | clade_218 | N | N |
| Methylobacterium radiotolerans | 1225 | GU294320 | clade_218 | N | N |
| Methylobacterium sp. 1sub | 1226 | AY468371 | clade_218 | N | N |
| Methylobacterium sp. MM4 | 1227 | AY468370 | clade_218 | N | N |
| Achromobacter denitrificans | 18 | NR_042021 | clade_224 | N | N |
| Achromobacter piechaudii | 19 | ADMS01000149 | clade_224 | N | N |
| Achromobacter xylosoxidans | 20 | ACRC01000072 | clade_224 | N | N |
| Bordetella bronchiseptica | 384 | NR_025949 | clade_224 | N | OP |
| Bordetella holmesii | 385 | AB683187 | clade_224 | N | OP |
| Bordetella parapertussis | 386 | NR_025950 | clade_224 | N | OP |
| Bordetella pertussis | 387 | BX640418 | clade_224 | N | OP |
| Microbacterium chocolatum | 1230 | NR_037045 | clade_225 | N | N |
| Microbacterium flavescens | 1231 | EU714363 | clade_225 | N | N |
| Microbacterium lacticum | 1233 | EU714351 | clade_225 | N | N |
| Microbacterium oleivorans | 1234 | EU714381 | clade_225 | N | N |
| Microbacterium oxydans | 1235 | EU714348 | clade_225 | N | N |
| Microbacterium paraoxydans | 1236 | AJ491806 | clade_225 | N | N |
| Microbacterium phyllosphaerae | 1237 | EU714359 | clade_225 | N | N |
| Microbacterium schleiferi | 1238 | NR_044936 | clade_225 | N | N |
| Microbacterium sp. 768 | 1239 | EU714378 | clade_225 | N | N |
| Microbacterium sp. oral strain C24KA | 1240 | AF287752 | clade_225 | N | N |
| Microbacterium testaceum | 1241 | EU714365 | clade_225 | N | N |
| Corynebacterium atypicum | 686 | NR_025540 | clade_229 | N | N |
| Corynebacterium mastitidis | 708 | AB359395 | clade_229 | N | N |
| Corynebacterium sp. NML 97_0186 | 725 | GU238411 | clade_229 | N | N |
| Mycobacterium elephantis | 1275 | AF385898 | clade_237 | N | OP |
| Mycobacterium paraterrae | 1288 | EU919229 | clade_237 | N | OP |
| Mycobacterium phlei | 1289 | GU142920 | clade_237 | N | OP |
| Mycobacterium sp. 1776 | 1293 | EU703152 | clade_237 | N | N |
| Mycobacterium sp. 1781 | 1294 | EU703147 | clade_237 | N | N |
| Mycobacterium sp. AQ1GA4 | 1297 | HM210417 | clade_237 | N | N |
| Mycobacterium sp. GN_10546 | 1299 | FJ497243 | clade_237 | N | N |
| Mycobacterium sp. GN_10827 | 1300 | FJ497247 | clade_237 | N | N |
| Mycobacterium sp. GN_11124 | 1301 | FJ652846 | clade_237 | N | N |
| Mycobacterium sp. GN_9188 | 1302 | FJ497240 | clade_237 | N | N |
| Mycobacterium sp. GR_2007_210 | 1303 | FJ555538 | clade_237 | N | N |
| Anoxybacillus contaminans | 172 | NR_029006 | clade_238 | N | N |
| Bacillus aeolius | 195 | NR_025557 | clade_238 | N | N |
| Brevibacterium frigoritolerans | 422 | NR_042639 | clade_238 | N | N |
| Geobacillus sp. E263 | 934 | DQ647387 | clade_238 | N | N |
| Geobacillus sp. WCH70 | 935 | CP001638 | clade_238 | N | N |
| Geobacillus thermocatenulatus | 937 | NR_043020 | clade_238 | N | N |
| Geobacillus thermoleovorans | 940 | NR_074931 | clade_238 | N | N |
| Lysinibacillus fusiformis | 1192 | FN397522 | clade_238 | N | N |
| Planomicrobium koreense | 1468 | NR_025011 | clade_238 | N | N |
| Sporosarcina newyorkensis | 1754 | AFPZ01000142 | clade_238 | N | N |
| Sporosarcina sp. 2681 | 1755 | GU994081 | clade_238 | N | N |
| Ureibacillus composti | 1968 | NR_043746 | clade_238 | N | N |
| Ureibacillus suwonensis | 1969 | NR_043232 | clade_238 | N | N |
| Ureibacillus terrenus | 1970 | NR_025394 | clade_238 | N | N |
| Ureibacillus thermophilus | 1971 | NR_043747 | clade_238 | N | N |
| Ureibacillus thermosphaericus | 1972 | NR_040961 | clade_238 | N | N |
| Prevotella micans | 1507 | AGWK01000061 | clade_239 | N | N |
| Prevotella sp. oral clone DA058 | 1533 | AY005065 | clade_239 | N | N |
| Prevotella sp. SEQ053 | 1523 | JN867222 | clade_239 | N | N |
| Treponema socranskii | 1937 | NR_024868 | clade_240 | N | OP |
| Treponema sp. 6:H:D15A_4 | 1938 | AY005083 | clade_240 | N | N |
| Treponema sp. oral taxon 265 | 1953 | GU408850 | clade_240 | N | N |
| Treponema sp. oral taxon G85 | 1958 | GU432215 | clade_240 | N | N |
| Porphyromonas endodontalis | 1472 | ACNN01000021 | clade_241 | N | N |
| Porphyromonas sp. oral clone BB134 | 1478 | AY005068 | clade_241 | N | N |
| Porphyromonas sp. oral clone F016 | 1479 | AY005069 | clade_241 | N | N |
| Porphyromonas sp. oral clone P2PB_52 P1 | 1480 | AY207054 | clade_241 | N | N |
| Porphyromonas sp. oral clone P4GB_100 P2 | 1481 | AY207057 | clade_241 | N | N |
| Acidovorax sp. 98_63833 | 26 | AY258065 | clade_245 | N | N |
| Comamonadaceae bacterium NML000135 | 663 | JN585335 | clade_245 | N | N |
| Comamonadaceae bacterium NML790751 | 664 | JN585331 | clade_245 | N | N |
| Comamonadaceae bacterium NML910035 | 665 | JN585332 | clade_245 | N | N |
| Comamonadaceae bacterium NML910036 | 666 | JN585333 | clade_245 | N | N |
| Comamonas sp. NSP5 | 668 | AB076850 | clade_245 | N | N |
| Delftia acidovorans | 748 | CP000884 | clade_245 | N | N |
| Xenophilus aerolatus | 2018 | JN585329 | clade_245 | N | N |
| Oribacterium sp. oral taxon 078 | 1380 | ACIQ02000009 | clade_246 | N | N |
| Oribacterium sp. oral taxon 102 | 1381 | GQ422713 | clade_246 | N | N |
| Weissella cibaria | 2007 | NR_036924 | clade_247 | N | N |
| Weissella confusa | 2008 | NR_040816 | clade_247 | N | N |
| Weissella hellenica | 2009 | AB680902 | clade_247 | N | N |
| Weissella kandleri | 2010 | NR_044659 | clade_247 | N | N |
| Weissella koreensis | 2011 | NR_075058 | clade_247 | N | N |
| Weissella paramesenteroides | 2012 | ACKU01000017 | clade_247 | N | N |
| Weissella sp. KLDS 7.0701 | 2013 | EU600924 | clade_247 | N | N |
| Mobiluncus curtisii | 1251 | AEPZ01000013 | clade_249 | N | N |
| Enhydrobacter aerosaccus | 785 | ACYI01000081 | clade_256 | N | N |
| Moraxella osloensis | 1262 | JN175341 | clade_256 | N | N |
| Moraxella sp. GM2 | 1264 | JF837191 | clade_256 | N | N |
| Brevibacterium casei | 420 | JF951998 | clade_257 | N | N |
| Brevibacterium epidermidis | 421 | NR_029262 | clade_257 | N | N |
| Brevibacterium sanguinis | 426 | NR_028016 | clade_257 | N | N |
| Brevibacterium sp. H15 | 427 | AB177640 | clade_257 | N | N |
| Acinetobacter radioresistens | 35 | ACVR01000010 | clade_261 | N | N |
| Lactobacillus alimentarius | 1068 | NR_044701 | clade_263 | N | N |
| Lactobacillus farciminis | 1082 | NR_044707 | clade_263 | N | N |
| Lactobacillus kimchii | 1097 | NR_025045 | clade_263 | N | N |
| Lactobacillus nodensis | 1101 | NR_041629 | clade_263 | N | N |
| Lactobacillus tucceti | 1138 | NR_042194 | clade_263 | N | N |
| Pseudomonas mendocina | 1595 | AAUL01000021 | clade_265 | N | N |
| Pseudomonas pseudoalcaligenes | 1598 | NR_037000 | clade_265 | N | N |
| Pseudomonas sp. NP522b | 1602 | EU723211 | clade_265 | N | N |
| Pseudomonas stutzeri | 1603 | AM905854 | clade_265 | N | N |
| Paenibacillus barcinonensis | 1390 | NR_042272 | clade_270 | N | N |
| Paenibacillus barengoltzii | 1391 | NR_042756 | clade_270 | N | N |
| Paenibacillus chibensis | 1392 | NR_040885 | clade_270 | N | N |
| Paenibacillus cookii | 1393 | NR_025372 | clade_270 | N | N |
| Paenibacillus durus | 1394 | NR_037017 | clade_270 | N | N |
| Paenibacillus glucanolyticus | 1395 | D78470 | clade_270 | N | N |
| Paenibacillus lactis | 1396 | NR_025739 | clade_270 | N | N |
| Paenibacillus pabuli | 1398 | NR_040853 | clade_270 | N | N |
| Paenibacillus popilliae | 1400 | NR_040888 | clade_270 | N | N |
| Paenibacillus sp. CIP 101062 | 1401 | HM212646 | clade_270 | N | N |
| Paenibacillus sp. JC66 | 1404 | JF824808 | clade_270 | N | N |
| Paenibacillus sp. R_27413 | 1405 | HE586333 | clade_270 | N | N |
| Paenibacillus sp. R_27422 | 1406 | HE586338 | clade_270 | N | N |
| Paenibacillus timonensis | 1408 | NR_042844 | clade_270 | N | N |
| Rothia mucilaginosa | 1651 | ACVO01000020 | clade_271 | N | N |
| Rothia nasimurium | 1652 | NR_025310 | clade_271 | N | N |
| Prevotella sp. oral taxon 302 | 1550 | ACZK01000043 | clade_280 | N | N |
| Prevotella sp. oral taxon F68 | 1556 | HM099652 | clade_280 | N | N |
| Prevotella tannerae | 1563 | ACIJ02000018 | clade_280 | N | N |
| Prevotellaceae bacterium P4P_62 P1 | 1566 | AY207061 | clade_280 | N | N |
| Porphyromonas asaccharolytica | 1471 | AENO01000048 | clade_281 | N | N |
| Porphyromonas gingivalis | 1473 | AE015924 | clade_281 | N | N |
| Porphyromonas macacae | 1475 | NR_025908 | clade_281 | N | N |
| Porphyromonas sp. UQD 301 | 1477 | EU012301 | clade_281 | N | N |
| Porphyromonas uenonis | 1482 | ACLR01000152 | clade_281 | N | N |
| Leptotrichia buccalis | 1165 | CP001685 | clade_282 | N | N |
| Leptotrichia hofstadii | 1168 | ACVB02000032 | clade_282 | N | N |
| Leptotrichia sp. oral clone HE012 | 1173 | AY349386 | clade_282 | N | N |
| Leptotrichia sp. oral taxon 223 | 1176 | GU408547 | clade_282 | N | N |
| Bacteroides fluxus | 278 | AFBN01000029 | clade_285 | N | N |
| Bacteroides helcogenes | 281 | CP002352 | clade_285 | N | N |
| Parabacteroides johnsonii | 1419 | ABYH01000014 | clade_286 | N | N |
| Parabacteroides merdae | 1420 | EU136685 | clade_286 | N | N |
| Treponema denticola | 1926 | ADEC01000002 | clade_288 | N | OP |
| Treponema genomosp. P5 oral clone MB3_P23 | 1929 | DQ003624 | clade_288 | N | N |
| Treponema putidum | 1935 | AJ543428 | clade_288 | N | OP |
| Treponema sp. oral clone P2PB_53 P3 | 1942 | AY207055 | clade_288 | N | N |
| Treponema sp. oral taxon 247 | 1949 | GU408748 | clade_288 | N | N |
| Treponema sp. oral taxon 250 | 1950 | GU408776 | clade_288 | N | N |
| Treponema sp. oral taxon 251 | 1951 | GU408781 | clade_288 | N | N |
| Anaerococcus hydrogenalis | 144 | ABXA01000039 | clade_289 | N | N |
| Anaerococcus sp. 8404299 | 148 | HM587318 | clade_289 | N | N |
| Anaerococcus sp. gpac215 | 156 | AM176540 | clade_289 | N | N |
| Anaerococcus vaginalis | 158 | ACXU01000016 | clade_289 | N | N |
| Propionibacterium acidipropionici | 1569 | NC_019395 | clade_290 | N | N |
| Propionibacterium avidum | 1571 | AJ003055 | clade_290 | N | N |
| Propionibacterium granulosum | 1573 | FJ785716 | clade_290 | N | N |
| Propionibacterium jensenii | 1574 | NR_042269 | clade_290 | N | N |
| Propionibacterium propionicum | 1575 | NR_025277 | clade_290 | N | N |
| Propionibacterium sp. H456 | 1577 | AB177643 | clade_290 | N | N |
| Propionibacterium thoenii | 1581 | NR_042270 | clade_290 | N | N |
| Bifidobacterium bifidum | 349 | ABQP01000027 | clade_293 | N | N |
| Leuconostoc mesenteroides | 1183 | ACKV01000113 | clade_295 | N | N |
| Leuconostoc pseudomesenteroides | 1184 | NR_040814 | clade_295 | N | N |
| Johnsonella ignava | 1016 | X87152 | clade_298 | N | N |
| Propionibacterium acnes | 1570 | ADJM01000010 | clade_299 | N | N |
| Propionibacterium sp. 434_HC2 | 1576 | AFIL01000035 | clade_299 | N | N |
| Propionibacterium sp. LG | 1578 | AY354921 | clade_299 | N | N |
| Propionibacterium sp. S555a | 1579 | AB264622 | clade_299 | N | N |
| Alicyclobacillus sp. CCUG 53762 | 128 | HE613268 | clade_301 | N | N |
| Actinomyces cardiffensis | 53 | GU470888 | clade_303 | N | N |
| Actinomyces funkei | 55 | HQ906497 | clade_303 | N | N |
| Actinomyces sp. HKU31 | 74 | HQ335393 | clade_303 | N | N |
| Actinomyces sp. oral taxon C55 | 94 | HM099646 | clade_303 | N | N |
| Kerstersia gyiorum | 1018 | NR_025669 | clade_307 | N | N |
| Pigmentiphaga daeguensis | 1467 | JN585327 | clade_307 | N | N |
| Aeromonas allosaccharophila | 104 | S39232 | clade_308 | N | N |
| Aeromonas enteropelogenes | 105 | X71121 | clade_308 | N | N |
| Aeromonas hydrophila | 106 | NC_008570 | clade_308 | N | N |
| Aeromonas jandaei | 107 | X60413 | clade_308 | N | N |
| Aeromonas salmonicida | 108 | NC_009348 | clade_308 | N | N |
| Aeromonas trota | 109 | X60415 | clade_308 | N | N |
| Aeromonas veronii | 110 | NR_044845 | clade_308 | N | N |
| Marvinbryantia formatexigens | 1196 | AJ505973 | clade_309 | N | N |
| Rhodobacter sp. oral taxon C30 | 1620 | HM099648 | clade_310 | N | N |
| Rhodobacter sphaeroides | 1621 | CP000144 | clade_310 | N | N |
| Lactobacillus antri | 1071 | ACLL01000037 | clade_313 | N | N |
| Lactobacillus coleohominis | 1076 | ACOH01000030 | clade_313 | N | N |
| Lactobacillus fermentum | 1083 | CP002033 | clade_313 | N | N |
| Lactobacillus gastricus | 1085 | AICN01000060 | clade_313 | N | N |
| Lactobacillus mucosae | 1099 | FR693800 | clade_313 | N | N |
| Lactobacillus oris | 1103 | AEKL01000077 | clade_313 | N | N |
| Lactobacillus pontis | 1111 | HM218420 | clade_313 | N | N |
| Lactobacillus reuteri | 1112 | ACGW02000012 | clade_313 | N | N |
| Lactobacillus sp. KLDS 1.0707 | 1127 | EU600911 | clade_313 | N | N |
| Lactobacillus sp. KLDS 1.0709 | 1128 | EU600913 | clade_313 | N | N |
| Lactobacillus sp. KLDS 1.0711 | 1129 | EU600915 | clade_313 | N | N |
| Lactobacillus sp. KLDS 1.0713 | 1131 | EU600917 | clade_313 | N | N |
| Lactobacillus sp. KLDS 1.0716 | 1132 | EU600921 | clade_313 | N | N |
| Lactobacillus sp. KLDS 1.0718 | 1133 | EU600922 | clade_313 | N | N |
| Lactobacillus sp. oral taxon 052 | 1137 | GQ422710 | clade_313 | N | N |
| Lactobacillus vaginalis | 1140 | ACGV01000168 | clade_313 | N | N |
| Brevibacterium aurantiacum | 419 | NR_044854 | clade_314 | N | N |
| Brevibacterium linens | 423 | AJ315491 | clade_314 | N | N |
| Lactobacillus pentosus | 1108 | JN813103 | clade_315 | N | N |
| Lactobacillus plantarum | 1110 | ACGZ02000033 | clade_315 | N | N |
| Lactobacillus sp. KLDS 1.0702 | 1123 | EU600906 | clade_315 | N | N |
| Lactobacillus sp. KLDS 1.0703 | 1124 | EU600907 | clade_315 | N | N |
| Lactobacillus sp. KLDS 1.0704 | 1125 | EU600908 | clade_315 | N | N |
| Lactobacillus sp. KLDS 1.0705 | 1126 | EU600909 | clade_315 | N | N |
| Agrobacterium radiobacter | 115 | CP000628 | clade_316 | N | N |
| Agrobacterium tumefaciens | 116 | AJ389893 | clade_316 | N | N |
| Corynebacterium argentoratense | 685 | EF463055 | clade_317 | N | N |
| Corynebacterium diphtheriae | 693 | NC_002935 | clade_317 | N | OP |
| Corynebacterium pseudotuberculosis | 715 | NR_037070 | clade_317 | N | N |
| Corynebacterium renale | 717 | NR_037069 | clade_317 | N | N |
| Corynebacterium ulcerans | 731 | NR_074467 | clade_317 | N | N |
| Aurantimonas coralicida | 191 | AY065627 | clade_318 | N | N |
| Aureimonas altamirensis | 192 | FN658986 | clade_318 | N | N |
| Lactobacillus acidipiscis | 1066 | NR_024718 | clade_320 | N | N |
| Lactobacillus salivarius | 1117 | AEBA01000145 | clade_320 | N | N |
| Lactobacillus sp. KLDS 1.0719 | 1134 | EU600923 | clade_320 | N | N |
| Lactobacillus buchneri | 1073 | ACGH01000101 | clade_321 | N | N |
| Lactobacillus genomosp. C1 | 1086 | AY278619 | clade_321 | N | N |
| Lactobacillus genomosp. C2 | 1087 | AY278620 | clade_321 | N | N |
| Lactobacillus hilgardii | 1089 | ACGP01000200 | clade_321 | N | N |
| Lactobacillus kefiri | 1096 | NR_042230 | clade_321 | N | N |
| Lactobacillus parabuchneri | 1105 | NR_041294 | clade_321 | N | N |
| Lactobacillus parakefiri | 1107 | NR_029039 | clade_321 | N | N |
| Lactobacillus curvatus | 1079 | NR_042437 | clade_322 | N | N |
| Lactobacillus sakei | 1116 | DQ989236 | clade_322 | N | N |
| Aneurinibacillus aneurinilyticus | 167 | AB101592 | clade_323 | N | N |
| Aneurinibacillus danicus | 168 | NR_028657 | clade_323 | N | N |
| Aneurinibacillus migulanus | 169 | NR_036799 | clade_323 | N | N |
| Aneurinibacillus terranovensis | 170 | NR_042271 | clade_323 | N | N |
| Staphylococcus aureus | 1757 | CP002643 | clade_325 | N | Category-B |
| Staphylococcus auricularis | 1758 | JQ624774 | clade_325 | N | N |
| Staphylococcus capitis | 1759 | ACFR01000029 | clade_325 | N | N |
| Staphylococcus caprae | 1760 | ACRH01000033 | clade_325 | N | N |
| Staphylococcus carnosus | 1761 | NR_075003 | clade_325 | N | N |
| Staphylococcus cohnii | 1762 | JN175375 | clade_325 | N | N |
| Staphylococcus condimenti | 1763 | NR_029345 | clade_325 | N | N |
| Staphylococcus epidermidis | 1764 | ACHE01000056 | clade_325 | N | N |
| Staphylococcus equorum | 1765 | NR_027520 | clade_325 | N | N |
| Staphylococcus haemolyticus | 1767 | NC_007168 | clade_325 | N | N |
| Staphylococcus hominis | 1768 | AM157418 | clade_325 | N | N |
| Staphylococcus lugdunensis | 1769 | AEQA01000024 | clade_325 | N | N |
| Staphylococcus pasteuri | 1770 | FJ189773 | clade_325 | N | N |
| Staphylococcus pseudintermedius | 1771 | CP002439 | clade_325 | N | N |
| Staphylococcus saccharolyticus | 1772 | NR_029158 | clade_325 | N | N |
| Staphylococcus saprophyticus | 1773 | NC_007350 | clade_325 | N | N |
| Staphylococcus sp. clone bottae7 | 1777 | AF467424 | clade_325 | N | N |
| Staphylococcus sp. H292 | 1775 | AB177642 | clade_325 | N | N |
| Staphylococcus sp. H780 | 1776 | AB177644 | clade_325 | N | N |
| Staphylococcus succinus | 1778 | NR_028667 | clade_325 | N | N |
| Staphylococcus warneri | 1780 | ACPZ01000009 | clade_325 | N | N |
| Staphylococcus xylosus | 1781 | AY395016 | clade_325 | N | N |
| Cardiobacterium hominis | 490 | ACKY01000036 | clade_326 | N | N |
| Cardiobacterium valvarum | 491 | NR_028847 | clade_326 | N | N |
| Pseudomonas fluorescens | 1593 | AY622220 | clade_326 | N | N |
| Pseudomonas gessardii | 1594 | FJ943496 | clade_326 | N | N |
| Pseudomonas monteilii | 1596 | NR_024910 | clade_326 | N | N |
| Pseudomonas poae | 1597 | GU188951 | clade_326 | N | N |
| Pseudomonas putida | 1599 | AF094741 | clade_326 | N | N |
| Pseudomonas sp. G1229 | 1601 | DQ910482 | clade_326 | N | N |
| Pseudomonas tolaasii | 1604 | AF320988 | clade_326 | N | N |
| Pseudomonas viridiflava | 1605 | NR_042764 | clade_326 | N | N |
| Listeria grayi | 1185 | ACCR02000003 | clade_328 | N | OP |
| Listeria innocua | 1186 | JF967625 | clade_328 | N | N |
| Listeria ivanovii | 1187 | X56151 | clade_328 | N | N |
| Listeria monocytogenes | 1188 | CP002003 | clade_328 | N | Category-B |
| Listeria welshimeri | 1189 | AM263198 | clade_328 | N | OP |
| Capnocytophaga sp. oral clone ASCH05 | 484 | AY923149 | clade_333 | N | N |
| Capnocytophaga sputigena | 489 | ABZV01000054 | clade_333 | N | N |
| Leptotrichia genomosp. C1 | 1166 | AY278621 | clade_334 | N | N |
| Leptotrichia shahii | 1169 | AY029806 | clade_334 | N | N |
| Leptotrichia sp. neutropenicPatient | 1170 | AF189244 | clade_334 | N | N |
| Leptotrichia sp. oral clone GT018 | 1171 | AY349384 | clade_334 | N | N |
| Leptotrichia sp. oral clone GT020 | 1172 | AY349385 | clade_334 | N | N |
| Bacteroides sp. 20_3 | 296 | ACRQ01000064 | clade_335 | N | N |
| Bacteroides sp. 3_1_19 | 307 | ADCJ01000062 | clade_335 | N | N |
| Bacteroides sp. 3_2_5 | 311 | ACIB01000079 | clade_335 | N | N |
| Parabacteroides distasonis | 1416 | CP000140 | clade_335 | N | N |
| Parabacteroides goldsteinii | 1417 | AY974070 | clade_335 | N | N |
| Parabacteroides gordonii | 1418 | AB470344 | clade_335 | N | N |
| Parabacteroides sp. D13 | 1421 | ACPW01000017 | clade_335 | N | N |
| Capnocytophaga genomosp. C1 | 477 | AY278613 | clade_336 | N | N |
| Capnocytophaga ochracea | 480 | AEOH01000054 | clade_336 | N | N |
| Capnocytophaga sp. GEJ8 | 481 | GU561335 | clade_336 | N | N |
| Capnocytophaga sp. oral strain A47ROY | 486 | AY005077 | clade_336 | N | N |
| Capnocytophaga sp. S1b | 482 | U42009 | clade_336 | N | N |
| Paraprevotella clara | 1426 | AFFY01000068 | clade_336 | N | N |
| Bacteroides heparinolyticus | 282 | JN867284 | clade_338 | N | N |
| Prevotella heparinolytica | 1500 | GQ422742 | clade_338 | N | N |
| Treponema genomosp. P4 oral clone MB2_G19 | 1928 | DQ003618 | clade_339 | N | N |
| Treponema genomosp. P6 oral clone MB4_G11 | 1930 | DQ003625 | clade_339 | N | N |
| Treponema sp. oral taxon 254 | 1952 | GU408803 | clade_339 | N | N |
| Treponema sp. oral taxon 508 | 1956 | GU413616 | clade_339 | N | N |
| Treponema sp. oral taxon 518 | 1957 | GU413640 | clade_339 | N | N |
| Chlamydia muridarum | 502 | AE002160 | clade_341 | N | OP |
| Chlamydia trachomatis | 504 | U68443 | clade_341 | N | OP |
| Chlamydia psittaci | 503 | NR_036864 | clade_342 | N | Category-B |
| Chlamydophila pneumoniae | 509 | NC_002179 | clade_342 | N | OP |
| Chlamydophila psittaci | 510 | D85712 | clade_342 | N | OP |
| Anaerococcus octavius | 146 | NR_026360 | clade_343 | N | N |
| Anaerococcus sp. 8405254 | 149 | HM587319 | clade_343 | N | N |
| Anaerococcus sp. 9401487 | 150 | HM587322 | clade_343 | N | N |
| Anaerococcus sp. 9403502 | 151 | HM587325 | clade_343 | N | N |
| Gardnerella vaginalis | 923 | CP001849 | clade_344 | N | N |
| Campylobacter lari | 466 | CP000932 | clade_346 | N | OP |
| Anaerobiospirillum succiniciproducens | 142 | NR_026075 | clade_347 | N | N |
| Anaerobiospirillum thomasii | 143 | AJ420985 | clade_347 | N | N |
| Ruminobacter amylophilus | 1654 | NR_026450 | clade_347 | N | N |
| Succinatimonas hippei | 1897 | AEVO01000027 | clade_347 | N | N |
| Actinomyces europaeus | 54 | NR_026363 | clade_348 | N | N |
| Actinomyces sp. oral clone GU009 | 82 | AY349361 | clade_348 | N | N |
| Moraxella catarrhalis | 1260 | CP002005 | clade_349 | N | N |
| Moraxella lincolnii | 1261 | FR822735 | clade_349 | N | N |
| Moraxella sp. 16285 | 1263 | JF682466 | clade_349 | N | N |
| Psychrobacter sp. 13983 | 1613 | HM212668 | clade_349 | N | N |
| Actinobaculum massiliae | 49 | AF487679 | clade_350 | N | N |
| Actinobaculum schaalii | 50 | AY957507 | clade_350 | N | N |
| Actinobaculum sp. BM#101342 | 51 | AY282578 | clade_350 | N | N |
| Actinobaculum sp. P2P_19 P1 | 52 | AY207066 | clade_350 | N | N |
| Actinomyces sp. oral clone IO076 | 84 | AY349363 | clade_350 | N | N |
| Actinomyces sp. oral taxon 848 | 93 | ACUY01000072 | clade_350 | N | N |
| Actinomyces neuii | 65 | X71862 | clade_352 | N | N |
| Mobiluncus mulieris | 1252 | ACKW01000035 | clade_352 | N | N |
| Blastomonas natatoria | 372 | NR_040824 | clade_356 | N | N |
| Novosphingobium aromaticivorans | 1357 | AAAV03000008 | clade_356 | N | N |
| Sphingomonas sp. oral clone FI012 | 1745 | AY349411 | clade_356 | N | N |
| Sphingopyxis alaskensis | 1749 | CP000356 | clade_356 | N | N |
| Oxalobacter formigenes | 1389 | ACDQ01000020 | clade_357 | N | N |
| Veillonella atypica | 1974 | AEDS01000059 | clade_358 | N | N |
| Veillonella dispar | 1975 | ACIK02000021 | clade_358 | N | N |
| Veillonella genomosp. P1 oral clone MB5_P17 | 1976 | DQ003631 | clade_358 | N | N |
| Veillonella parvula | 1978 | ADFU01000009 | clade_358 | N | N |
| Veillonella sp. 3_1_44 | 1979 | ADCV01000019 | clade_358 | N | N |
| Veillonella sp. 6_1_27 | 1980 | ADCW01000016 | clade_358 | N | N |
| Veillonella sp. ACP1 | 1981 | HQ616359 | clade_358 | N | N |
| Veillonella sp. AS16 | 1982 | HQ616365 | clade_358 | N | N |
| Veillonella sp. BS32b | 1983 | HQ616368 | clade_358 | N | N |
| Veillonella sp. ICM51a | 1984 | HQ616396 | clade_358 | N | N |
| Veillonella sp. MSA12 | 1985 | HQ616381 | clade_358 | N | N |
| Veillonella sp. NVG 100cf | 1986 | EF108443 | clade_358 | N | N |
| Veillonella sp. OK11 | 1987 | JN695650 | clade_358 | N | N |
| Veillonella sp. oral clone ASCG01 | 1990 | AY923144 | clade_358 | N | N |
| Veillonella sp. oral clone ASCG02 | 1991 | AY953257 | clade_358 | N | N |
| Veillonella sp. oral clone OH1A | 1992 | AY947495 | clade_358 | N | N |
| Veillonella sp. oral taxon 158 | 1993 | AENU01000007 | clade_358 | N | N |
| Kocuria marina | 1040 | GQ260086 | clade_365 | N | N |
| Kocuria rhizophila | 1042 | AY030315 | clade_365 | N | N |
| Kocuria rosea | 1043 | X87756 | clade_365 | N | N |
| Kocuria varians | 1044 | AF542074 | clade_365 | N | N |
| Clostridiaceae bacterium END_2 | 531 | EF451053 | clade_368 | N | N |
| Micrococcus antarcticus | 1242 | NR_025285 | clade_371 | N | N |
| Micrococcus luteus | 1243 | NR_075062 | clade_371 | N | N |
| Micrococcus lylae | 1244 | NR_026200 | clade_371 | N | N |
| Micrococcus sp. 185 | 1245 | EU714334 | clade_371 | N | N |
| Lactobacillus brevis | 1072 | EU194349 | clade_372 | N | N |
| Lactobacillus parabrevis | 1104 | NR_042456 | clade_372 | N | N |
| Pediococcus acidilactici | 1436 | ACXB01000026 | clade_372 | N | N |
| Pediococcus pentosaceus | 1437 | NR_075052 | clade_372 | N | N |
| Lactobacillus dextrinicus | 1081 | NR_036861 | clade_373 | N | N |
| Lactobacillus perolens | 1109 | NR_029360 | clade_373 | N | N |
| Lactobacillus rhamnosus | 1113 | ABWJ01000068 | clade_373 | N | N |
| Lactobacillus saniviri | 1118 | AB602569 | clade_373 | N | N |
| Lactobacillus sp. BT6 | 1121 | HQ616370 | clade_373 | N | N |
| Mycobacterium mageritense | 1282 | FR798914 | clade_374 | N | OP |
| Mycobacterium neoaurum | 1286 | AF268445 | clade_374 | N | OP |
| Mycobacterium smegmatis | 1291 | CP000480 | clade_374 | N | OP |
| Mycobacterium sp. HE5 | 1304 | AJ012738 | clade_374 | N | N |
| Dysgonomonas gadei | 775 | ADLV01000001 | clade_377 | N | N |
| Dysgonomonas mossii | 776 | ADLW01000023 | clade_377 | N | N |
| Porphyromonas levii | 1474 | NR_025907 | clade_377 | N | N |
| Porphyromonas somerae | 1476 | AB547667 | clade_377 | N | N |
| Bacteroides barnesiae | 267 | NR_041446 | clade_378 | N | N |
| Bacteroides coprocola | 272 | ABIY02000050 | clade_378 | N | N |
| Bacteroides coprophilus | 273 | ACBW01000012 | clade_378 | N | N |
| Bacteroides dorei | 274 | ABWZ01000093 | clade_378 | N | N |
| Bacteroides massiliensis | 284 | AB200226 | clade_378 | N | N |
| Bacteroides plebeius | 289 | AB200218 | clade_378 | N | N |
| Bacteroides sp. 3_1_33FAA | 309 | ACPS01000085 | clade_378 | N | N |
| Bacteroides sp. 3_1_40A | 310 | ACRT01000136 | clade_378 | N | N |
| Bacteroides sp. 4_3_47FAA | 313 | ACDR02000029 | clade_378 | N | N |
| Bacteroides sp. 9_1_42FAA | 314 | ACAA01000096 | clade_378 | N | N |
| Bacteroides sp. NB_8 | 323 | AB117565 | clade_378 | N | N |
| Bacteroides vulgatus | 331 | CP000139 | clade_378 | N | N |
| Bacteroides ovatus | 287 | ACWH01000036 | clade_38 | N | N |
| Bacteroides sp. 1_1_30 | 294 | ADCL01000128 | clade_38 | N | N |
| Bacteroides sp. 2_1_22 | 297 | ACPQ01000117 | clade_38 | N | N |
| Bacteroides sp. 2_2_4 | 299 | ABZZ01000168 | clade_38 | N | N |
| Bacteroides sp. 3_1_23 | 308 | ACRS01000081 | clade_38 | N | N |
| Bacteroides sp. D1 | 318 | ACAB02000030 | clade_38 | N | N |
| Bacteroides sp. D2 | 321 | ACGA01000077 | clade_38 | N | N |
| Bacteroides sp. D22 | 320 | ADCK01000151 | clade_38 | N | N |
| Bacteroides xylanisolvens | 332 | ADKP01000087 | clade_38 | N | N |
| Treponema lecithinolyticum | 1931 | NR_026247 | clade_380 | N | OP |
| Treponema parvum | 1933 | AF302937 | clade_380 | N | OP |
| Treponema sp. oral clone JU025 | 1940 | AY349417 | clade_380 | N | N |
| Treponema sp. oral taxon 270 | 1954 | GQ422733 | clade_380 | N | N |
| Parascardovia denticolens | 1428 | ADEB01000020 | clade_381 | N | N |
| Scardovia inopinata | 1688 | AB029087 | clade_381 | N | N |
| Scardovia wiggsiae | 1689 | AY278626 | clade_381 | N | N |
| Clostridiales bacterium 9400853 | 533 | HM587320 | clade_384 | N | N |
| Mogibacterium diversum | 1254 | NR_027191 | clade_384 | N | N |
| Mogibacterium neglectum | 1255 | NR_027203 | clade_384 | N | N |
| Mogibacterium pumilum | 1256 | NR_028608 | clade_384 | N | N |
| Mogibacterium timidum | 1257 | Z36296 | clade_384 | N | N |
| Borrelia burgdorferi | 389 | ABGI01000001 | clade_386 | N | OP |
| Borrelia garinii | 392 | ABJV01000001 | clade_386 | N | OP |
| Borrelia sp. NE49 | 397 | AJ224142 | clade_386 | N | OP |
| Caldimonas manganoxidans | 457 | NR_040787 | clade_387 | N | N |
| Comamonadaceae bacterium oral taxon F47 | 667 | HM099651 | clade_387 | N | N |
| Lautropia mirabilis | 1149 | AEQP01000026 | clade_387 | N | N |
| Lautropia sp. oral clone AP009 | 1150 | AY005030 | clade_387 | N | N |
| Peptoniphilus asaccharolyticus | 1441 | D14145 | clade_389 | N | N |
| Peptoniphilus duerdenii | 1442 | EU526290 | clade_389 | N | N |
| Peptoniphilus harei | 1443 | NR_026358 | clade_389 | N | N |
| Peptoniphilus indolicus | 1444 | AY153431 | clade_389 | N | N |
| Peptoniphilus lacrimalis | 1446 | ADDO01000050 | clade_389 | N | N |
| Peptoniphilus sp. gpac077 | 1450 | AM176527 | clade_389 | N | N |
| Peptoniphilus sp. JC140 | 1447 | JF824803 | clade_389 | N | N |
| Peptoniphilus sp. oral taxon 386 | 1452 | ADCS01000031 | clade_389 | N | N |
| Peptoniphilus sp. oral taxon 836 | 1453 | AEAA01000090 | clade_389 | N | N |
| Peptostreptococcaceae bacterium ph1 | 1454 | JN837495 | clade_389 | N | N |
| Dialister pneumosintes | 765 | HM596297 | clade_390 | N | N |
| Dialister sp. oral taxon 502 | 767 | GQ422739 | clade_390 | N | N |
| Cupriavidus metallidurans | 741 | GU230889 | clade_391 | N | N |
| Herbaspirillum seropedicae | 1001 | CP002039 | clade_391 | N | N |
| Herbaspirillum sp. JC206 | 1002 | JN657219 | clade_391 | N | N |
| Janthinobacterium sp. SY12 | 1015 | EF455530 | clade_391 | N | N |
| Massilia sp. CCUG 43427A | 1197 | FR773700 | clade_391 | N | N |
| Ralstonia pickettii | 1615 | NC_010682 | clade_391 | N | N |
| Ralstonia sp. 5_7_47FAA | 1616 | ACUF01000076 | clade_391 | N | N |
| Francisella novicida | 889 | ABSS01000002 | clade_392 | N | N |
| Francisella philomiragia | 890 | AY928394 | clade_392 | N | N |
| Francisella tularensis | 891 | ABAZ01000082 | clade_392 | N | Category-A |
| Ignatzschineria indica | 1009 | HQ823562 | clade_392 | N | N |
| Ignatzschineria sp. NML 95_0260 | 1010 | HQ823559 | clade_392 | N | N |
| Streptococcus mutans | 1814 | AP010655 | clade_394 | N | N |
| Lactobacillus gasseri | 1084 | ACOZ01000018 | clade_398 | N | N |
| Lactobacillus hominis | 1090 | FR681902 | clade_398 | N | N |
| Lactobacillus iners | 1091 | AEKJ01000002 | clade_398 | N | N |
| Lactobacillus johnsonii | 1093 | AE017198 | clade_398 | N | N |
| Lactobacillus senioris | 1119 | AB602570 | clade_398 | N | N |
| Lactobacillus sp. oral clone HT002 | 1135 | AY349382 | clade_398 | N | N |
| Weissella beninensis | 2006 | EU439435 | clade_398 | N | N |
| Sphingomonas echinoides | 1744 | NR_024700 | clade_399 | N | N |
| Sphingomonas sp. oral taxon A09 | 1747 | HM099639 | clade_399 | N | N |
| Sphingomonas sp. oral taxon F71 | 1748 | HM099645 | clade_399 | N | N |
| Zymomonas mobilis | 2032 | NR_074274 | clade_399 | N | N |
| Arcanobacterium haemolyticum | 174 | NR_025347 | clade_400 | N | N |
| Arcanobacterium pyogenes | 175 | GU585578 | clade_400 | N | N |
| Trueperella pyogenes | 1962 | NR_044858 | clade_400 | N | N |
| Lactococcus garvieae | 1144 | AF061005 | clade_401 | N | N |
| Lactococcus lactis | 1145 | CP002365 | clade_401 | N | N |
| Brevibacterium mcbrellneri | 424 | ADNU01000076 | clade_402 | N | N |
| Brevibacterium paucivorans | 425 | EU086796 | clade_402 | N | N |
| Brevibacterium sp. JC43 | 428 | JF824806 | clade_402 | N | N |
| Selenomonas artemidis | 1692 | HM596274 | clade_403 | N | N |
| Selenomonas sp. FOBRC9 | 1704 | HQ616378 | clade_403 | N | N |
| Selenomonas sp. oral taxon 137 | 1715 | AENV01000007 | clade_403 | N | N |
| Desmospora activa | 751 | AM940019 | clade_404 | N | N |
| Desmospora sp. 8437 | 752 | AFHT01000143 | clade_404 | N | N |
| Paenibacillus sp. oral taxon F45 | 1407 | HM099647 | clade_404 | N | N |
| Corynebacterium ammoniagenes | 682 | ADNS01000011 | clade_405 | N | N |
| Corynebacterium aurimucosum | 687 | ACLH01000041 | clade_405 | N | N |
| Corynebacterium bovis | 688 | AF537590 | clade_405 | N | N |
| Corynebacterium canis | 689 | GQ871934 | clade_405 | N | N |
| Corynebacterium casei | 690 | NR_025101 | clade_405 | N | N |
| Corynebacterium durum | 694 | Z97069 | clade_405 | N | N |
| Corynebacterium efficiens | 695 | ACLI01000121 | clade_405 | N | N |
| Corynebacterium falsenii | 696 | Y13024 | clade_405 | N | N |
| Corynebacterium flavescens | 697 | NR_037040 | clade_405 | N | N |
| Corynebacterium glutamicum | 701 | BA000036 | clade_405 | N | N |
| Corynebacterium jeikeium | 704 | ACYW01000001 | clade_405 | N | OP |
| Corynebacterium kroppenstedtii | 705 | NR_026380 | clade_405 | N | N |
| Corynebacterium lipophiloflavum | 706 | ACHJ01000075 | clade_405 | N | N |
| Corynebacterium matruchotii | 709 | ACSH02000003 | clade_405 | N | N |
| Corynebacterium minutissimum | 710 | X82064 | clade_405 | N | N |
| Corynebacterium resistens | 718 | ADGN01000058 | clade_405 | N | N |
| Corynebacterium simulans | 720 | AF537604 | clade_405 | N | N |
| Corynebacterium singulare | 721 | NR_026394 | clade_405 | N | N |
| Corynebacterium sp. 1 ex sheep | 722 | Y13427 | clade_405 | N | N |
| Corynebacterium sp. NML 99_0018 | 726 | GU238413 | clade_405 | N | N |
| Corynebacterium striatum | 727 | ACGE01000001 | clade_405 | N | OP |
| Corynebacterium urealyticum | 732 | X81913 | clade_405 | N | OP |
| Corynebacterium variabile | 734 | NR_025314 | clade_405 | N | N |
| Aerococcus sanguinicola | 98 | AY837833 | clade_407 | N | N |
| Aerococcus urinae | 99 | CP002512 | clade_407 | N | N |
| Aerococcus urinaeequi | 100 | NR_043443 | clade_407 | N | N |
| Aerococcus viridans | 101 | ADNT01000041 | clade_407 | N | N |
| Fusobacterium naviforme | 898 | HQ223106 | clade_408 | N | N |
| Moryella indoligenes | 1268 | AF527773 | clade_408 | N | N |
| Selenomonas genomosp. P5 | 1697 | AY341820 | clade_410 | N | N |
| Selenomonas sp. oral clone IQ048 | 1710 | AY349408 | clade_410 | N | N |
| Selenomonas sputigena | 1717 | ACKP02000033 | clade_410 | N | N |
| Hyphomicrobium sulfonivorans | 1007 | AY468372 | clade_411 | N | N |
| Methylocella silvestris | 1228 | NR_074237 | clade_411 | N | N |
| Legionella pneumophila | 1153 | NC_002942 | clade_412 | N | OP |
| Lactobacillus coryniformis | 1077 | NR_044705 | clade_413 | N | N |
| Arthrobacter agilis | 178 | NR_026198 | clade_414 | N | N |
| Arthrobacter arilaitensis | 179 | NR_074608 | clade_414 | N | N |
| Arthrobacter bergerei | 180 | NR_025612 | clade_414 | N | N |
| Arthrobacter globiformis | 181 | NR_026187 | clade_414 | N | N |
| Arthrobacter nicotianae | 182 | NR_026190 | clade_414 | N | N |
| Mycobacterium abscessus | 1269 | AGQU01000002 | clade_418 | N | OP |
| Mycobacterium chelonae | 1273 | AB548610 | clade_418 | N | OP |
| Bacteroides salanitronis | 291 | CP002530 | clade_419 | N | N |
| Paraprevotella xylaniphila | 1427 | AFBR01000011 | clade_419 | N | N |
| Barnesiella intestinihominis | 336 | AB370251 | clade_420 | N | N |
| Barnesiella viscericola | 337 | NR_041508 | clade_420 | N | N |
| Parabacteroides sp. NS31_3 | 1422 | JN029805 | clade_420 | N | N |
| Porphyromonadaceae bacterium NML 060648 | 1470 | EF184292 | clade_420 | N | N |
| Tannerella forsythia | 1913 | CP003191 | clade_420 | N | N |
| Tannerella sp. 6_1_58FAA_CT1 | 1914 | ACWX01000068 | clade_420 | N | N |
| Mycoplasma amphoriforme | 1311 | AY531656 | clade_421 | N | N |
| Mycoplasma genitalium | 1317 | L43967 | clade_421 | N | N |
| Mycoplasma pneumoniae | 1322 | NC_000912 | clade_421 | N | N |
| Mycoplasma penetrans | 1321 | NC_004432 | clade_422 | N | N |
| Ureaplasma parvum | 1966 | AE002127 | clade_422 | N | N |
| Ureaplasma urealyticum | 1967 | AAYN01000002 | clade_422 | N | N |
| Treponema genomosp. P1 | 1927 | AY341822 | clade_425 | N | N |
| Treponema sp. oral taxon 228 | 1943 | GU408580 | clade_425 | N | N |
| Treponema sp. oral taxon 230 | 1944 | GU408603 | clade_425 | N | N |
| Treponema sp. oral taxon 231 | 1945 | GU408631 | clade_425 | N | N |
| Treponema sp. oral taxon 232 | 1946 | GU408646 | clade_425 | N | N |
| Treponema sp. oral taxon 235 | 1947 | GU408673 | clade_425 | N | N |
| Treponema sp. ovine footrot | 1959 | AJ010951 | clade_425 | N | N |
| Treponema vincentii | 1960 | ACYH01000036 | clade_425 | N | OP |
| Burkholderiales bacterium 1_1_47 | 452 | ADCQ01000066 | clade_432 | N | OP |
| Parasutterella excrementihominis | 1429 | AFBP01000029 | clade_432 | N | N |
| Parasutterella secunda | 1430 | AB491209 | clade_432 | N | N |
| Sutterella morbirenis | 1898 | AJ832129 | clade_432 | N | N |
| Sutterella sanguinus | 1900 | AJ748647 | clade_432 | N | N |
| Sutterella sp. YIT 12072 | 1901 | AB491210 | clade_432 | N | N |
| Sutterella stercoricanis | 1902 | NR_025600 | clade_432 | N | N |
| Sutterella wadsworthensis | 1903 | ADMF01000048 | clade_432 | N | N |
| Propionibacterium freudenreichii | 1572 | NR_036972 | clade_433 | N | N |
| Propionibacterium sp. oral taxon 192 | 1580 | GQ422728 | clade_433 | N | N |
| Tessaracoccus sp. oral taxon F04 | 1917 | HM099640 | clade_433 | N | N |
| Peptoniphilus ivorii | 1445 | Y07840 | clade_434 | N | N |
| Peptoniphilus sp. gpac007 | 1448 | AM176517 | clade_434 | N | N |
| Peptoniphilus sp. gpac018A | 1449 | AM176519 | clade_434 | N | N |
| Peptoniphilus sp. gpac148 | 1451 | AM176535 | clade_434 | N | N |
| Flexispira rappini | 887 | AY126479 | clade_436 | N | N |
| Helicobacter bilis | 993 | ACDN01000023 | clade_436 | N | N |
| Helicobacter cinaedi | 995 | ABQT01000054 | clade_436 | N | N |
| Helicobacter sp. None | 998 | U44756 | clade_436 | N | N |
| Brevundimonas subvibrioides | 429 | CP002102 | clade_438 | N | N |
| Hyphomonas neptunium | 1008 | NR_074092 | clade_438 | N | N |
| Phenylobacterium zucineum | 1465 | AY628697 | clade_438 | N | N |
| Streptococcus downei | 1793 | AEKN01000002 | clade_441 | N | N |
| Streptococcus sp. SHV515 | 1848 | Y07601 | clade_441 | N | N |
| Acinetobacter sp. CIP 53.82 | 40 | JQ638584 | clade_443 | N | N |
| Halomonas elongata | 990 | NR_074782 | clade_443 | N | N |
| Halomonas johnsoniae | 991 | FR775979 | clade_443 | N | N |
| Butyrivibrio fibrisolvens | 456 | U41172 | clade_444 | N | N |
| Roseburia sp. 11SE37 | 1640 | FM954975 | clade_444 | N | N |
| Roseburia sp. 11SE38 | 1641 | FM954976 | clade_444 | N | N |
| Shuttleworthia satelles | 1728 | ACIP02000004 | clade_444 | N | N |
| Shuttleworthia sp. MSX8B | 1729 | HQ616383 | clade_444 | N | N |
| Shuttleworthia sp. oral taxon G69 | 1730 | GU432167 | clade_444 | N | N |
| Bdellovibrio sp. MPA | 344 | AY294215 | clade_445 | N | N |
| Desulfobulbus sp. oral clone CH031 | 755 | AY005036 | clade_445 | N | N |
| Desulfovibrio desulfuricans | 757 | DQ092636 | clade_445 | N | N |
| Desulfovibrio fairfieldensis | 758 | U42221 | clade_445 | N | N |
| Desulfovibrio piger | 759 | AF192152 | clade_445 | N | N |
| Desulfovibrio sp. 3_1_syn3 | 760 | ADDR01000239 | clade_445 | N | N |
| Geobacter bemidjiensis | 941 | CP001124 | clade_445 | N | N |
| Brachybacterium alimentarium | 401 | NR_026269 | clade_446 | N | N |
| Brachybacterium conglomeratum | 402 | AB537169 | clade_446 | N | N |
| Brachybacterium tyrofermentans | 403 | NR_026272 | clade_446 | N | N |
| Dermabacter hominis | 749 | FJ263375 | clade_446 | N | N |
| Aneurinibacillus thermoaerophilus | 171 | NR_029303 | clade_448 | N | N |
| Brevibacillus agri | 409 | NR_040983 | clade_448 | N | N |
| Brevibacillus centrosporus | 411 | NR_043414 | clade_448 | N | N |
| Brevibacillus choshinensis | 412 | NR_040980 | clade_448 | N | N |
| Brevibacillus invocatus | 413 | NR_041836 | clade_448 | N | N |
| Brevibacillus parabrevis | 415 | NR_040981 | clade_448 | N | N |
| Brevibacillus reuszeri | 416 | NR_040982 | clade_448 | N | N |
| Brevibacillus sp. phR | 417 | JN837488 | clade_448 | N | N |
| Brevibacillus thermoruber | 418 | NR_026514 | clade_448 | N | N |
| Lactobacillus murinus | 1100 | NR_042231 | clade_449 | N | N |
| Lactobacillus oeni | 1102 | NR_043095 | clade_449 | N | N |
| Lactobacillus ruminis | 1115 | ACGS02000043 | clade_449 | N | N |
| Lactobacillus vini | 1141 | NR_042196 | clade_449 | N | N |
| Gemella haemolysans | 924 | ACDZ02000012 | clade_450 | N | N |
| Gemella morbillorum | 925 | NR_025904 | clade_450 | N | N |
| Gemella morbillorum | 926 | ACRX01000010 | clade_450 | N | N |
| Gemella sanguinis | 927 | ACRY01000057 | clade_450 | N | N |
| Gemella sp. oral clone ASCE02 | 929 | AY923133 | clade_450 | N | N |
| Gemella sp. oral clone ASCF04 | 930 | AY923139 | clade_450 | N | N |
| Gemella sp. oral clone ASCF12 | 931 | AY923143 | clade_450 | N | N |
| Gemella sp. WAL 1945J | 928 | EU427463 | clade_450 | N | N |
| Sporolactobacillus nakayamae | 1753 | NR_042247 | clade_451 | N | N |
| Gluconacetobacter entanii | 945 | NR_028909 | clade_452 | N | N |
| Gluconacetobacter europaeus | 946 | NR_026513 | clade_452 | N | N |
| Gluconacetobacter hansenii | 947 | NR_026133 | clade_452 | N | N |
| Gluconacetobacter oboediens | 949 | NR_041295 | clade_452 | N | N |
| Gluconacetobacter xylinus | 950 | NR_074338 | clade_452 | N | N |
| Auritibacter ignavus | 193 | FN554542 | clade_453 | N | N |
| Dermacoccus sp. Ellin185 | 750 | AEIQ01000090 | clade_453 | N | N |
| Janibacter limosus | 1013 | NR_026362 | clade_453 | N | N |
| Janibacter melonis | 1014 | EF063716 | clade_453 | N | N |
| Acetobacter aceti | 7 | NR_026121 | clade_454 | N | N |
| Acetobacter fabarum | 8 | NR_042678 | clade_454 | N | N |
| Acetobacter lovaniensis | 9 | NR_040832 | clade_454 | N | N |
| Acetobacter malorum | 10 | NR_025513 | clade_454 | N | N |
| Acetobacter orientalis | 11 | NR_028625 | clade_454 | N | N |
| Acetobacter pasteurianus | 12 | NR_026107 | clade_454 | N | N |
| Acetobacter pomorum | 13 | NR_042112 | clade_454 | N | N |
| Acetobacter syzygii | 14 | NR_040868 | clade_454 | N | N |
| Acetobacter tropicalis | 15 | NR_036881 | clade_454 | N | N |
| Gluconacetobacter azotocaptans | 943 | NR_028767 | clade_454 | N | N |
| Gluconacetobacter diazotrophicus | 944 | NR_074292 | clade_454 | N | N |
| Gluconacetobacter johannae | 948 | NR_024959 | clade_454 | N | N |
| Nocardia brasiliensis | 1351 | AIHV01000038 | clade_455 | N | N |
| Nocardia cyriacigeorgica | 1352 | HQ009486 | clade_455 | N | N |
| Nocardia puris | 1354 | NR_028994 | clade_455 | N | N |
| Nocardia sp. 01_Je_025 | 1355 | GU574059 | clade_455 | N | N |
| Rhodococcus equi | 1623 | ADNW01000058 | clade_455 | N | N |
| Oceanobacillus caeni | 1358 | NR_041533 | clade_456 | N | N |
| Oceanobacillus sp. Ndiop | 1359 | CAER01000083 | clade_456 | N | N |
| Ornithinibacillus bavariensis | 1384 | NR_044923 | clade_456 | N | N |
| Ornithinibacillus sp. 7_10AIA | 1385 | FN397526 | clade_456 | N | N |
| Virgibacillus proomii | 2005 | NR_025308 | clade_456 | N | N |
| Corynebacterium amycolatum | 683 | ABZU01000033 | clade_457 | N | OP |
| Corynebacterium hansenii | 702 | AM946639 | clade_457 | N | N |
| Corynebacterium xerosis | 735 | FN179330 | clade_457 | N | OP |
| Staphylococcaceae bacterium NML 92_0017 | 1756 | AY841362 | clade_458 | N | N |
| Staphylococcus fleurettii | 1766 | NR_041326 | clade_458 | N | N |
| Staphylococcus sciuri | 1774 | NR_025520 | clade_458 | N | N |
| Staphylococcus vitulinus | 1779 | NR_024670 | clade_458 | N | N |
| Stenotrophomonas maltophilia | 1782 | AAVZ01000005 | clade_459 | N | N |
| Stenotrophomonas sp. FG_6 | 1783 | EF017810 | clade_459 | N | N |
| Mycobacterium africanum | 1270 | AF480605 | clade_46 | N | OP |
| Mycobacterium alsiensis | 1271 | AJ938169 | clade_46 | N | OP |
| Mycobacterium avium | 1272 | CP000479 | clade_46 | N | OP |
| Mycobacterium colombiense | 1274 | AM062764 | clade_46 | N | OP |
| Mycobacterium gordonae | 1276 | GU142930 | clade_46 | N | OP |
| Mycobacterium intracellulare | 1277 | GQ153276 | clade_46 | N | OP |
| Mycobacterium kansasii | 1278 | AF480601 | clade_46 | N | OP |
| Mycobacterium lacus | 1279 | NR_025175 | clade_46 | N | OP |
| Mycobacterium leprae | 1280 | FM211192 | clade_46 | N | OP |
| Mycobacterium lepromatosis | 1281 | EU203590 | clade_46 | N | OP |
| Mycobacterium mantenii | 1283 | FJ042897 | clade_46 | N | OP |
| Mycobacterium marinum | 1284 | NC_010612 | clade_46 | N | OP |
| Mycobacterium microti | 1285 | NR_025234 | clade_46 | N | OP |
| Mycobacterium parascrofulaceum | 1287 | ADNV01000350 | clade_46 | N | OP |
| Mycobacterium seoulense | 1290 | DQ536403 | clade_46 | N | OP |
| Mycobacterium sp. 1761 | 1292 | EU703150 | clade_46 | N | N |
| Mycobacterium sp. 1791 | 1295 | EU703148 | clade_46 | N | N |
| Mycobacterium sp. 1797 | 1296 | EU703149 | clade_46 | N | N |
| Mycobacterium sp. B10_07.09.0206 | 1298 | HQ174245 | clade_46 | N | N |
| Mycobacterium sp. NLA001000736 | 1305 | HM627011 | clade_46 | N | N |
| Mycobacterium sp. W | 1306 | DQ437715 | clade_46 | N | N |
| Mycobacterium tuberculosis | 1307 | CP001658 | clade_46 | N | Category-C |
| Mycobacterium ulcerans | 1308 | AB548725 | clade_46 | N | OP |
| Mycobacterium vulneris | 1309 | EU834055 | clade_46 | N | OP |
| Xanthomonas campestris | 2016 | EF101975 | clade_461 | N | N |
| Xanthomonas sp. kmd_489 | 2017 | EU723184 | clade_461 | N | N |
| Dietzia natronolimnaea | 769 | GQ870426 | clade_462 | N | N |
| Dietzia sp. BBDP51 | 770 | DQ337512 | clade_462 | N | N |
| Dietzia sp. CA149 | 771 | GQ870422 | clade_462 | N | N |
| Dietzia timorensis | 772 | GQ870424 | clade_462 | N | N |
| Gordonia bronchialis | 951 | NR_027594 | clade_463 | N | N |
| Gordonia polyisoprenivorans | 952 | DQ385609 | clade_463 | N | N |
| Gordonia sp. KTR9 | 953 | DQ068383 | clade_463 | N | N |
| Gordonia sputi | 954 | FJ536304 | clade_463 | N | N |
| Gordonia terrae | 955 | GQ848239 | clade_463 | N | N |
| Leptotrichia goodfellowii | 1167 | ADAD01000110 | clade_465 | N | N |
| Leptotrichia sp. oral clone IK040 | 1174 | AY349387 | clade_465 | N | N |
| Leptotrichia sp. oral clone P2PB_51 P1 | 1175 | AY207053 | clade_465 | N | N |
| Bacteroidales genomosp. P7 oral clone MB3_P19 | 264 | DQ003623 | clade_466 | N | N |
| Butyricimonas virosa | 454 | AB443949 | clade_466 | N | N |
| Odoribacter laneus | 1363 | AB490805 | clade_466 | N | N |
| Odoribacter splanchnicus | 1364 | CP002544 | clade_466 | N | N |
| Capnocytophaga gingivalis | 478 | ACLQ01000011 | clade_467 | N | N |
| Capnocytophaga granulosa | 479 | X97248 | clade_467 | N | N |
| Capnocytophaga sp. oral clone AH015 | 483 | AY005074 | clade_467 | N | N |
| Capnocytophaga sp. oral strain S3 | 487 | AY005073 | clade_467 | N | N |
| Capnocytophaga sp. oral taxon 338 | 488 | AEXX01000050 | clade_467 | N | N |
| Capnocytophaga canimorsus | 476 | CP002113 | clade_468 | N | N |
| Capnocytophaga sp. oral clone ID062 | 485 | AY349368 | clade_468 | N | N |
| Lactobacillus catenaformis | 1075 | M23729 | clade_469 | N | N |
| Lactobacillus vitulinus | 1142 | NR_041305 | clade_469 | N | N |
| Cetobacterium somerae | 501 | AJ438155 | clade_470 | N | N |
| Fusobacterium gonidiaformans | 896 | ACET01000043 | clade_470 | N | N |
| Fusobacterium mortiferum | 897 | ACDB02000034 | clade_470 | N | N |
| Fusobacterium necrogenes | 899 | X55408 | clade_470 | N | N |
| Fusobacterium necrophorum | 900 | AM905356 | clade_470 | N | N |
| Fusobacterium sp. 12_1B | 905 | AGWJ01000070 | clade_470 | N | N |
| Fusobacterium sp. 3_1_5R | 911 | ACDD01000078 | clade_470 | N | N |
| Fusobacterium sp. D12 | 918 | ACDG02000036 | clade_470 | N | N |
| Fusobacterium ulcerans | 921 | ACDH01000090 | clade_470 | N | N |
| Fusobacterium varium | 922 | ACIE01000009 | clade_470 | N | N |
| Mycoplasma arthritidis | 1312 | NC_011025 | clade_473 | N | N |
| Mycoplasma faucium | 1314 | NR_024983 | clade_473 | N | N |
| Mycoplasma hominis | 1318 | AF443616 | clade_473 | N | N |
| Mycoplasma orale | 1319 | AY796060 | clade_473 | N | N |
| Mycoplasma salivarium | 1324 | M24661 | clade_473 | N | N |
| Mitsuokella jalaludinii | 1247 | NR_028840 | clade_474 | N | N |
| Mitsuokella multacida | 1248 | ABWK02000005 | clade_474 | N | N |
| Mitsuokella sp. oral taxon 521 | 1249 | GU413658 | clade_474 | N | N |
| Mitsuokella sp. oral taxon G68 | 1250 | GU432166 | clade_474 | N | N |
| Selenomonas genomosp. C1 | 1695 | AY278627 | clade_474 | N | N |
| Selenomonas genomosp. P8 oral clone MB5_P06 | 1700 | DQ003628 | clade_474 | N | N |
| Selenomonas ruminantium | 1703 | NR_075026 | clade_474 | N | N |
| Veillonellaceae bacterium oral taxon 131 | 1994 | GU402916 | clade_474 | N | N |
| Alloscardovia omnicolens | 139 | NR_042583 | clade_475 | N | N |
| Alloscardovia sp. OB7196 | 140 | AB425070 | clade_475 | N | N |
| Bifidobacterium urinalis | 366 | AJ278695 | clade_475 | N | N |
| Prevotella loescheii | 1503 | JN867231 | clade_48 | N | N |
| Prevotella sp. oral clone ASCG12 | 1530 | DQ272511 | clade_48 | N | N |
| Prevotella sp. oral clone GU027 | 1540 | AY349398 | clade_48 | N | N |
| Prevotella sp. oral taxon 472 | 1553 | ACZS01000106 | clade_48 | N | N |
| Selenomonas dianae | 1693 | GQ422719 | clade_480 | N | N |
| Selenomonas flueggei | 1694 | AF287803 | clade_480 | N | N |
| Selenomonas genomosp. C2 | 1696 | AY278628 | clade_480 | N | N |
| Selenomonas genomosp. P6 oral clone MB3_C41 | 1698 | DQ003636 | clade_480 | N | N |
| Selenomonas genomosp. P7 oral clone MB5_C08 | 1699 | DQ003627 | clade_480 | N | N |
| Selenomonas infelix | 1701 | AF287802 | clade_480 | N | N |
| Selenomonas noxia | 1702 | GU470909 | clade_480 | N | N |
| Selenomonas sp. oral clone FT050 | 1705 | AY349403 | clade_480 | N | N |
| Selenomonas sp. oral clone GI064 | 1706 | AY349404 | clade_480 | N | N |
| Selenomonas sp. oral clone GT010 | 1707 | AY349405 | clade_480 | N | N |
| Selenomonas sp. oral clone HU051 | 1708 | AY349406 | clade_480 | N | N |
| Selenomonas sp. oral clone IK004 | 1709 | AY349407 | clade_480 | N | N |
| Selenomonas sp. oral clone JI021 | 1711 | AY349409 | clade_480 | N | N |
| Selenomonas sp. oral clone JS031 | 1712 | AY349410 | clade_480 | N | N |
| Selenomonas sp. oral clone OH4A | 1713 | AY947498 | clade_480 | N | N |
| Selenomonas sp. oral clone P2PA_80 P4 | 1714 | AY207052 | clade_480 | N | N |
| Selenomonas sp. oral taxon 149 | 1716 | AEEJ01000007 | clade_480 | N | N |
| Veillonellaceae bacterium oral taxon 155 | 1995 | GU470897 | clade_480 | N | N |
| Agrococcus jenensis | 117 | NR_026275 | clade_484 | N | N |
| Microbacterium gubbeenense | 1232 | NR_025098 | clade_484 | N | N |
| Pseudoclavibacter sp. Timone | 1590 | FJ375951 | clade_484 | N | N |
| Tropheryma whipplei | 1961 | BX251412 | clade_484 | N | N |
| Zimmermannella bifida | 2031 | AB012592 | clade_484 | N | N |
| Legionella hackeliae | 1151 | M36028 | clade_486 | N | OP |
| Legionella longbeachae | 1152 | M36029 | clade_486 | N | OP |
| Legionella sp. D3923 | 1154 | JN380999 | clade_486 | N | OP |
| Legionella sp. D4088 | 1155 | JN381012 | clade_486 | N | OP |
| Legionella sp. H63 | 1156 | JF831047 | clade_486 | N | OP |
| Legionella sp. NML 93L054 | 1157 | GU062706 | clade_486 | N | OP |
| Legionella steelei | 1158 | HQ398202 | clade_486 | N | OP |
| Tatlockia micdadei | 1915 | M36032 | clade_486 | N | N |
| Helicobacter pullorum | 996 | ABQU01000097 | clade_489 | N | N |
| Acetobacteraceae bacterium AT 5844 | 16 | AGEZ01000040 | clade_490 | N | N |
| Roseomonas cervicalis | 1643 | ADVL01000363 | clade_490 | N | N |
| Roseomonas mucosa | 1644 | NR_028857 | clade_490 | N | N |
| Roseomonas sp. NML94_0193 | 1645 | AF533357 | clade_490 | N | N |
| Roseomonas sp. NML97_0121 | 1646 | AF533359 | clade_490 | N | N |
| Roseomonas sp. NML98_0009 | 1647 | AF533358 | clade_490 | N | N |
| Roseomonas sp. NML98_0157 | 1648 | AF533360 | clade_490 | N | N |
| Rickettsia akari | 1627 | CP000847 | clade_492 | N | OP |
| Rickettsia conorii | 1628 | AE008647 | clade_492 | N | OP |
| Rickettsia prowazekii | 1629 | M21789 | clade_492 | N | Category-B |
| Rickettsia rickettsii | 1630 | NC_010263 | clade_492 | N | OP |
| Rickettsia slovaca | 1631 | L36224 | clade_492 | N | OP |
| Rickettsia typhi | 1632 | AE017197 | clade_492 | N | OP |
| Anaeroglobus geminatus | 160 | AGCJ01000054 | clade_493 | N | N |
| Megasphaera genomosp. C1 | 1201 | AY278622 | clade_493 | N | N |
| Megasphaera micronuciformis | 1203 | AECS01000020 | clade_493 | N | N |
| Clostridiales genomosp. BVAB3 | 540 | CP001850 | clade_495 | N | N |
| Tsukamurella paurometabola | 1963 | X80628 | clade_496 | N | N |
| Tsukamurella tyrosinosolvens | 1964 | AB478958 | clade_496 | N | N |
| Abiotrophia para_adiacens | 2 | AB022027 | clade_497 | N | N |
| Carnobacterium divergens | 492 | NR_044706 | clade_497 | N | N |
| Carnobacterium maltaromaticum | 493 | NC_019425 | clade_497 | N | N |
| Enterococcus avium | 800 | AF133535 | clade_497 | N | N |
| Enterococcus caccae | 801 | AY943820 | clade_497 | N | N |
| Enterococcus casseliflavus | 802 | AEWT01000047 | clade_497 | N | N |
| Enterococcus durans | 803 | AJ276354 | clade_497 | N | N |
| Enterococcus faecalis | 804 | AE016830 | clade_497 | N | N |
| Enterococcus faecium | 805 | AM157434 | clade_497 | N | N |
| Enterococcus gallinarum | 806 | AB269767 | clade_497 | N | N |
| Enterococcus gilvus | 807 | AY033814 | clade_497 | N | N |
| Enterococcus hawaiiensis | 808 | AY321377 | clade_497 | N | N |
| Enterococcus hirae | 809 | AF061011 | clade_497 | N | N |
| Enterococcus italicus | 810 | AEPV01000109 | clade_497 | N | N |
| Enterococcus mundtii | 811 | NR_024906 | clade_497 | N | N |
| Enterococcus raffinosus | 812 | FN600541 | clade_497 | N | N |
| Enterococcus sp. BV2CASA2 | 813 | JN809766 | clade_497 | N | N |
| Enterococcus sp. CCRI_16620 | 814 | GU457263 | clade_497 | N | N |
| Enterococcus sp. F95 | 815 | FJ463817 | clade_497 | N | N |
| Enterococcus sp. RfL6 | 816 | AJ133478 | clade_497 | N | N |
| Enterococcus thailandicus | 817 | AY321376 | clade_497 | N | N |
| Fusobacterium canifelinum | 893 | AY162222 | clade_497 | N | N |
| Fusobacterium genomosp. C1 | 894 | AY278616 | clade_497 | N | N |
| Fusobacterium genomosp. C2 | 895 | AY278617 | clade_497 | N | N |
| Fusobacterium periodonticum | 902 | ACJY01000002 | clade_497 | N | N |
| Fusobacterium sp. 1_1_41FAA | 906 | ADGG01000053 | clade_497 | N | N |
| Fusobacterium sp. 11_3_2 | 904 | ACUO01000052 | clade_497 | N | N |
| Fusobacterium sp. 2_1_31 | 907 | ACDC02000018 | clade_497 | N | N |
| Fusobacterium sp. 3_1_27 | 908 | ADGF01000045 | clade_497 | N | N |
| Fusobacterium sp. 3_1_33 | 909 | ACQE01000178 | clade_497 | N | N |
| Fusobacterium sp. 3_1_36A2 | 910 | ACPU01000044 | clade_497 | N | N |
| Fusobacterium sp. AC18 | 912 | HQ616357 | clade_497 | N | N |
| Fusobacterium sp. ACB2 | 913 | HQ616358 | clade_497 | N | N |
| Fusobacterium sp. AS2 | 914 | HQ616361 | clade_497 | N | N |
| Fusobacterium sp. CM1 | 915 | HQ616371 | clade_497 | N | N |
| Fusobacterium sp. CM21 | 916 | HQ616375 | clade_497 | N | N |
| Fusobacterium sp. CM22 | 917 | HQ616376 | clade_497 | N | N |
| Fusobacterium sp. oral clone ASCF06 | 919 | AY923141 | clade_497 | N | N |
| Fusobacterium sp. oral clone ASCF11 | 920 | AY953256 | clade_497 | N | N |
| Granulicatella adiacens | 959 | ACKZ01000002 | clade_497 | N | N |
| Granulicatella elegans | 960 | AB252689 | clade_497 | N | N |
| Granulicatella paradiacens | 961 | AY879298 | clade_497 | N | N |
| Granulicatella sp. oral clone ASC02 | 963 | AY923126 | clade_497 | N | N |
| Granulicatella sp. oral clone ASCA05 | 964 | DQ341469 | clade_497 | N | N |
| Granulicatella sp. oral clone ASCB09 | 965 | AY953251 | clade_497 | N | N |
| Granulicatella sp. oral clone ASCG05 | 966 | AY923146 | clade_497 | N | N |
| Tetragenococcus halophilus | 1918 | NR_075020 | clade_497 | N | N |
| Tetragenococcus koreensis | 1919 | NR_043113 | clade_497 | N | N |
| Vagococcus fluvialis | 1973 | NR_026489 | clade_497 | N | N |
| Chryseobacterium anthropi | 514 | AM982793 | clade_498 | N | N |
| Chryseobacterium gleum | 515 | ACKQ02000003 | clade_498 | N | N |
| Chryseobacterium hominis | 516 | NR_042517 | clade_498 | N | N |
| Treponema refringens | 1936 | AF426101 | clade_499 | N | OP |
| Treponema sp. oral clone JU031 | 1941 | AY349416 | clade_499 | N | N |
| Treponema sp. oral taxon 239 | 1948 | GU408738 | clade_499 | N | N |
| Treponema sp. oral taxon 271 | 1955 | GU408871 | clade_499 | N | N |
| Alistipes finegoldii | 129 | NR_043064 | clade_500 | N | N |
| Alistipes onderdonkii | 131 | NR_043318 | clade_500 | N | N |
| Alistipes putredinis | 132 | ABFK02000017 | clade_500 | N | N |
| Alistipes shahii | 133 | FP929032 | clade_500 | N | N |
| Alistipes sp. HGB5 | 134 | AENZ01000082 | clade_500 | N | N |
| Alistipes sp. JC50 | 135 | JF824804 | clade_500 | N | N |
| Alistipes sp. RMA 9912 | 136 | GQ140629 | clade_500 | N | N |
| Mycoplasma agalactiae | 1310 | AF010477 | clade_501 | N | N |
| Mycoplasma bovoculi | 1313 | NR_025987 | clade_501 | N | N |
| Mycoplasma fermentans | 1315 | CP002458 | clade_501 | N | N |
| Mycoplasma flocculare | 1316 | X62699 | clade_501 | N | N |
| Mycoplasma ovipneumoniae | 1320 | NR_025989 | clade_501 | N | N |
| Arcobacter butzleri | 176 | AEPT01000071 | clade_502 | N | N |
| Arcobacter cryaerophilus | 177 | NR_025905 | clade_502 | N | N |
| Campylobacter curvus | 461 | NC_009715 | clade_502 | N | OP |
| Campylobacter rectus | 467 | ACFU01000050 | clade_502 | N | OP |
| Campylobacter showae | 468 | ACVQ01000030 | clade_502 | N | OP |
| Campylobacter sp. FOBRC14 | 469 | HQ616379 | clade_502 | N | OP |
| Campylobacter sp. FOBRC15 | 470 | HQ616380 | clade_502 | N | OP |
| Campylobacter sp. oral clone BB120 | 471 | AY005038 | clade_502 | N | OP |
| Campylobacter sputorum | 472 | NR_044839 | clade_502 | N | OP |
| Bacteroides ureolyticus | 330 | GQ167666 | clade_504 | N | N |
| Campylobacter gracilis | 463 | ACYG01000026 | clade_504 | N | OP |
| Campylobacter hominis | 464 | NC_009714 | clade_504 | N | OP |
| Dialister invisus | 762 | ACIM02000001 | clade_506 | N | N |
| Dialister micraerophilus | 763 | AFBB01000028 | clade_506 | N | N |
| Dialister microaerophilus | 764 | AENT01000008 | clade_506 | N | N |
| Dialister propionicifaciens | 766 | NR_043231 | clade_506 | N | N |
| Dialister succinatiphilus | 768 | AB370249 | clade_506 | N | N |
| Megasphaera elsdenii | 1200 | AY038996 | clade_506 | N | N |
| Megasphaera genomosp. type_1 | 1202 | ADGP01000010 | clade_506 | N | N |
| Megasphaera sp. BLPYG_07 | 1204 | HM990964 | clade_506 | N | N |
| Megasphaera sp. UPII 199_6 | 1205 | AFIJ01000040 | clade_506 | N | N |
| Chromobacterium violaceum | 513 | NC_005085 | clade_507 | N | N |
| Laribacter hongkongensis | 1148 | CP001154 | clade_507 | N | N |
| Methylophilus sp. ECd5 | 1229 | AY436794 | clade_507 | N | N |
| Finegoldia magna | 883 | ACHM02000001 | clade_509 | N | N |
| Parvimonas micra | 1431 | AB729072 | clade_509 | N | N |
| Parvimonas sp. oral taxon 110 | 1432 | AFII01000002 | clade_509 | N | N |
| Peptostreptococcus micros | 1456 | AM176538 | clade_509 | N | N |
| Peptostreptococcus sp. oral clone FJ023 | 1460 | AY349390 | clade_509 | N | N |
| Peptostreptococcus sp. P4P_31 P3 | 1458 | AY207059 | clade_509 | N | N |
| Helicobacter pylori | 997 | CP000012 | clade_510 | N | OP |
| Anaplasma marginale | 165 | ABOR01000019 | clade_511 | N | N |
| Anaplasma phagocytophilum | 166 | NC_007797 | clade_511 | N | N |
| Ehrlichia chaffeensis | 783 | AAIF01000035 | clade_511 | N | OP |
| Neorickettsia risticii | 1349 | CP001431 | clade_511 | N | N |
| Neorickettsia sennetsu | 1350 | NC_007798 | clade_511 | N | N |
| Pseudoramibacter alactolyticus | 1606 | AB036759 | clade_512 | N | N |
| Veillonella montpellierensis | 1977 | AF473836 | clade_513 | N | N |
| Veillonella sp. oral clone ASCA08 | 1988 | AY923118 | clade_513 | N | N |
| Veillonella sp. oral clone ASCB03 | 1989 | AY923122 | clade_513 | N | N |
| Inquilinus limosus | 1012 | NR_029046 | clade_514 | N | N |
| Sphingomonas sp. oral clone FZ016 | 1746 | AY349412 | clade_514 | N | N |
| Anaerococcus lactolyticus | 145 | ABYO01000217 | clade_515 | N | N |
| Anaerococcus prevotii | 147 | CP001708 | clade_515 | N | N |
| Anaerococcus sp. gpac104 | 152 | AM176528 | clade_515 | N | N |
| Anaerococcus sp. gpac126 | 153 | AM176530 | clade_515 | N | N |
| Anaerococcus sp. gpac155 | 154 | AM176536 | clade_515 | N | N |
| Anaerococcus sp. gpac199 | 155 | AM176539 | clade_515 | N | N |
| Anaerococcus tetradius | 157 | ACGC01000107 | clade_515 | N | N |
| Bacteroides coagulans | 271 | AB547639 | clade_515 | N | N |
| Clostridiales bacterium 9403326 | 534 | HM587324 | clade_515 | N | N |
| Clostridiales bacterium ph2 | 539 | JN837487 | clade_515 | N | N |
| Peptostreptococcus sp. 9succ1 | 1457 | X90471 | clade_515 | N | N |
| Peptostreptococcus sp. oral clone AP24 | 1459 | AB175072 | clade_515 | N | N |
| Tissierella praeacuta | 1924 | NR_044860 | clade_515 | N | N |
| Helicobacter canadensis | 994 | ABQS01000108 | clade_518 | N | N |
| Peptostreptococcus anaerobius | 1455 | AY326462 | clade_520 | N | N |
| Peptostreptococcus stomatis | 1461 | ADGQ01000048 | clade_520 | N | N |
| Bilophila wadsworthia | 367 | ADCP01000166 | clade_521 | N | N |
| Desulfovibrio vulgaris | 761 | NR_074897 | clade_521 | N | N |
| Actinomyces nasicola | 64 | AJ508455 | clade_523 | N | N |
| Cellulosimicrobium funkei | 500 | AY501364 | clade_523 | N | N |
| Lactococcus raffinolactis | 1146 | NR_044359 | clade_524 | N | N |
| Bacteroidales genomosp. P1 | 258 | AY341819 | clade_529 | N | N |
| Bacteroidales genomosp. P2 oral clone MB1_G13 | 259 | DQ003613 | clade_529 | N | N |
| Bacteroidales genomosp. P3 oral clone MB1_G34 | 260 | DQ003615 | clade_529 | N | N |
| Bacteroidales genomosp. P4 oral clone MB2_G17 | 261 | DQ003617 | clade_529 | N | N |
| Bacteroidales genomosp. P5 oral clone MB2_P04 | 262 | DQ003619 | clade_529 | N | N |
| Bacteroidales genomosp. P6 oral clone MB3_C19 | 263 | DQ003634 | clade_529 | N | N |
| Bacteroidales genomosp. P8 oral clone MB4_G15 | 265 | DQ003626 | clade_529 | N | N |
| Bacteroidetes bacterium oral taxon D27 | 333 | HM099638 | clade_530 | N | N |
| Bacteroidetes bacterium oral taxon F31 | 334 | HM099643 | clade_530 | N | N |
| Bacteroidetes bacterium oral taxon F44 | 335 | HM099649 | clade_530 | N | N |
| Flavobacterium sp. NF2_1 | 885 | FJ195988 | clade_530 | N | N |
| Myroides odoratimimus | 1326 | NR_042354 | clade_530 | N | N |
| Myroides sp. MY15 | 1327 | GU253339 | clade_530 | N | N |
| Chlamydiales bacterium NS16 | 507 | JN606076 | clade_531 | N | N |
| Chlamydophila pecorum | 508 | D88317 | clade_531 | N | OP |
| Parachlamydia sp. UWE25 | 1423 | BX908798 | clade_531 | N | N |
| Fusobacterium russii | 903 | NR_044687 | clade_532 | N | N |
| Streptobacillus moniliformis | 1784 | NR_027615 | clade_532 | N | N |
| Eubacteriaceae bacterium P4P_50 P4 | 833 | AY207060 | clade_533 | N | N |
| Abiotrophia defectiva | 1 | ACIN02000016 | clade_534 | N | N |
| Abiotrophia sp. oral clone P4PA_155 P1 | 3 | AY207063 | clade_534 | N | N |
| Catonella genomosp. P1 oral clone MB5_P12 | 496 | DQ003629 | clade_534 | N | N |
| Catonella morbi | 497 | ACIL02000016 | clade_534 | N | N |
| Catonella sp. oral clone FL037 | 498 | AY349369 | clade_534 | N | N |
| Eremococcus coleocola | 818 | AENN01000008 | clade_534 | N | N |
| Facklamia hominis | 879 | Y10772 | clade_534 | N | N |
| Granulicatella sp. M658_99_3 | 962 | AJ271861 | clade_534 | N | N |
| Campylobacter coli | 459 | AAFL01000004 | clade_535 | N | OP |
| Campylobacter concisus | 460 | CP000792 | clade_535 | N | OP |
| Campylobacter fetus | 462 | ACLG01001177 | clade_535 | N | OP |
| Campylobacter jejuni | 465 | AL139074 | clade_535 | N | Category-B |
| Campylobacter upsaliensis | 473 | AEPU01000040 | clade_535 | N | OP |
| Atopobium minutum | 183 | HM007583 | clade_539 | N | N |
| Atopobium parvulum | 184 | CP001721 | clade_539 | N | N |
| Atopobium rimae | 185 | ACFE01000007 | clade_539 | N | N |
| Atopobium sp. BS2 | 186 | HQ616367 | clade_539 | N | N |
| Atopobium sp. F0209 | 187 | EU592966 | clade_539 | N | N |
| Atopobium sp. ICM42b10 | 188 | HQ616393 | clade_539 | N | N |
| Atopobium sp. ICM57 | 189 | HQ616400 | clade_539 | N | N |
| Atopobium vaginae | 190 | AEDQ01000024 | clade_539 | N | N |
| Coriobacteriaceae bacterium BV3Ac1 | 677 | JN809768 | clade_539 | N | N |
| Actinomyces naeslundii | 63 | X81062 | clade_54 | N | N |
| Actinomyces oricola | 67 | NR_025559 | clade_54 | N | N |
| Actinomyces oris | 69 | BABV01000070 | clade_54 | N | N |
| Actinomyces sp. 7400942 | 70 | EU484334 | clade_54 | N | N |
| Actinomyces sp. ChDC B197 | 72 | AF543275 | clade_54 | N | N |
| Actinomyces sp. GEJ15 | 73 | GU561313 | clade_54 | N | N |
| Actinomyces sp. M2231_94_1 | 79 | AJ234063 | clade_54 | N | N |
| Actinomyces sp. oral clone GU067 | 83 | AY349362 | clade_54 | N | N |
| Actinomyces sp. oral clone IO077 | 85 | AY349364 | clade_54 | N | N |
| Actinomyces sp. oral clone IP073 | 86 | AY349365 | clade_54 | N | N |
| Actinomyces sp. oral clone JA063 | 88 | AY349367 | clade_54 | N | N |
| Actinomyces sp. oral taxon 170 | 89 | AFBL01000010 | clade_54 | N | N |
| Actinomyces sp. oral taxon 171 | 90 | AECW01000034 | clade_54 | N | N |
| Actinomyces urogenitalis | 95 | ACFH01000038 | clade_54 | N | N |
| Actinomyces viscosus | 96 | ACRE01000096 | clade_54 | N | N |
| Orientia tsutsugamushi | 1383 | AP008981 | clade_541 | N | OP |
| Megamonas funiformis | 1198 | AB300988 | clade_542 | N | N |
| Megamonas hypermegale | 1199 | AJ420107 | clade_542 | N | N |
| Aeromicrobium marinum | 102 | NR_025681 | clade_544 | N | N |
| Aeromicrobium sp. JC14 | 103 | JF824798 | clade_544 | N | N |
| Luteococcus sanguinis | 1190 | NR_025507 | clade_544 | N | N |
| Propionibacteriaceae bacterium NML 02_0265 | 1568 | EF599122 | clade_544 | N | N |
| Rhodococcus corynebacterioides | 1622 | X80615 | clade_546 | N | N |
| Rhodococcus erythropolis | 1624 | ACNO01000030 | clade_546 | N | N |
| Rhodococcus fascians | 1625 | NR_037021 | clade_546 | N | N |
| Segniliparus rotundus | 1690 | CP001958 | clade_546 | N | N |
| Segniliparus rugosus | 1691 | ACZI01000025 | clade_546 | N | N |
| Exiguobacterium acetylicum | 878 | FJ970034 | clade_547 | N | N |
| Macrococcus caseolyticus | 1194 | NR_074941 | clade_547 | N | N |
| Streptomyces sp. 1 AIP_2009 | 1890 | FJ176782 | clade_548 | N | N |
| Streptomyces sp. SD 524 | 1892 | EU544234 | clade_548 | N | N |
| Streptomyces sp. SD 528 | 1893 | EU544233 | clade_548 | N | N |
| Streptomyces thermoviolaceus | 1895 | NR_027616 | clade_548 | N | N |
| Borrelia afzelii | 388 | ABCU01000001 | clade_549 | N | OP |
| Borrelia crocidurae | 390 | DQ057990 | clade_549 | N | OP |
| Borrelia duttonii | 391 | NC_011229 | clade_549 | N | OP |
| Borrelia hermsii | 393 | AY597657 | clade_549 | N | OP |
| Borrelia hispanica | 394 | DQ057988 | clade_549 | N | OP |
| Borrelia persica | 395 | HM161645 | clade_549 | N | OP |
| Borrelia recurrentis | 396 | AF107367 | clade_549 | N | OP |
| Borrelia spielmanii | 398 | ABKB01000002 | clade_549 | N | OP |
| Borrelia turicatae | 399 | NC_008710 | clade_549 | N | OP |
| Borrelia valaisiana | 400 | ABCY01000002 | clade_549 | N | OP |
| Providencia alcalifaciens | 1586 | ABXW01000071 | clade_55 | N | N |
| Providencia rettgeri | 1587 | AM040492 | clade_55 | N | N |
| Providencia rustigianii | 1588 | AM040489 | clade_55 | N | N |
| Providencia stuartii | 1589 | AF008581 | clade_55 | N | N |
| Treponema pallidum | 1932 | CP001752 | clade_550 | N | OP |
| Treponema phagedenis | 1934 | AEFH01000172 | clade_550 | N | N |
| Treponema sp. clone DDKL_4 | 1939 | Y08894 | clade_550 | N | N |
| Acholeplasma laidlawii | 17 | NR_074448 | clade_551 | N | N |
| Mycoplasma putrefaciens | 1323 | U26055 | clade_551 | N | N |
| Mycoplasmataceae genomosp. P1 oral clone MB1_G23 | 1325 | DQ003614 | clade_551 | N | N |
| Spiroplasma insolitum | 1750 | NR_025705 | clade_551 | N | N |
| Collinsella intestinalis | 660 | ABXH02000037 | clade_553 | N | N |
| Collinsella stercoris | 661 | ABXJ01000150 | clade_553 | N | N |
| Collinsella tanakaei | 662 | AB490807 | clade_553 | N | N |
| Caminicella sporogenes | 458 | NR_025485 | clade_554 | N | N |
| Acidaminococcus fermentans | 21 | CP001859 | clade_556 | N | N |
| Acidaminococcus intestini | 22 | CP003058 | clade_556 | N | N |
| Acidaminococcus sp. D21 | 23 | ACGB01000071 | clade_556 | N | N |
| Phascolarctobacterium faecium | 1462 | NR_026111 | clade_556 | N | N |
| Phascolarctobacterium sp. YIT 12068 | 1463 | AB490812 | clade_556 | N | N |
| Phascolarctobacterium succinatutens | 1464 | AB490811 | clade_556 | N | N |
| Acidithiobacillus ferrivorans | 25 | NR_074660 | clade_557 | N | N |
| Xanthomonadaceae bacterium NML 03_0222 | 2015 | EU313791 | clade_557 | N | N |
| Catabacter hongkongensis | 494 | AB671763 | clade_558 | N | N |
| Christensenella minuta | 512 | AB490809 | clade_558 | N | N |
| Clostridiales bacterium oral clone P4PA_66 P1 | 536 | AY207065 | clade_558 | N | N |
| Clostridiales bacterium oral taxon 093 | 537 | GQ422712 | clade_558 | N | N |
| Heliobacterium modesticaldum | 1000 | NR_074517 | clade_560 | N | N |
| Alistipes indistinctus | 130 | AB490804 | clade_561 | N | N |
| Bacteroidales bacterium ph 8 | 257 | JN837494 | clade_561 | N | N |
| Candidates Sulcia muelleri | 475 | CP002163 | clade_561 | N | N |
| Cytophaga xylanolytica | 742 | FR733683 | clade_561 | N | N |
| Flavobacteriaceae genomosp. C1 | 884 | AY278614 | clade_561 | N | N |
| Gramella forsetii | 958 | NR_074707 | clade_561 | N | N |
| Sphingobacterium faecium | 1740 | NR_025537 | clade_562 | N | N |
| Sphingobacterium mizutaii | 1741 | JF708889 | clade_562 | N | N |
| Sphingobacterium multivorum | 1742 | NR_040953 | clade_562 | N | N |
| Sphingobacterium spiritivorum | 1743 | ACHA02000013 | clade_562 | N | N |
| Jonquetella anthropi | 1017 | ACOO02000004 | clade_563 | N | N |
| Pyramidobacter piscolens | 1614 | AY207056 | clade_563 | N | N |
| Synergistes genomosp. C1 | 1904 | AY278615 | clade_563 | N | N |
| Synergistes sp. RMA 14551 | 1905 | DQ412722 | clade_563 | N | N |
| Synergistetes bacterium ADV897 | 1906 | GQ258968 | clade_563 | N | N |
| Candidates Arthromitus sp. | 474 | NR_074460 | clade_564 | N | N |
| SFB_mouse_Yit | | | | | |
| Gracilibacter thermotolerans | 957 | NR_043559 | clade_564 | N | N |
| Brachyspira aalborgi | 404 | FM178386 | clade_565 | N | N |
| Brachyspira sp. HIS3 | 406 | FM178387 | clade_565 | N | N |
| Brachyspira sp. HIS4 | 407 | FM178388 | clade_565 | N | N |
| Brachyspira sp. HIS5 | 408 | FM178389 | clade_565 | N | N |
| Adlercreutzia equolifaciens | 97 | AB306661 | clade_566 | N | N |
| Coriobacteriaceae bacterium JC110 | 678 | CAEM01000062 | clade_566 | N | N |
| Coriobacteriaceae bacterium phI | 679 | JN837493 | clade_566 | N | N |
| Cryptobacterium curtum | 740 | GQ422741 | clade_566 | N | N |
| Eggerthella sinensis | 779 | AY321958 | clade_566 | N | N |
| Eggerthella sp. 1_3_56FAA | 780 | ACWN01000099 | clade_566 | N | N |
| Eggerthella sp. HGA1 | 781 | AEXR01000021 | clade_566 | N | N |
| Eggerthella sp. YY7918 | 782 | AP012211 | clade_566 | N | N |
| Gordonibacter pamelaeae | 680 | AM886059 | clade_566 | N | N |
| Gordonibacter pamelaeae | 956 | FP929047 | clade_566 | N | N |
| Slackia equolifaciens | 1732 | EU377663 | clade_566 | N | N |
| Slackia exigua | 1733 | ACUX01000029 | clade_566 | N | N |
| Slackia faecicanis | 1734 | NR_042220 | clade_566 | N | N |
| Slackia heliotrinireducens | 1735 | NR_074439 | clade_566 | N | N |
| Slackia isoflavoniconvertens | 1736 | AB566418 | clade_566 | N | N |
| Slackia piriformis | 1737 | AB490806 | clade_566 | N | N |
| Slackia sp. NATTS | 1738 | AB505075 | clade_566 | N | N |
| Chlamydiales bacterium NS13 | 506 | JN606075 | clade_567 | N | N |
| Victivallaceae bacterium NML 080035 | 2003 | FJ394915 | clade_567 | N | N |
| Victivallis vadensis | 2004 | ABDE02000010 | clade_567 | N | N |
| Streptomyces griseus | 1889 | NR_074787 | clade_573 | N | N |
| Streptomyces sp. SD 511 | 1891 | EU544231 | clade_573 | N | N |
| Streptomyces sp. SD 534 | 1894 | EU544232 | clade_573 | N | N |
| Cloacibacillus evryensis | 530 | GQ258966 | clade_575 | N | N |
| Deferribacteres sp. oral clone JV001 | 743 | AY349370 | clade_575 | N | N |
| Deferribacteres sp. oral clone JV023 | 745 | AY349372 | clade_575 | N | N |
| Synergistetes bacterium LBVCM1157 | 1907 | GQ258969 | clade_575 | N | N |
| Synergistetes bacterium oral taxon 362 | 1909 | GU410752 | clade_575 | N | N |
| Synergistetes bacterium oral taxon D48 | 1910 | GU430992 | clade_575 | N | N |
| Peptococcus sp. oral clone JM048 | 1439 | AY349389 | clade_576 | N | N |
| Helicobacter winghamensis | 999 | ACDO01000013 | clade_577 | N | N |
| Wolinella succinogenes | 2014 | BX571657 | clade_577 | N | N |
| Olsenella genomosp. C1 | 1368 | AY278623 | clade_578 | N | N |
| Olsenella profusa | 1369 | FN178466 | clade_578 | N | N |
| Olsenella sp. F0004 | 1370 | EU592964 | clade_578 | N | N |
| Olsenella sp. oral taxon 809 | 1371 | ACVE01000002 | clade_578 | N | N |
| Olsenella uli | 1372 | CP002106 | clade_578 | N | N |
| Nocardiopsis dassonvillei | 1356 | CP002041 | clade_579 | N | N |
| Peptococcus niger | 1438 | NR_029221 | clade_580 | N | N |
| Peptococcus sp. oral taxon 167 | 1440 | GQ422727 | clade_580 | N | N |
| Akkermansia muciniphila | 118 | CP001071 | clade_583 | N | N |
| Opitutus terrae | 1373 | NR_074978 | clade_583 | N | N |
| Clostridiales bacterium oral taxon F32 | 538 | HM099644 | clade_584 | N | N |
| Leptospira borgpetersenii | 1161 | NC_008508 | clade_585 | N | OP |
| Leptospira broomii | 1162 | NR_043200 | clade_585 | N | OP |
| Leptospira interrogans | 1163 | NC_005823 | clade_585 | N | OP |
| Methanobrevibacter gottschalkii | 1213 | NR_044789 | clade_587 | N | N |
| Methanobrevibacter millerae | 1214 | NR_042785 | clade_587 | N | N |
| Methanobrevibacter oralis | 1216 | HE654003 | clade_587 | N | N |
| Methanobrevibacter thaueri | 1219 | NR_044787 | clade_587 | N | N |
| Methanobrevibacter smithii | 1218 | ABYV02000002 | clade_588 | N | N |
| Deinococcus radiodurans | 746 | AE000513 | clade_589 | N | N |
| Deinococcus sp. R_43890 | 747 | FR682752 | clade_589 | N | N |
| Thermus aquaticus | 1923 | NR_025900 | clade_589 | N | N |
| Actinomyces sp. c109 | 81 | AB167239 | clade_590 | N | N |
| Syntrophomonadaceae genomosp. P1 | 1912 | AY341821 | clade_590 | N | N |
| Anaerobaculum hydrogeniformans | 141 | ACJX02000009 | clade_591 | N | N |
| Microcystis aeruginosa | 1246 | NC_010296 | clade_592 | N | N |
| Prochlorococcus marinus | 1567 | CP000551 | clade_592 | N | N |
| Methanobrevibacter acididurans | 1208 | NR_028779 | clade_593 | N | N |
| Methanobrevibacter arboriphilus | 1209 | NR_042783 | clade_593 | N | N |
| Methanobrevibacter curvatus | 1210 | NR_044796 | clade_593 | N | N |
| Methanobrevibacter cuticularis | 1211 | NR_044776 | clade_593 | N | N |
| Methanobrevibacter filiformis | 1212 | NR_044801 | clade_593 | N | N |
| Methanobrevibacter woesei | 1220 | NR_044788 | clade_593 | N | N |
| Roseiflexus castenholzii | 1642 | CP000804 | clade_594 | N | N |
| Methanobrevibacter olleyae | 1215 | NR_043024 | clade_595 | N | N |
| Methanobrevibacter ruminantium | 1217 | NR_042784 | clade_595 | N | N |
| Methanobrevibacter wolinii | 1221 | NR_044790 | clade_595 | N | N |
| Methanosphaera stadtmanae | 1222 | AY196684 | clade_595 | N | N |
| Chloroflexi genomosp. P1 | 511 | AY331414 | clade_596 | N | N |
| Halorubrum lipolyticum | 992 | AB477978 | clade_597 | N | N |
| Methanobacterium formicicum | 1207 | NR_025028 | clade_597 | N | N |
| Acidilobus saccharovorans | 24 | AY350586 | clade_598 | N | N |
| Hyperthermus butylicus | 1006 | CP000493 | clade_598 | N | N |
| Ignicoccus islandicus | 1011 | X99562 | clade_598 | N | N |
| Metallosphaera sedula | 1206 | D26491 | clade_598 | N | N |
| Thermofilum pendens | 1922 | X14835 | clade_598 | N | N |
| Prevotella melaninogenica | 1506 | CP002122 | clade_6 | N | N |
| Prevotella sp. ICM1 | 1520 | HQ616385 | clade_6 | N | N |
| Prevotella sp. oral clone FU048 | 1535 | AY349393 | clade_6 | N | N |
| Prevotella sp. oral clone GI030 | 1537 | AY349395 | clade_6 | N | N |
| Prevotella sp. SEQ116 | 1526 | JN867246 | clade_6 | N | N |
| Streptococcus anginosus | 1787 | AECT01000011 | clade_60 | N | N |
| Streptococcus milleri | 1812 | X81023 | clade_60 | N | N |
| Streptococcus sp. 16362 | 1829 | JN590019 | clade_60 | N | N |
| Streptococcus sp. 69130 | 1832 | X78825 | clade_60 | N | N |
| Streptococcus sp. AC15 | 1833 | HQ616356 | clade_60 | N | N |
| Streptococcus sp. CM7 | 1839 | HQ616373 | clade_60 | N | N |
| Streptococcus sp. OBRC6 | 1847 | HQ616352 | clade_60 | N | N |
| Burkholderia ambifaria | 442 | AAUZ01000009 | clade_61 | N | OP |
| Burkholderia cenocepacia | 443 | AAHI01000060 | clade_61 | N | OP |
| Burkholderia cepacia | 444 | NR_041719 | clade_61 | N | OP |
| Burkholderia mallei | 445 | CP000547 | clade_61 | N | Category-B |
| Burkholderia multivorans | 446 | NC_010086 | clade_61 | N | OP |
| Burkholderia oklahomensis | 447 | DQ108388 | clade_61 | N | OP |
| Burkholderia pseudomallei | 448 | CP001408 | clade_61 | N | Category-B |
| Burkholderia rhizoxinica | 449 | HQ005410 | clade_61 | N | OP |
| Burkholderia sp. 383 | 450 | CP000151 | clade_61 | N | OP |
| Burkholderia xenovorans | 451 | U86373 | clade_61 | N | OP |
| Prevotella buccae | 1488 | ACRB01000001 | clade_62 | N | N |
| Prevotella genomosp. P8 oral clone MB3_P13 | 1498 | DQ003622 | clade_62 | N | N |
| Prevotella sp. oral clone FW035 | 1536 | AY349394 | clade_62 | N | N |
| Prevotella bivia | 1486 | ADFO01000096 | clade_63 | N | N |
| Prevotella disiens | 1494 | AEDO01000026 | clade_64 | N | N |
| Bacteroides faecis | 276 | GQ496624 | clade_65 | N | N |
| Bacteroides fragilis | 279 | AP006841 | clade_65 | N | N |
| Bacteroides nordii | 285 | NR_043017 | clade_65 | N | N |
| Bacteroides salyersiae | 292 | EU136690 | clade_65 | N | N |
| Bacteroides sp. 1_1_14 | 293 | ACRP01000155 | clade_65 | N | N |
| Bacteroides sp. 1_1_6 | 295 | ACIC01000215 | clade_65 | N | N |
| Bacteroides sp. 2_1_56FAA | 298 | ACWI01000065 | clade_65 | N | N |
| Bacteroides sp. AR29 | 316 | AF139525 | clade_65 | N | N |
| Bacteroides sp. B2 | 317 | EU722733 | clade_65 | N | N |
| Bacteroides thetaiotaomicron | 328 | NR_074277 | clade_65 | N | N |
| Actinobacillus minor | 45 | ACFT01000025 | clade_69 | N | N |
| Haemophilias parasuis | 978 | GU226366 | clade_69 | N | N |
| Vibrio cholerae | 1996 | AAUR01000095 | clade_71 | N | Category-B |
| Vibrio fluvialis | 1997 | X76335 | clade_71 | N | Category-B |
| Vibrio furnissii | 1998 | CP002377 | clade_71 | N | Category-B |
| Vibrio mimicus | 1999 | ADAF01000001 | clade_71 | N | Category-B |
| Vibrio parahaemolyticus | 2000 | AAWQ01000116 | clade_71 | N | Category-B |
| Vibrio sp. RC341 | 2001 | ACZT01000024 | clade_71 | N | Category-B |
| Vibrio vulnificus | 2002 | AE016796 | clade_71 | N | Category-B |
| Lactobacillus acidophilus | 1067 | CP000033 | clade_72 | N | N |
| Lactobacillus amylolyticus | 1069 | ADNY01000006 | clade_72 | N | N |
| Lactobacillus amylovorus | 1070 | CP002338 | clade_72 | N | N |
| Lactobacillus crispatus | 1078 | ACOG01000151 | clade_72 | N | N |
| Lactobacillus delbrueckii | 1080 | CP002341 | clade_72 | N | N |
| Lactobacillus helveticus | 1088 | ACLM01000202 | clade_72 | N | N |
| Lactobacillus kalixensis | 1094 | NR_029083 | clade_72 | N | N |
| Lactobacillus kefiranofaciens | 1095 | NR_042440 | clade_72 | N | N |
| Lactobacillus leichmannii | 1098 | JX986966 | clade_72 | N | N |
| Lactobacillus sp. 66c | 1120 | FR681900 | clade_72 | N | N |
| Lactobacillus sp. KLDS 1.0701 | 1122 | EU600905 | clade_72 | N | N |
| Lactobacillus sp. KLDS 1.0712 | 1130 | EU600916 | clade_72 | N | N |
| Lactobacillus sp. oral clone HT070 | 1136 | AY349383 | clade_72 | N | N |
| Lactobacillus ultunensis | 1139 | ACGU01000081 | clade_72 | N | N |
| Prevotella intermedia | 1502 | AF414829 | clade_81 | N | N |
| Prevotella nigrescens | 1511 | AFPX01000069 | clade_81 | N | N |
| Prevotella pallens | 1515 | AFPY01000135 | clade_81 | N | N |
| Prevotella sp. oral taxon 310 | 1551 | GQ422737 | clade_81 | N | N |
| Prevotella genomosp. C1 | 1495 | AY278624 | clade_82 | N | N |
| Prevotella sp. CM38 | 1519 | HQ610181 | clade_82 | N | N |
| Prevotella sp. oral taxon 317 | 1552 | ACQH01000158 | clade_82 | N | N |
| Prevotella sp. SG12 | 1527 | GU561343 | clade_82 | N | N |
| Prevotella denticola | 1493 | CP002589 | clade_83 | N | N |
| Prevotella genomosp. P7 oral clone MB2_P31 | 1497 | DQ003620 | clade_83 | N | N |
| Prevotella histicola | 1501 | JN867315 | clade_83 | N | N |
| Prevotella multiformis | 1508 | AEWX01000054 | clade_83 | N | N |
| Prevotella sp. JCM 6330 | 1522 | AB547699 | clade_83 | N | N |
| Prevotella sp. oral clone GI059 | 1539 | AY349397 | clade_83 | N | N |
| Prevotella sp. oral taxon 782 | 1555 | GQ422745 | clade_83 | N | N |
| Prevotella sp. oral taxon G71 | 1559 | GU432180 | clade_83 | N | N |
| Prevotella sp. SEQ065 | 1524 | JN867234 | clade_83 | N | N |
| Prevotella veroralis | 1565 | ACVA01000027 | clade_83 | N | N |
| Bacteroides acidifaciens | 266 | NR_028607 | clade_85 | N | N |
| Bacteroides cellulosilyticus | 269 | ACCH01000108 | clade_85 | N | N |
| Bacteroides clarus | 270 | AFBM01000011 | clade_85 | N | N |
| Bacteroides eggerthii | 275 | ACWG01000065 | clade_85 | N | N |
| Bacteroides oleiciplenus | 286 | AB547644 | clade_85 | N | N |
| Bacteroides pyogenes | 290 | NR_041280 | clade_85 | N | N |
| Bacteroides sp. 315_5 | 300 | FJ848547 | clade_85 | N | N |
| Bacteroides sp. 31SF15 | 301 | AJ583248 | clade_85 | N | N |
| Bacteroides sp. 31SF18 | 302 | AJ583249 | clade_85 | N | N |
| Bacteroides sp. 35AE31 | 303 | AJ583244 | clade_85 | N | N |
| Bacteroides sp. 35AE37 | 304 | AJ583245 | clade_85 | N | N |
| Bacteroides sp. 35BE34 | 305 | AJ583246 | clade_85 | N | N |
| Bacteroides sp. 35BE35 | 306 | AJ583247 | clade_85 | N | N |
| Bacteroides sp. WH2 | 324 | AY895180 | clade_85 | N | N |
| Bacteroides sp. XB12B | 325 | AM230648 | clade_85 | N | N |
| Bacteroides stercoris | 327 | ABFZ02000022 | clade_85 | N | N |
| Actinobacillus pleuropneumoniae | 46 | NR_074857 | clade_88 | N | N |
| Actinobacillus ureae | 48 | AEVG01000167 | clade_88 | N | N |
| Haemophilus aegyptius | 969 | AFBC01000053 | clade_88 | N | N |
| Haemophilus ducreyi | 970 | AE017143 | clade_88 | N | OP |
| Haemophilus haemolyticus | 973 | JN175335 | clade_88 | N | N |
| Haemophilus influenzae | 974 | AADP01000001 | clade_88 | N | OP |
| Haemophilus parahaemolyticus | 975 | GU561425 | clade_88 | N | N |
| Haemophilus parainfluenzae | 976 | AEWU01000024 | clade_88 | N | N |
| Haemophilus paraphrophaemolyticus | 977 | M75076 | clade_88 | N | N |
| Haemophilus somnus | 979 | NC_008309 | clade_88 | N | N |
| Haemophilus sp. 70334 | 980 | HQ680854 | clade_88 | N | N |
| Haemophilus sp. HK445 | 981 | FJ685624 | clade_88 | N | N |
| Haemophilus sp. oral clone ASCA07 | 982 | AY923117 | clade_88 | N | N |
| Haemophilus sp. oral clone ASCG06 | 983 | AY923147 | clade_88 | N | N |
| Haemophilus sp. oral clone BJ021 | 984 | AY005034 | clade_88 | N | N |
| Haemophilus sp. oral clone BJ095 | 985 | AY005033 | clade_88 | N | N |
| Haemophilus sp. oral taxon 851 | 987 | AGRK01000004 | clade_88 | N | N |
| Haemophilus sputorum | 988 | AFNK01000005 | clade_88 | N | N |
| Histophilus somni | 1003 | AF549387 | clade_88 | N | N |
| Mannheimia haemolytica | 1195 | ACZX01000102 | clade_88 | N | N |
| Pasteurella bettyae | 1433 | L06088 | clade_88 | N | N |
| Moellerella wisconsensis | 1253 | JN175344 | clade_89 | N | N |
| Morganella morganii | 1265 | AJ301681 | clade_89 | N | N |
| Morganella sp. JB_T16 | 1266 | AJ781005 | clade_89 | N | N |
| Proteus mirabilis | 1582 | ACLE01000013 | clade_89 | N | N |
| Proteus penneri | 1583 | ABVP01000020 | clade_89 | N | N |
| Proteus sp. HS7514 | 1584 | DQ512963 | clade_89 | N | N |
| Proteus vulgaris | 1585 | AJ233425 | clade_89 | N | N |
| Oribacterium sinus | 1374 | ACKX1000142 | clade_90 | N | N |
| Oribacterium sp. ACB1 | 1375 | HM120210 | clade_90 | N | N |
| Oribacterium sp. ACB7 | 1376 | HM120211 | clade_90 | N | N |
| Oribacterium sp. CM12 | 1377 | HQ616374 | clade_90 | N | N |
| Oribacterium sp. ICM51 | 1378 | HQ616397 | clade_90 | N | N |
| Oribacterium sp. OBRC12 | 1379 | HQ616355 | clade_90 | N | N |
| Oribacterium sp. oral taxon 108 | 1382 | AFIH01000001 | clade_90 | N | N |
| Actinobacillus actinomycetemcomitans | 44 | AY362885 | clade_92 | N | N |
| Actinobacillus succinogenes | 47 | CP000746 | clade_92 | N | N |
| Aggregatibacter actinomycetemcomitans | 112 | CP001733 | clade_92 | N | N |
| Aggregatibacter aphrophilus | 113 | CP001607 | clade_92 | N | N |
| Aggregatibacter segnis | 114 | AEPS01000017 | clade_92 | N | N |
| Averyella dalhousiensis | 194 | DQ481464 | clade_92 | N | N |
| Bisgaard Taxon | 368 | AY683487 | clade_92 | N | N |
| Bisgaard Taxon | 369 | AY683489 | clade_92 | N | N |
| Bisgaard Taxon | 370 | AY683491 | clade_92 | N | N |
| Bisgaard Taxon | 371 | AY683492 | clade_92 | N | N |
| Buchnera aphidicola | 440 | NR_074609 | clade_92 | N | N |
| Cedecea davisae | 499 | AF493976 | clade_92 | N | N |
| Citrobacter amalonaticus | 517 | FR870441 | clade_92 | N | N |
| Citrobacter braakii | 518 | NR_028687 | clade_92 | N | N |
| Citrobacter farmeri | 519 | AF025371 | clade_92 | N | N |
| Citrobacter freundii | 520 | NR_028894 | clade_92 | N | N |
| Citrobacter gillenii | 521 | AF025367 | clade_92 | N | N |
| Citrobacter koseri | 522 | NC_009792 | clade_92 | N | N |
| Citrobacter murliniae | 523 | AF025369 | clade_92 | N | N |
| Citrobacter rodentium | 524 | NR_074903 | clade_92 | N | N |
| Citrobacter sedlakii | 525 | AF025364 | clade_92 | N | N |
| Citrobacter sp. 30_2 | 526 | ACDJ01000053 | clade_92 | N | N |
| Citrobacter sp. KMSI_3 | 527 | GQ468398 | clade_92 | N | N |
| Citrobacter werkmanii | 528 | AF025373 | clade_92 | N | N |
| Citrobacter youngae | 529 | ABWL02000011 | clade_92 | N | N |
| Cronobacter malonaticus | 737 | GU122174 | clade_92 | N | N |
| Cronobacter sakazakii | 738 | NC_009778 | clade_92 | N | N |
| Cronobacter turicensis | 739 | FN543093 | clade_92 | N | N |
| Enterobacter aerogenes | 786 | AJ251468 | clade_92 | N | N |
| Enterobacter asburiae | 787 | NR_024640 | clade_92 | N | N |
| Enterobacter cancerogenus | 788 | Z96078 | clade_92 | N | N |
| Enterobacter cloacae | 789 | FP929040 | clade_92 | N | N |
| Enterobacter cowanii | 790 | NR_025566 | clade_92 | N | N |
| Enterobacter hormaechei | 791 | AFHR01000079 | clade_92 | N | N |
| Enterobacter sp. 247BMC | 792 | HQ122932 | clade_92 | N | N |
| Enterobacter sp. 638 | 793 | NR_074777 | clade_92 | N | N |
| Enterobacter sp. JC163 | 794 | JN657217 | clade_92 | N | N |
| Enterobacter sp. SCSS | 795 | HM007811 | clade_92 | N | N |
| Enterobacter sp. TSE38 | 796 | HM156134 | clade_92 | N | N |
| Enterobacteriaceae bacterium 9_2_54FAA | 797 | ADCU01000033 | clade_92 | N | N |
| Enterobacteriaceae bacterium CF01Ent_1 | 798 | AJ489826 | clade_92 | N | N |
| Enterobacteriaceae bacterium Smarlab 3302238 | 799 | AY538694 | clade_92 | N | N |
| Escherichia albertii | 824 | ABKX01000012 | clade_92 | N | N |
| Escherichia coli | 825 | NC_008563 | clade_92 | N | Category-B |
| Escherichia fergusonii | 826 | CU928158 | clade_92 | N | N |
| Escherichia hermannii | 827 | HQ407266 | clade_92 | N | N |
| Escherichia sp. 1_1_43 | 828 | ACID01000033 | clade_92 | N | N |
| Escherichia sp. 4_1_40B | 829 | ACDM02000056 | clade_92 | N | N |
| Escherichia sp. B4 | 830 | EU722735 | clade_92 | N | N |
| Escherichia vulneris | 831 | NR_041927 | clade_92 | N | N |
| Ewingella americana | 877 | JN175329 | clade_92 | N | N |
| Haemophilus genomosp. P2 oral clone MB3_C24 | 971 | DQ003621 | clade_92 | N | N |
| Haemophilus genomosp. P3 oral clone MB3_C38 | 972 | DQ003635 | clade_92 | N | N |
| Haemophilus sp. oral clone JM053 | 986 | AY349380 | clade_92 | N | N |
| Hafnia alvei | 989 | DQ412565 | clade_92 | N | N |
| Klebsiella oxytoca | 1024 | AY292871 | clade_92 | N | OP |
| Klebsiella pneumoniae | 1025 | CP000647 | clade_92 | N | OP |
| Klebsiella sp. AS10 | 1026 | HQ616362 | clade_92 | N | N |
| Klebsiella sp. Co9935 | 1027 | DQ068764 | clade_92 | N | N |
| Klebsiella sp. enrichment culture clone SRC_DSD25 | 1036 | HM195210 | clade_92 | N | N |
| Klebsiella sp. OBRC7 | 1028 | HQ616353 | clade_92 | N | N |
| Klebsiella sp. SP_BA | 1029 | FJ999767 | clade_92 | N | N |
| Klebsiella sp. SRC_DSD1 | 1033 | GU797254 | clade_92 | N | N |
| Klebsiella sp. SRC_DSD11 | 1030 | GU797263 | clade_92 | N | N |
| Klebsiella sp. SRC_DSD12 | 1031 | GU797264 | clade_92 | N | N |
| Klebsiella sp. SRC_DSD15 | 1032 | GU797267 | clade_92 | N | N |
| Klebsiella sp. SRC_DSD2 | 1034 | GU797253 | clade_92 | N | N |
| Klebsiella sp. SRC_DSD6 | 1035 | GU797258 | clade_92 | N | N |
| Klebsiella variicola | 1037 | CP001891 | clade_92 | N | N |
| Kluyvera ascorbata | 1038 | NR_028677 | clade_92 | N | N |
| Kluyvera cryocrescens | 1039 | NR_028803 | clade_92 | N | N |
| Leminorella grimontii | 1159 | AJ233421 | clade_92 | N | N |
| Leminorella richardii | 1160 | HF558368 | clade_92 | N | N |
| Pantoea agglomerans | 1409 | AY335552 | clade_92 | N | N |
| Pantoea ananatis | 1410 | CP001875 | clade_92 | N | N |
| Pantoea brenneri | 1411 | EU216735 | clade_92 | N | N |
| Pantoea citrea | 1412 | EF688008 | clade_92 | N | N |
| Pantoea conspicua | 1413 | EU216737 | clade_92 | N | N |
| Pantoea septica | 1414 | EU216734 | clade_92 | N | N |
| Pasteurella dagmatis | 1434 | ACZR01000003 | clade_92 | N | N |
| Pasteurella multocida | 1435 | NC_002663 | clade_92 | N | N |
| Plesiomonas shigelloides | 1469 | X60418 | clade_92 | N | N |
| Raoultella ornithinolytica | 1617 | AB364958 | clade_92 | N | N |
| Raoultella planticola | 1618 | AF129443 | clade_92 | N | N |
| Raoultella terrigena | 1619 | NR_037085 | clade_92 | N | N |
| Salmonella bongori | 1683 | NR_041699 | clade_92 | N | Category-B |
| Salmonella enterica | 1672 | NC_011149 | clade_92 | N | Category-B |
| Salmonella enterica | 1673 | NC_011205 | clade_92 | N | Category-B |
| Salmonella enterica | 1674 | DQ344532 | clade_92 | N | Category-B |
| Salmonella enterica | 1675 | ABEH02000004 | clade_92 | N | Category-B |
| Salmonella enterica | 1676 | ABAK02000001 | clade_92 | N | Category-B |
| Salmonella enterica | 1677 | NC_011080 | clade_92 | N | Category-B |
| Salmonella enterica | 1678 | EU118094 | clade_92 | N | Category-B |
| Salmonella enterica | 1679 | NC_011094 | clade_92 | N | Category-B |
| Salmonella enterica | 1680 | AE014613 | clade_92 | N | Category-B |
| Salmonella enterica | 1682 | ABFH02000001 | clade_92 | N | Category-B |
| Salmonella enterica | 1684 | ABEM01000001 | clade_92 | N | Category-B |
| Salmonella enterica | 1685 | ABAM02000001 | clade_92 | N | Category-B |
| Salmonella typhimurium | 1681 | DQ344533 | clade_92 | N | Category-B |
| Salmonella typhimurium | 1686 | AF170176 | clade_92 | N | Category-B |
| Serratia fonticola | 1718 | NR_025339 | clade_92 | N | N |
| Serratia liquefaciens | 1719 | NR_042062 | clade_92 | N | N |
| Serratia marcescens | 1720 | GU826157 | clade_92 | N | N |
| Serratia odorifera | 1721 | ADBY01000001 | clade_92 | N | N |
| Serratia proteamaculans | 1722 | AAUN01000015 | clade_92 | N | N |
| Shigella boydii | 1724 | AAKA01000007 | clade_92 | N | Category-B |
| Shigella dysenteriae | 1725 | NC_007606 | clade_92 | N | Category-B |
| Shigella flexneri | 1726 | AE005674 | clade_92 | N | Category-B |
| Shigella sonnei | 1727 | NC_007384 | clade_92 | N | Category-B |
| Tatumella ptyseos | 1916 | NR_025342 | clade_92 | N | N |
| Trabulsiella guamensis | 1925 | AYS73830 | clade_92 | N | N |
| Yersinia aldovae | 2019 | AJ871363 | clade_92 | N | OP |
| Yersinia aleksiciae | 2020 | AJ627597 | clade_92 | N | OP |
| Yersinia bercovieri | 2021 | AF366377 | clade_92 | N | OP |
| Yersinia enterocolitica | 2022 | FR729477 | clade_92 | N | Category-B |
| Yersinia frederiksenii | 2023 | AF366379 | clade_92 | N | OP |
| Yersinia intermedia | 2024 | AF366380 | clade_92 | N | OP |
| Yersinia kristensenii | 2025 | ACCA01000078 | clade_92 | N | OP |
| Yersinia mollaretii | 2026 | NR_027546 | clade_92 | N | OP |
| Yersinia pestis | 2027 | AE013632 | clade_92 | N | Category-A |
| Yersinia pseudotuberculosis | 2028 | NC_009708 | clade_92 | N | OP |
| Yersinia rohdei | 2029 | ACCD01000071 | clade_92 | N | OP |
| Yokenella regensburgei | 2030 | AB273739 | clade_92 | N | N |
| Conchiformibius kuhniae | 669 | NR_041821 | clade_94 | N | N |
| Morococcus cerebrosus | 1267 | JN175352 | clade_94 | N | N |
| Neisseria bacilliformis | 1328 | AFAY01000058 | clade_94 | N | N |
| Neisseria cinerea | 1329 | ACDY01000037 | clade_94 | N | N |
| Neisseria flavescens | 1331 | ACQV01000025 | clade_94 | N | N |
| Neisseria gonorrhoeae | 1333 | CP002440 | clade_94 | N | OP |
| Neisseria lactamica | 1334 | ACEQ01000095 | clade_94 | N | N |
| Neisseria macacae | 1335 | AFQE01000146 | clade_94 | N | N |
| Neisseria meningitidis | 1336 | NC_003112 | clade_94 | N | OP |
| Neisseria mucosa | 1337 | ACDX01000110 | clade_94 | N | N |
| Neisseria pharyngis | 1338 | AJ239281 | clade_94 | N | N |
| Neisseria polysaccharea | 1339 | ADBE01000137 | clade_94 | N | N |
| Neisseria sicca | 1340 | ACKO02000016 | clade_94 | N | N |
| Neisseria sp. KEM232 | 1341 | GQ203291 | clade_94 | N | N |
| Neisseria sp. oral clone AP132 | 1344 | AY005027 | clade_94 | N | N |
| Neisseria sp. oral strain B33KA | 1346 | AY005028 | clade_94 | N | N |
| Neisseria sp. oral taxon 014 | 1347 | ADEA01000039 | clade_94 | N | N |
| Neisseria sp. TM10_1 | 1343 | DQ279352 | clade_94 | N | N |
| Neisseria subflava | 1348 | ACEO01000067 | clade_94 | N | N |
| Okadaella gastrococcus | 1365 | HQ699465 | clade_98 | N | N |
| Streptococcus agalactiae | 1785 | AAJO01000130 | clade_98 | N | N |
| Streptococcus alactolyticus | 1786 | NR_041781 | clade_98 | N | N |
| Streptococcus australis | 1788 | AEQR01000024 | clade_98 | N | N |
| Streptococcus bovis | 1789 | AEEL01000030 | clade_98 | N | N |
| Streptococcus canis | 1790 | AJ413203 | clade_98 | N | N |
| Streptococcus constellatus | 1791 | AY277942 | clade_98 | N | N |
| Streptococcus cristatus | 1792 | AEVC01000028 | clade_98 | N | N |
| Streptococcus dysgalactiae | 1794 | AP010935 | clade_98 | N | N |
| Streptococcus equi | 1795 | CP001129 | clade_98 | N | N |
| Streptococcus equinus | 1796 | AEVB01000043 | clade_98 | N | N |
| Streptococcus gallolyticus | 1797 | FR824043 | clade_98 | N | N |
| Streptococcus genomosp. C1 | 1798 | AY278629 | clade_98 | N | N |
| Streptococcus genomosp. C2 | 1799 | AY278630 | clade_98 | N | N |
| Streptococcus genomosp. C3 | 1800 | AY278631 | clade_98 | N | N |
| Streptococcus genomosp. C4 | 1801 | AY278632 | clade_98 | N | N |
| Streptococcus genomosp. C5 | 1802 | AY278633 | clade_98 | N | N |
| Streptococcus genomosp. C6 | 1803 | AY278634 | clade_98 | N | N |
| Streptococcus genomosp. C7 | 1804 | AY278635 | clade_98 | N | N |
| Streptococcus genomosp. C8 | 1805 | AY278609 | clade_98 | N | N |
| Streptococcus gordonii | 1806 | NC_009785 | clade_98 | N | N |
| Streptococcus infantarius | 1807 | ABJK02000017 | clade_98 | N | N |
| Streptococcus infantis | 1808 | AFNN01000024 | clade_98 | N | N |
| Streptococcus intermedius | 1809 | NR_028736 | clade_98 | N | N |
| Streptococcus lutetiensis | 1810 | NR_037096 | clade_98 | N | N |
| Streptococcus massiliensis | 1811 | AY769997 | clade_98 | N | N |
| Streptococcus mitis | 1813 | AM157420 | clade_98 | N | N |
| Streptococcus oligofermentans | 1815 | AY099095 | clade_98 | N | N |
| Streptococcus oralis | 1816 | ADMV01000001 | clade_98 | N | N |
| Streptococcus parasanguinis | 1817 | AEKM01000012 | clade_98 | N | N |
| Streptococcus pasteurianus | 1818 | AP012054 | clade_98 | N | N |
| Streptococcus peroris | 1819 | AEVF01000016 | clade_98 | N | N |
| Streptococcus pneumoniae | 1820 | AE008537 | clade_98 | N | N |
| Streptococcus porcinus | 1821 | EF121439 | clade_98 | N | N |
| Streptococcus pseudopneumoniae | 1822 | FJ827123 | clade_98 | N | N |
| Streptococcus pseudoporcinus | 1823 | AENS01000003 | clade_98 | N | N |
| Streptococcus pyogenes | 1824 | AE006496 | clade_98 | N | OP |
| Streptococcus ratti | 1825 | X58304 | clade_98 | N | N |
| Streptococcus sanguinis | 1827 | NR_074974 | clade_98 | N | N |
| Streptococcus sinensis | 1828 | AF432857 | clade_98 | N | N |
| Streptococcus sp. 2_1_36FAA | 1831 | ACOI01000028 | clade_98 | N | N |
| Streptococcus sp. 2285_97 | 1830 | AJ131965 | clade_98 | N | N |
| Streptococcus sp. ACS2 | 1834 | HQ616360 | clade_98 | N | N |
| Streptococcus sp. AS20 | 1835 | HQ616366 | clade_98 | N | N |
| Streptococcus sp. BS35a | 1836 | HQ616369 | clade_98 | N | N |
| Streptococcus sp. C150 | 1837 | ACRI01000045 | clade_98 | N | N |
| Streptococcus sp. CM6 | 1838 | HQ616372 | clade_98 | N | N |
| Streptococcus sp. ICM10 | 1840 | HQ616389 | clade_98 | N | N |
| Streptococcus sp. ICM12 | 1841 | HQ616390 | clade_98 | N | N |
| Streptococcus sp. ICM2 | 1842 | HQ616386 | clade_98 | N | N |
| Streptococcus sp. ICM4 | 1844 | HQ616387 | clade_98 | N | N |
| Streptococcus sp. ICM45 | 1843 | HQ616394 | clade_98 | N | N |
| Streptococcus sp. M143 | 1845 | ACRK01000025 | clade_98 | N | N |
| Streptococcus sp. M334 | 1846 | ACRL01000052 | clade_98 | N | N |
| Streptococcus sp. oral clone ASB02 | 1849 | AY923121 | clade_98 | N | N |
| Streptococcus sp. oral clone ASCA03 | 1850 | DQ272504 | clade_98 | N | N |
| Streptococcus sp. oral clone ASCA04 | 1851 | AY923116 | clade_98 | N | N |
| Streptococcus sp. oral clone ASCA09 | 1852 | AY923119 | clade_98 | N | N |
| Streptococcus sp. oral clone ASCB04 | 1853 | AY923123 | clade_98 | N | N |
| Streptococcus sp. oral clone ASCB06 | 1854 | AY923124 | clade_98 | N | N |
| Streptococcus sp. oral clone ASCC04 | 1855 | AY923127 | clade_98 | N | N |
| Streptococcus sp. oral clone ASCC05 | 1856 | AY923128 | clade_98 | N | N |
| Streptococcus sp. oral clone ASCC12 | 1857 | DQ272507 | clade_98 | N | N |
| Streptococcus sp. oral clone ASCD01 | 1858 | AY923129 | clade_98 | N | N |
| Streptococcus sp. oral clone ASCD09 | 1859 | AY923130 | clade_98 | N | N |
| Streptococcus sp. oral clone ASCD10 | 1860 | DQ272509 | clade_98 | N | N |
| Streptococcus sp. oral clone ASCE03 | 1861 | AY923134 | clade_98 | N | N |
| Streptococcus sp. oral clone ASCE04 | 1862 | AY953253 | clade_98 | N | N |
| Streptococcus sp. oral clone ASCE05 | 1863 | DQ272510 | clade_98 | N | N |
| Streptococcus sp. oral clone ASCE06 | 1864 | AY923135 | clade_98 | N | N |
| Streptococcus sp. oral clone ASCE09 | 1865 | AY923136 | clade_98 | N | N |
| Streptococcus sp. oral clone ASCE10 | 1866 | AY923137 | clade_98 | N | N |
| Streptococcus sp. oral clone ASCE12 | 1867 | AY923138 | clade_98 | N | N |
| Streptococcus sp. oral clone ASCF05 | 1868 | AY923140 | clade_98 | N | N |
| Streptococcus sp. oral clone ASCF07 | 1869 | AY953255 | clade_98 | N | N |
| Streptococcus sp. oral clone ASCF09 | 1870 | AY923142 | clade_98 | N | N |
| Streptococcus sp. oral clone ASCG04 | 1871 | AY923145 | clade_98 | N | N |
| Streptococcus sp. oral clone BW009 | 1872 | AY005042 | clade_98 | N | N |
| Streptococcus sp. oral clone CH016 | 1873 | AY005044 | clade_98 | N | N |
| Streptococcus sp. oral clone GK051 | 1874 | AY349413 | clade_98 | N | N |
| Streptococcus sp. oral clone GM006 | 1875 | AY349414 | clade_98 | N | N |
| Streptococcus sp. oral clone P2PA_41 P2 | 1876 | AY207051 | clade_98 | N | N |
| Streptococcus sp. oral clone P4PA_30 P4 | 1877 | AY207064 | clade_98 | N | N |
| Streptococcus sp. oral taxon 071 | 1878 | AEEP01000019 | clade_98 | N | N |
| Streptococcus sp. oral taxon G59 | 1879 | GU432132 | clade_98 | N | N |
| Streptococcus sp. oral taxon G62 | 1880 | GU432146 | clade_98 | N | N |
| Streptococcus sp. oral taxon G63 | 1881 | GU432150 | clade_98 | N | N |
| Streptococcus suis | 1882 | FM252032 | clade_98 | N | N |
| Streptococcus thermophilus | 1883 | CP000419 | clade_98 | N | N |
| Streptococcus salivarius | 1826 | AGBV01000001 | clade_98 | N | N |
| Streptococcus uberis | 1884 | HQ391900 | clade_98 | N | N |
| Streptococcus urinalis | 1885 | DQ303194 | clade_98 | N | N |
| Streptococcus vestibularis | 1886 | AEKO01000008 | clade_98 | N | N |
| Streptococcus viridans | 1887 | AF076036 | clade_98 | N | N |
| Synergistetes bacterium oral clone 03 5 D05 | 1908 | GU227192 | clade_98 | N | N |

TABLE 2
Species isolated from ethanol treated spore preparation preparation
before (left) and after (right) CsCI gradient step

| | ethanol | ethanol treated, |
|---|---|---|
| | treated spore | gradient purified |
| Isolates | preparation | spore preparation |
| Bacillus coagulans | 7 | 2 |
| Blautia luti | 1 | 1 |
| Blautia sp | 14 | 13 |
| Blautia wexlerae | 3 | 1 |
| Ruminococcus obeum | 4 | 2 |
| Clostridiales sp | 1 | 2 |
| Clostridium aerotolerans | 1 | 2 |
| Clostridium disporicum | 0 | 1 |
| Clostridium sp | 1 | 1 |
| Clostridium symbiosum | 0 | 1 |
| Dorea longicatena | 8 | 6 |
| Eubacterium cellulosolvens | 1 | 0 |
| Eubacterium ventriosum | 2 | 2 |
| Gemmiger formicilis | 0 | 1 |
| Robinsoniella peoriensis | 0 | 1 |
| Roseburia hominis | 3 | 6 |
| Roseburia intestinalis | 9 | 7 |
| Ruminococcus sp | 5 | 2 |
| Syntrophococcus sucromutans | 1 | 1 |
| Turicibacter sanguinis | 3 | 4 |
| Clostridiales sp | 7 | 9 |
| Clostridium bartlettii | 8 | 11 |
| Clostridium irregulare | 0 | 1 |
| Clostridium sordellii | 4 | 6 |
| Lachnospiraceae sp | 1 | 0 |

TABLE 3
Representative diseases, disorders and conditions
potentially associated with a dysbiosis, which are
suitable for treatment with the compositions and
methods of the invention.

Abdominal abscess
Abdominal cavity inflammation
Abscess
Absidia infection
Achromatopsia
Acidemia
Acinetobacter baumanii infection
Acinetobacter infection
Acinetobacter lwoffii infection
Acne
Acne vulgaris
Acoustic neuroma
Acromegaly
Actinomyces israelii infection
Acute bronchitis
Acute chest syndrome
Acute coronary syndrome
Acute decompensated heart failure
Acute external otitis
Acute lung injury
Acute lymphoblastic leukemia
Acute myelogenous leukemia
Acute promyelocytic leukemia
Acute sinusitis
Addison's disease
Adenocarcinoma
Adenoid hypertrophy
Adenoma
Adenomyosis
Adenosine deaminase deficiency
Adenovirus infection
Adhesive capsulitis
Adrenal cortical carcinoma
Adrenal gland hypofunction
Adrenomyeloneuropathy
Adult respiratory distress syndrome
Adult T-cell lymphoma
Adult varicella zoster virus infection
Advanced solid tumor
Agammaglobulinemia
Age related macular degeneration
Aggression
Aggressive fibromatosis
Aging
Agitation
AL amyloidosis
Albuminuria
Alcoholism (and effects)
Alkaptonuria
Allergic conjunctivitis
Allergic rhinitis
Allergy
Alopecia
Alopecia areata
Alpha mannosidosis
Alpha-1 antitrypsin deficiency
ALS
Alzheimers disease
Amenorrhea
Amnesia
Amoeba infection
Amoebic liver abscess
Amphetamine dependence
Amyloidosis
Anal cancer
Anal fissure
Anal fistula
Anal tumor
Anaplastic astrocytoma
Anaplastic large cell lymphoma
Anemia
Anesthesia
Angina
Angioedema
Angiomyolipoma
Angiosarcoma
Angle closure glaucoma
Ankylosing spondylitis
Anorexia nervosa
Anovulation
Antibiotic treatment
Antiphospholipid syndrome
Antitbiotic associated diarrhea
Anus pain
Anxiety disorder
Aortic aneurysm
Aortic stenosis
Aplastic anemia
Apnea
Appendicitis
Appetite loss
Arterial occlusive disease
Arterial thrombosis
Arteriosclerosis
Arteriovenous malformation
Artery disease
Arthralgia
Arthritis
Ascites
Asperger syndrome
Aspergillus fumigatus infection
Aspergillus infection
Asthma
Astrocytoma
Ataxia telangiectasia
Atherosclerosis
Atopic dermatitis
Atopy/Allergic Sensitivity
Atrial fibrillation
Atrial flutter
Atrophic vaginitis
Atrophy
Attention deficit hyperactivity disorder
Autism
Autoimmune disease
Autoimmune hemolytic anemia
Autosomal dominant polycystic kidney disease
B-cell acute lymphoblastic leukemia
B-cell lymphoma
Bacillus anthracis infection
Bacillus infection
Back pain
Bacteremia
Bacterial endocarditis
Bacterial eye infection
Bacterial infection
Bacterial meningitis
Bacterial pneumonia
Bacterial respiratory tract infection
Bacterial skin infection
Bacterial susceptibility
Bacterial urinary tract infection
Bacterial vaginosis
Bacteroides caccae infection
Bacteroides fragilis infection
Bacteroides infection
Bacteroides thetaiotaomicron infection
Bacteroides uniformis infection
Bacteroides vulgatus infection
Balanitis
Bariatric surgery
Bartholinitis
Bartonella bacilliformis infection
Bartonella infection
Basal cell carcinoma
Becker muscular dystrophy
Behcets disease
Beta thalassemia
Bifidobacterium infection
Biliary cancer
Biliary cirrhosis
Biliary tract disease
Biliary tract infection
Biliary tumor
Binge eating disorder
Bipolar disorder
Bipolar I disorder
Birth complication
BK virus infection
Bladder cancer
Bladder tumor
Blastomyces infection
Bleeding
Blepharitis
Blepharospasm
Blood clotting disorder
Blood clotting factor deficiency
Blood transfusion
Bone and joint infection
Bone disease
Bone infection
Bone injury
Bone marrow disease
Bone marrow failure
Bone marrow transplantation
Bone metastases
Bone resorption
Bone tumor
Bordetella pertussis infection
Borrelia burgdorferi infection
Borrelia recurrentis infection
Brain disease
Brain hemorrhage
Brain infarction
Brain injury
Brain ischemia
Brain tumor
Breast cancer
Breast disease
Breast tumor
Bronchiectasis
Bronchiolitis
Bronchitis
Bronchospasm
Brucella infection
Budd Chiari syndrome
Bulimia nervosa
Burkholderia infection
Burkitts lymphoma
Bursitis
Cachexia
Campylobacter fetus infection
Campylobacter infection
Campylobacter jejuni infection
Cancer
Cancer pain
Candida albicans infection
Candida infection
Candida krusei infection
Capillary leak syndrome
Carbamoyl phosphate synthase I deficiency
Carcinoid syndrome
Carcinoid tumor
Carcinoma
Cardiac edema
Cardiac failure
Cardiac hypertrophy
Cardiac reperfusion injury
Cardiotoxicity drug-induced
Cardiovascular disease
Carnitine palmitoyltransferase deficiency
Cartilage disease
Castlemans disease
Cataplexy
Cataract
Celiac Disease
Cellulitis
Central nervous system disease
Central nervous system injury
Central nervous system tumor
Cerebral edema
Cerebral hemorrhage
Cerebral hypoxia
Cerebral infarction
Cerebral palsy
Cerebrotendinous xanthomatosis
Cerebrovascular disease
Cerebrovascular ischemia
Cervical dysplasia
Cervical dystonia
Cervicitis
Cervix disease
Cervix infection
Cervix injury
Chemotherapy induced nausea and vomiting
Chemotherapy-induced diarrhea
Chemotherapy-induced emesis
Chlamydia infection
Chlamydia pneumoniae infection
Chlamydia trachomatis infection
Chlamydiae infection
Cholangiocarcinoma
Cholangitis
Cholecystitis
Cholelithiasis
Cholesteryl ester storage disease
Chondrosarcoma
Chorioretinitis
Choroidal neovascularization
Choroiditis
Chromoblastomycosis
Chronic bronchitis
Chronic fatigue syndrome
Chronic granulomatous disease
Chronic infection
Chronic inflammatory demyelinating polyneuropathy
Chronic lymphocytic leukemia
Chronic myelocytic leukemia
Chronic obstructive airway disease
Chronic obstructive pulmonary disease
Chronic Polio Shedders
Chronic thromboembolic pulmonary hypertension
Churg-Strauss syndrome
CINCA syndrome
Circadian rhythm sleep disorder
Cirrhosis
Citrobacter infection
Citrullinuria
Cladophialophora infection
Clostridiaceae infection
Clostridium botulinum infection
Clostridium difficile infection
Clostridium infection
Clostridium tetani infection
Cluster headache
CMV retinitis
Coats disease
Cocaine addiction
Coccidioides infection
Cognitive disorder
Cold agglutinin disease
Colitis
Colon cancer
Colon polyps
Colon tumor
Colorectal cancer
Colorectal tumor
Common cold
Compensated liver cirrhosis
Complement diseases
Complex partial seizure
Complex regional pain syndrome
Complicated skin and skin structure infection
Complicated urinary tract infection
Compulsive gambling
Condyloma
Congenital adrenal hyperplasia
Congenital heart defect
Congenital hemophilia
Congestive heart failure
Conjunctiva disease
Conjunctival neoplasm
Conjunctivitis
Connective tissue disease
Constipation
Constipation predominant irritable bowel syndrome
Contact dermatitis
Contraception
Copper metabolism disorder
Corneal disease
Corneal injury
Corneal transplant rejection
Corneal transplantation
Corneal ulcer
Coronary artery disease
Coronary thrombosis
Corynebacterium diphtheriae infection
Corynebacterium infection
Cough
Coxiella infection
CRE infection
Crohns disease
Cryopyrin associated periodic syndrome
Cryptococcus infection
Cryptococcus neoformans infection
Cryptococcus neoformans meningitis
Cryptosporidium infection
Cushings disease
Cutaneous lupus erythematosus
Cutaneous T-cell lymphoma
Cystic fibrosis
Cystic hygroma
Cystinosis
Cystinuria
Cystitis
Cytomegalovirus infection
Dacryocystitis
Deafness
Decubitus ulcer
Deep vein thrombosis
Degenerative disc disease
Dementia
Dengue virus infection
Depression
Dermatitis
Dermatofibrosarcoma
Dermatological disease
Dermatomycosis
Dermatomyositis
Dermatophytosis
Diabetes insipidus
Diabetes mellitus
Diabetic complication
Diabetic foot ulcer
Diabetic ketoacidosis
Diabetic macular edema
Diabetic nephropathy
Diabetic neuropathy
Diabetic retinopathy
Diarrhea
Diarrhea predominant irritable bowel syndrome
Diastolic heart failure
Diffuse large B-cell lymphoma
Digoxin overdose
Discoid lupus erythematosus
Disseminated intravascular coagulation
Diverticulitis
Diverticulosis
DNA virus infection
Drug withdrawal syndrome
Dry age related macular degeneration
Duchenne dystrophy
Dumping syndrome
Duodenal ulcer
Dupuytren contracture
Dysgeusia
Dysmenorrhea
Dyspareunia
Dyspepsia
Dystonia
Dysuria
Ebola virus infection
Eczema
Edema
Eisenmenger syndrome
Embolism
Emesis
Emotional lability
Emphysema
Empyema
End stage renal disease
Endocarditis
Endocrine disease
Endocrine tumor
Endometrioid carcinoma
Endometriosis
Endophthalmitis
Entamoeba histolytica infection
Enteritis
Enterobacter aerogenes infection
Enterobacter cloacae infection
Enterobacter infection
Enterobacteriaceae infection
Enterococcus faecalis infection
Enterococcus faecium infection
Enterococcus infection
Enterocolitis
Enterotoxigenic Escherichia coli (ETEC)
Enterovirus 71 infection
Enuresis
Eosinophilic esophagitis
Ependymoma
Epidermolysis bullosa
Epidermophyton infection
Epididymitis
Epididymo-orchitis
Epilepsy
Epstein Barr virus infection
Erectile dysfunction
Erysipelas
Erythema
Erythema multiforme
Erythema nodosum leprae
Erythropoietic protoporphyria
ESBL (Extended Spectrum Beta Lactamase)
Producing Bacterial Infection
Escherichia coli infection
Esophageal cancer
Esophageal varices
Esophagitis
Esophagus tumor
Essential hypertension
Estrogen deficiency
Ewing sarcoma
Exocrine pancreatic insufficiency
Exophiala infection
Extrinsic allergic alveolitis
Fabry disease
Factor I deficiency
Factor II deficiency
Factor IX deficiency
Factor VII deficiency
Factor VIII deficiency
Factor XIII deficiency
Fallopian tube cancer
Fallopian tube tumor
Familial adenomatous polyposis
Familial amyloid neuropathy
Familial cold autoinflammatory syndrome
Familial hypercholesterolemia
Familial mediterranean fever
Fasciola hepatica infection
Fatigue
Febrile neutropenia
Fecal incontinence
Female contraception
Female genital tract infection
Female genital tract tumor
Female infertility
Female sexual dysfunction
Fever
Fibromyalgia
Fibrosis
Fistula
Flavivirus infection
Folic acid deficiency
Follicle center lymphoma
Folliculitis
Food Allergy
Food poisoning
Fragile X syndrome
Francisella tularensis infection
Friedreich ataxia
Frontotemporal dementia
Functional bowel disorder
Fungal infection
Fungal meningitis
Fungal respiratory tract infection
Fungal urinary tract infection
Fusarium infection
Fusobacterium infection
Galactorrhea
Gallbladder tumor
GALT
Gastric injury
Gastric motility disorder
Gastric ulcers
Gastritis
Gastroesophageal reflux
Gastrointestinal disease
Gastrointestinal infection
Gastrointestinal pain
Gastrointestinal stromal tumor
Gastrointestinal tumor
Gastrointestinal ulcer
Gastroparesis
Gaucher disease
Gauchers disease type I
Gauchers disease type III
General anesthesia
Generalized anxiety disorder
Genetic disorder
Genital herpes
Genital system disease
Genital tract infection
Genital ulcer
Genitourinary disease
Genitourinary tract tumor
Germ cell and embryonic cancer
Gestational diabetes
Giant bone cell tumor
Giardia lamblia infection
Gigantism
Gingivitis
Glaucoma
Glaucoma surgical procedure
Glioblastoma
Glioma
Gliosarcoma
Glomerulonephritis
Glossitis
Glucose intolerance
Goiter
Gout
Graft versus host disease
Gram negative bacterium infection
Gram positive bacterium infection
Granulomatosis
Growth disorder
Growth hormone deficiency
Guillain Barre syndrome
Gynecological disorder
Gynecomastia
Haemophilus aegyptus infection
Haemophilus ducreyi infection
Haemophilus infection
Haemophilus influenzae infection
Haemophilus parainfluenzae infection
Hairy cell leukemia
Hallervorden-Spatz syndrome
Hantavirus infection
Head and neck tumor
Headache
Hearing loss
Heart arrhythmia
Heart disease
Heart transplant rejection
Helicobacter pylori infection
Helminth infection
Hemangioma
Hematological disease
Hematological neoplasm
Hematoma
Hematopoietic stem cell transplantation
Hemiplegia
Hemochromatosis
Hemoglobinopathy
Hemolytic anemia
Hemolytic uremic syndrome
Hemophilia
Hemorrhagic shock
Hemorrhoids
Heparin induced thrombocytopenia
Hepatic edema
Hepatic encephalopathy
Hepatic porphyria
Hepatitis
Hepatitis A virus infection
Hepatitis B virus infection
Hepatitis C virus infection
Hepatitis D virus infection
Hepatitis E virus infection
Hepatitis virus infection
Hepatobiliary disease
Hepatobiliary system tumor
Hepatocellular carcinoma
Hepatorenal syndrome
Hereditary angioedema
Herpes simplex virus infection
Herpesvirus infection
Herpetic keratitis
Hidradenitis
Hidradenitis suppurativa
Hippel Lindau syndrome
Histoplasma infection
HIV infection
HIV-1 infection
HIV-2 infection
HIV-associated lipodystrophy
Hodgkins disease
Homocystinuria
Hormone deficiency
Hormone dependent prostate cancer
Hormone refractory prostate cancer
Hot flashes
HSV-1 infection
HSV-2 infection
Human T cell leukemia virus 1 infection
Hunter syndrome
Huntingtons chorea
Hydrocephalus
Hyperactivity
Hyperammonemia
Hyperandrogenism
Hyperbilirubinemia
Hypercalcemia
Hypercholesterolemia
Hypereosinophilic syndrome
Hyperglycemia
Hyperhidrosis
Hyperkalemia
Hyperlipidemia
Hyperlipoproteinemia type I
Hypernatremia
Hyperoxaluria
Hyperparathyroidism
Hyperphosphatemia
Hyperpigmentation
Hyperprolactinemia
Hypersensitivity
Hypersomnia
Hypertension
Hyperthyroidism
Hypertriglyceridemia
Hypertrophic cardiomyopathy
Hyperuricemia
Hypoalbuminemia
Hypocalcemia
Hypogammaglobulinemia
Hypoglycemia
Hypogonadism
Hyponatremia
Hypoparathyroidism
Hypophosphatasia
Hypophosphatemia
Hypotension
Hypothyroidism
Hypovolemia
Ichthyosis vulgaris
Idiopathic pulmonary fibrosis
IgA nephropathy
Ileus
Immediate type hypersensitivity
Immune complex disease
Immune deficiency
Immune disorder
Immune thrombocytopenic purpura
Immunocompromised
Immunoglobulin G deficiency
Impetigo
Inappropriate ADH syndrome
Inborn error of metabolism
Inclusion body myositis
Indigestion
Infantile spasms
Infectious arthritis
Infectious disease
Infectious endocarditis
Infertility
Inflammatory bowel disease
Inflammatory disease
Influenza virus A infection
Influenza virus B infection
Influenza virus infection
Injury
Insomnia
Insulin dependent diabetes
Intermittent claudication
Interstitial cystitis
Interstitial lung disease
Intervertebral disc disease
Intestine disease
Intestine infection
Intoxication
Iritis
Iron deficiency anemia
Iron metabolism disorder
Iron overload
Irritable bowel syndrome
Ischemia
Ischemic heart disease
Ischemic stroke
Islet cell transplant rejection
Japanese encephalitis virus infection
Jaundice
Jaw disease
Joint disease
Joint infection
Juvenile myoclonic epilepsy
Juvenile rheumatoid arthritis
Kaposis sarcoma
Kawasaki disease
Keratitis
Keratoconjunctivitis
Keratosis
Kidney dialysis
Kidney transplant rejection
Klebsiella granulomatis infection
Klebsiella infection
Klebsiella pneumoniae infection
Labor complication
Lacrimal gland disease
Lactation disorder
Lambert-Eaton syndrome
Large intestine tumor
Laron syndrome
Laryngitis
Laryngopharyngitis
Lebers hereditary optic atrophy
Left heart disease pulmonary hypertension
Leg ulcer
Legionella infection
Legionella pneumophila infection
Leigh disease
Leishmania braziliensis infection
Leishmania donovani infection
Leishmania infection
Leishmania tropica infection
Lennox Gastaut syndrome
Leprosy
Leptospiraceae infection
Lesch Nyhan syndrome
Leukemia
Leukopenia
Leukopenia drug induced
Leukoplakia
Lewy body dementia
Lichen
Lipid metabolism disorder
Lipodystrophy
Lipoma
Lipoprotein lipase deficiency
Liposarcoma
Listeria monocytogenes infection
Listerosis
Liver abscess
Liver cancer
Liver cirrhosis
Liver disease
Liver fibrosis
Liver injury
Liver transplant rejection
Liver tumor
Lower back pain
Lower limb fracture
Lower respiratory tract infection
Lung abscess
Lung cancer
Lung disease
Lung disease pulmonary hypertension
Lung embolism
Lung infection
Lung inflammation
Lung malformation
Lung transplant rejection
Lung tumor
Lupus erythematosus panniculitis
Lupus nephritis
Lyme disease
Lymphadenitis
Lymphadenopathy
Lymphangioleiomyomatosis
Lymphangitis
Lymphatic system disease
Lymphedema
Lymphoblastic leukemia
Lymphoid leukemia
Lymphoma
Lysosomal acid lipase deficiency
Macroglobulinemia
Macular disease
Macular edema
Macular telangiectasia 2
Major depressive disorder
Male contraception
Male hypogonadism
Male infertility
Male osteoporosis
Male sexual dysfunction
Mania
Mantle cell lymphoma
Marburg virus infection
Marginal zone B-cell lymphoma
Maroteaux-Lamy syndrome
Mastalgia
Mastitis
Mastocytosis
Mastoiditis
Measles virus infection
Meconium aspiration syndrome
Medullary thyroid cancer
Medulloblastoma
Melanoma
Membranoproliferative glomerulonephritis
Membranous glomerulonephritis
MEN1
MEN2
Meningioma
Meningitis
Menopause
Menorrhagia
Menstruation disorder
Mental deficiency
Merkel cell carcinoma
Mesothelioma
Metabolic bone disease
Metabolic disorder
Metabolic Syndrome
Metabolic syndrome X
Metal intoxication
Metastasis
Metastatic brain cancer
Metastatic breast cancer
Metastatic colon cancer
Metastatic colorectal cancer
Metastatic esophageal cancer
Metastatic gastrointestinal cancer
Metastatic head and neck cancer
Metastatic liver cancer
Metastatic lung cancer
Metastatic non small cell lung cancer
Metastatic ovary cancer
Metastatic pancreas cancer
Metastatic prostate cancer
Metastatic renal cancer
Metastatic stomach cancer
Methylmalonic acidemia
Metrorrhagia
Micrococcaceae infection
Micrococcus infection
Microsporidial infection
Microsporum infection
Migraine
Mild cognitive impairment
Mitochondrial disease
Molluscum contagiosum infection
Monkeypox virus infection
Moraxella catarrhalis infection
Moraxella infection
Morganella infection
Morganella morganii infection
Morning sickness
Morquio syndrome
Motion sickness
Motor neurone disease
Mouth tumor
Mouth ulcer
Movement disorder
MRSA infection
Muckle Wells syndrome
Mucopolysaccharidosis type I
Mucor infection
Mucositis
Multidrug resistant infection
Multiple myeloma
Multiple sclerosis
Mumps virus infection
Muscle hypertonia
Muscle injury
Muscle spasm
Muscle weakness
Muscular dystrophy
Musculoskeletal disease
Musculoskeletal pain
Musculoskeletal system inflammation
Myalgia
Myasthenia gravis
Mycobacterium infection
Mycobacterium leprae infection
Mycobacterium tuberculosis infection
Mycoplasma infection
Mycoplasma pneumoniae infection
Mycosis fungoides
Mydriasis
Myelitis
Myelodysplastic syndrome
Myelofibrosis
Myeloid leukemia
Myeloproliferative disorder
Myocardial disease
Myocardial infarction
N-acetylglutamate synthase deficiency
Nail disease
Narcolepsy
Nasal polyps
Nasopharyngeal carcinoma
Nausea
Nausea drug-induced
Necrotizing enterocolitis
Necrotizing Pancreatitis
Neisseria gonorrhoeae infection
Neisseria infection
Neisseria meningitidis infection
Neisseria meningitidis meningitis
Nelson syndrome
Nematode infection
Neonatal respiratory distress syndrome
Neoplasm
Neoplastic meningitis
Nephritis
Nephroblastoma
Nephropathic cystinosis
Nephrotic syndrome
Nerve injury
Nerve tumor
Neuroblastoma
Neurodegenerative disease
Neuroendocrine tumor
Neurofibromatosis
Neurofibromatosis type I
Neurological disease
Neuromuscular blockade
Neuromuscular disease
Neuromyelitis optica
Neuropathic pain
Neuropathy
Neutropenia
Nicotine dependence
Niemann Pick disease type C
Nocturia
Non alcoholic fatty liver disease
Non segmental vitiligo
Non small cell lung cancer
Non-alcoholic steatohepatitis
Non-Hodgkin lymphoma
Non-insulin dependent diabetes
Non-small-cell lung cancer
Obesity
Obsessive compulsive disorder
Obstructive sleep apnea
Occlusive coronary artery disease
Ocular cystinosis
Ocular disease
Ocular hypertension
Ocular infection
Ocular inflammation
Ocular melanoma
Ocular pain
Ocular surgical procedure
Onychomycosis
Open angle glaucoma
Opiate dependence
Optic neuritis
Oral mucositis
Orbital inflammatory disease
Organ transplant rejection
Organ transplantation
Ornithine transcarbamylase deficiency
Orthostatic hypotension
Ostealgia
Osteoarthritis
Osteogenesis imperfecta
Osteolysis
Osteomalacia
Osteomyelitis
Osteonecrosis
Osteopenia
Osteopetrosis
Osteoporosis
Osteosarcoma
Otitis
Otitis externa
Otitis media
Otorhinolaryngological disease
Otorhinolaryngological infection
Ovarian hyperstimulation syndrome
Ovary cancer
Ovary tumor
Overactive bladder
Overgrowth syndrome
Pagets bone disease
Pain
Pancreas disease
Pancreas transplant rejection
Pancreas tumor
Pancreatic ductal adenocarcinoma
Pancreatic endocrine tumor
Pancreatic ulcers
Pancreatitis
Panic disorder
Panophthalmitis
Papillary thyroid tumor
Papillomavirus infection
Papular skin disease
Papulosis
Paracetamol overdose
Paralysis
Paraproteinemia
Parasitic infection
Parkinsons disease
Paroxysmal nocturnal hemoglobinuria
Partial seizure
Parturition
Patent ductus arteriosus
Pediatric varicella zoster virus infection
Pediculosis capitis infestation
Pelvic inflammatory disease
Pemphigoid
Pemphigus
Penile induration
Penis tumor
Peptic ulcer
Peptostreptococcus infection
Perennial allergic rhinitis
Perianal fistula
Periarthritis
Pericoronitis
Periodontal disease
Periodontitis
Peripheral arterial occlusive disease
Peripheral neuropathy
Peripheral T-cell lymphoma
Peripheral vascular disease
Peritoneal tumor
Peritonitis
Peritonsillitis
Pervasive child developmental disorder
Pharyngitis
Phenylketonuria
Phlebothrombosis
Pink eye infection
Pituitary tumor
Pityriasis
Planned abortion
Plasmodium falciparum infection
Plasmodium infection
Plasmodium malariae infection
Plasmodium vivax infection
Pleural disease
Pneumocystis carinii infection
Pneumonia
POEMS syndrome
Poison intoxication
Poliovirus infection
Polyarteritis nodosa
Polychondritis
Polycystic kidney disease
Polycystic ovary syndrome
Polycythemia
Polycythemia vera
Polymorphus light eruption
Polymyalgia rheumatica
Polymyositis
Polyomavirus infection
Pompes disease
Port wine stain
Portal hypertension
Post traumatic stress disorder
Post-surgical bacterial leakage
Postherpetic neuralgia
Postmenopausal osteoporosis
Postnatal depression
Pouchitis
Pre-eclampsia
Precocious puberty
Premature ejaculation
Premature labor
Premature ovarian failure
Premenstrual syndrome
Prevotella infection
Priapism
Primary biliary cirrhosis
Primary sclerosing cholangitis
Proctitis
Progestogen deficiency
Progressive supranuclear palsy
Proliferative diabetic retinopathy
Prolymphocytic leukemia
Prophylaxis
Propionibacterium acnes infection
Propionibacterium infection
Prostate cancer
Prostate hyperplasia
Prostate tumor
Prostatitis
Protein C deficiency
Proteinuria
Proteus infection
Proteus mirabilis infection
Protozoal infection
Providencia infection
Prurigo
Pruritus
Pseudomembranous colitis
Pseudomonas aeruginosa infection
Pseudomonas infection
Psoriasis
Psoriatic arthritis
Psychiatric disorder
Psychotic disorder
Pterygium
Pulmonary artery hypertension
Pulmonary fibrosis
Pulmonary heart disease
Pulmonary hypertension
Purpura
Pyelonephritis
Pyoderma gangrenosum
Rabies virus infection
Radiation sickness
Radiotherapy induced emesis
Raynauds disease
Rectal cancer
Rectal tumor
Rectosigmoiditis
Renal cell carcinoma
Renal disease
Renal failure
Renal injury
Renal osteodystrophy
Renal tubule disease
Renal tumor
Renovascular hypertension
Reperfusion injury
Reproductive disorder
Respiratory congestion
Respiratory disease
Respiratory disorder
Respiratory distress syndrome
Respiratory insufficiency
Respiratory syncytial virus infection
Respiratory tract infection
Respiratory tract inflammation
Restenosis
Restless legs syndrome
Retinal dystrophy
Retinal venous occlusion
Retinitis pigmentosa
Retinoblastoma
Retinopathy
Retinopathy of prematurity
Rett syndrome
Rheumatoid arthritis
Rhinitis
Rhinopharyngitis
Rhizomucor infection
Rhizopus infection
Rickets
Rickettsia infection
Rocky mountain spotted fever
Rosacea
Ross River virus infection
Rotavirus infection
Rotavirus Vaccine Adjunct
Rubella virus infection
Salmonella infection
Salmonella typhi infection
Salpingitis
Sandhoff disease
Sarcoidosis
Sarcoma
Sarcopenia
SARS coronavirus infection
Scabies infection
Scar tissue
Scedosporium infection
Schistosomiasis
Schizoaffective disorder
Schizophrenia
Scleritis
Scleroderma
Seasonal affective disorder
Seasonal allergic rhinitis
Sebaceous gland disease
Seborrheic dermatitis
Secondary hyperparathyroidism
Seizure disorder
Seminoma
Sepsis
Septic shock
Serratia infection
Serratia marcescens infection
Serum sickness
Severe combined immunodeficiency syndrome
Sexual deviation
Sexual dysfunction
Sezary syndrome
Shigella boydii infection
Shigella dysenteriae infection
Shigella flexneri infection
Shigella infection
Shigella sonnei infection
Shock
Short bowel syndrome
Sialadenitis
Sialorrhea
Sickle beta 0 thalassemia
Sickle cell anemia
Sickle cell crisis
Sinusitis
Sitosterolemia
Sjoegrens syndrome
Skin allergy
Skin burns
Skin cancer
Skin infection
Skin Inflammatory disease
Skin lymphoma
Skin tumor
Skin ulcer
Sleep apnea
Sleep disorder
Small-cell lung cancer
Social phobia
Soft tissue sarcoma
Solid tumor
Spinal cord disease
Spinal cord injury
Spinal cord tumor
Spinal muscular atrophy
Spinal stenosis
Spinocerebellar ataxia
Spinocerebellar degenerations
Splenomegaly
Spondylarthritis
Spondylosyndesis
Spontaneous abortion
Squamous cell carcinoma
Stage III melanoma
Stage IV melanoma
Staphylococcus aureus infection
Staphylococcus epidermidis infection
Staphylococcus infection
Staphylococcus saprophyticus infection
Stem cell transplantation
Stenotrophomonas maltophilia infection
Stevens Johnson syndrome
Stomach cancer
Stomach disease
Stomach infection
Stomach tumor
Stomach ulcer
Stomatitis
Strabismus
Streptococcus agalactiae infection
Streptococcus constellatus infection
Streptococcus infection
Streptococcus intermedius infection
Streptococcus mitis infection
Streptococcus oralis infection
Streptococcus pneumoniae infection
Streptococcus pyogenes infection
Stress urinary incontinence
Stroke
Stutter
Subcortical vascular dementia
Sucrase isomaltase deficiency
Sunburn
Surgery
Surgical procedure
Synovitis
Systemic lupus erythematosus
Systemic mastocytosis
Systolic heart failure
T-cell acute lymphoblastic leukemia
T-cell lymphoma
Tachycardia
Tardive dyskinesia
Tay Sachs disease
Temporal arteritis
Tendon injury
Tenosynovitis
Testis tumor
Testosterone deficiency
Thalassemia major
Thrombasthenia
Thromboangiitis obliterans
Thrombocyte disorder
Thrombocythemia
Thrombocytopenia
Thrombocytopenic purpura
Thrombocytosis
Thromboembolism
Thrombosis
Thymoma
Thyroid associated ophthalmopathy
Thyroid tumor
Tinea
Tinea capitis
Tinea corporis
Tinea cruris
Tinea pedis
Tinnitus
Tonic clonic epilepsy
Tonsillitis
Tooth disease
Topical anesthesia
Torticollis
Tourette syndrome
Toxic epidermal necrolysis
Toxicity
Transitional cell carcinoma
Transplant rejection
Transplantation
Traumatic brain injury
Travelers diarrhea
Trench mouth
Treponema infection
Treponema pallidum infection
Trichomonas infection
Trichophyton infection
Trigeminal neuralgia
Trypanosoma brucei infection
Trypanosoma cruzi infection
Trypanosomiasis
Tuberous sclerosis
Tumor lysis syndrome
Turners syndrome
Type 1 Diabetes
Type 2 Diabetes
Type I tyrosinemia
Ulcer
Ulcerative colitis
Ulcerative proctitis
Umbilical cord stem cell transplantation
Unidentified indication
Unstable angina
Upper respiratory tract infection
Ureaplasma urealyticum infection
Uremia
Urethritis
Urinary dysfunction
Urinary incontinence
Urinary retention
Urinary tract disease
Urinary tract infection
Urinary tract tumor
Urogenital tract infection
Urolithiasis
Urticaria
Uterine cervix tumor
Uterine fibroids
Uterus infection
Uterus tumor
Uveal melanoma
Uveitis
Vaccination
Vaccinia virus infection
Vaginal infection
Vaginitis
Variant angina pectoris
Varicella zoster virus infection
Varices
Varicosis ulcer
Variola virus infection
Vascular dementia
Vascular disease
Vascular neoplasm
Vascular occlusive disease
Vasculitis
Venous occlusive disease
Ventricular arrhythmia
Ventricular fibrillation
Ventricular tachycardia
Verruca vulgaris
Vertigo
Vibrio cholerae infection
Viral encephalitis
Viral eye infection
Viral hemorrhagic fever
Viral infection
Viral respiratory tract infection
Viral susceptibility
Viridans group Streptococcus infection
Vitamin B12 deficiency
Vitamin D deficiency
Vitamin E deficiency
Vitamin K deficiency
Vitiligo
Vitreous hemorrhage
Von Willebrands disease
Vancomycin-Resistant Enterococcus Infection
Vulva disease
Vulvovaginitis
Wasting disease
Wasting Syndrome
Wegener granulomatosis
Weight loss
West Nile virus infection
Wet age related macular degeneration
Whipple's disease
Wilson disease
Wound healing
X linked dominant hypophosphatemic rickets
Xenobiotic metabolism
Xerophthalmia
Xerostomia
Yellow fever virus infection
Yersinia pestis infection
Zollinger-Ellison syndrome
Flatulence
Gastrointestinal Disorder
General Inflammation

TABLE 4
Representative diseases, disorders and conditions
potentially associated with a dysbiosis, which are
suitable for treatment with the compositions and
methods of the invention

Abdominal cavity inflammation
Absidia infection
Acinetobacter baumanii infection
Acinetobacter infection
Acinetobacter lwoffii infection
Acne
Actinomyces israelii infection
Adenovirus infection
Adult varicella zoster virus infection
Aging
Alcoholism (and effects)
Allergic conjunctivitis
Allergic rhinitis
Allergy
ALS
Alzheimers disease
Amoeba infection
Anal cancer
Antibiotic treatment
Antitbiotic associated diarrhea
Arteriosclerosis
Arthritis
Aspergillus fumigatus infection
Aspergillus infection
Asthma
Atherosclerosis
Atopic dermatitis
Atopy/Allergic Sensitivity
Autism
Autoimmune disease
Bacillus anthracis infection
Bacillus infection
Bacterial endocarditis
Bacterial eye infection
Bacterial infection
Bacterial meningitis
Bacterial pneumonia
Bacterial respiratory tract infection
Bacterial skin infection
Bacterial susceptibility
Bacterial urinary tract infection
Bacterial vaginosis
Bacteroides caccae infection
Bacteroides fragilis infection
Bacteroides infection
Bacteroides thetaiotaomicron infection
Bacteroides uniformis infection
Bacteroides vulgatus infection
Bartonella bacilliformis infection
Bartonella infection
Bifidobacterium infection
Biliary cancer
Biliary cirrhosis
Biliary tract disease
Biliary tract infection
Biliary tumor
BK virus infection
Blastomyces infection
Bone and joint infection
Bone infection
Bordetella pertussis infection
Borrelia burgdorferi infection
Borrelia recurrentis infection
Brucella infection
Burkholderia infection
Cachexia
Campylobacter fetus infection
Campylobacter infection
Campylobacter jejuni infection
Cancer
Candida albicans infection
Candida infection
Candida krusei infection
Celiac Disease
Cervix infection
Chemotherapy-induced diarrhea
Chlamydia infection
Chlamydia pneumoniae infection
Chlamydia trachomatis infection
Chlamydiae infection
Chronic fatigue syndrome
Chronic infection
Chronic inflammatory demyelinating
polyneuropathy
Chronic Polio Shedders
Circadian rhythm sleep disorder
Cirrhosis
Citrobacter infection
Cladophialophora infection
Clostridiaceae infection
Clostridium botulinum infection
Clostridium difficile infection
Clostridium infection
Clostridium tetani infection
Coccidioides infection
Colitis
Colon cancer
Colorectal cancer
Common cold
Compensated liver cirrhosis
Complicated skin and skin structure infection
Complicated urinary tract infection
Constipation
Constipation predominant irritable bowel
syndrome
Corynebacterium diphtheriae infection
Corynebacterium infection
Coxiella infection
CRE infection
Crohns disease
Cryptococcus infection
Cryptococcus neoformans infection
Cryptosporidium infection
Cutaneous lupus erythematosus
Cystic fibrosis
Cystitis
Cytomegalovirus infection
Dementia
Dengue virus infection
Depression
Dermatitis
Diabetes mellitus
Diabetic complication
Diabetic foot ulcer
Diarrhea
Diarrhea predominant irritable bowel
syndrome
Discoid lupus erythematosus
Diverticulitis
DNA virus infection
Duodenal ulcer
Ebola virus infection
Entamoeba histolytica infection
Enterobacter aerogenes infection
Enterobacter cloacae infection
Enterobacter infection
Enterobacteriaceae infection
Enterococcus faecalis infection
Enterococcus faecium infection
Enterococcus infection
Enterocolitis
Enterovirus 71 infection
Epidermophyton infection
Epstein Barr virus infection
ESBL (Extended Spectrum Beta Lactamase)
Producing Bacterial Infection
Escherichia coli infection
Esophageal cancer
Exophiala infection
Familial cold autoinflammatory syndrome
Fasciola hepatica infection
Female genital tract infection
Female genital tract tumor
Female infertility
Fibrosis
Flavivirus infection
Food Allergy
Francisella tularensis infection
Functional bowel disorder
Fungal infection
Fungal respiratory tract infection
Fungal urinary tract infection
Fusarium infection
Fusobacterium infection
Gastric ulcers
Gastrointestinal infection
Gastrointestinal pain
Gastrointestinal ulcer
Genital tract infection
Genitourinary disease
Genitourinary tract tumor
Gestational diabetes
Giardia lamblia infection
Gingivitis
Gram negative bacterium infection
Gram positive bacterium infection
Haemophilus aegyptus infection
Haemophilus ducreyi infection
Haemophilus infection
Haemophilus influenzae infection
Haemophilus parainfluenzae infection
Hantavirus infection
Helicobacter pylori infection
Helminth infection
Hepatitis A virus infection
Hepatitis B virus infection
Hepatitis C virus infection
Hepatitis D virus infection
Hepatitis E virus infection
Hepatitis virus infection
Herpes simplex virus infection
Herpesvirus infection
Histoplasma infection
HIV infection
HIV-1 infection
HIV-2 infection
HSV-1 infection
HSV-2 infection
Human T cell leukemia virus 1 infection
Hypercholesterolemia
Hyperoxaluria
Hypertension
Infectious arthritis
Infectious disease
Infectious endocarditis
Infertility
Inflammatory bowel disease
Inflammatory disease
Influenza virus A infection
Influenza virus B infection
Influenza virus infection
Insomnia
Insulin dependent diabetes
Intestine infection
Irritable bowel syndrome
Japanese encephalitis virus infection
Joint infection
Juvenile rheumatoid arthritis
Klebsiella granulomatis infection
Klebsiella infection
Klebsiella pneumoniae infection
Legionella infection
Legionella pneumophila infection
Leishmania braziliensis infection
Leishmania donovani infection
Leishmania infection
Leishmania tropica infection
Leptospiraceae infection
Listeria monocytogenes infection
Listerosis
Liver cirrhosis
Liver fibrosis
Lower respiratory tract infection
Lung infection
Lung inflammation
Lupus erythematosus panniculitis
Lupus nephritis
Lyme disease
Male infertility
Marburg virus infection
Measles virus infection
Metabolic disorder
Metabolic Syndrome
Metastatic colon cancer
Metastatic colorectal cancer
Metastatic esophageal cancer
Metastatic gastrointestinal cancer
Metastatic stomach cancer
Micrococcaceae infection
Micrococcus infection
Microsporidial infection
Microsporum infection
Molluscum contagiosum infection
Monkeypox virus infection
Moraxella catarrhalis infection
Moraxella infection
Morganella infection
Morganella morganii infection
MRSA infection
Mucor infection
Multidrug resistant infection
Multiple sclerosis
Mumps virus infection
Musculoskeletal system inflammation
Mycobacterium infection
Mycobacterium leprae infection
Mycobacterium tuberculosis infection
Mycoplasma infection
Mycoplasma pneumoniae infection
Necrotizing enterocolitis
Necrotizing Pancreatitis
Neisseria gonorrhoeae infection
Neisseria infection
Neisseria meningitidis infection
Nematode infection
Non alcoholic fatty liver disease
Non-alcoholic steatohepatitis
Non-insulin dependent diabetes
Obesity
Ocular infection
Ocular inflammation
Orbital inflammatory disease
Osteoarthritis
Otorhinolaryngological infection
Pain
Papillomavirus infection
Parasitic infection
Parkinsons disease
Pediatric varicella zoster virus infection
Pelvic inflammatory disease
Peptostreptococcus infection
Perennial allergic rhinitis
Periarthritis
Pink eye infection
Plasmodium falciparum infection
Plasmodium infection
Plasmodium malariae infection
Plasmodium vivax infection
Pneumocystis carinii infection
Poliovirus infection
Polyomavirus infection
Post-surgical bacterial leakage
Pouchitis
Prevotella infection
Primary biliary cirrhosis
Primary sclerosing cholangitis
Propionibacterium acnes infection
Propionibacterium infection
Prostate cancer
Proteus infection
Proteus mirabilis infection
Protozoal infection
Providencia infection
Pseudomonas aeruginosa infection
Pseudomonas infection
Psoriasis
Psoriatic arthritis
Pulmonary fibrosis
Rabies virus infection
Rectal cancer
Respiratory syncytial virus infection
Respiratory tract infection
Respiratory tract inflammation
Rheumatoid arthritis
Rhinitis
Rhizomucor infection
Rhizopus infection
Rickettsia infection
Ross River virus infection
Rotavirus infection
Rubella virus infection
Salmonella infection
Salmonella typhi infection
Sarcopenia
SARS coronavirus infection
Scabies infection
Scedosporium infection
Scleroderma
Seasonal allergic rhinitis
Serratia infection
Serratia marcescens infection
Shigella boydii infection
Shigella dysenteriae infection
Shigella flexneri infection
Shigella infection
Shigella sonnei infection
Short bowel syndrome
Skin allergy
Skin cancer
Skin infection
Skin Inflammatory disease
Sleep disorder
Spondylarthritis
Staphylococcus aureus infection
Staphylococcus epidermidis infection
Staphylococcus infection
Staphylococcus saprophyticus infection
Stenotrophomonas maltophilia infection
Stomach cancer
Stomach infection
Stomach ulcer
Streptococcus agalactiae infection
Streptococcus constellatus infection
Streptococcus infection
Streptococcus intermedius infection
Streptococcus mitis infection
Streptococcus oralis infection
Streptococcus pneumoniae infection
Streptococcus pyogenes infection
Systemic lupus erythematosus
Travelers diarrhea
Trench mouth
Treponema infection
Treponema pallidum infection
Trichomonas infection
Trichophyton infection
Trypanosoma brucei infection
Trypanosoma cruzi infection
Type 1 Diabetes
Type 2 Diabetes
Ulcerative colitis
Upper respiratory tract infection
Ureaplasma urealyticum infection
Urinary tract disease
Urinary tract infection
Urinary tract tumor
Urogenital tract infection
Uterus infection
Vaccinia virus infection
Vaginal infection
Varicella zoster virus infection
Variola virus infection
Vibrio cholerae infection
Viral eye infection
Viral infection
Viral respiratory tract infection
Viridans group Streptococcus infection
Vancomycin-Resistant Enterococcus
Infection
Wasting Syndrome
Weight loss
West Nile virus infection
Whipple's disease
Xenobiotic metabolism
Yellow fever virus infection
Yersinia pestis infection
Flatulence
Gastrointestinal Disorder
General Inflammation

TABLE 5
Mortality and weight change in mice challenged with
C. difficile with or without ethanol treated,
spore preparation treatment.

| | mortality | % weight change |
|---|---|---|
| Test article | (n = 10) | on Day 3 |
| vehicle (negative control) | 20% | −10.5% |
| Donor feces (positive | 0 | −0.1% |
| control) | | |
| EtOH-treated spore | 0 | 2.3% |
| preparation 1× | | |
| EtOH-treated spore | 0 | 2.4% |
| preparation 0.1× | | |
| EtOH-treated spore | 0 | −3% |
| preparation 0.01× | | |
| heat-treated spore | 0 | 0.1% |
| preparation | | |

TABLE 6
Microbial compositions administered via oral gavage on Day −1

| | OTU | Clade |
|---|---|---|
| Microbial | Clostridium_butyricum | clade_252 |
| Compo- | Clostridium_disporicum | clade_253 |
| sition 1 | Clostridium_hylemonae | clade_260 |
| | Clostridium_orbiscindens | clade_494 |
| | Clostridium_symbiosum | clade_408 |
| | Collinsella_aerofaciens | clade_553 |
| | Coprococcus_comes | clade_262 |
| | Lachnospiraceae_bacterium_5_1_57FAA | clade_260 |
| | Ruminococcus_bromii | clade_537 |
| | Blautia_producta | clade_309 |
| | Clostridium_bolteae | clade_408 |
| | Clostridium_innocuum | clade_351 |
| | Clostridium_mayombei | clade_354 |
| | Clostridium_tertium | clade_252 |
| | Ruminococcus_gnavus | clade_360 |
| Microbial | Clostridium_disporicum | clade_253 |
| Compo- | Clostridium_orbiscindens | clade_494 |
| sition 2 | Clostridium_symbiosum | clade_408 |
| | Collinsella_aerofaciens | clade_553 |
| | Eubacterium_rectale | clade_444 |
| | Lachnospiraceae_bacterium_5_1_57FAA | clade_260 |
| | Blautia_producta | clade_309 |
| | Clostridium_innocuum | clade_351 |
| | Clostridium_mayombei | clade_354 |

TABLE 7
Population of OTUs on Days 2, 3 and 4 following dosing with Microbial Compositions

| | 1 × 10⁹ per OTU | | | | 1× 10⁸ per OTU | | | | 1 × 10⁷ per OTU | | | |
|---|---|---|---|---|---|---|---|---|---|---|---|---|
| Microbial comp 1 | −1 | 2 | 3 | 4 | −1 | 2 | 3 | 4 | −1 | 2 | 3 | 4 |
| Cl_butyricum | 0 | 106 | 51 | 32 | 0 | 10 | 0 | 34 | 195 | 0 | 0 | 0 |
| Cl_disporicum | 10 | 1746 | 1190 | 887 | 0 | 1746 | 769 | 1011 | 201 | 11175 | 1531 | 1152 |
| Cl_hylemonae | 0 | 258 | 258 | 84 | 0 | 203 | 164 | 77 | 0 | 265 | 214 | 90 |
| Cl_orbiscindens | 0 | 188 | 192 | 471 | 0 | 188 | 138 | 276 | 0 | 221 | 174 | 341 |
| Cl_symbiosum | 0 | 485 | 482 | 486 | 0 | 444 | 379 | 447 | 0 | 562 | 427 | 775 |
| Co_aerofaciens | 0 | 0 | 0 | 0 | 0 | 0 | 0 | 0 | 0 | 0 | 0 | 0 |
| C_comes | 0 | 0 | 0 | 0 | 0 | 0 | 0 | 0 | 0 | 0 | 0 | 0 |
| L_bacterium_5_1_ | 0 | 341 | 336 | 354 | 0 | 351 | 182 | 356 | 0 | 256 | 240 | 300 |
| 57FAA | | | | | | | | | | | | |
| R_bromii | 0 | 0 | 0 | 0 | 0 | 0 | 0 | 0 | 0 | 0 | 0 | 0 |
| B_producta | 0 | 0 | 0 | 0 | 0 | 0 | 0 | 0 | 0 | 0 | 0 | 0 |
| Cl_bolteae | 0 | 0 | 0 | 0 | 0 | 0 | 0 | 0 | 0 | 0 | 0 | 0 |
| Cl_innocuum | 0 | 0 | 0 | 0 | 0 | 0 | 0 | 0 | 0 | 0 | 0 | 0 |
| Cl_mayombei | 0 | 0 | 0 | 0 | 0 | 0 | 0 | 0 | 0 | 0 | 0 | 0 |
| Cl_tertium | 0 | 0 | 0 | 0 | 0 | 0 | 0 | 0 | 0 | 0 | 0 | 0 |
| R_gnavus | 0 | 0 | 0 | 0 | 0 | 0 | 0 | 0 | 0 | 0 | 0 | 0 |
| Microbial comp 2 | −1 | 2 | 3 | 4 | −1 | 2 | 3 | 4 | −1 | 2 | 3 | 4 |
| Cl_disporicum | 29 | 11810 | 10948 | 14672 | 0 | 11349 | 13978 | 3942 | 0 | 11995 | 7005 | 6268 |
| Cl_orbiscindens | 0 | 510 | 408 | 764 | 0 | 332 | 545 | 544 | 0 | 310 | 319 | 432 |
| Cl_symbiosum | 0 | 559 | 508 | 375 | 0 | 665 | 494 | 450 | 0 | 396 | 639 | 650 |
| Co_aerofaciens | 0 | 0 | 0 | 0 | 0 | 0 | 1172 | 0 | 0 | 0 | 247 | 0 |
| E_rectale | 0 | 0 | 0 | 0 | 0 | 0 | 0 | 12 | 0 | 0 | 0 | 261 |
| L_bacterium_5_1_ | 0 | 972 | 801 | 596 | 0 | 860 | 962 | 844 | 0 | 636 | 1901 | 1269 |
| 57FAA | | | | | | | | | | | | |
| B_producta | 0 | 0 | 0 | 0 | 0 | 0 | 0 | 0 | 0 | 0 | 0 | 0 |
| Cl_innocuum | 0 | 0 | 0 | 0 | 0 | 0 | 0 | 0 | 0 | 0 | 0 | 0 |
| Cl_mayombei | 0 | 0 | 0 | 0 | 0 | 0 | 0 | 0 | 0 | 0 | 0 | 0 |

TABLE 8
Population of Clades on Days 2, 3 and 4 following dosing with Microbial Compositions

| | 1 × 10⁹ per OTU | | | | 1 × 10⁸ per OTU | | | | 1 × 10⁷ per OTU | | | |
|---|---|---|---|---|---|---|---|---|---|---|---|---|
| Microbial | | | | | | | | | | | | |
| comp 1 | −1 | 2 | 3 | 4 | −1 | 2 | 3 | 4 | −1 | 2 | 3 | 4 |
| clade_252 | 0 | 444 | 252 | 87 | 0 | 198 | 122 | 125 | 209 | 394 | 231 | 88 |
| clade_253 | 10 | 1746 | 1190 | 887 | 0 | 1746 | 769 | 1011 | 201 | 11175 | 1531 | 1152 |
| clade_260 | 0 | 599 | 594 | 438 | 0 | 554 | 346 | 433 | 0 | 521 | 454 | 390 |
| clade_262 | 0 | 14 | 151 | 51 | 0 | 0 | 0 | 0 | 0 | 12 | 21 | 57 |
| clade_309 | 0 | 11093 | 9750 | 4023 | 0 | 9991 | 5208 | 5145 | 19 | 9311 | 6369 | 4951 |
| clade_351 | 0 | 9064 | 10647 | 7751 | 0 | 6528 | 7259 | 8213 | 0 | 8903 | 10049 | 8701 |
| clade_354 | 0 | 0 | 0 | 0 | 0 | 0 | 0 | 31 | 173 | 0 | 0 | 0 |
| clade_360 | 0 | 14300 | 10220 | 11036 | 0 | 12553 | 12989 | 6889 | 0 | 9308 | 13483 | 9292 |
| clade_408 | 13 | 8892 | 12985 | 12101 | 23 | 3952 | 7260 | 10652 | 43 | 4079 | 8581 | 14929 |
| clade_494 | 0 | 226 | 227 | 565 | 0 | 188 | 184 | 411 | 0 | 221 | 200 | 351 |
| clade_537 | 0 | 0 | 68 | 225 | 0 | 0 | 0 | 0 | 0 | 0 | 0 | 55 |
| clade_553 | 0 | 0 | 0 | 0 | 0 | 0 | 0 | 0 | 0 | 0 | 0 | 0 |
| Microbial | | | | | | | | | | | | |
| comp 2 | −1 | 2 | 3 | 4 | −1 | 2 | 3 | 4 | −1 | 2 | 3 | 4 |
| clade_253 | 29 | 11810 | 10948 | 14672 | 0 | 11349 | 13978 | 3942 | 0 | 11995 | 7005 | 6268 |
| clade_260 | 0 | 1125 | 1312 | 854 | 0 | 1049 | 1295 | 1250 | 0 | 792 | 2121 | 1637 |
| clade_309 | 54 | 12513 | 13731 | 7849 | 0 | 11610 | 12004 | 12672 | 0 | 7407 | 14111 | 10858 |
| clade_351 | 0 | 7651 | 9939 | 5936 | 0 | 8495 | 9724 | 9207 | 0 | 6005 | 9833 | 7655 |
| clade_354 | 149 | 0 | 127 | 429 | 0 | 0 | 0 | 39 | 12 | 0 | 0 | 0 |
| clade_408 | 18 | 2242 | 4989 | 10480 | 12 | 1688 | 5580 | 3789 | 0 | 1068 | 1561 | 6281 |
| clade_444 | 41 | 0 | 49 | 202 | 0 | 18 | 0 | 12 | 0 | 14 | 82 | 1578 |
| clade_494 | 0 | 510 | 465 | 1054 | 0 | 332 | 565 | 596 | 0 | 310 | 319 | 476 |
| clade_553 | 0 | 0 | 0 | 0 | 0 | 0 | 1172 | 0 | 0 | 0 | 247 | 0 |

TABLE 9
Mortality by experimental group in mice
challenged with 10^{4.5} C. difficile spores on Day 0

| | Dose | Deaths |
|---|---|---|
| Group | (CFU per OTU) | (% mortality) |
| Vehicle control | N/A | 10 (100%) |
| Microbial | 10⁹ | 1 (10%) |
| composition 1 | 10⁸ | 1 (10%) |
| | 10⁷ | 0 (0%) |
| Microbial | 10⁹ | 0 (0%) |
| composition 2 | 10⁸ | 1 (10%) |
| | 10⁷ | 1 (10%) |

TABLE 10
Spore quantitation for ethanol treated spore preparations
using spore CFU (SCFU) assay and DPA assay

| | SCFU/30 | DPA SEq/30 | Ratio SCFU/ |
|---|---|---|---|
| Preparation | capsules | capsules | DPA |
| Preparation 1 | 4.0 × 10⁵ | 6.8 × 10⁷ | 5.9 x 10⁻³ |
| Preparation 2 | 2.1 × 10⁷ | 9.2 × 10⁸ | 0.023 |
| Preparation 3 | 6.9 × 10⁹ | 9.6 × 10⁹ | 0.72 |

TABLE 11
DPA doses in Table AC when normalized to 4 × 10⁵ SCFU per dose

| | | | Fraction of |
|---|---|---|---|
| | SCFU/30 | DPA SEq/30 | Preparation 1 |
| Preparation | capsules | capsules | Dose |
| Preparation 1 | 4.0 × 10⁵ | 6.8 × 10⁷ | 1.0 |
| Preparation 2 | 4.0 × 10⁵ | 1.8 × 10⁷ | 0.26 |
| Preparation 3 | 4.0 × 10⁵ | 5.6 × 10⁵ | 0.0082 |

### Table 12

Bacterial OTUs associated with engraftment and ecological augmentation and establishment of a more diverse microbial ecology in patients treated with an ethanol treated spore preparation. OTUs that comprise an augmented ecology are not present in the patient prior to treatment and/or exist at extremely low frequencies such that they do not comprise a significant fraction of the total microbial carriage and are not detectable by genomic and/or microbiological assay methods. OTUs that are members of the engrafting and augmented ecologies were identified by characterizing the OTUs that increase in their relative abundance post treatment and that respectively are: (i) present in the ethanol treated spore preparation and absent in the patient pretreatment, or (ii) absent in the ethanol treated spore preparation, but increase in their relative abundance through time post treatment with the preparation due to the formation of favorable growth conditions by the treatment. Notably, the latter OTUs can grow from low frequency reservoirs in the patient, or be introduced from exogenous sources such as diet. OTUs that comprise a “core” augmented or engrafted ecology can be defined by the percentage of total patients in which they are observed to engraft and/or augment; the greater this percentage the more likely they are to be part of a core ecology responsible for catalyzing a shift away from a dysbiotic ecology. The dominant OTUs in an ecology can be identified using several methods including but not limited to defining the OTUs that have the greatest relative abundance in either the augmented or engrafted ecologies and defining a total relative abundance threshold. As example, the dominant OTUs in the augmented ecology of Patient-1 were identified by defining the OTUs with the greatest relative abundance, which together comprise 60% of the microbial carriage in this patient's augmented ecology.

| | | | Dominant |
|---|---|---|---|
| | | Spore | OTU |
| | Phylogenetic | Forming | Augmented |
| OTU | Clade | OTU | Ecology |
| Bacteroides sp. 2_1_22 | clade38 | N | Y |
| Streptococcus anginosus | clade60 | N | |
| Prevotella intermedia | clade81 | N | |
| Prevotella nigrescens | clade81 | N | |
| Oribacterium sp. ACB7 | clade90 | N | |
| Prevotella salivae | clade104 | N | |
| Bacteroides intestinalis | clade171 | N | Y |
| Bifidobacterium dentium | clade172 | N | |
| Alcaligenes faecalis | clade183 | N | |
| Rothia dentocariosa | clade194 | N | |
| Peptoniphilus lacrimalis | clade291 | N | |
| Anaerococcus sp. gpac155 | clade294 | N | |
| Sutterella stercoricanis | clade302 | N | Y |
| Bacteroides sp. 3_1_19 | clade335 | N | Y |
| Parabacteroides goldsteinii | clade335 | N | |
| Bacteroides dorei | clade378 | N | Y |
| Bacteroides massiliensis | clade378 | N | |
| Lactobacillus iners | clade398 | N | |
| Granulicatella adiacens | clade460 | N | |
| Eggerthella sp. 1_3_56FAA | clade477 | N | |
| Gordonibacter pamelaeae | clade477 | N | |
| Finegoldia magna | clade509 | N | |
| Actinomyces nasicola | clade523 | N | |
| Streptobacillus moniliformis | clade532 | N | |
| Oscillospira guilliermondii | clade540 | N | |
| Orientia tsutsugamushi | clade541 | N | |
| Christensenella minuta | clade558 | N | |
| Clostridium oroticum | clade96 | Y | |
| Clostridium sp. D5 | clade96 | Y | |
| Clostridium glycyrrhizinilyticum | clade147 | Y | |
| Coprococcus comes | clade147 | Y | |
| Ruminococcus lactaris | clade147 | Y | |
| Ruminococcus torques | clade147 | Y | Y |
| Clostridiales sp. SS3/4 | clade246 | Y | |
| Clostridium hylemonae | clade260 | Y | |
| Clostridium aerotolerans | clade269 | Y | |
| Clostridium asparagiforme | clade300 | Y | Y |
| Clostridium sp. M62/1 | clade300 | Y | |
| Clostridium symbiosum | clade300 | Y | |
| Lachnospiraceae genomo sp. C1 | clade300 | Y | |
| Blautia sp. M25 | clade304 | Y | Y |
| Blautia stercoris | clade304 | Y | |
| Ruminococcus hansenii | clade304 | Y | |
| Ruminococcus obeum | clade304 | Y | |
| Ruminococcus sp. 5_1_39BFAA | clade304 | Y | |
| Bryantella formatexigens | clade309 | Y | |
| Eubacterium cellulosolvens | clade309 | Y | |
| Clostridium sp. HGF2 | clade351 | Y | |
| Clostridium bartlettii | clade354 | Y | |
| Clostridium bifermentans | clade354 | Y | |
| Clostridium glycolicum | clade354 | Y | |
| Eubacterium tenue | clade354 | Y | |
| Dorea formicigenerans | clade360 | Y | |
| Dorea longicatena | clade360 | Y | |
| Lachnospiraceae bacterium | clade360 | Y | |
| 2_1_46FAA | | | |
| Lachnospiraceae bacterium | clade360 | Y | Y |
| 9_1_43BFAA | | | |
| Ruminococcus gnavus | clade360 | Y | |
| Clostridium hathewayi | clade362 | Y | |
| Blautia hydrogenotrophica | clade368 | Y | |
| Clostridiaceae bacterium END-2 | clade368 | Y | |
| Roseburia faecis | clade369 | Y | |
| Roseburia hominis | clade370 | Y | |
| Roseburia intestinalis | clade370 | Y | |
| Eubacterium sp. WAL 14571 | clade384 | Y | |
| Erysipelotrichaceae bacterium | clade385 | Y | |
| 5_2_54FAA | | | |
| Eubacterium biforme | clade385 | Y | |
| Eubacterium dolichum | clade385 | Y | |
| Coprococcus catus | clade393 | Y | |
| Acetivibrio ethanolgignens | clade396 | Y | |
| Anaerosporobacter mobilis | clade396 | Y | |
| Bacteroides pectinophilus | clade396 | Y | |
| Eubacterium hallii | clade396 | Y | |
| Eubacterium xylanophilum | clade396 | Y | |
| Anaerostipes caccae | clade408 | Y | |
| Clostridiales bacterium | clade408 | Y | |
| 1_7_47FAA | | | |
| Clostridium aldenense | clade408 | Y | |
| Clostridium citroniae | clade408 | Y | |
| Eubacterium hadrum | clade408 | Y | Y |
| Acetanaerobacterium elongatum | clade439 | Y | |
| Faecalibacterium prausnitzii | clade478 | Y | |
| Gemmiger formicilis | clade478 | Y | Y |
| Eubacterium ramulus | clade482 | Y | |
| Lachnospiraceae bacterium | clade483 | Y | |
| 3_1_57FAA_CT1 | | | |
| Lachnospiraceae bacterium A4 | clade483 | Y | Y |
| Lachnospiraceae bacterium DJF | clade483 | Y | |
| VP30 | | | |
| Holdemania filiformis | clade485 | Y | |
| Clostridium orbiscindens | clade494 | Y | |
| Pseudoflavonifractor capillosus | clade494 | Y | |
| Ruminococcaceae bacterium D16 | clade494 | Y | |
| Acetivibrio cellulolyticus | clade495 | Y | |
| Eubacterium limosum | clade512 | Y | |
| Anaerotruncus colihominis | clade516 | Y | |
| Clostridium methylpentosum | clade516 | Y | |
| Clostridium sp. YIT 12070 | clade516 | Y | |
| Hydrogenoanaerobacterium | clade516 | Y | |
| saccharovorans | | | |
| Eubacterium ventriosum | clade519 | Y | |
| Eubacterium eligens | clade522 | Y | |
| Lachnospira pectinoschiza | clade522 | Y | |
| Lactobacillus rogosae | clade522 | Y | Y |
| Clostridium leptum | clade537 | Y | |
| Eubacterium coprostanoligenes | clade537 | Y | |
| Ruminococcus bromii | clade537 | Y | |
| Clostridium viride | clade540 | Y | |
| Butyrivibrio crossotus | clade543 | Y | |
| Coprococcus eutactus | clade543 | Y | |
| Eubacterium ruminantium | clade543 | Y | |
| Eubacterium rectale | clade568 | Y | Y |
| Roseburia inulinivorans | clade568 | Y | |
| Butyricicoccus pullicaecorum | clade572 | Y | |
| Eubacterium desmolans | clade572 | Y | |
| Papillibacter cinnamivorans | clade572 | Y | |
| Sporobacter termitidis | clade572 | Y | |
| Clostridium lactatifermentans | clade576 | Y | |

TABLE 13
Reduction in the opportunistic pathogen or
pathobiont load by ethanol treated spores.

| | Pretreatment | Day 5 | Day 14 | Day 25 |
|---|---|---|---|---|
| Klebsiella | 20.27% | 1.32% | 7.62% | 0.00% |
| (% of total reads) | | | | |
| Fusobacterium | 19.14% | 3.01% | 0.01% | 0.00% |
| (% total of reads) | | | | |

TABLE 14
Changes in Enterobacteria as a function of treatment
measured on Simmons Citrate Agar

| | | Pretreatment titer | Day 25 titer |
|---|---|---|---|
| Patient | Organism | (cfu/g) | (cfu/g) |
| 1 | Klebsiella pneumoniae | 9 × 10⁶ | 1 × 10³ |
| 1 | Klebsiella sp. Co9935 | 4 × 10⁶ | 1 × 10³ |
| 1 | Escherichia coli | 7 × 10⁶ | 1 × 10⁶ |
| 2 | Klebsiella sp. Co9935 | 4 × 10⁶ | 1 × 10³ |
| 4 | Klebsiella pneumoniae | 3 × 10⁸ | <1 × 10⁴ |
| 4 | Klebsiella sp. Co9935 | 6 × 10⁷ | <1 × 10⁴ |
| 5 | Klebsiella pneumoniae | 1 × 10⁶ | <1 × 10⁴ |

TABLE 15
Augmentation of Bacteroides as a function of
bacterial composition treatment of Patient 1.

| | Bacteroides | Pretreatment titer | Day 25 titer |
|---|---|---|---|
| Media | species | (cfu/g) | (cfu/g) |
| BBE | B. fragilis group | <2 × 10⁴ | 3 × 10⁸ |
| PFA | All Bacteroides | <2 × 10⁷ | 2 × 10¹⁰ |

TABLE 16
Bacteroides spp. in Patient 1 post-treatment with the ethanol treated spore
preparation based full-length 16S rDNA sequences of isolated strains.

| | % of total Bacteroides cfu |
|---|---|
| Species | (1.58E10 cfu/g) |
| Bacteroides sp. 4_1_36 | 63% |
| Bacteroides cellulosilyticus | 14% |
| Bacteroides sp. 1_1_30 | 14% |
| Bacteroides uniformis | 4.8% |
| Bacteroides ovatus | 1.7% |
| Bacteroides dorei | 0.91% |
| Bacteroides xylanisolvens | 0.83% |
| Bacteroides sp. 3_1_19 | 0.23% |

TABLE 17
Titers (in cfu/g) of imipenem-resistant M. morganii,
P. rettgeri and P. pennerii from Patients 2, 4 & 5

| Patient | Organism | Pretreatment titer | Day 28 titer* |
|---|---|---|---|
| Patient 2 | M. morganii | 1 × 10⁴ | 6 × 10² |
| Patient 2 | P. rettgeri | 9 × 10³ | <5 × 10¹ |
| Patient 4 | M. morganii | 2 × 10⁴ | <5 × 10¹ |
| Patient 4 | P. pennerii | 2 × 10⁴ | <5 × 10¹ |
| Patient 5 | M. morganii | 5 × 10³ | <5 × 10¹ |

*Limit of detection based on plating 200 uL of 10% wt/vol suspension is 5 × 10¹

TABLE 18

| | | % of | | | |
|---|---|---|---|---|---|
| | | Spore | % of | | |
| | | Preps | Patients | | Key- |
| | | with | OTU | Spore | stone |
| OTU | Clade | OTU | Engrafts | Former | OTU |
| Prevotella_maculosa | clade_104 | 10% | 0% | N | N |
| Prevotella_copri | clade_168 | 20% | 0% | N | N |
| Bacteroides_caccae | clade_170 | 30% | 0% | N | Y |
| Bifidobacterium_sp_TM_7* | clade_172 | 90% | 60% | N | N |
| Bifidobacterium_gallicum | clade_172 | 70% | 20% | N | N |
| Bifidobacterium_dentium | clade_172 | 50% | 0% | N | N |
| Lactobacillus_casei | clade_198 | 20% | 10% | N | N |
| Actinomyces_odontolyticus | clade_212 | 20% | 30% | N | N |
| Clostridium_colicanis | clade_223 | 10% | 10% | Y | N |
| Clostridiales_sp_SS3_4* | clade_246 | 100% | 70% | Y | N |
| Clostridium_sporogenes | clade_252 | 40% | 40% | Y | N |
| Clostridium_butyricum | clade_252 | 20% | 20% | Y | N |
| Clostridium_disporicum | clade_253 | 40% | 30% | Y | N |
| Clostridium_hylemonae* | clade_260 | 100% | 50% | Y | N |
| Clostridium_scindens | clade_260 | 10% | 60% | Y | N |
| Coprococcus_comes* | clade_262 | 90% | 80% | Y | Y |
| Lachnospiraceae_bacterium_ | clade_262 | 90% | 80% | Y | Y |
| 1_4_56FAA* | | | | | |
| Ruminococcus_torgues | clade_262 | 30% | 70% | Y | Y |
| Parabacteroides_merdae | clade_286 | 30% | 20% | N | Y |
| Bifidobacterium_bifidum | clade_293 | 10% | 0% | N | N |
| Johnsonella_ignava | clade_298 | 10% | 10% | N | N |
| Blautia_glucerasea* | clade_309 | 100% | 80% | Y | N |
| Blautia_sp_M25* | clade_309 | 100% | 70% | Y | Y |
| Lachnospiraceae_bacterium_ | clade_309 | 100% | 60% | Y | N |
| 6_1_63FAA* | | | | | |
| Eubacterium_cellulosolvens | clade_309 | 10% | 30% | Y | Y |
| Lactobacillus_fermentum | clade_313 | 10% | 0% | N | N |
| Sarcina_ventriculi | clade_353 | 10% | 10% | Y | N |
| Clostridium_bartlettii* | clade_354 | 90% | 70% | Y | N |
| Clostridium_bifermentans | clade_354 | 70% | 70% | Y | N |
| Clostridium_mayombei | clade_354 | 50% | 50% | Y | N |
| Dorea_longicatena* | clade_360 | 100% | 60% | Y | Y |
| Lachnospiraceae_bacterium_ | clade_360 | 100% | 30% | Y | N |
| 9_1_43BFAA | | | | | |
| Lachnospiraceae_bacterium | clade_360 | 80% | 80% | Y | N |
| 2_1_58FAA* | | | | | |
| Lachnospiraceae_bacterium_ | clade_360 | 50% | 50% | Y | N |
| 2_1_46FAA | | | | | |
| Lactobacillus_perolens | clade_373 | 10% | 0% | N | N |
| Bacteroides_dorei | clade_378 | 60% | 50% | N | Y |
| Eubacterium_biforme | clade_385 | 10% | 0% | Y | N |
| Peptoniphilus_sp_gpac077 | clade_389 | 10% | 20% | N | N |
| Coprococcus_catus* | clade_393 | 100% | 70% | Y | Y |
| Eubacterium_hallii* | clade_396 | 90% | 60% | Y | Y |
| Anaerosporobacter_mobilis | clade_396 | 40% | 60% | Y | N |
| Bacteroides_pectinophilus | clade_396 | 10% | 60% | Y | N |
| Lactobacillus_hominis | clade_398 | 10% | 0% | N | N |
| Lactococcus_lactis | clade_401 | 40% | 40% | N | N |
| Ruminococcus_ | clade_406 | 80% | 50% | Y | N |
| champanellensis* | | | | | |
| Ruminococcus_callidus | clade_406 | 10% | 10% | Y | N |
| Clostridium_clostridioforme* | clade_408 | 100% | 60% | Y | Y |
| Eubacterium_hadrum* | clade_408 | 100% | 90% | Y | Y |
| Clostridium_symbiosum | clade_408 | 30% | 50% | Y | Y |
| Anaerostipes_caccae | clade_408 | 10% | 50% | Y | N |
| Parasutterella_ | clade_432 | 10% | 0% | N | N |
| excrementihominis | | | | | |
| Sutterella_stercoricanis | clade_432 | 10% | 0% | N | N |
| Eubacterium_rectale* | clade_444 | 100% | 80% | Y | Y |
| Lachnobacterium_bovis* | clade_444 | 100% | 80% | Y | N |
| Desulfovibrio_desulfuricans | clade_445 | 10% | 0% | N | Y |
| Eubacterium_sp_oral_clone_ | clade_476 | 80% | 70% | Y | N |
| JS001* | | | | | |
| Faecalibacterium_prausnitzii* | clade_478 | 100% | 60% | Y | Y |
| Subdoligranulum_variabile* | clade_478 | 100% | 80% | Y | Y |
| Coprobacillus_sp_D7* | clade_481 | 90% | 60% | Y | N |
| Clostridium_cocleatum | clade_481 | 60% | 20% | Y | N |
| Clostridium_spiroforme | clade_481 | 40% | 50% | Y | N |
| Eubacterium_ramulus* | clade_482 | 80% | 60% | Y | N |
| Flavonifractor_plautii | clade_494 | 70% | 60% | Y | Y |
| Pseudoflavonifractor_ | clade_494 | 60% | 60% | Y | Y |
| capillosus | | | | | |
| Ruminococcaceae_bacterium_ | clade_494 | 30% | 50% | Y | Y |
| D16 | | | | | |
| Acetivibrio_cellulolyticus* | clade_495 | 70% | 80% | Y | N |
| Clostridium_stercorarium | clade_495 | 40% | 50% | Y | N |
| Enterococcus_durans | clade_497 | 10% | 10% | N | N |
| Enterococcus_faecium | clade_497 | 10% | 10% | N | N |
| Dialister_invisus | clade_506 | 50% | 10% | N | N |
| Eubacterium_limosum | clade_512 | 20% | 0% | Y | N |
| Ruminococcus_flavefaciens | clade_516 | 60% | 60% | Y | N |
| Eubacterium_ventriosum | clade_519 | 30% | 60% | Y | Y |
| Bilophila_wadsworthia | clade_521 | 90% | 0% | N | Y |
| Lachnospira_pectinoschiza | clade_522 | 40% | 60% | Y | N |
| Eubacterium_eligens | clade_522 | 30% | 50% | Y | Y |
| Catonella_morbi | clade_534 | 20% | 0% | N | N |
| Clostridium_ | clade_537 | 100% | 80% | Y | N |
| sporosphaeroides* | | | | | |
| Ruminococcus_bromii | clade_537 | 60% | 30% | Y | Y |
| Clostridium_leptum | clade_537 | 40% | 70% | Y | Y |
| Clostridium_sp_YIT_12069 | clade_537 | 40% | 60% | Y | N |
| Clostridium_viride | clade_540 | 10% | 10% | Y | N |
| Megamonas_funiformis | clade_542 | 50% | 0% | N | N |
| Eubacterium_ruminantium* | clade_543 | 80% | 90% | Y | N |
| Coprococcus_eutactus | clade_543 | 20% | 20% | Y | N |
| Collinsella_aerofaciens | clade_553 | 50% | 10% | Y | Y |
| Alkaliphilus_metalliredigenes | clade_554 | 40% | 10% | Y | N |
| Turicibacter_sanguinis | clade_555 | 80% | 40% | Y | N |
| Phascolarctobacterium_ | clade_556 | 20% | 0% | N | N |
| faecium | | | | | |
| Clostridiales_bacterium_oral | clade_558 | 80% | 50% | N | N |
| clone_P4PA* | | | | | |
| Lutispora_thermophila | clade_564 | 100% | 0% | Y | N |
| Coriobacteriaceae_bacterium_ | clade_566 | 70% | 0% | N | N |
| JC110 | | | | | |
| Eggerthella_sp_1_3_56FAA | clade_566 | 70% | 30% | N | N |
| Adlercreutzia_equolifaciens | clade_566 | 40% | 0% | N | N |
| Gordonibacter_pamelaeae | clade_566 | 30% | 0% | N | Y |
| Slackia_isoflavoniconvertens | clade_566 | 10% | 0% | N | N |
| Eubacterium_desmolans* | clade_572 | 90% | 70% | Y | N |
| Papillibacter_cinnamivorans* | clade_572 | 90% | 80% | Y | N |
| Clostridium_colinum | clade_576 | 30% | 30% | Y | N |
| Akkermansia_muciniphila | clade_583 | 60% | 10% | N | Y |
| Clostridiales_bacterium_ | clade_584 | 60% | 30% | N | N |
| oral_taxon_F32 | | | | | |
| Prochlorococcus_marinus | clade_592 | 30% | 0% | N | N |
| Methanobrevibacter_wolinii | clade_595 | 30% | 0% | N | N |
| Bacteroides_fragilis | clade_65 | 20% | 30% | N | Y |
| Lactobacillus_delbrueckii | clade_72 | 10% | 0% | N | N |
| Escherichia_coli | clade_92 | 50% | 0% | N | Y |
| Clostridium_sp_D5 | clade_96 | 80% | 60% | Y | N |
| Streptococcus_thermophilus | clade_98 | 90% | 20% | N | Y |
| Streptococcus_sp_CM6 | clade_98 | 20% | 10% | N | N |
| Streptococcus_sp_oral_ | clade_98 | 10% | 0% | N | N |
| clone_ASCE05 | | | | | |

OTUs detected by a minimum of ten 16S-V4 sequence reads in at least a one ethanol treated spore preparation (pan-microbiome).
OTUs that engraft in a treated patients and the percentage of patients in which they engraft are denoted, as are the clades, spore forming status, and Keystone OTU status.
Starred OTUs occur in ≥80% of the ethanol preps and engraft in ≥ 50% of the treated patients.

TABLE 19
Top 20 OTUs ranked by CES

| | | | Spore | Keystone |
|---|---|---|---|---|
| OTU | Clade | CES | Former | OTU |
| Eubacterium_hadrum | clade_408 | 4.2 | Y | Y |
| Eubacterium_rectale | clade_444 | 4.2 | Y | Y |
| Subdoligranulum_variabile | clade_478 | 4.2 | Y | Y |
| Blautia_sp_M25 | clade_309 | 4.2 | Y | Y |
| Coprococcus_catus | clade_393 | 4.2 | Y | Y |
| Lachnospiraceae_bacterium_ | clade_262 | 4.2 | Y | Y |
| 1_4_56FAA | | | | |
| Coprococcus_comes | clade_262 | 4.2 | Y | Y |
| Blautia_glucerasea | clade_309 | 4.0 | Y | N |
| Lachnobacterium_bovis | clade_444 | 4.0 | Y | N |
| Clostridium_sporosphaeroides | clade_537 | 4.0 | Y | N |
| Clostridiales_sp_SS3_4 | clade_246 | 4.0 | Y | N |
| Papillibacter_cinnamivorans | clade_572 | 4.0 | Y | N |
| Clostridium_bartlettii | clade_354 | 4.0 | Y | N |
| Eubacterium_desmolans | clade_572 | 4.0 | Y | N |
| Clostridium_clostridioforme | clade_408 | 3.2 | Y | Y |
| Dorea_longicatena | clade_360 | 3.2 | Y | Y |
| Faecalibacterium_prausnitzii | clade_478 | 3.2 | Y | Y |
| Eubacterium_hallii | clade_396 | 3.2 | Y | Y |
| Clostridium_leptum | clade_537 | 3.2 | Y | Y |
| Lachnospiraceae_bacterium_ | clade_309 | 3.0 | Y | N |
| 6_1_63FAA | | | | |

TABLE 20
Subsets of the Core Ecology tested in the C. difficile mouse model

| | | Substitute For OTU |
|---|---|---|
| Subset | OTU | in Table 1 (Clade) |
| Subset 1 | Collinsella aerofaciens | none (Clade_553) |
| | Clostridium tertium | C. sporogenes (Clade_252) |
| | Clostridium disporicum | none (Clade_253) |
| | Clostridium innocuum | Clostridium_sp_HGF2 |
| | | (Clade_351) |
| | Clostridium mayombei | none (Clade_354) |
| | Clostridium butyricum | none (Clade_252) |
| | Coprococcus comes | none (Clade_262) |
| | Clostridium hylemonae | none (Clade_260) |
| | Clostridium bolteae | E. hadrum (Clade_408) |
| | Clostridium symbiosum | C. clostridioforme |
| | | (Clade_408) |
| | Clostridium orbiscindens | R._bacterium_D16 |
| | | (Clade_494) |
| | Lachnospiraceae | C. scindens (Clade_260) |
| | bacterium_5_1_57FAA | |
| | Blautia producta | Blautia_sp_M25 (Clade_309) |
| | Ruminococcus gnavus | D. longicatena(Clade_360) |
| | Ruminococcus bromii | none (Clade_537) |
| Subset 2 | Collinsella aerofaciens | none (Clade_553) |
| | Clostridium butyricum | none (Clade_252) |
| | Clostridium hylemonae | none (Clade_260) |
| | Blautia producta | Blautia_sp_M25 (Clade_309) |
| Subset 3 | Collinsella aerofaciens | none (Clade_553) |
| | Clostridium innocuum | Clostridium_sp_HGF2 |
| | | (Clade_351) |
| | Coprococcus comes | none (Clade_262) |
| | Ruminococcus bromii | none (Clade_537) |
| Subset 4 | Clostridium butyricum | none (Clade_252) |
| | Clostridium hylemonae | none (Clade_260) |
| | Blautia producta | Blautia_sp_M25 (Clade_309) |
| Subset 5 | Clostridium butyricum | none (Clade_252) |
| | Clostridium hylemonae | none (Clade_260) |
| Subset 6 | Blautia producta | Blautia_sp_M25 (Clade_309) |
| | Clostridium butyricum | none (Clade_252) |
| Subset 7 | Clostridium orbiscindens | R._bacterium_D16 |
| | | (Clade_494) |
| | Lachnospiraceae | C. scindens (Clade_260) |
| | bacterium_5_1_57FAA | |
| | Eubacterium rectale | none (Clade_444) |

TABLE 21
Results of bacterial compositions tested
in a C. difficile mouse model

| | | | Avg. | Avg. |
|---|---|---|---|---|
| | | Cumulative | Minimum | Maximum |
| | | Mortality | Relative | Clinical Score |
| Group | Dose | (%) | Weight | (Death = 4) |
| Vehicle | — | 40 | 0.87 | 2.8 |
| Control | | | | |
| Feces | 5.8e8 | 0 | 0.99 | 0 |
| Control | cfu total | | | |
| Subset 1 | 1e8 | 0 | 0.98 | 0 |
| | cfu/OTU | | | |
| Subset 2 | 1e8 | 10 | 0.84 | 2.1 |
| | cfu/OTU | | | |
| Subset 3 | 1e8 | 10 | 0.84 | 2.2 |
| | cfu/OTU | | | |
| Subset 4 | 1e8 | 0 | 0.87 | 2 |
| | cfu/OTU | | | |
| Subset 5 | 1e8 | 20 | 0.91 | 1.7 |
| | cfu/OTU | | | |
| Subset 6 | 1e8 | 40 | 0.82 | 2.8 |
| | cfu/OTU | | | |
| Subset 7 | 1e8 | 0 | 0.90 | 1 |
| | cfu/OTU | | | |

TABLE 22
OTUs and their clade assignments tested in ternary
combinations with results in the in vitro inhibition assay

| OTU1 | Clade1 | OTU2 | Clade2 | OTU3 | Clade3 | Results |
|---|---|---|---|---|---|---|
| Clostridium_ | clade_408 | Blautia_producta | clade_309 | Eubacterium_ | clade_444 | ++++ |
| bolteae | | | | rectale | | |
| Clostridium_ | clade_408 | Clostridium_ | clade_408 | Blautia_producta | clade_309 | ++++ |
| bolteae | | symbiosum | | | | |
| Clostridium_ | clade_408 | Clostridium_ | clade_408 | Eubacterium_ | clade_444 | −− |
| bolteae | | symbiosum | | rectale | | |
| Clostridium_ | clade_408 | Clostridium_ | clade_408 | Faecalibacterium_ | clade_478 | − |
| bolteae | | symbiosum | | prausnzii | | |
| Clostridium_ | clade_408 | Clostridium_ | clade_408 | Lachnospiraceae_ | clade_260 | |
| bolteae | | symbiosum | | bacterium_5_1_ | | |
| | | | | 57FAA | | |
| Clostridium_ | clade_408 | Faecalibacterium_ | clade_478 | Blautia_producta | clade_309 | ++++ |
| bolteae | | prausnzii | | | | |
| Clostridium_ | clade_408 | Faecalibacterium_ | clade_478 | Eubacterium_ | clade_444 | |
| bolteae | | prausnzii | | rectale | | |
| Clostridium_ | clade_408 | Faecalibacterium_ | clade_478 | Lachnospiraceae_ | clade_260 | ++++ |
| bolteae | | prausnzii | | bacterium_5_1_ | | |
| | | | | 57FAA | | |
| Clostridium_ | clade_408 | Lachnospiraceae_ | clade_260 | Blautia_producta | clade_309 | ++++ |
| bolteae | | bacterium_5_1_ | | | | |
| | | 57FAA | | | | |
| Clostridium_ | clade_408 | Lachnospiraceae_ | clade_260 | Eubacterium_ | clade_444 | + |
| bolteae | | bacterium_5_1_ | | rectale | | |
| | | 57FAA | | | | |
| Clostridium_ | clade_408 | Blautia_producta | clade_309 | Eubacterium_ | clade_444 | ++++ |
| symbiosum | | | | rectale | | |
| Clostridium_ | clade_408 | Faecalibacterium_ | clade_478 | Blautia_producta | clade_309 | ++++ |
| symbiosum | | prausnzii | | | | |
| Clostridium_ | clade_408 | Faecalibacterium_ | clade_478 | Eubacterium_ | clade_444 | |
| symbiosum | | prausnzii | | rectale | | |
| Clostridium_ | clade_408 | Faecalibacterium_ | clade_478 | Lachnospiraceae_ | clade_260 | + |
| symbiosum | | prausnzii | | bacterium_5_1_ | | |
| | | | | 57FAA | | |
| Clostridium_ | clade_408 | Lachnospiraceae_ | clade_260 | Blautia_producta | clade_309 | ++++ |
| symbiosum | | bacterium_5_1_ | | | | |
| | | 57FAA | | | | |
| Clostridium_ | clade_408 | Lachnospiraceae_ | clade_260 | Eubacterium_ | clade_444 | |
| symbiosum | | bacterium_5_1_ | | rectale | | |
| | | 57FAA | | | | |
| Collinsella_ | clade_553 | Blautia_producta | clade_309 | Eubacterium_ | clade_444 | ++++ |
| aerofaciens | | | | rectale | | |
| Collinsella_ | clade_553 | Clostridium_ | clade_408 | Blautia_producta | clade_309 | ++++ |
| aerofaciens | | bolteae | | | | |
| Collinsella_ | clade_553 | Clostridium_ | clade_408 | Clostridium_ | clade_408 | ++++ |
| aerofaciens | | bolteae | | symbiosum | | |
| Collinsella_ | clade_553 | Clostridium_ | clade_408 | Eubacterium_ | clade_444 | ++++ |
| aerofaciens | | bolteae | | rectale | | |
| Collinsella_ | clade_553 | Clostridium_ | clade_408 | Faecalibacterium_ | clade_478 | ++++ |
| aerofaciens | | bolteae | | prausnzii | | |
| Collinsella_ | clade_553 | Clostridium_ | clade_408 | Lachnospiraceae_ | clade_260 | ++++ |
| aerofaciens | | bolteae | | bacterium_5_1_ | | |
| | | | | 57FAA | | |
| Collinsella_ | clade_553 | Clostridium_ | clade_408 | Blautia_producta | clade_309 | ++++ |
| aerofaciens | | symbiosum | | | | |
| Collinsella_ | clade_553 | Clostridium_ | clade_408 | Eubacterium_ | clade_444 | |
| aerofaciens | | symbiosum | | rectale | | |
| Collinsella_ | clade_553 | Clostridium_ | clade_408 | Faecalibacterium_ | clade_478 | |
| aerofaciens | | symbiosum | | prausnzii | | |
| Collinsella_ | clade_553 | Clostridium_ | clade_408 | Lachnospiraceae_ | clade_260 | + |
| aerofaciens | | symbiosum | | bacterium_5_1_ | | |
| | | | | 57FAA | | |
| Collinsella_ | clade_553 | Coprococcus_ | clade_262 | Blautia_producta | clade_309 | ++++ |
| aerofaciens | | comes | | | | |
| Collinsella_ | clade_553 | Coprococcus_ | clade_262 | Clostridium_ | clade_408 | ++++ |
| aerofaciens | | comes | | bolteae | | |
| Collinsella_ | clade_553 | Coprococcus_ | clade_262 | Clostridium_ | clade_408 | +++ |
| aerofaciens | | comes | | symbiosum | | |
| Collinsella_ | clade_553 | Coprococcus_ | clade_262 | Eubacterium_ | clade_444 | +++ |
| aerofaciens | | comes | | rectale | | |
| Collinsella_ | clade_553 | Coprococcus_ | clade_262 | Faecalibacterium_ | clade_478 | ++++ |
| aerofaciens | | comes | | prausnzii | | |
| Collinsella_ | clade_553 | Coprococcus_ | clade_262 | Lachnospiraceae_ | clade_260 | +++ |
| aerofaciens | | comes | | bacterium_5_1_ | | |
| | | | | 57FAA | | |
| Collinsella_ | clade_553 | Faecalibacterium_ | clade_478 | Blautia_producta | clade_309 | ++++ |
| aerofaciens | | prausnzii | | | | |
| Collinsella_ | clade_553 | Faecalibacterium_ | clade_478 | Eubacterium_ | clade_444 | +++ |
| aerofaciens | | prausnzii | | rectale | | |
| Collinsella_ | clade_553 | Faecalibacterium_ | clade_478 | Lachnospiraceae_ | clade_260 | +++ |
| aerofaciens | | prausnzii | | bacterium_5_1_ | | |
| | | | | 57FAA | | |
| Collinsella_ | clade_553 | Lachnospiraceae_ | clade_260 | Blautia_producta | clade_309 | ++++ |
| aerofaciens | | bacterium_5_1_ | | | | |
| | | 57FAA | | | | |
| Collinsella_ | | Lachnospiraceae_ | clade_260 | Eubacterium_ | clade_444 | ++++ |
| aerofaciens | | bacterium_5_1_ | | rectale | | |
| | | 57FAA | | | | |
| Coprococcus_ | clade_262 | Blautia_producta | clade_309 | Eubacterium_ | clade_444 | ++++ |
| comes | | | | rectale | | |
| Coprococcus_ | clade_262 | Clostridium_ | clade_408 | Blautia_producta | clade_309 | ++++ |
| comes | | bolteae | | | | |
| Coprococcus_ | clade_262 | Clostridium_ | clade_408 | Clostridium_ | clade_408 | |
| comes | | bolteae | | symbiosum | | |
| Coprococcus_ | clade_262 | Clostridium_ | clade_408 | Eubacterium_ | clade_444 | −− |
| comes | | bolteae | | rectale | | |
| Coprococcus_ | clade_262 | Clostridium_ | clade_408 | Faecalibacterium_ | clade_478 | +++ |
| comes | | bolteae | | prausnzii | | |
| Coprococcus_ | clade_262 | Clostridium_ | clade_408 | Lachnospiraceae_ | clade_260 | +++ |
| comes | | bolteae | | bacterium_5_1_ | | |
| | | | | 57FAA | | |
| Coprococcus_ | clade_262 | Clostridium_ | clade_408 | Blautia_producta | clade_309 | ++++ |
| comes | | symbiosum | | | | |
| Coprococcus_ | clade_262 | Clostridium_ | clade_408 | Eubacterium_ | clade_444 | −−− |
| comes | | symbiosum | | rectale | | |
| Coprococcus_ | clade_262 | Clostridium_ | clade_408 | Faecalibacterium_ | clade_478 | |
| comes | | symbiosum | | prausnzii | | |
| Coprococcus_ | clade_262 | Clostridium_ | clade_408 | Lachnospiraceae_ | clade_260 | |
| comes | | symbiosum | | bacterium_5_1_ | | |
| | | | | 57FAA | | |
| Coprococcus_ | clade_262 | Faecalibacterium_ | clade_478 | Blautia_producta | clade_309 | ++++ |
| comes | | prausnzii | | | | |
| Coprococcus_ | clade_262 | Faecalibacterium_ | clade_478 | Eubacterium_ | clade_444 | − |
| comes | | prausnzii | | rectale | | |
| Coprococcus_ | clade_262 | Faecalibacterium_ | clade_478 | Lachnospiraceae__ | clade_260 | |
| comes | | prausnzii | | bacterium_5_1_ | | |
| | | | | 57FAA | | |
| Coprococcus_ | clade_262 | Lachnospiraceae_ | clade_260 | Blautia_producta | clade_309 | ++++ |
| comes | | bacterium_5_1_ | | | | |
| | | 57FAA | | | | |
| Coprococcus_ | clade_262 | Lachnospiraceae_ | clade_260 | Eubacterium_ | clade_444 | |
| comes | | bacterium_5_1_ | | rectale | | |
| | | 57FAA | | | | |
| Faecalibacterium_ | clade_478 | Blautia_producta | clade_309 | Eubacterium_ | clade_444 | ++++ |
| prausnzii | | | | rectale | | |
| Faecalibacterium_ | clade_478 | Lachnospiraceae_ | | | | |
| prausnzii | | bacterium_5_1_ | clade_260 | Blautia_producta | clade_309 | ++++ |
| | | 57FAA | | | | |
| Faecalibacterium_ | clade_478 | Lachnospiraceae_ | | | | |
| prausnzii | | bacterium_5_1_ | clade_260 | Eubacterium_ | clade_444 | |
| | | 57FAA | | rectale | | |
| Lachnospiraceae_ | clade_260 | Blautia_producta | clade_309 | Eubacterium_ | clade_444 | ++++ |
| bacterium_5_1_ | | | | rectale | | |
| 57FAA | | | | | | |

1-22. (canceled)

23. A therapeutic composition comprising a purified population of spore-forming bacteria or bacteria having a substantial activity of spore-forming bacteria, in an amount effective to populate the gastrointestinal tract of a mammalian subject to whom the therapeutic composition is administered.

24. The therapeutic composition of claim 23, wherein the population is effective to i) treat a gastrointestinal dysbiosis, and/or ii) engraft at least one type of bacteria present in the therapeutic composition but not present in a mammalian subject prior to treatment; and/or iii) augment at least one type of bacteria not present in the therapeutic composition in a mammalian subject to whom the therapeutic composition is administered.

25. (canceled)

26. The therapeutic composition of claim 23, wherein the populating of the gastrointestinal tract is effective to either: i) engraft at least one type of bacteria present in the therapeutic composition but not present in a mammalian subject, or ii) augment at least one type of bacteria not present in the therapeutic composition in the mammalian recipient.

27. The therapeutic composition of claim 23, wherein the population comprises a population of bacterial spores.

28. (canceled)

29. The therapeutic composition of claim 23, wherein the population is purified from a fecal material obtained from a mammalian donor subject.

30-36. (canceled)

37. The therapeutic composition of claim 23, wherein the dysbiosis comprises a gastrointestinal disease, disorder or condition selected from the group consisting of Clostridium difficile-induced diarrhea, irritable bowel syndrome (IBS), colonization with a pathogen or pathobiont, infection by a drug-resistant pathogen or pathobiont, colitis, and Crohn's Disease.

38-39. (canceled)

40. The therapeutic composition of claim 23, wherein the purified population is substantially reduced in a residual habitat product.

41-52. (canceled)

53. A therapeutic composition comprising a purified population of spore-forming bacteria, in an amount effective to populate the gastrointestinal tract of a mammalian subject to whom the therapeutic composition is administered, wherein the purified population is obtained by the steps of a) providing a fecal material comprising spore-forming bacteria and b) subjecting the spore-forming bacteria to a treatment step resulting in purification of the spore-forming bacteria.

54. (canceled)

55. The therapeutic composition of claim 53, wherein the purified population is obtained from a heat treatment, mechanical treatment, or solvent treatment of the spore-forming bacteria.

56. The therapeutic composition of claim 53, wherein the composition optionally comprises a germinant.

57-76. (canceled)

77. The therapeutic composition of claim 27, wherein the bacterial spores comprise bacteria from the genera provided in Table 1.

78-80. (canceled)

81. A therapeutic composition comprising a purified bacterial spore population comprising at least about 1×10³, 1×10⁴, 1×10⁵, or 1×10⁶ spores, wherein the composition does not exceed about 1 gram in weight, formulated for oral administration to populate the gastrointestinal tract in a mammalian subject in need thereof, in order to i) engraft in the mammalian recipient and/or ii) augment a bacterial population in the mammalian recipient and thereby treat or prevent a dysbiosis.

82-83. (canceled)

84. The therapeutic composition of claim 81, in an amount effective to treat or prevent a metabolic disorder in a mammalian recipient subject suffering from or at risk of developing such disorder to whom the therapeutic composition is administered.

85-106. (canceled)

107. The therapeutic composition of claim 23, comprising a first type of isolated bacteria and a second type of isolated bacteria, wherein the first type and the second type are not identical, wherein the first type of isolated bacteria and the second type of isolated bacteria comprise an operational taxonomic unit (OTU) distinction.

108. The therapeutic composition of claim 107, wherein OTU distinction comprises 16S rRNA sequence similarity below about 97% identity.

109-120. (canceled)
